SEQUENCE LISTING (620 sequences)

SEQ ID NO: 1 - DNA, 18 bp, Artificial Sequence
  primer
aaggcagatcaacacagg

SEQ ID NO: 2 - DNA, 18 bp, Artificial Sequence
  primer
agcagattgcaaagcctg

SEQ ID NO: 3 - DNA, 20 bp, Artificial Sequence
  primer
caacacagggatagaataac

SEQ ID NO: 4 - DNA, 19 bp, Artificial Sequence
  primer
atctgccttagcagattgc

SEQ ID NO: 5 - DNA, 21 bp, Artificial Sequence
  primer
tgcagaaattgcccctcgact

SEQ ID NO: 6 - DNA, 23 bp, Artificial Sequence
  primer
aacaatggggaagaagtcaaagg

SEQ ID NO: 7 - DNA, 18 bp, Artificial Sequence
  primer
aaggcagatcaacacagg

SEQ ID NO: 8 - DNA, 18 bp, Artificial Sequence
  primer
agcagattgcaaagcctg

SEQ ID NO: 9 - DNA, 20 bp, Artificial Sequence
  primer
caacacagggatagaataac

SEQ ID NO: 10 - DNA, 19 bp, Artificial Sequence
  primer
atctgccttagcagattgc

SEQ ID NO: 11 - DNA, 2296 bp, Artificial Sequence
  isolate
aaggcagatcaacacagggatagaataacacgttttgcgactttgaaacatagagctaagaatcaggaaaattatttatttaccctggctaagtttaaagagaactatgaaaaagacgttaaaaacgaagaatctatcaaagctctaaaatctgctcagaaattgaatgaatgcggaaactatctgctattcaaaaatttttacacaataggcgaagttaaactttccaagcttcgtacctgcggacagcatttgctttgtcctttctgtgctgccattcgtgcatctcgtgctattcaaaaatacgttgaacgtattgaccaagtcttaaaagaaaatcgcaagctcaagcctgttctaatcacgctcacagttaaaaacggctctaacctagcagaacgctctgaacatcttatgaagtcatttagaacgcttctagagcgtcgtagggactatgaaaagaaaggacgaggttttaacgagttctgtaaggttcaaggtgctatgtattcatatgagaatacattcaatgagaaaacaggcgaatggcatccgcatattcatatgtttgcactggttgaccagtggattgatcaacaagaattttcagaatactggcacagtgttactggcgattctatggttgttgatgtccgtagggctaagagagaaaagggacatggatatagcaaagctgctgctgaagtctgtaaatatgctttgaagttttgcgatctgtctgttgaaaagacttgggaggctttcaaagttctcaaaggtaaacgcttaacaggctcttttgggcttctgtggggtgtcaaaattcctgacacaatgacagacgatatgccagaagaagatttgccttatcttgaaatgctttacaagtttacatacggcaaaaagtcttactacgacttaccgataacaagacacgtagaagcgcaggcgggagagcgcgcggagttaggaggagctacgacgacgcccgacgagagatatagcgcgggtgcttcaagtgttaaacaaggttcggaatggagcgtggcgaccccgggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattggctttcattggttagtctgtttcttgagaaacgcggaagcgtgcattctgaaaaactcaccacttttgaaaaaagtgtagtgagtacacttctccagtagccgaacgagtgtctacgagtgacgactcctaaataagaatttcaaaaccttcctttctctgattacaagctccccttagatttctaagcgggagtttttagccatcccgaacgggtggcgacgcaatactatttgcatctttttcgataatgatatataaaagattcgtctagactctaagttaatttcaaaaatatattaggtatatcactatgtcagagccaacaaacctacagttattcgttgctgaacttaaaaaaactggtcgttcttcttatcacggtgcttatttccaagttccgttccgtgtccaaatgcaccttcacgcaaaagttgaagcccttactaaacacttgggtagtacacgaaataaggtcttaaacgatcttttatcaattgcccttgatcaggtttatcaatctttggatctcgatgaagataccttgcacgaaatccaagtagaggaaggtcgcattctccatgaattacttgaaaaccgtaaagatattaaatctggagatatggcagatgattaatttacaggggactttaatcaatgctttccgtgtggatggtggtaaaggtaaagacggcaaagaatatgaagcgcgtgacaaggtgcaaattcttggttcgttggaactcccaaacggggaaattaaacacgaacttgttgacctaacagttgaggattctcgactatttgaaccattcaaaaatcaggttattagcatctcgtgtggtgctatggctatcggtcgtaatgttgttttttatgttcgtaaaggcgcaaaaccgattttagcagaaagtatttgatactcgttaaaaagcagtgagttagcgaagctactcgctgttttttcttttacttgatactattgattaaagtggggacaaaaattgcttaaaaaagacaataaaaaagcccatatttcggacactttggcgggtgatgaaatacaggctttgcaatctgct

SEQ ID NO: 12 - protein, 89 aa, Artificial Sequence
  orf 1
MKRKDEVLTSSVRFKVLCIHMRIHSMRKQANGIRIFICLHWLTSGLINKNFQNTGTVLLAILWLLMSVGLREKRDMDIAKLLLKSVNML

SEQ ID NO: 13 - protein, 401 aa, Artificial Sequence
  orf 2
MTRFATLKHRAKNQENYLFTLAKFKENYEKDVKNEESIKALKSAQKLNECGNYLLFKNFYTIGEVKLSKLRTCGQHLLCPFCAAIRASRAIQKYVERIDQVLKENRKLKPVLITLTVKNGSNLAERSEHLMKSFRTLLERRRDYEKKGRGFNEFCKVQGAMYSYENTFNEKTGEWHPHIHMFALVDQWIDQQEFSEYWHSVTGDSMVVDVRRAKREKGHGYSKAAAEVCKYALKFCDLSVEKTWEAFKVLKGKRLTGSFGLLWGVKIPDTMTDDMPEEDLPYLEMLYKFTYGKKSYYDLPITRHVEAQAGERAELGGATTTPDERYSAGASSVKQGSEWSVATPGRTALCSVPKFQRKKAHWRIPPSTRVRVRQRIRRWDGIMYNIDLFPYVEHRLLAFIG

SEQ ID NO: 14 - protein, 107 aa, Artificial Sequence
  orf 3
MRDIARVLQVLNKVRNGAWRPRVEQRSVLYRSFNVKKPTGESHLLPVFVFGKESEDGTGLCITLIFSHMSNIGYWLSLVSLFLEKRGSVHSEKLTTFEKSVVSTLLQ

SEQ ID NO: 15 - protein, 61 aa, Artificial Sequence
  orf 4
MLGISLCQSQQTYSYSLLNLKKLVVLLITVLISKFRSVSKCTFTQKLKPLLNTWVVHEIRS

SEQ ID NO: 16 - protein, 112 aa, Artificial Sequence
  orf 5
MRYITMSEPTNLQLFVAELKKTGRSSYHGAYFQVPFRVQMHLHAKVEALTKHLGSTRNKVLNDLLSIALDQVYQSLDLDEDTLHEIQVEEGRILHELLENRKDIKSGDMADD

SEQ ID NO: 17 - protein, 112 aa, Artificial Sequence
  orf 6
MNYLKTVKILNLEIWQMINLQGTLINAFRVDGGKGKDGKEYEARDKVQILGSLELPNGEIKHELVDLTVEDSRLFEPFKNQVISISCGAMAIGRNVVFYVRKGAKPILAESI

SEQ ID NO: 18 - protein, 435 aa, Artificial Sequence
  orf 7
MLKKDNKKAHISDTLAGDEIQALQSAKADQHRDRITRFATLKHRAKNQENYLFTLAKFKENYEKDVKNEESIKALKSAQKLNECGNYLLFKNFYTIGEVKLSKLRTCGQHLLCPFCAAIRASRAIQKYVERIDQVLKENRKLKPVLITLTVKNGSNLAERSEHLMKSFRTLLERRRDYEKKGRGFNEFCKVQGAMYSYENTFNEKTGEWHPHIHMFALVDQWIDQQEFSEYWHSVTGDSMVVDVRRAKREKGHGYSKAAAEVCKYALKFCDLSVEKTWEAFKVLKGKRLTGSFGLLWGVKIPDTMTDDMPEEDLPYLEMLYKFTYGKKSYYDLPITRHVEAQAGERAELGGATTTPDERYSAGASSVKQGSEWSVATPGRTALCSVPKFQRKKAHWRIPPSTRVRVRQRIRRWDGIMYNIDLFPYVEHRLLAFIG

SEQ ID NO: 19 - protein, 76 aa, Artificial Sequence
  orf 8
MNGSNSRESSTVRSTSSCLISPFGSSNEPRICTLSRASYSLPSLPLPPSTRKALIKVPCKLIICHISRFNIFTVFK

SEQ ID NO: 20 - protein, 63 aa, Artificial Sequence
  orf 9
MFDIWEKINVIHNPVPSSDSLPNTNTGRRWDSPVGFFTLKLRYRTERCSTRGRHAPFRTLFNT

SEQ ID NO: 21 - protein, 84 aa, Artificial Sequence
  orf
MSSVIVSGILTPHRSPKEPVKRLPLRTLKASQVFSTDRSQNFKAYLQTSAAALLYPCPFSLLALRTSTTIESPVTLCQYSENSC

SEQ ID NO: 22 - protein, 56 aa, Artificial Sequence
  orf
MIFLILSSMFQSRKTCYSIPVLICLSRLQSLYFITRQSVRNMGFFIVFFKQFLSPL

SEQ ID NO: 23 - DNA, 267 bp, Artificial Sequence
  orf sequence
atgaaaagaaaggacgaggttttaacgagttctgtaaggttcaaggtgctatgtattcatatgagaatacattcaatgagaaaacaggcgaatggcatccgcatattcatatgtttgcactggttgaccagtggattgatcaacaagaattttcagaatactggcacagtgttactggcgattctatggttgttgatgtccgtagggctaagagagaaaagggacatggatatagcaaagctgctgctgaagtctgtaaatatgctt

SEQ ID NO: 24 - DNA, 1203 bp, Artificial Sequence
  orf
ataacacgttttgcgactttgaaacatagagctaagaatcaggaaaattatttatttaccctggctaagtttaaagagaactatgaaaaagacgttaaaaacgaagaatctatcaaagctctaaaatctgctcagaaattgaatgaatgcggaaactatctgctattcaaaaatttttacacaataggcgaagttaaactttccaagcttcgtacctgcggacagcatttgctttgtcctttctgtgctgccattcgtgcatctcgtgctattcaaaaatacgttgaacgtattgaccaagtcttaaaagaaaatcgcaagctcaagcctgttctaatcacgctcacagttaaaaacggctctaacctagcagaacgctctgaacatcttatgaagtcatttagaacgcttctagagcgtcgtagggactatgaaaagaaaggacgaggttttaacgagttctgtaaggttcaaggtgctatgtattcatatgagaatacattcaatgagaaaacaggcgaatggcatccgcatattcatatgtttgcactggttgaccagtggattgatcaacaagaattttcagaatactggcacagtgttactggcgattctatggttgttgatgtccgtagggctaagagagaaaagggacatggatatagcaaagctgctgctgaagtctgtaaatatgctttgaagttttgcgatctgtctgttgaaaagacttgggaggctttcaaagttctcaaaggtaaacgcttaacaggctcttttgggcttctgtggggtgtcaaaattcctgacacaatgacagacgatatgccagaagaagatttgccttatcttgaaatgctttacaagtttacatacggcaaaaagtcttactacgacttaccgataacaagacacgtagaagcgcaggcgggagagcgcgcggagttaggaggagctacgacgacgcccgacgagagatatagcgcgggtgcttcaagtgttaaacaaggttcggaatggagcgtggcgaccccgggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattggctttcattggt

SEQ ID NO: 25 - DNA, 321 bp, Artificial Sequence
  orf
acgagagatatagcgcgggtgcttcaagtgttaaacaaggttcggaatggagcgtggcgaccccgggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattggctttcattggttagtctgtttcttgagaaacgcggaagcgtgcattctgaaaaactcaccacttttgaaaaaagtgtagtgagtacacttctccag

SEQ ID NO: 26 - DNA, 183 bp, Artificial Sequence
  orf
atattaggtatatcactatgtcagagccaacaaacctacagttattcgttgctgaacttaaaaaaactggtcgttcttcttatcacggtgcttatttccaagttccgttccgtgtccaaatgcaccttcacgcaaaagttgaagcccttactaaacacttgggtagtacacgaaataaggtct

SEQ ID NO: 27 - DNA, 336 bp, Artificial Sequence
  orf
attaggtatatcactatgtcagagccaacaaacctacagttattcgttgctgaacttaaaaaaactggtcgttcttcttatcacggtgcttatttccaagttccgttccgtgtccaaatgcaccttcacgcaaaagttgaagcccttactaaacacttgggtagtacacgaaataaggtcttaaacgatcttttatcaattgcccttgatcaggtttatcaatctttggatctcgatgaagataccttgcacgaaatccaagtagaggaaggtcgcattctccatgaattacttgaaaaccgtaaagatattaaatctggagatatggcagatgat

SEQ ID NO: 28 - DNA, 336 bp, Artificial Sequence
  orf
atgaattacttgaaaaccgtaaagatattaaatctggagatatggcagatgattaatttacaggggactttaatcaatgctttccgtgtggatggtggtaaaggtaaagacggcaaagaatatgaagcgcgtgacaaggtgcaaattcttggttcgttggaactcccaaacggggaaattaaacacgaacttgttgacctaacagttgaggattctcgactatttgaaccattcaaaaatcaggttattagcatctcgtgtggtgctatggctatcggtcgtaatgttgttttttatgttcgtaaaggcgcaaaaccgattttagcagaaagtatt

SEQ ID NO: 29 - DNA, 1305 bp, Artificial Sequence
  orf
ttgcttaaaaaagacaataaaaaagcccatatttcggacactttggcgggtgatgaaatacaggctttgcaatctgctaaggcagatcaacacagggatagaataacacgttttgcgactttgaaacatagagctaagaatcaggaaaattatttatttaccctggctaagtttaaagagaactatgaaaaagacgttaaaaacgaagaatctatcaaagctctaaaatctgctcagaaattgaatgaatgcggaaactatctgctattcaaaaatttttacacaataggcgaagttaaactttccaagcttcgtacctgcggacagcatttgctttgtcctttctgtgctgccattcgtgcatctcgtgctattcaaaaatacgttgaacgtattgaccaagtcttaaaagaaaatcgcaagctcaagcctgttctaatcacgctcacagttaaaaacggctctaacctagcagaacgctctgaacatcttatgaagtcatttagaacgcttctagagcgtcgtagggactatgaaaagaaaggacgaggttttaacgagttctgtaaggttcaaggtgctatgtattcatatgagaatacattcaatgagaaaacaggcgaatggcatccgcatattcatatgtttgcactggttgaccagtggattgatcaacaagaattttcagaatactggcacagtgttactggcgattctatggttgttgatgtccgtagggctaagagagaaaagggacatggatatagcaaagctgctgctgaagtctgtaaatatgctttgaagttttgcgatctgtctgttgaaaagacttgggaggctttcaaagttctcaaaggtaaacgcttaacaggctcttttgggcttctgtggggtgtcaaaattcctgacacaatgacagacgatatgccagaagaagatttgccttatcttgaaatgctttacaagtttacatacggcaaaaagtcttactacgacttaccgataacaagacacgtagaagcgcaggcgggagagcgcgcggagttaggaggagctacgacgacgcccgacgagagatatagcgcgggtgcttcaagtgttaaacaaggttcggaatggagcgtggcgaccccgggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattggctttcattggt

SEQ ID NO: 30 - DNA, 228 bp, Artificial Sequence
  orf
ttgaatggttcaaatagtcgagaatcctcaactgttaggtcaacaagttcgtgtttaatttccccgtttgggagttccaacgaaccaagaatttgcaccttgtcacgcgcttcatattctttgccgtctttacctttaccaccatccacacggaaagcattgattaaagtcccctgtaaattaatcatctgccatatctccagatttaatatctttacggttttcaag

SEQ ID NO: 31 - DNA, 189 bp, Artificial Sequence
  orf
atgttcgacatatgggaaaagatcaatgttatacataatcccgtcccatcttcggattctttgccgaacacgaacacgggtagaaggtgggattctccagtgggcttttttacgttgaaacttcggtacagaacagagcgctgttcgacccggggtcgccacgctccattccgaaccttgtttaacact

SEQ ID NO: 32 - DNA, 252 bp, Artificial Sequence
  orf
atatcgtctgtcattgtgtcaggaattttgacaccccacagaagcccaaaagagcctgttaagcgtttacctttgagaactttgaaagcctcccaagtcttttcaacagacagatcgcaaaacttcaaagcatatttacagacttcagcagcagctttgctatatccatgtcccttttctctcttagccctacggacatcaacaaccatagaatcgccagtaacactgtgccagtattctgaaaattcttgt

SEQ ID NO: 33 - DNA, 168 bp, Artificial Sequence
  orf
ataattttcctgattcttagctctatgtttcaaagtcgcaaaacgtgttattctatccctgtgttgatctgccttagcagattgcaaagcctgtatttcatcacccgccaaagtgtccgaaatatgggcttttttattgtcttttttaagcaatttttgtccccactt

SEQ ID NO: 34 - DNA, 2356 bp, Artificial Sequence
  isolate
aaggcagatcaacacagggatagaataacacgttttgcgactttgaaacatagggctaagaatcaggaaaattatttatttaccctggctaagtttaaagagaactatgaaaaagacgttaaaaacgaagaatctatcaaagctctaaaatctgctcagaaattgaatgaatgcggaaactatctgctattcaaaaatttttacacaataggcgaagttaaactttctaagctccgtacctgcggacagcatttgctttgtcctttctgcgctgctattcgtgcatctcgtgctattcaaaaatacgttgaacgtattgatcaagtcttacaagaaaatcgcaagcttaagcctgttctaatcacgctcactgttaaaaacggttctgatctagcagaacgctctgaacatcttatgaagtcctttagaaagctcctagatcgtcgtagggactatgaaaagaaagggcgaggttttaacgagttctgtaaggttcaaggcgctatgtattcatatgaaaatacatttaatgaaaaaacaggcgaatggcatcctcatattcatatgttcgctttagttgataattggattgatcagcaagaattttcagaatattggcatagccttactggcgactctatggttgtcgatgttcgtagggcaaaaaaagaaaaaggtcacggttatagcaaagcggctgctgaagtctgtaagtatgctttgaagtttggcgatctgtccgttgaaaagacttgggaagcgtttaaggttctcaagggcaagcgtttaacaggttcttttggtctgctatggggtgtcaaaatccctgacacaatgacagacgatatgccattagaagatttgccatatctcgaaatgctctacaagttcgcctacagtaaaaaatcctactatgacttactaatcacacgacacgtagagccacatagcaaggacaacgacgacgaggaggagcttcgacaacgaggagaaggcgtacgaagcgatttggcgataacggacgagggaggagcggagcggattgagagcgagggcacgcagcgattgacacgcactgcaccgcaacgaggacgtaaaaaagcccactggagaatcccaccttctacccgtgttcgtgttcggcaacgaatcagaagatgggacggatatttatataatattgacctattcccgtatgttgagcataggctattagccttcatagaagactaaagattacttatataatctttagttccttctctccagtagccgagcgagtgtctacgagcgacgactcctaaatcagaatttcaaaaccttcctttccctgattacaaagctccccttagatttctaagcgggagtttttagccatcccgaaagggtggcgacgcattgacaattataatttaatctgcaatattcataagacattcataaataatctgattggaatattaaatatgctttttaaatctctaggcgttgcaatcgctacaaataatgcttttaactctgacgaagacttgaaaccaattactattcgtttagctgatagtgaaattcaggtgtatgacactgttgcaaaatcaatgggtctaactcgtcaagattttctatcacacgttattcgttccaacttccgacaggctttaaaagagtttgtagtgggttatacaagttcaaatccttctacgcctttgtctcttttatttaattcaaatacagacaatgacgaagttaagctcaaactttccaaccttctttcatctatttctcaggaattaatggacgaagaagaaaaagaaattcatgaagcaatcgaaaaccatatgaatacttcctatgcatatgcaactcgtccaaaaggcatctttgttgagaaggatgattcaaaatgattaagcttgagggaatcgttttaaacgtatttacccagcaaggcggtaaagataaaaaaacaggcgatcaatttgaagaccgtgacaaggtacaaattttaggtgctatggctctgcctaatggtgacgtaaaaaatgaactttttacgttatcagtagaaaattatcgggactttaaagactacctaaatcagaaaatttcgattgctgttggtgcaatggcaagtggtcgtaatgtgattttttatgttgctaaaggtgcaaaacctgttttagcagagacaatgtgaaacgtgaaaaaaaacagtgagttagcgaagcttctcgctgttttttcttttacttgatactattgattaaagtggggacaaaaattgcttaaaaaagacaataaaaaagcccatatttcagaagctttggcgggcggagaaatacaggctttgcaatctgct

SEQ ID NO: 35 - protein, 402 aa, Artificial Sequence
  orf
MTRFATLKHRAKNQENYLFTLAKFKENYEKDVKNEESIKALKSAQKLNECGNYLLFKNFYTIGEVKLSKLRTCGQHLLCPFCAAIRASRAIQKYVERIDQVLQENRKLKPVLITLTVKNGSDLAERSEHLMKSFRKLLDRRRDYEKKGRGFNEFCKVQGAMYSYENTFNEKTGEWHPHIHMFALVDNWIDQQEFSEYWHSLTGDSMVVDVRRAKKEKGHGYSKAAAEVCKYALKFGDLSVEKTWEAFKVLKGKRLTGSFGLLWGVKIPDTMTDDMPLEDLPYLEMLYKFAYSKKSYYDLLITRHVEPHSKDNDDEEELRQRGEGVRSDLAITDEGGAERIESEGTQRLTRTAPQRGRKKAHWRIPPSTRVRVRQRIRRWDGYLYNIDLFPYVEHRLLAFIED

SEQ ID NO: 36 - protein, 183 aa, Artificial Sequence
  orf
MSKREFLAIPKGWRRIDNYNLICNIHKTFINNLIGILNMLFKSLGVAIATNNAFNSDEDLKPITIRLADSEIQVYDTVAKSMGLTRQDFLSHVIRSNFRQALKEFVVGYTSSNPSTPLSLLFNSNTDNDEVKLKLSNLLSSISQELMDEEEKEIHEAIENHMNTSYAYATRPKGIFVEKDDSK

SEQ ID NO: 37 - protein, 97 aa, Artificial Sequence
  orf
MIKLEGIVLNVFTQQGGKDKKTGDQFEDRDKVQILGAMALPNGDVKNELFTLSVENYRDFKDYLNQKISIAVGAMASGRNVIFYVAKGAKPVLAETM

SEQ ID NO: 38 - protein, 436 aa, Artificial Sequence
  orf
MLKKDNKKAHISEALAGGEIQALQSAKADQHRDRITRFATLKHRAKNQENYLFTLAKFKENYEKDVKNEESIKALKSAQKLNECGNYLLFKNFYTIGEVKLSKLRTCGQHLLCPFCAAIRASRAIQKYVERIDQVLQENRKLKPVLITLTVKNGSDLAERSEHLMKSFRKLLDRRRDYEKKGRGFNEFCKVQGAMYSYENTFNEKTGEWHPHIHMFALVDNWIDQQEFSEYWHSLTGDSMVVDVRRAKKEKGHGYSKAAAEVCKYALKFGDLSVEKTWEAFKVLKGKRLTGSFGLLWGVKIPDTMTDDMPLEDLPYLEMLYKFAYSKKSYYDLLITRHVEPHSKDNDDEEELRQRGEGVRSDLAITDEGGAERIESEGTQRLTRTAPQRGRKKAHWRIPPSTRVRVRQRIRRWDGYLYNIDLFPYVEHRLLAFIED

SEQ ID NO: 39 - protein, 52 aa, Artificial Sequence
  orf
MFKTIPSSLIILNHPSQQRCLLDELHMHRKYSYGFRLLHEFLFLLRPLIPEK

SEQ ID NO: 40 - protein, 64 aa, Artificial Sequence
  orf
MGFSSGLFYVLVAVQCVSIAACPRSQSAPLLPRPLSPNRFVRLLLVVEAPPRRRCPCYVALRVV

SEQ ID NO: 41 - protein, 82 aa, Artificial Sequence
  orf
MSVPSSDSLPNTNTGRRWDSPVGFFTSSLRCSACQSLRALALNPLRSSLVRYRQIASYAFSSLSKLLLVVVVLAMWLYVSCD

SEQ ID NO: 42 - protein, 103 aa, Artificial Sequence
  orf
MKANSLCSTYGNRSILYKYPSHLLIRCRTRTRVEGGILQWAFLRPRCGAVRVNRCVPSLSIRSAPPSSVIAKSLRTPSPRCRSSSSSSLSLLCGSTCRVISKS

SEQ ID NO: 43 - protein, 64 aa, Artificial Sequence
  orf
MSRYGKSSNGISSVIVSGILTPHSRPKEPVKRLPLRTLNASQVFSTDRSPNFKAYLQTSAAALL

SEQ ID NO: 44 - protein, 59 aa, Artificial Sequence
  orf
MLALENLKRFPSLFNGQIAKLQSILTDFSSRFAITVTFFFFCPTNIDNHRVASKAMPIF

SEQ ID NO: 45 - DNA, 1206 bp, Artificial Sequence
  orf
ataacacgttttgcgactttgaaacatagggctaagaatcaggaaaattatttatttaccctggctaagtttaaagagaactatgaaaaagacgttaaaaacgaagaatctatcaaagctctaaaatctgctcagaaattgaatgaatgcggaaactatctgctattcaaaaatttttacacaataggcgaagttaaactttctaagctccgtacctgcggacagcatttgctttgtcctttctgcgctgctattcgtgcatctcgtgctattcaaaaatacgttgaacgtattgatcaagtcttacaagaaaatcgcaagcttaagcctgttctaatcacgctcactgttaaaaacggttctgatctagcagaacgctctgaacatcttatgaagtcctttagaaagctcctagatcgtcgtagggactatgaaaagaaagggcgaggttttaacgagttctgtaaggttcaaggcgctatgtattcatatgaaaatacatttaatgaaaaaacaggcgaatggcatcctcatattcatatgttcgctttagttgataattggattgatcagcaagaattttcagaatattggcatagccttactggcgactctatggttgtcgatgttcgtagggcaaaaaaagaaaaaggtcacggttatagcaaagcggctgctgaagtctgtaagtatgctttgaagtttggcgatctgtccgttgaaaagacttgggaagcgtttaaggttctcaagggcaagcgtttaacaggttcttttggtctgctatggggtgtcaaaatccctgacacaatgacagacgatatgccattagaagatttgccatatctcgaaatgctctacaagttcgcctacagtaaaaaatcctactatgacttactaatcacacgacacgtagagccacatagcaaggacaacgacgacgaggaggagcttcgacaacgaggagaaggcgtacgaagcgatttggcgataacggacgagggaggagcggagcggattgagagcgagggcacgcagcgattgacacgcactgcaccgcaacgaggacgtaaaaaagcccactggagaatcccaccttctacccgtgttcgtgttcggcaacgaatcagaagatgggacggatatttatataatattgacctattcccgtatgttgagcataggctattagccttcatagaagac

SEQ ID NO: 46 - DNA, 549 bp, Artificial Sequence
  orf
atttctaagcgggagtttttagccatcccgaaagggtggcgacgcattgacaattataatttaatctgcaatattcataagacattcataaataatctgattggaatattaaatatgctttttaaatctctaggcgttgcaatcgctacaaataatgcttttaactctgacgaagacttgaaaccaattactattcgtttagctgatagtgaaattcaggtgtatgacactgttgcaaaatcaatgggtctaactcgtcaagattttctatcacacgttattcgttccaacttccgacaggctttaaaagagtttgtagtgggttatacaagttcaaatccttctacgcctttgtctcttttatttaattcaaatacagacaatgacgaagttaagctcaaactttccaaccttctttcatctatttctcaggaattaatggacgaagaagaaaaagaaattcatgaagcaatcgaaaaccatatgaatacttcctatgcatatgcaactcgtccaaaaggcatctttgttgagaaggatgattcaaaa

SEQ ID NO: 47 - protein, 291 aa, Artificial Sequence
  orf
ATGATTAAGCTTGAGGGAATCGTTTTAAACGTATTTACCCAGCAAGGCGGTAAAGATAAAAAAACAGGCGATCAATTTGAAGACCGTGACAAGGTACAAATTTTAGGTGCTATGGCTCTGCCTAATGGTGACGTAAAAAATGAACTTTTTACGTTATCAGTAGAAAATTATCGGGACTTTAAAGACTACCTAAATCAGAAAATTTCGATTGCTGTTGGTGCAATGGCAAGTGGTCGTAATGTGATTTTTTATGTTGCTAAAGGTGCAAAACCTGTTTTAGCAGAGACAATG

SEQ ID NO: 48 - DNA, 1308 bp, Artificial Sequence
  orf
ttgcttaaaaaagacaataaaaaagcccatatttcagaagctttggcgggcggagaaatacaggctttgcaatctgctaaggcagatcaacacagggatagaataacacgttttgcgactttgaaacatagggctaagaatcaggaaaattatttatttaccctggctaagtttaaagagaactatgaaaaagacgttaaaaacgaagaatctatcaaagctctaaaatctgctcagaaattgaatgaatgcggaaactatctgctattcaaaaatttttacacaataggcgaagttaaactttctaagctccgtacctgcggacagcatttgctttgtcctttctgcgctgctattcgtgcatctcgtgctattcaaaaatacgttgaacgtattgatcaagtcttacaagaaaatcgcaagcttaagcctgttctaatcacgctcactgttaaaaacggttctgatctagcagaacgctctgaacatcttatgaagtcctttagaaagctcctagatcgtcgtagggactatgaaaagaaagggcgaggttttaacgagttctgtaaggttcaaggcgctatgtattcatatgaaaatacatttaatgaaaaaacaggcgaatggcatcctcatattcatatgttcgctttagttgataattggattgatcagcaagaattttcagaatattggcatagccttactggcgactctatggttgtcgatgttcgtagggcaaaaaaagaaaaaggtcacggttatagcaaagcggctgctgaagtctgtaagtatgctttgaagtttggcgatctgtccgttgaaaagacttgggaagcgtttaaggttctcaagggcaagcgtttaacaggttcttttggtctgctatggggtgtcaaaatccctgacacaatgacagacgatatgccattagaagatttgccatatctcgaaatgctctacaagttcgcctacagtaaaaaatcctactatgacttactaatcacacgacacgtagagccacatagcaaggacaacgacgacgaggaggagcttcgacaacgaggagaaggcgtacgaagcgatttggcgataacggacgagggaggagcggagcggattgagagcgagggcacgcagcgattgacacgcactgcaccgcaacgaggacgtaaaaaagcccactggagaatcccaccttctacccgtgttcgtgttcggcaacgaatcagaagatgggacggatatttatataatattgacctattcccgtatgttgagcataggctattagccttcatagaagac

SEQ ID NO: 49 - DNA, 156 bp, Artificial Sequence
  orf
acgtttaaaacgattccctcaagcttaatcattttgaatcatccttctcaacaaagatgccttttggacgagttgcatatgcataggaagtattcatatggttttcgattgcttcatgaatttctttttcttcttcgtccattaattcctgagaaa

SEQ ID NO: 50 - DNA, 192 bp, Artificial Sequence
  orf
gtgggattctccagtgggcttttttacgtcctcgttgcggtgcagtgcgtgtcaatcgctgcgtgccctcgctctcaatccgctccgctcctccctcgtccgttatcgccaaatcgcttcgtacgccttctcctcgttgtcgaagctcctcctcgtcgtcgttgtccttgctatgtggctctacgtgtcgtg

SEQ ID NO: 51 - DNA, 309 bp, Artificial Sequence
  orf
atgaaggctaatagcctatgctcaacatacgggaataggtcaatattatataaatatccgtcccatcttctgattcgttgccgaacacgaacacgggtagaaggtgggattctccagtgggcttttttacgtcctcgttgcggtgcagtgcgtgtcaatcgctgcgtgccctcgctctcaatccgctccgctcctccctcgtccgttatcgccaaatcgcttcgtacgccttctcctcgttgtcgaagctcctcctcgtcgtcgttgtccttgctatgtggctctacgtgtcgtgtgattagtaagtca

SEQ ID NO: 52 - DNA, 192 bp, Artificial Sequence
  orf
atttcgagatatggcaaatcttctaatggcatatcgtctgtcattgtgtcagggattttgacaccccatagcagaccaaaagaacctgttaaacgcttgcccttgagaaccttaaacgcttcccaagtcttttcaacggacagatcgccaaacttcaaagcatacttacagacttcagcagccgctttgcta

SEQ ID NO: 53 - DNA, 246 bp, Artificial Sequence
  orf
atatccgtcccatcttctgattcgttgccgaacacgaacacgggtagaaggtgggattctccagtgggcttttttacgtcctcgttgcggtgcagtgcgtgtcaatcgctgcgtgccctcgctctcaatccgctccgctcctccctcgtccgttatcgccaaatcgcttcgtacgccttctcctcgttgtcgaagctcctcctcgtcgtcgttgtccttgctatgtggctctacgtgtcgtgtgat

SEQ ID NO: 54 - DNA, 177 bp, Artificial Sequence
  orf
acgcttgcccttgagaaccttaaacgcttcccaagtcttttcaacggacagatcgccaaacttcaaagcatacttacagacttcagcagccgctttgctataaccgtgacctttttctttttttgccctacgaacatcgacaaccatagagtcgccagtaaggctatgccaatattc

SEQ ID NO: 55 - DNA, 2661 bp, Artificial Sequence
  isolate
caacacagggatagaataacacgttttgcgactttgaaacatagagctaagaatcaggaaaattatttatttaccctggcacaattcaaagacaactatgaaaaagatgttaaaaacgaggaatctatcaaggctctaaaatctgctcagaaattgaatgaatgcggaaactatttgctattcaaaaatttttacacaataggcgaagttaagctctccaagctccgcacctgtggacagcatttgctttgtcctttctgcgctgctatccgtgcatctcgtgcaattcaaaaatacgttgaacgtatcaatcaggttcttcaagagaatcgcaagctcaagcctgttttaatcacgcttaccgtcaaaaatggttcagaccttgccgaaaggtcagaacaccttatgaaatcgtttagaacgcttctagagcgtcgtagggactatttaaaaaagggttggggttatagcgagttctgtaaggttcaaggtgccatgtactcttacgagaatacatacaacgaaaaaacaggtgagtggcatccgcatattcatatgtttgcattggttgatcagtggattgatcagcaggaattttcagaatattggcatagccttactggggactcgatggttgttgacgtccgcagggcgagaaaagaaaaaggacacggctatagtaaagcggctgccgaagtctgtaagtatgctttgaagtttggcgatctatccgttgaaaaaacttgggaagcattcaaggtactcaagggcaagcgtttaacaggttctttcggtcttttatggggtgtcaaaattcctgaaactatgactgatgatatgcctgatgatgaaacactcccttatatggaaatgctgtacaagttcgtttacggcaaaaagtcacattatgacctagccattactcgtatggttgagccaaaggctaaggacgacaacgaggagaaggaggcaccgacgtccgaccgaggcggagaacgtagccgacaggctcaaaaggacgatgcaatcgcatccgacatcccgactcgcgggtgcggtgcgaagcgaggacgcaaaaaagcccactggagaatcccaccttctacccgtgttcgggttcgtcaacgaatccgaagatgggacggatatttatataatattgacctattcccttatgttgagcataggctattagccctcattggctagtagccgtttcggagaaacgcggaagcgtgcattctgaaaattctgctaaagattactcatataatctttagttccttctccccagtagctaggaatttaatatgactgaaattgaaaaattgaagaaagagattgaagacttaaaactgattattgaaagtaaaaataatcaggttgaatctaaaaataagcatattaaaagacttgagagaatcatctcgaatatgaaaaaagaacaatctcagcagtagccgagggggcggagtgcccccgaactgacataataaaaatttcaaaaccttccttccctgattacaagctccccttagattctaagcgggagcttttagccatcccgaatgggtggcgacgcaaagaaaatttgacaaagattctaggtgtattattattcgtattaccaatagaaaatatgcaccataatttgaggaataatctatgtgtcaatcattcggtactatgggacttgcaaaagctgaagctgaaattaacgctcatgaacaagacagcgcatctgttgtttcttctgaaatcaaaccttttacaactaaagcgcctactcacgttattgaaggtttagaccttcttgctggtcattttggaatgactcgcaatgcattggttttaaagctcatcaatcaatatttgggtcaggcattcgctgaatactctactggctataactctgtttttcacaatcctgatgtttcagatgttcagcaaatcattaatgacattgaaaatctaactgaacacagtgacgtttcagaacaagcgaaagaatacgtaaatcgtttggctgtagatcatttggtgaatacctaatgactgagatgcctaaagaacatttagaacaggcgaaagaccttttcgcctacatcattcaagaacatatgaaagacgaccgatgtgtcacacacatcaagatatttaaacaagatggtcgattaaaaatattagaggtaacagaagaatgctaaaccttactggaacagtgattaacgtctttactcaacaaggcggaacaaacaaaaaaggcgagtcttttgaagaccgtgacaaaattcagctactcggtgctttagatctcccaaacgggggaatcaaaaacgagttagttactcttacagtcgaaaattatcgtgacttcaaagatcttctaaatcaaaaaatttctatccctgttggtgttatggcaatgggtcgcaacgtgattttttacgttgcaaaaggcgcacaacctcaaattgtgcaggcataagaattcgttaaaaaaacagtgagttagcgaagcaactcgctgttttttcttttacttgatactattgattaaagtggggatgaaaattgcttaaaaaagacaataaaaaagcccatatttcgaacactttggcgggtggagaaatacaggctttgcaatctgctaaggcagat

SEQ ID NO: 56 - protein, 82 aa, Artificial Sequence
  orf
MASSVRFKVPCTLTRIHTTKKQVSGIRIFICLHWLISGLISRNFQNIGIALLGTRWLLTSAGREKKKDTAIVKRLPKSVSML

SEQ ID NO: 57 - protein, 87 aa, Artificial Sequence
  orf
MSQRLRTTTRRRRHRRPTEAENVADRLKRTMQSHPTSRLAGAVRSEDAKKPTGESHLLPVFGFVNESEDGTDIYIILTYSLMLSIGY

SEQ ID NO: 58 - protein, 397 aa, Artificial Sequence
  orf
MTRFATLKHRAKNQENYLFTLAQFKDNYEKDVKNEESIKALKSAQKLNECGNYLLFKNFYTIGEVKLSKLRTCGQHLLCPFCAAIRASRAIQKYVERINQVLQENRKLKPVLITLTVKNGSDLAERSEHLMKSFRTLLERRRDYLKKGWGYSEFCKVQGAMYSYENTYNEKTGEWHPHIHMFALVDQWIDQQEFSEYWHSLTGDSMVVDVRRARKEKGHGYSKAAAEVCKYALKFGDLSVEKTWEAFKVLKGKRLTGSFGLLWGVKIPETMTDDMPDDETLPYMEMLYKFVYGKKSHYDLAITRMVEPKAKDDNEEKEAPTSDRGGERSRQAQKDDAIASDIPTRGCGAKRGRKKAHWRIPPSTRVRVRQRIRRWDGYLYNIDLFPYVEHRLLALIG

SEQ ID NO: 59 - protein, 119 aa, Artificial Sequence
  orf
MSAVAEGAECPRTDIIKISKPSFPDYKLPLDSKRELLAIPNGWRRKENLTKILGVLLFVLPIENMHHNLRNNLCVNHSVLWDLQKLKLKLTLMNKTAHLLFLLKSNLLQLKRLLTLLKV

SEQ ID NO: 60 - protein, 128 aa, Artificial Sequence
  orf
MCQSFGTMGLAKAEAEINAHEQDSASVVSSEIKPFTTKAPTHVIEGLDLLAGHFGMTRNALVLKLINQYLGQAFAEYSTGYNSVFHNPDVSDVQQIINDIENLTEHSDVSEQAKEYVNRLAVDHLVNT

SEQ ID NO: 61 - protein, 65 aa, Artificial Sequence
  orf
MRKSFGCRSFGEYLMTEMPKEHLEQAKDLFAYIIQEHMKDDRCVTHIKIFKQDGRLKILEVTEEC

SEQ ID NO: 62 - protein, 104 aa, Artificial Sequence
  orf
MKNIRGNRRMLNLTGTVINVFTQQGGTNKKGESFEDRDKIQLLGALDLPNGGIKNELVTLTVENYRDFKDLLNQKISIPVGVMAMGRNVIFYVAKGAQPQIVQA

SEQ ID NO: 63 - protein, 431 aa, Artificial Sequence
  orf
MLKKDNKKAHISNTLAGGEIQALQSAKADQHRDRITRFATLKHRAKNQENYLFTLAQFKDNYEKDVKNEESIKALKSAQKLNECGNYLLFKNFYTIGEVKLSKLRTCGQHLLCPFCAAIRASRAIQKYVERINQVLQENRKLKPVLITLTVKNGSDLAERSEHLMKSFRTLLERRRDYLKKGWGYSEFCKVQGAMYSYENTYNEKTGEWHPHIHMFALVDQWIDQQEFSEYWHSLTGDSMVVDVRRARKEKGHGYSKAAAEVCKYALKFGDLSVEKTWEAFKVLKGKRLTGSFGLLWGVKIPETMTDDMPDDETLPYMEMLYKFVYGKKSHYDLAITRMVEPKAKDDNEEKEAPTSDRGGERSRQAQKDDAIASDIPTRGCGAKRGRKKAHWRIPPSTRVRVRQRIRRWDGYLYNIDLFPYVEHRLLALIG

SEQ ID NO: 64 - protein, 50 aa, Artificial Sequence
  orf
MTHRLFLKLWCIFSIGNTNNNTPRIFVKFSLRRHPFGMAKSSRLESKGSL

SEQ ID NO: 65 - protein, 51 aa, Artificial Sequence
  orf
MKFLLCQFGGTPPPRLLLRLFFFHIRDDSLKSFNMLIFRFNLIIFTFNNQF

SEQ ID NO: 66 - protein, 51 aa, Artificial Sequence
  orf
MGFSSGLFCVLASHRTRESGCRMRLHRPFEPVGYVLRLGRTSVPPSPRCRP

SEQ ID NO: 67 - protein, 78 aa, Artificial Sequence
  orf
MSVPSSDSLTNPNTGRRWDSPVGFFASSLRTAPASRDVGCDCIVLLSLSATFSASVGRRCLLLLVVVLSLWLNHTSNG

SEQ ID NO: 68 - protein, 54 aa, Artificial Sequence
  orf
MSSVIVSGILTPHKRPKEPVKRLPLSTLNASQVFSTDRSPNFKAYLQTSAAALL

SEQ ID NO: 69 - protein, 62 aa, Artificial Sequence
  orf
MLKIPADQSTDQPMQTYEYADATHLFFRCMYSRKSTWHLEPYRTRYNPNPFLNSPYDALEAF

SEQ ID NO: 70 - protein, 50 aa, Artificial Sequence
  orf
MLLKNLIDTFNVFLNCTRCTDSSAERTKQMLSTGAELGELNFAYCVKIFE

SEQ ID NO: 71 - protein, 56 aa, Artificial Sequence
  orf
MIFLILSSMFQSRKTCYSIPVLICLSRLQSLYFSTRQSVRNMGFFIVFFKQFSSPL

SEQ ID NO: 72 - DNA, 246 bp, Artificial Sequence
  orf
atagcgagttctgtaaggttcaaggtgccatgtactcttacgagaatacatacaacgaaaaaacaggtgagtggcatccgcatattcatatgtttgcattggttgatcagtggattgatcagcaggaattttcagaatattggcatagccttactggggactcgatggttgttgacgtccgcagggcgagaaaagaaaaaggacacggctatagtaaagcggctgccgaagtctgtaagtatgctt

SEQ ID NO: 73 - DNA, 261 bp, Artificial Sequence
  orf
ttgagccaaaggctaaggacgacaacgaggagaaggaggcaccgacgtccgaccgaggcggagaacgtagccgacaggctcaaaaggacgatgcaatcgcatccgacatcccgactcgcgggtgcggtgcgaagcgaggacgcaaaaaagcccactggagaatcccaccttctacccgtgttcgggttcgtcaacgaatccgaagatgggacggatatttatataatattgacctattcccttatgttgagcataggctat

SEQ ID NO: 74 - DNA, 1191 bp, Artificial Sequence
  orf
ataacacgttttgcgactttgaaacatagagctaagaatcaggaaaattatttatttaccctggcacaattcaaagacaactatgaaaaagatgttaaaaacgaggaatctatcaaggctctaaaatctgctcagaaattgaatgaatgcggaaactatttgctattcaaaaatttttacacaataggcgaagttaagctctccaagctccgcacctgtggacagcatttgctttgtcctttctgcgctgctatccgtgcatctcgtgcaattcaaaaatacgttgaacgtatcaatcaggttcttcaagagaatcgcaagctcaagcctgttttaatcacgcttaccgtcaaaaatggttcagaccttgccgaaaggtcagaacaccttatgaaatcgtttagaacgcttctagagcgtcgtagggactatttaaaaaagggttggggttatagcgagttctgtaaggttcaaggtgccatgtactcttacgagaatacatacaacgaaaaaacaggtgagtggcatccgcatattcatatgtttgcattggttgatcagtggattgatcagcaggaattttcagaatattggcatagccttactggggactcgatggttgttgacgtccgcagggcgagaaaagaaaaaggacacggctatagtaaagcggctgccgaagtctgtaagtatgctttgaagtttggcgatctatccgttgaaaaaacttgggaagcattcaaggtactcaagggcaagcgtttaacaggttctttcggtcttttatggggtgtcaaaattcctgaaactatgactgatgatatgcctgatgatgaaacactcccttatatggaaatgctgtacaagttcgtttacggcaaaaagtcacattatgacctagccattactcgtatggttgagccaaaggctaaggacgacaacgaggagaaggaggcaccgacgtccgaccgaggcggagaacgtagccgacaggctcaaaaggacgatgcaatcgcatccgacatcccgactcgcgggtgcggtgcgaagcgaggacgcaaaaaagcccactggagaatcccaccttctacccgtgttcgggttcgtcaacgaatccgaagatgggacggatatttatataatattgacctattcccttatgttgagcataggctattagccctcattggc

SEQ ID NO: 75 - DNA, 357 bp, Artificial Sequence
  orf
atctcagcagtagccgagggggcggagtgcccccgaactgacataataaaaatttcaaaaccttccttccctgattacaagctccccttagattctaagcgggagcttttagccatcccgaatgggtggcgacgcaaagaaaatttgacaaagattctaggtgtattattattcgtattaccaatagaaaatatgcaccataatttgaggaataatctatgtgtcaatcattcggtactatgggacttgcaaaagctgaagctgaaattaacgctcatgaacaagacagcgcatctgttgtttcttctgaaatcaaaccttttacaactaaagcgcctactcacgttattgaaggtt

SEQ ID NO: 76 - DNA, 384 bp, Artificial Sequence
  orf
atgtgtcaatcattcggtactatgggacttgcaaaagctgaagctgaaattaacgctcatgaacaagacagcgcatctgttgtttcttctgaaatcaaaccttttacaactaaagcgcctactcacgttattgaaggtttagaccttcttgctggtcattttggaatgactcgcaatgcattggttttaaagctcatcaatcaatatttgggtcaggcattcgctgaatactctactggctataactctgtttttcacaatcctgatgtttcagatgttcagcaaatcattaatgacattgaaaatctaactgaacacagtgacgtttcagaacaagcgaaagaatacgtaaatcgtttggctgtagatcatttggtgaatacc

SEQ ID NO: 77 - DNA, 195 bp, Artificial Sequence
  orf
atacgtaaatcgtttggctgtagatcatttggtgaatacctaatgactgagatgcctaaagaacatttagaacaggcgaaagaccttttcgcctacatcattcaagaacatatgaaagacgaccgatgtgtcacacacatcaagatatttaaacaagatggtcgattaaaaatattagaggtaacagaagaatgc

SEQ ID NO: 78 - DNA, 312 bp, Artificial Sequence
  orf
attaaaaatattagaggtaacagaagaatgctaaaccttactggaacagtgattaacgtctttactcaacaaggcggaacaaacaaaaaaggcgagtcttttgaagaccgtgacaaaattcagctactcggtgctttagatctcccaaacgggggaatcaaaaacgagttagttactcttacagtcgaaaattatcgtgacttcaaagatcttctaaatcaaaaaatttctatccctgttggtgttatggcaatgggtcgcaacgtgattttttacgttgcaaaaggcgcacaacctcaaattgtgcaggca

SEQ ID NO: 79 - DNA, 1293 bp, Artificial Sequence
  orf
ttgcttaaaaaagacaataaaaaagcccatatttcgaacactttggcgggtggagaaatacaggctttgcaatctgctaaggcagatcaacacagggatagaataacacgttttgcgactttgaaacatagagctaagaatcaggaaaattatttatttaccctggcacaattcaaagacaactatgaaaaagatgttaaaaacgaggaatctatcaaggctctaaaatctgctcagaaattgaatgaatgcggaaactatttgctattcaaaaatttttacacaataggcgaagttaagctctccaagctccgcacctgtggacagcatttgctttgtcctttctgcgctgctatccgtgcatctcgtgcaattcaaaaatacgttgaacgtatcaatcaggttcttcaagagaatcgcaagctcaagcctgttttaatcacgcttaccgtcaaaaatggttcagaccttgccgaaaggtcagaacaccttatgaaatcgtttagaacgcttctagagcgtcgtagggactatttaaaaaagggttggggttatagcgagttctgtaaggttcaaggtgccatgtactcttacgagaatacatacaacgaaaaaacaggtgagtggcatccgcatattcatatgtttgcattggttgatcagtggattgatcagcaggaattttcagaatattggcatagccttactggggactcgatggttgttgacgtccgcagggcgagaaaagaaaaaggacacggctatagtaaagcggctgccgaagtctgtaagtatgctttgaagtttggcgatctatccgttgaaaaaacttgggaagcattcaaggtactcaagggcaagcgtttaacaggttctttcggtcttttatggggtgtcaaaattcctgaaactatgactgatgatatgcctgatgatgaaacactcccttatatggaaatgctgtacaagttcgtttacggcaaaaagtcacattatgacctagccattactcgtatggttgagccaaaggctaaggacgacaacgaggagaaggaggcaccgacgtccgaccgaggcggagaacgtagccgacaggctcaaaaggacgatgcaatcgcatccgacatcccgactcgcgggtgcggtgcgaagcgaggacgcaaaaaagcccactggagaatcccaccttctacccgtgttcgggttcgtcaacgaatccgaagatgggacggatatttatataatattgacctattcccttatgttgagcataggctattagccctcattggc

SEQ ID NO: 80 - DNA, 150 bp, Artificial Sequence
  orf
ttgacacatagattattcctcaaattatggtgcatattttctattggtaatacgaataataatacacctagaatctttgtcaaattttctttgcgtcgccacccattcgggatggctaaaagctcccgcttagaatctaaggggagcttg

SEQ ID NO: 81 - DNA, 153 bp, Artificial Sequence
  orf
ttgaaatttttattatgtcagttcgggggcactccgccccctcggctactgctgagattgttcttttttcatattcgagatgattctctcaagtcttttaatatgcttatttttagattcaacctgattatttttactttcaataatcagttt

SEQ ID NO: 82 - DNA, 152 bp, Artificial Sequence
  orf
gtgggattctccagtgggcttttttgcgtcctcgcttcgcaccgcacccgcgagtcgggatgtcggatgcgattgcatcgtccttttgagcctgtcggctacgttctccgcctcggtcggacgtcggtgcctccttctcctcgttgtcgtcc

SEQ ID NO: 83 - DNA, 234 bp, Artificial Sequence
  orf
atatccgtcccatcttcggattcgttgacgaacccgaacacgggtagaaggtgggattctccagtgggcttttttgcgtcctcgcttcgcaccgcacccgcgagtcgggatgtcggatgcgattgcatcgtccttttgagcctgtcggctacgttctccgcctcggtcggacgtcggtgcctccttctcctcgttgtcgtccttagcctttggctcaaccatacgagtaatggc

SEQ ID NO: 84 - DNA, 162 bp, Artificial Sequence
  orf
atatcatcagtcatagtttcaggaattttgacaccccataaaagaccgaaagaacctgttaaacgcttgcccttgagtaccttgaatgcttcccaagttttttcaacggatagatcgccaaacttcaaagcatacttacagacttcggcagccgctttacta

SEQ ID NO: 85 - DNA, 186 bp, Artificial Sequence
  orf
attctgaaaattcctgctgatcaatccactgatcaaccaatgcaaacatatgaatatgcggatgccactcacctgttttttcgttgtatgtattctcgtaagagtacatggcaccttgaaccttacagaactcgctataaccccaacccttttttaaatagtccctacgacgctctagaagcgttc

SEQ ID NO: 86 - DNA, 150 bp, Artificial Sequence
  orf
attctcttgaagaacctgattgatacgttcaacgtatttttgaattgcacgagatgcacggatagcagcgcagaaaggacaaagcaaatgctgtccacaggtgcggagcttggagagcttaacttcgcctattgtgtaaaaatttttgaa

SEQ ID NO: 87 - DNA, 168 bp, Artificial Sequence
  orf
ataattttcctgattcttagctctatgtttcaaagtcgcaaaacgtgttattctatccctgtgttgatctgccttagcagattgcaaagcctgtatttctccacccgccaaagtgttcgaaatatgggcttttttattgtcttttttaagcaattttcatccccactt

SEQ ID NO: 88 - DNA, 2362 bp, Artificial Sequence
  isolate
aaggcagatcaacacagggatagaataacacgttttgggattttgaaacatagagcgaagctacaagaacaatatttgtggacacaagttgatttcaaatctgaaggcgaagacgatctatccaataaggctcttaaggctgcaacaaaattaaaaggttgtggccagtttcttatattcagaaattactacaccatcgaccaagttaagctcgaaaaattccacgtatgtggacagcatttgctatgcccaatgtgtgcaggtattcgcgctgcacgttcaatgaagcgctatttagaccgtatccatgaattaatgcgtcagaaacgcggtttaaagcccgttcttatcactttaacggtcaagaatggagaagacctagaagaacgctataatcaccttacaagctcatttagaacgcttctatcccgttatagggattataagaaaaaaggttggggatttaatcaattctgtaagattgatggcgctttctatactactgaatacacttacaacgataaaaccaagcaatggcatccgcatattcatattttcgctttactcaatgaatggatagaccaggaagaacttgccgagacttggcacgacattaccctggattcttatatcgttgatattcgcagagttaaaaaaaccaaagaacacggttatagcaaagctgttgcagaggtttgtaaatacgctcttaagtttagcgatctgtcactggagaacacatgggaagcctttttgactcttaaaggtaaacgccttacaggttctttcggttctatgcatggagttaaaattccagataaggccacggatgacatgccactggaggagctgccgtacttagaaatgttctatcgttttgtatttgataaaaaatcctactacaacctagaaataacgaaagacgtaaagccacaaactaaggaatgaggagtgaggaggagctgcgacgaccgacacgaccgaatgactttgtgagtggcgataaggacgaagcaatcgcatccgacatcccggcttgcgggtgcggtgcgaagcgaggacgttaagcaagcctacctgtatatacagacttgtaaggggttcgaccccttagaccccaaagggcgcacttagccattgagcaaagctcaacggcagtgctcccccaatcaacagagggggtgtctacccccgaactgactcactcgaatttcaaaacttccttccctgattacaagctccccttagatttctaagcgggagtttttagcaatcccgcagggttgcgacgcaaagataatttgacaaagattctagatgtattattattcgtattacgaatagaaaatatgcaccataatttgaggaataatctatgtgtcaatcattcggtactatgggacttgcaaaagctgaagctgaaattaacgctcatgaacaagacagcgcatctgctgtttcttctgaaatcaaaccttttacaactaaagcgcctactcacgttattgaaggtttagaccttcttgctggtcattttggaatgactcgcaatgcattggttttaaagctcatcaatcaatatttgggtcaggcattcgctgaatactctactggctataactctgtttttcacaatcccgatgtttcagatgttcagcaaatcattaatgacattgaaaatctaactgaacacagtgacgtttcagaacaagcgaaagaatacgtaaatcgtttggttgtagatcatttggtgaatacctaatgactgagatgcctaaagaacatttggaacaagcgaaagaccttttcgctcggattatccaagaacacatgaaagacaaccgcacacttacagccataaaaatctttaaagaagatggtcgtttaaaaattttggagttatcagatgattaaattagaaggcacagtattaaacgtttttactcaacaaggcggtaaagacaaaaagacaggtgatcaatttgctgaccgtgacaaggtgcaaattttgggtgctatggatctgcctaacggtgacattaaaaatgagttatttacgttatctgtagaaaactatcgggactttaaagacttcttaaatcagaaaatctatattgctgttggtgcaatggcaagtggtcgaaatgttattttttacgttgctaaaggcgcacagcctgttttagcagaaacaatgtgaaacatgaaaaaaaacagtgagttagccttggcttctcgctgttttttctttctcttgatactatcaactaaagtggggatgaaattgactaaaaaagacaataaaaaggcccataattctgacactttggcgggtgatgaattacaggctttgcaatctgct

SEQ ID NO: 89 - protein, 62 aa, Artificial Sequence
  orf
MITLQAHLERFYPVIGIIRKKVGDLINSVRLMALSILLNTLTTIKPSNGIRIFIFSLYSMNG

SEQ ID NO: 90 - protein, 54 aa, Artificial Sequence
  orf
MPRLGTTLPWILISLIFAELKKPKNTVIAKLLQRFVNTLLSLAICHWRTHGKPF

SEQ ID NO: 91 - protein, 307 aa, Artificial Sequence
  orf
MTRFGILKHRAKLQEQYLWTQVDFKSEGEDDLSNKALKAATKLKGCGQFLIFRNYYTIDQVKLEKFHVCGQHLLCPMCAGIRAARSMKRYLDRIHELMRQKRGLKPVLITLTVKNGEDLEERYNHLTSSFRTLLSRYRDYKKKGWGFNQFCKIDGAFYTTEYTYNDKTKQWHPHIHIFALLNEWIDQEELAETWHDITLDSYIVDIRRVKKTKEHGYSKAVAEVCKYALKFSDLSLENTWEAFLTLKGKRLTGSFGSMHGVKIPDKATDDMPLEELPYLEMFYRFVFDKKSYYNLEITKDVKPQTKE

SEQ ID NO: 92 - protein, 77 aa, Artificial Sequence
  orf 4
MRRKDNLTKILDVLLFVLRIENMHHNLRNNLCVNHSVLWDLQKLKLKLTLMNKTAHLLFLLKSNLLQLKRLLTLLKV

SEQ ID NO: 93 - protein, 128 aa, Artificial Sequence
  orf
MCQSFGTMGLAKAEAEINAHEQDSASAVSSEIKPFTTKAPTHVIEGLDLLAGHFGMTRNALVLKLINQYLGQAFAEYSTGYNSVFHNPDVSDVQQIINDIENLTEHSDVSEQAKEYVNRLVVDHLVNT

SEQ ID NO: 94 - protein, 64 aa, Artificial Sequence
  orf
MRKSFGCRSFGEYLMTEMPKEHLEQAKDLFARIIQEHMKDNRTLTAIKIFKEDGRLKILELSDD

SEQ ID NO: 95 - protein, 97 aa, Artificial Sequence
  orf
MIKLEGTVLNVFTQQGGKDKKTGDQFADRDKVQILGAMDLPNGDIKNELFTLSVENYRDFKDFLNQKIYIAVGAMASGRNVIFYVAKGAQPVLAETM

SEQ ID NO: 96 - protein, 368 aa, Artificial Sequence
  orf
MKKNSELALASRCFFFLLILSTKVGMKLTKKDNKKAHNSDTLAGDELQALQSAKADQHRDRITRFGILKHRAKLQEQYLWTQVDFKSEGEDDLSNKALKAATKLKGCGQFLIFRNYYTIDQVKLEKFHVCGQHLLCPMCAGIRAARSMKRYLDRIHELMRQKRGLKPVLITLTVKNGEDLEERYNHLTSSFRTLLSRYRDYKKKGWGFNQFCKIDGAFYTTEYTYNDKTKQWHPHIHIFALLNEWIDQEELAETWHDITLDSYIVDIRRVKKTKEHGYSKAVAEVCKYALKFSDLSLENTWEAFLTLKGKRLTGSFGSMHGVKIPDKATDDMPLEELPYLEMFYRFVFDKKSYYNLEITKDVKPQTKE

SEQ ID NO: 97 - protein, 56 aa, Artificial Sequence
  orf
MSELWAFLLSFLVNFIPTLVDSIKRKKKQREAKANSLFFFMFHIVSAKTGCAPLAT

SEQ ID NO: 98 - protein, 75 aa, Artificial Sequence
  orf
MFKRPSSLKIFMAVSVRLSFMCSWIIRAKRSFACSKCSLGISVIRYSPNDLQPNDLRILSLVLKRHCVQLDFQCH

SEQ ID NO: 99 - protein, 60 aa, Artificial Sequence
  orf
MYIQVGLLNVLASHRTRKPGCRMRLLRPYRHSQSHSVVSVVAAPPHSSFLSLWLYVFRYF

SEQ ID NO: 100 - protein, 54 aa, Artificial Sequence
  orf
MSSLRTAPASRDVGCDCFVLIATHKVIRSCRSSQLLLTPHSLVCGFTSFVISRL

SEQ ID NO: 101 - protein, 64 aa, Artificial Sequence
  orf
MSKYGSSSSGMSSVALSGILTPCIEPKEPVRRLPLRVKKASHVFSSDRSLNLRAYLQTSATALL

SEQ ID NO: 102 - protein, 53 aa, Artificial Sequence
  orf
MAKLKSVFTNLCNSFAITVFFGFFNSANINDIRIQGNVVPSLGKFFLVYPFIE

SEQ ID NO: 103 - protein, 57 aa, Artificial Sequence
  orf
MNIRNWPQPFNFVAALRALLDRSSSPSDLKSTCVHKYCSCSFALCFKIPKRVILSLC

SEQ ID NO: 104 - DNA, 186 bp, Artificial Sequence
  orf
ataatcaccttacaagctcatttagaacgcttctatcccgttatagggattataagaaaaaaggttggggatttaatcaattctgtaagattgatggcgctttctatactactgaatacacttacaacgataaaaccaagcaatggcatccgcatattcatattttcgctttactcaatgaatgga

SEQ ID NO: 105 - DNA, 162 bp, Artificial Sequence
  orf
ttgccgagacttggcacgacattaccctggattcttatatcgttgatattcgcagagttaaaaaaaccaaagaacacggttatagcaaagctgttgcagaggtttgtaaatacgctcttaagtttagcgatctgtcactggagaacacatgggaagcctttt

SEQ ID NO: 106 - DNA, 921 bp, Artificial Sequence
  orf
ataacacgttttgggattttgaaacatagagcgaagctacaagaacaatatttgtggacacaagttgatttcaaatctgaaggcgaagacgatctatccaataaggctcttaaggctgcaacaaaattaaaaggttgtggccagtttcttatattcagaaattactacaccatcgaccaagttaagctcgaaaaattccacgtatgtggacagcatttgctatgcccaatgtgtgcaggtattcgcgctgcacgttcaatgaagcgctatttagaccgtatccatgaattaatgcgtcagaaacgcggtttaaagcccgttcttatcactttaacggtcaagaatggagaagacctagaagaacgctataatcaccttacaagctcatttagaacgcttctatcccgttatagggattataagaaaaaaggttggggatttaatcaattctgtaagattgatggcgctttctatactactgaatacacttacaacgataaaaccaagcaatggcatccgcatattcatattttcgctttactcaatgaatggatagaccaggaagaacttgccgagacttggcacgacattaccctggattcttatatcgttgatattcgcagagttaaaaaaaccaaagaacacggttatagcaaagctgttgcagaggtttgtaaatacgctcttaagtttagcgatctgtcactggagaacacatgggaagcctttttgactcttaaaggtaaacgccttacaggttctttcggttctatgcatggagttaaaattccagataaggccacggatgacatgccactggaggagctgccgtacttagaaatgttctatcgttttgtatttgataaaaaatcctactacaacctagaaataacgaaagacgtaaagccacaaactaaggaa

SEQ ID NO: 107 - DNA, 231 bp, Artificial Sequence
  orf
ttgcgacgcaaagataatttgacaaagattctagatgtattattattcgtattacgaatagaaaatatgcaccataatttgaggaataatctatgtgtcaatcattcggtactatgggacttgcaaaagctgaagctgaaattaacgctcatgaacaagacagcgcatctgctgtttcttctgaaatcaaaccttttacaactaaagcgcctactcacgttattgaaggtt

SEQ ID NO: 108 - DNA, 384 bp, Artificial Sequence
  orf
atgtgtcaatcattcggtactatgggacttgcaaaagctgaagctgaaattaacgctcatgaacaagacagcgcatctgctgtttcttctgaaatcaaaccttttacaactaaagcgcctactcacgttattgaaggtttagaccttcttgctggtcattttggaatgactcgcaatgcattggttttaaagctcatcaatcaatatttgggtcaggcattcgctgaatactctactggctataactctgtttttcacaatcccgatgtttcagatgttcagcaaatcattaatgacattgaaaatctaactgaacacagtgacgtttcagaacaagcgaaagaatacgtaaatcgtttggttgtagatcatttggtgaatacc

SEQ ID NO: 109 - DNA, 192 bp, Artificial Sequence
  orf
atacgtaaatcgtttggttgtagatcatttggtgaatacctaatgactgagatgcctaaagaacatttggaacaagcgaaagaccttttcgctcggattatccaagaacacatgaaagacaaccgcacacttacagccataaaaatctttaaagaagatggtcgtttaaaaattttggagttatcagatgat

SEQ ID NO: 110 - DNA, 291 bp, Artificial Sequence
  orf
atgattaaattagaaggcacagtattaaacgtttttactcaacaaggcggtaaagacaaaaagacaggtgatcaatttgctgaccgtgacaaggtgcaaattttgggtgctatggatctgcctaacggtgacattaaaaatgagttatttacgttatctgtagaaaactatcgggactttaaagacttcttaaatcagaaaatctatattgctgttggtgcaatggcaagtggtcgaaatgttattttttacgttgctaaaggcgcacagcctgttttagcagaaacaatg

SEQ ID NO: 111 - DNA, 1104 bp, Artificial Sequence
  orf
atgaaaaaaaacagtgagttagccttggcttctcgctgttttttctttctcttgatactatcaactaaagtggggatgaaattgactaaaaaagacaataaaaaggcccataattctgacactttggcgggtgatgaattacaggctttgcaatctgctaaggcagatcaacacagggatagaataacacgttttgggattttgaaacatagagcgaagctacaagaacaatatttgtggacacaagttgatttcaaatctgaaggcgaagacgatctatccaataaggctcttaaggctgcaacaaaattaaaaggttgtggccagtttcttatattcagaaattactacaccatcgaccaagttaagctcgaaaaattccacgtatgtggacagcatttgctatgcccaatgtgtgcaggtattcgcgctgcacgttcaatgaagcgctatttagaccgtatccatgaattaatgcgtcagaaacgcggtttaaagcccgttcttatcactttaacggtcaagaatggagaagacctagaagaacgctataatcaccttacaagctcatttagaacgcttctatcccgttatagggattataagaaaaaaggttggggatttaatcaattctgtaagattgatggcgctttctatactactgaatacacttacaacgataaaaccaagcaatggcatccgcatattcatattttcgctttactcaatgaatggatagaccaggaagaacttgccgagacttggcacgacattaccctggattcttatatcgttgatattcgcagagttaaaaaaaccaaagaacacggttatagcaaagctgttgcagaggtttgtaaatacgctcttaagtttagcgatctgtcactggagaacacatgggaagcctttttgactcttaaaggtaaacgccttacaggttctttcggttctatgcatggagttaaaattccagataaggccacggatgacatgccactggaggagctgccgtacttagaaatgttctatcgttttgtatttgataaaaaatcctactacaacctagaaataacgaaagacgtaaagccacaaactaaggaa

SEQ ID NO: 112 - DNA, 168 bp, Artificial Sequence
  orf
gtgtcagaattatgggcctttttattgtcttttttagtcaatttcatccccactttagttgatagtatcaagagaaagaaaaaacagcgagaagccaaggctaactcactgttttttttcatgtttcacattgtttctgctaaaacaggctgtgcgcctttagcaacg

SEQ ID NO: 113 - DNA, 225 bp, Artificial Sequence
  orf
atttttaaacgaccatcttctttaaagatttttatggctgtaagtgtgcggttgtctttcatgtgttcttggataatccgagcgaaaaggtctttcgcttgttccaaatgttctttaggcatctcagtcattaggtattcaccaaatgatctacaaccaaacgatttacgtattctttcgcttgttctgaaacgtcactgtgttcagttagattttcaatgtcat

SEQ ID NO: 114 - DNA, 180 bp, Artificial Sequence
  orf
ctgtatatacaggtaggcttgcttaacgtcctcgcttcgcaccgcacccgcaagccgggatgtcggatgcgattgcttcgtccttatcgccactcacaaagtcattcggtcgtgtcggtcgtcgcagctcctcctcactcctcattccttagtttgtggctttacgtctttcgttatttc

SEQ ID NO: 115 - DNA, 162 bp, Artificial Sequence
  orf
acgtcctcgcttcgcaccgcacccgcaagccgggatgtcggatgcgattgcttcgtccttatcgccactcacaaagtcattcggtcgtgtcggtcgtcgcagctcctcctcactcctcattccttagtttgtggctttacgtctttcgttatttctaggttg

SEQ ID NO: 116 - DNA, 192 bp, Artificial Sequence
  orf
atttctaagtacggcagctcctccagtggcatgtcatccgtggccttatctggaattttaactccatgcatagaaccgaaagaacctgtaaggcgtttacctttaagagtcaaaaaggcttcccatgtgttctccagtgacagatcgctaaacttaagagcgtatttacaaacctctgcaacagctttgcta

SEQ ID NO: 117 - DNA, 159 bp, Artificial Sequence
  orf
atcgctaaacttaagagcgtatttacaaacctctgcaacagctttgctataaccgtgttctttggtttttttaactctgcgaatatcaacgatataagaatccagggtaatgtcgtgccaagtctcggcaagttcttcctggtctatccattcattgag

SEQ ID NO: 118 - DNA, 171 bp, Artificial Sequence
  orf
ctgaatataagaaactggccacaaccttttaattttgttgcagccttaagagccttattggatagatcgtcttcgccttcagatttgaaatcaacttgtgtccacaaatattgttcttgtagcttcgctctatgtttcaaaatcccaaaacgtgttattctatccctgtgt

SEQ ID NO: 119 - DNA, 2376 bp, Artificial Sequence
  isolate
caacacagggatagaataacacgttttggtattttgaaacacagatcgaagcaacaagaaaactttttgtggactcttgcaaaattcaaagaaaactaccctcaagacctgccaagtgaagaaacaacaaaagctttaaagtctgctcaaaagttgcaaggctgtggcaattttttgctcttcaaaaatttttacacaatagatcaaactaagttagccaaatttcatgtctgtggtcagcatttgttatgtcctttttgtgctggtatccgtgcttcaaagtctattcagaaatattcagaacgtattgatgaagttctcaagaaaaaccgtaagctaaaagtcgctcttttgacgctgacggttaaaaacggttctgacctaggggaacgtgctaatcatctcttaacgtcgtttagaacgcttatcaagcgcagacaggactatttaaaaaagggtcgtgggtttaatgagttctgtaaaattgatggtgcgatgtattcctatgaaaatacctacaatgaaaaaacaggtgaatggcatcctcatgttcacatgcttgtacttctcaatgattggatagatcaggaagccttgggcgaaacttggcatgaaattactggtgattcttatatcgttgatgttcgaaaagttaaaaaatctaaagaattagggtatgccaaagcagcagcagaggtttgtaaatatgctcttaagtttagtgatctgactgttgaaaaaacttgggaagcatttaaaacccttaagggtaaaaaccttagtggctcatttggttccctgcgtggtgtcaaaatccctaaaaaccttgctgacgatatgcctgaagatcaggatttaccttatctcgaaatgctgtacaagttcgtatttggcaagcattcgcactatgacttagttatgactagacacgtagaagcgcaggcgggagagcgctcggagttaggaggagcttcgacgacgcccgacgagagatatagcgcgggtgcttcaagtgttaaacaaggttcggaatggagcgtggcgacccctggtcgaacagcgctctgttctgtaccgaggtttcaacgtaaaaaatcccactggagaatcccacctaaaacacgtgttagggttctgcaaagaattcgaagatgggacggcattatgtacaacattgatttattcccatatgtcgaacataggctattagctttcatcggttagtacctgtttcggagaaacgagtaagcgtgcattctgaaaaactcaccacttttgaaaatagtgtagtgagtacactttctccagtagccgaacgagtgtctacgagtgacgactccgaaacaagaatttcaaaaccttcctttccctgattacaaagctccccttagatttctaagcgggagtttttagcaatcccgcagggttgcgacacaatgatattgacaaaaaaatgatcggtattattatgggtattgcctagacactaaatacaaattttaaagggtaatactatgtgtgtttcattctctgatatgggtcattctaaagctgaacgagaaatcgaaagtcacgaaagtggcaagactgaaatcgaaactcgtttaaaaccgttcactatcaaaactcaagctcatcaaattgaagcgctggacattgtttccgaaatgatggaaatgagtcgaagtgctttagttgtaaagctcattaatcattatcttggtcaggctgtatatgacttcctttctagttacaactctgtcttctcacacgataagcctgaagaagttgttgtcttagatcatatcgaaggacttctcgctactaagtatcccgatctttctccagaagctaaaagcttcattatcaactctgtggcaaaaattgcttttgaaatgtagggaatcgaaaaatgattaagttagaaggaacagtattaaacgtcttcactcaacaaggtggtaaagacaaaaaaacaggcgatcaatttgctgatcgtgacaaggtacaaattttaggtgctatggatctgcctaatggtgacgtaaaaaatgaactttttacgttatcagtagaaaattatcgggactttaaagacttcttgaatcagaaaatctgtattgctgttggtgcaatggcaagtggtcgtaatgtgattttttacgttgctaaaggcgcaaagcctgttttagcagaacaaatgtgatttatataaaaaacagtgggttagcgaagctactcgctgttttttctttctcttgatactattaattaattcggggacaaaatttcatgaaaaatgacaataaaaaacccctgttatcagaagctttggcgggcgtagataacaagggttcgcaatctgctaaggcagat

SEQ ID NO: 120 - protein, 61 aa, Artificial Sequence
  orf
MLKKLGKHLKPLRVKTLVAHLVPCVVSKSLKTLLTICLKIRIYLISKCCTSSYLASIRTMT

SEQ ID NO: 121 - protein, 74 aa, Artificial Sequence
  orf
MRDIARVLQVLNKVRNGAWRPLVEQRSVLYRGFNVKNPTGESHLKHVLGFCKEFEDGTALCTTLIYSHMSNIGY

SEQ ID NO: 122 - protein, 402 aa, Artificial Sequence
  orf
MTRFGILKHRSKQQENFLWTLAKFKENYPQDLPSEETTKALKSAQKLQGCGNFLLFKNFYTIDQTKLAKFHVCGQHLLCPFCAGIRASKSIQKYSERIDEVLKKNRKLKVALLTLTVKNGSDLGERANHLLTSFRTLIKRRQDYLKKGRGFNEFCKIDGAMYSYENTYNEKTGEWHPHVHMLVLLNDWIDQEALGETWHEITGDSYIVDVRKVKKSKELGYAKAAAEVCKYALKFSDLTVEKTWEAFKTLKGKNLSGSFGSLRGVKIPKNLADDMPEDQDLPYLEMLYKFVFGKHSHYDLVMTRHVEAQAGERSELGGASTTPDERYSAGASSVKQGSEWSVATPGRTALCSVPRFQRKKSHWRIPPKTRVRVLQRIRRWDGIMYNIDLFPYVEHRLLAFIG

SEQ ID NO: 123 - protein, 55 aa, Artificial Sequence
  orf
MSFHRLVPVSEKRVSVHSEKLTTFENSVVSTLSPVAERVSTSDDSETRISKPSFP

SEQ ID NO: 124 - protein, 136 aa, Artificial Sequence
  orf
MQILKGNTMCVSFSDMGHSKAEREIESHESGKTEIETRLKPFTIKTQAHQIEALDIVSEMMEMSRSALVVKLINHYLGQAVYDFLSSYNSVFSHDKPEEVVVLDHIEGLLATKYPDLSPEAKSFIINSVAKIAFEM

SEQ ID NO: 125 - protein, 97 aa, Artificial Sequence
  orf
MIKLEGTVLNVFTQQGGKDKKTGDQFADRDKVQILGAMDLPNGDVKNELFTLSVENYRDFKDFLNQKICIAVGAMASGRNVIFYVAKGAKPVLAEQM

SEQ ID NO: 126 - protein, 441 aa, Artificial Sequence
  orf
MRGQNFMKNDNKKPLLSEALAGVDNKGSQSAKADQHRDRITRFGILKHRSKQQENFLWTLAKFKENYPQDLPSEETTKALKSAQKLQGCGNFLLFKNFYTIDQTKLAKFHVCGQHLLCPFCAGIRASKSIQKYSERIDEVLKKNRKLKVALLTLTVKNGSDLGERANHLLTSFRTLIKRRQDYLKKGRGFNEFCKIDGAMYSYENTYNEKTGEWHPHVHMLVLLNDWIDQEALGETWHEITGDSYIVDVRKVKKSKELGYAKAAAEVCKYALKFSDLTVEKTWEAFKTLKGKNLSGSFGSLRGVKIPKNLADDMPEDQDLPYLEMLYKFVFGKHSHYDLVMTRHVEAQAGERSELGGASTTPDERYSAGASSVKQGSEWSVATPGRTALCSVPRFQRKKSHWRIPPKTRVRVLQRIRRWDGIMYNIDLFPYVEHRLLAFIG

SEQ ID NO: 127 - protein, 56 aa, Artificial Sequence
  orf
MNVVHNAVPSSNSLQNPNTCFRWDSPVGFFTLKPRYRTERCSTRGRHAPFRTLFNT

SEQ ID NO: 128 - protein, 69 aa, Artificial Sequence
  orf
MSSARFLGILTPRREPNEPLRFLPLRVLNASQVFSTVRSLNLRAYLQTSAAALAYPNSLDFLTFRTSTI

SEQ ID NO: 129 - protein, 64 aa, Artificial Sequence
  orf
MNDIRITSNFMPSFAQGFLIYPIIEKYKHVNMRMPFTCFFIVGIFIGIHRTINFTELIKPTTLF

SEQ ID NO: 130 - protein, 52 aa, Artificial Sequence
  orf
MRVVFFEFCKSPQKVFLLLRSVFQNTKTCYSIPVLICLSRLRTLVIYARQSF

SEQ ID NO: 131 - DNA, 183 bp, Artificial Sequence
  orf
ctgttgaaaaaacttgggaagcatttaaaacccttaagggtaaaaaccttagtggctcatttggttccctgcgtggtgtcaaaatccctaaaaaccttgctgacgatatgcctgaagatcaggatttaccttatctcgaaatgctgtacaagttcgtatttggcaagcattcgcactatgact

SEQ ID NO: 132 - DNA, 222 bp, Artificial Sequence
  orf
acgagagatatagcgcgggtgcttcaagtgttaaacaaggttcggaatggagcgtggcgacccctggtcgaacagcgctctgttctgtaccgaggtttcaacgtaaaaaatcccactggagaatcccacctaaaacacgtgttagggttctgcaaagaattcgaagatgggacggcattatgtacaacattgatttattcccatatgtcgaacataggctat

SEQ ID NO: 133 - DNA, 1206 bp, Artificial Sequence
  orf
ataacacgttttggtattttgaaacacagatcgaagcaacaagaaaactttttgtggactcttgcaaaattcaaagaaaactaccctcaagacctgccaagtgaagaaacaacaaaagctttaaagtctgctcaaaagttgcaaggctgtggcaattttttgctcttcaaaaatttttacacaatagatcaaactaagttagccaaatttcatgtctgtggtcagcatttgttatgtcctttttgtgctggtatccgtgcttcaaagtctattcagaaatattcagaacgtattgatgaagttctcaagaaaaaccgtaagctaaaagtcgctcttttgacgctgacggttaaaaacggttctgacctaggggaacgtgctaatcatctcttaacgtcgtttagaacgcttatcaagcgcagacaggactatttaaaaaagggtcgtgggtttaatgagttctgtaaaattgatggtgcgatgtattcctatgaaaatacctacaatgaaaaaacaggtgaatggcatcctcatgttcacatgcttgtacttctcaatgattggatagatcaggaagccttgggcgaaacttggcatgaaattactggtgattcttatatcgttgatgttcgaaaagttaaaaaatctaaagaattagggtatgccaaagcagcagcagaggtttgtaaatatgctcttaagtttagtgatctgactgttgaaaaaacttgggaagcatttaaaacccttaagggtaaaaaccttagtggctcatttggttccctgcgtggtgtcaaaatccctaaaaaccttgctgacgatatgcctgaagatcaggatttaccttatctcgaaatgctgtacaagttcgtatttggcaagcattcgcactatgacttagttatgactagacacgtagaagcgcaggcgggagagcgctcggagttaggaggagcttcgacgacgcccgacgagagatatagcgcgggtgcttcaagtgttaaacaaggttcggaatggagcgtggcgacccctggtcgaacagcgctctgttctgtaccgaggtttcaacgtaaaaaatcccactggagaatcccacctaaaacacgtgttagggttctgcaaagaattcgaagatgggacggcattatgtacaacattgatttattcccatatgtcgaacataggctattagctttcatcggt

SEQ ID NO: 134 - DNA, 165 bp, Artificial Sequence
  orf
attagctttcatcggttagtacctgtttcggagaaacgagtaagcgtgcattctgaaaaactcaccacttttgaaaatagtgtagtgagtacactttctccagtagccgaacgagtgtctacgagtgacgactccgaaacaagaatttcaaaaccttcctttccc

SEQ ID NO: 135 - DNA, 408 bp, Artificial Sequence
  orf
atacaaattttaaagggtaatactatgtgtgtttcattctctgatatgggtcattctaaagctgaacgagaaatcgaaagtcacgaaagtggcaagactgaaatcgaaactcgtttaaaaccgttcactatcaaaactcaagctcatcaaattgaagcgctggacattgtttccgaaatgatggaaatgagtcgaagtgctttagttgtaaagctcattaatcattatcttggtcaggctgtatatgacttcctttctagttacaactctgtcttctcacacgataagcctgaagaagttgttgtcttagatcatatcgaaggacttctcgctactaagtatcccgatctttctccagaagctaaaagcttcattatcaactctgtggcaaaaattgcttttgaaatg

SEQ ID NO: 136 - DNA, 291 bp, Artificial Sequence
  orf
atgattaagttagaaggaacagtattaaacgtcttcactcaacaaggtggtaaagacaaaaaaacaggcgatcaatttgctgatcgtgacaaggtacaaattttaggtgctatggatctgcctaatggtgacgtaaaaaatgaactttttacgttatcagtagaaaattatcgggactttaaagacttcttgaatcagaaaatctgtattgctgttggtgcaatggcaagtggtcgtaatgtgattttttacgttgctaaaggcgcaaagcctgttttagcagaacaaatg

SEQ ID NO: 137 - DNA, 1323 bp, Artificial Sequence
  orf
attcggggacaaaatttcatgaaaaatgacaataaaaaacccctgttatcagaagctttggcgggcgtagataacaagggttcgcaatctgctaaggcagatcaacacagggatagaataacacgttttggtattttgaaacacagatcgaagcaacaagaaaactttttgtggactcttgcaaaattcaaagaaaactaccctcaagacctgccaagtgaagaaacaacaaaagctttaaagtctgctcaaaagttgcaaggctgtggcaattttttgctcttcaaaaatttttacacaatagatcaaactaagttagccaaatttcatgtctgtggtcagcatttgttatgtcctttttgtgctggtatccgtgcttcaaagtctattcagaaatattcagaacgtattgatgaagttctcaagaaaaaccgtaagctaaaagtcgctcttttgacgctgacggttaaaaacggttctgacctaggggaacgtgctaatcatctcttaacgtcgtttagaacgcttatcaagcgcagacaggactatttaaaaaagggtcgtgggtttaatgagttctgtaaaattgatggtgcgatgtattcctatgaaaatacctacaatgaaaaaacaggtgaatggcatcctcatgttcacatgcttgtacttctcaatgattggatagatcaggaagccttgggcgaaacttggcatgaaattactggtgattcttatatcgttgatgttcgaaaagttaaaaaatctaaagaattagggtatgccaaagcagcagcagaggtttgtaaatatgctcttaagtttagtgatctgactgttgaaaaaacttgggaagcatttaaaacccttaagggtaaaaaccttagtggctcatttggttccctgcgtggtgtcaaaatccctaaaaaccttgctgacgatatgcctgaagatcaggatttaccttatctcgaaatgctgtacaagttcgtatttggcaagcattcgcactatgacttagttatgactagacacgtagaagcgcaggcgggagagcgctcggagttaggaggagcttcgacgacgcccgacgagagatatagcgcgggtgcttcaagtgttaaacaaggttcggaatggagcgtggcgacccctggtcgaacagcgctctgttctgtaccgaggtttcaacgtaaaaaatcccactggagaatcccacctaaaacacgtgttagggttctgcaaagaattcgaagatgggacggcattatgtacaacattgatttattcccatatgtcgaacataggctattagctttcatcggt

SEQ ID NO: 138 - DNA, 168 bp, Artificial Sequence
  orf
atcaatgttgtacataatgccgtcccatcttcgaattctttgcagaaccctaacacgtgttttaggtgggattctccagtgggattttttacgttgaaacctcggtacagaacagagcgctgttcgaccaggggtcgccacgctccattccgaaccttgtttaacact

SEQ ID NO: 139 - DNA, 207 bp, Artificial Sequence
  orf
atatcgtcagcaaggtttttagggattttgacaccacgcagggaaccaaatgagccactaaggtttttacccttaagggttttaaatgcttcccaagttttttcaacagtcagatcactaaacttaagagcatatttacaaacctctgctgctgctttggcataccctaattctttagattttttaacttttcgaacatcaacgata

SEQ ID NO: 140 - DNA, 192 bp, Artificial Sequence
  orf
atcaacgatataagaatcaccagtaatttcatgccaagtttcgcccaaggcttcctgatctatccaatcattgagaagtacaagcatgtgaacatgaggatgccattcacctgttttttcattgtaggtattttcataggaatacatcgcaccatcaattttacagaactcattaaacccacgacccttttt

SEQ ID NO: 141 - DNA, 156 bp, Artificial Sequence
  orf
ttgagggtagttttctttgaattttgcaagagtccacaaaaagttttcttgttgcttcgatctgtgtttcaaaataccaaaacgtgttattctatccctgtgttgatctgccttagcagattgcgaacccttgttatctacgcccgccaaagcttc

SEQ ID NO: 142 - DNA, 2315 bp, Artificial Sequence
  isolate
aaggcagatcaacacagggatagaatatcacgttttggacttttgaaacatagagcgaagctacaagaacaatatttgtggacgcaggttgattttaaatccgaaggtgaaaatgagatatccaataaggctctataggctgcaaccaaattaaagggatgcggtcgatttctgctattccataattactacacaatagatcaagttaaacttgctaaggctcattattgttctcagcatttactttgccctatgtgtgctggtgtaagggctgctaagtcaatgagtaaatacgttcaacgtattgaagaattgatgcgtcagaatcgcaaattaaagcccgtattgatcactttaacggttaagaatggggaagacctagaagaacgctttaagcaccttagacgctcatttaggacgcttctcgatcgttataacgattataagaaaaaaggtcgtggttttaatcaattctgtaaaattgatggtgctttctattccactgaatatacctataactcaaaaacaaaagaatggcatccgcacattcatattttcgctttactcaatgaatggatagaccaggaagaactttctgagacctggcatgacattaccctggactcgtacatcgtagatattcgtagagttaaaaaaaccaaagaacacggctatagcaaagctgttgcagaggtttgtaaatacgccctcaaatttagcgatctgtcactggagaacacttgggaggcttatctttctttgaaaggtaatagactgactggctgttttggttctatgtatggcgttaaattacctgaaaaattaacagatgatttgccccttgatgatcttccatatatggaacttctttaccgatttgtctttggtaaaaaatcatattacgacttagaaataacaaaagacgtgaagccaaacaaaaggaatgaggaaggatgaacgaggagcttcgacgaggcccgacgcccgaatgacttgtgaaggcggtacgtcaatcggttgtctgtgatgtgacgttccgtgaacgtgcatcatcatcagacaatcgaataagcaagcctacctgtatatacagacttgtaaggggttcgaccccttagaccccaaagggcgcacttagccattgagcaaagctcaacggcagtgctcccaccagtagcgaggaattgaatatgactgaaattgaaaaattgaagaaagagattgaagacttaaagctgattattgaaagtaaaaataatcaggttgaatctaaaaataagcatattaaaagacttgagagaatcatctcgaatataaaaaaagaacaatcccagtagtaaccgagggggcggagtgcccccgaactgacatcatctaaatttcaaaaccttcccttcctgattacaagctccccttagatttctaagcgggagtttttagccatcccgaatgggtggcgtcgcaaatcataatttgcttttaaattaatagtggtttataagatggtttacatagtgaattatttaaaactcacccttaattaggaattatttatgacaaattatgatgacgaaccaaccaaagtagaaatgcttctttctgaaattaacaatactggaaagtctgcttatagcggtgttcttaaacccctgtctattcgtttacctattcaaacttatgcaaaagttgttgctattgagaattttattggtgctgaaaaaactagtaaaaataaaatcattaatgatttgttagaaattgcttttgatcagatttatccatccttaagtgaatctcaaaaacaagcgttcgattctttctcacaatctcttttagatggttcggagtcaggcaaactatgatcaaattagaaggaatcgttttaaatatctttacccagcaaggcggtaaagataaaaaaacaggtgatcaatttgctgatcgtgacaaggtgcaaattttaagtgctatggatctgcctaacggtgacgtaaaaaatgaattatttacgttatctgtagaaaactatcgggactttagcgacttcttaaatcaaaaaatttctattcctgttggggcaatggcaagcggtcgtaatgtgattttttatgtcgcaaaaggcgcacagcctgaattagcaaaaaccgtgtgaaacacgaaaaaaaaacagtgagttagccttggcttctcgctgttttttctttctcttgatactattaactaaagtggggatgaaattgactaaaaaagacaataaaaaagcccataattctgacactttggcgggtgatgaattacaggctttgcaatctgct

SEQ ID NO: 143 - protein, 50 aa, Artificial Sequence
  orf
MVITIIRKKVVVLINSVKLMVLSIPLNIPITQKQKNGIRTFIFSLYSMNG

SEQ ID NO: 144 - protein, 261 aa, Artificial Sequence
  orf
MLFHNYYTIDQVKLAKAHYCSQHLLCPMCAGVRAAKSMSKYVQRIEELMRQNRKLKPVLITLTVKNGEDLEERFKHLRRSFRTLLDRYNDYKKKGRGFNQFCKIDGAFYSTEYTYNSKTKEWHPHIHIFALLNEWIDQEELSETWHDITLDSYIVDIRRVKKTKEHGYSKAVAEVCKYALKFSDLSLENTWEAYLSLKGNRLTGCFGSMYGVKLPEKLTDDLPLDDLPYMELLYRFVFGKKSYYDLEITKDVKPNKRNEEG

SEQ ID NO: 145 - protein, 50 aa, Artificial Sequence
  orf
MNMTEIEKLKKEIEDLKLIIESKNNQVESKNKHIKRLERIISNIKKEQSQ

SEQ ID NO: 146 - protein, 51 aa, Artificial Sequence
  orf
MKLTILESLLIAVFLNPCLFVYLFKLMQKLLLLRILLVLKKLVKIKSLMIC

SEQ ID NO: 147 - protein, 102 aa, Artificial Sequence
  orf
MTNYDDEPTKVEMLLSEINNTGKSAYSGVLKPLSIRLPIQTYAKVVAIENFIGAEKTSKNKIINDLLEIAFDQIYPSLSESQKQAFDSFSQSLLDGSESGKL

SEQ ID NO: 148 - protein, 119 aa, Artificial Sequence
  orf
MSKTSVRFFLTISFRWFGVRQTMIKLEGIVLNIFTQQGGKDKKTGDQFADRDKVQILSAMDLPNGDVKNELFTLSVENYRDFSDFLNQKISIPVGAMASGRNVIFYVAKGAQPELAKTV

SEQ ID NO: 149 - protein, 90 aa, Artificial Sequence
  orf
MASRCFFFLLILLTKVGMKLTKKDNKKAHNSDTLAGDELQALQSAKADQHRDRISRFGLLKHRAKLQEQYLWTQVDFKSEGENEISNKAL

SEQ ID NO: 150 - protein, 50 aa, Artificial Sequence
  orf
MMMMHVHGTSHHRQPIDVPPSQVIRASGLVEAPRSSFLIPFVWLHVFCYF

SEQ ID NO: 151 - protein, 53 aa, Artificial Sequence
  orf
MAKFEGVFTNLCNSFAIAVFFGFFNSTNIYDVRVQGNVMPGLRKFFLVYPFIE

SEQ ID NO: 152 - DNA, 150 bp, Artificial Sequence
  orf
atcgttataacgattataagaaaaaaggtcgtggttttaatcaattctgtaaaattgatggtgctttctattccactgaatatacctataactcaaaaacaaaagaatggcatccgcacattcatattttcgctttactcaatgaatgga

SEQ ID NO: 153 - DNA, 783 bp, Artificial Sequence
  orf
ctgctattccataattactacacaatagatcaagttaaacttgctaaggctcattattgttctcagcatttactttgccctatgtgtgctggtgtaagggctgctaagtcaatgagtaaatacgttcaacgtattgaagaattgatgcgtcagaatcgcaaattaaagcccgtattgatcactttaacggttaagaatggggaagacctagaagaacgctttaagcaccttagacgctcatttaggacgcttctcgatcgttataacgattataagaaaaaaggtcgtggttttaatcaattctgtaaaattgatggtgctttctattccactgaatatacctataactcaaaaacaaaagaatggcatccgcacattcatattttcgctttactcaatgaatggatagaccaggaagaactttctgagacctggcatgacattaccctggactcgtacatcgtagatattcgtagagttaaaaaaaccaaagaacacggctatagcaaagctgttgcagaggtttgtaaatacgccctcaaatttagcgatctgtcactggagaacacttgggaggcttatctttctttgaaaggtaatagactgactggctgttttggttctatgtatggcgttaaattacctgaaaaattaacagatgatttgccccttgatgatcttccatatatggaacttctttaccgatttgtctttggtaaaaaatcatattacgacttagaaataacaaaagacgtgaagccaaacaaaaggaatgaggaagga

SEQ ID NO: 154 - DNA, 150 bp, Artificial Sequence
  orf
ttgaatatgactgaaattgaaaaattgaagaaagagattgaagacttaaagctgattattgaaagtaaaaataatcaggttgaatctaaaaataagcatattaaaagacttgagagaatcatctcgaatataaaaaaagaacaatcccag

SEQ ID NO: 155 - DNA, 153 bp, Artificial Sequence
  orf
ctgaaattaacaatactggaaagtctgcttatagcggtgttcttaaacccctgtctattcgtttacctattcaaacttatgcaaaagttgttgctattgagaattttattggtgctgaaaaaactagtaaaaataaaatcattaatgatttgt

SEQ ID NO: 156 - DNA, 306 bp, Artificial Sequence
  orf
atgacaaattatgatgacgaaccaaccaaagtagaaatgcttctttctgaaattaacaatactggaaagtctgcttatagcggtgttcttaaacccctgtctattcgtttacctattcaaacttatgcaaaagttgttgctattgagaattttattggtgctgaaaaaactagtaaaaataaaatcattaatgatttgttagaaattgcttttgatcagatttatccatccttaagtgaatctcaaaaacaagcgttcgattctttctcacaatctcttttagatggttcggagtcaggcaaacta

SEQ ID NO: 157 - DNA, 357 bp, Artificial Sequence
  orf
atctcaaaaacaagcgttcgattctttctcacaatctcttttagatggttcggagtcaggcaaactatgatcaaattagaaggaatcgttttaaatatctttacccagcaaggcggtaaagataaaaaaacaggtgatcaatttgctgatcgtgacaaggtgcaaattttaagtgctatggatctgcctaacggtgacgtaaaaaatgaattatttacgttatctgtagaaaactatcgggactttagcgacttcttaaatcaaaaaatttctattcctgttggggcaatggcaagcggtcgtaatgtgattttttatgtcgcaaaaggcgcacagcctgaattagcaaaaaccgtg

SEQ ID NO: 158 - DNA, 270 bp, Artificial Sequence
  orf
ttggcttctcgctgttttttctttctcttgatactattaactaaagtggggatgaaattgactaaaaaagacaataaaaaagcccataattctgacactttggcgggtgatgaattacaggctttgcaatctgctaaggcagatcaacacagggatagaatatcacgttttggacttttgaaacatagagcgaagctacaagaacaatatttgtggacgcaggttgattttaaatccgaaggtgaaaatgagatatccaataaggctcta

SEQ ID NO: 159 - DNA, 150 bp, Artificial Sequence
  orf
ctgatgatgatgcacgttcacggaacgtcacatcacagacaaccgattgacgtaccgccttcacaagtcattcgggcgtcgggcctcgtcgaagctcctcgttcatccttcctcattccttttgtttggcttcacgtcttttgttatttc

SEQ ID NO: 160 - DNA, 159 bp, Artificial Sequence
  orf
atcgctaaatttgagggcgtatttacaaacctctgcaacagctttgctatagccgtgttctttggtttttttaactctacgaatatctacgatgtacgagtccagggtaatgtcatgccaggtctcagaaagttcttcctggtctatccattcattgag

SEQ ID NO: 161 - DNA, 2460 bp, Artificial Sequence
  isolate
aaggcagatcaacacagggatagaataacacgttttgcgactttgaaacatagggctaagaatcaggaaaattatttatttacactggctcagtttaaagagaactatgaaaaagacgttaaaaacgaggaatctatcaacgccctaaaatctgctcagaagttgaatgaatgcggaaactatctgctattcaaaaatttttacacaataggcgaagttaaactctccaagctccacacctgcggacagcatttgctttgtcctttctgtgctgccattcgtgcatctcgtgctatccaaaaatacgttgaacgtattgatcaagtcctgcaagaaaatcgcaagcttaaacctgttttaattacgcttaccgttaaaaacggttctgacctagcggaacgctctgaacatcttatgaagtcctttagaaagctcctagatcgtcgtagggactatgaaaagaaagggcgaggttttaacgagttctgtaaggttcaaggtgctatgtattcatatgagaatacattcaatgagaaaacaggcgaatggcatccgcatattcatatgtttgcactggttgaccagtggattgatcatcaggaattttcagaatattggcatagccttactggtgactcgatggttgtcgatgttcgcagggcgagaaaagaaaaaggacacggctatagcaaagctgctgccgaagtctgtaagtatgctttgaagtttggcgatctatccgttgaaaaaacttgggaagctttcaaagttctcaaaggtaaacgcttaacaggctcttttgggcttctgtggggtgtcaaaattcctgacacaatgacagacgatatgccagaagaagatttaccttatcttgaaatgctttacaagtttacatacggcaaaaagtcttactacgacttactgataacaagacacgtagaagcgcaggcgggagagcgcgcggagttaggaggagctacgacgacgcccgacgagagatatagcgcgggtgcttcaagtgttaaacaaggttcggaatggagcgtggcgacccctggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattagcttttattagttagcctgtttcttgagaaacgcggaagcgtgcattctgaaaattccactaaagattactgacataatctttagttccttctctccagtagctaggaattgaatatgactgaaattgaaaaattgaagaaagagattgaagacttaaagctgattattgaaagtaaaaataatcaggttgaatctaaaaataagcatattaaaagacttgagagaatcatctcgaatatgaaaaaagaacaatctcagcagtagccgaacgagtgtctacgagtgacgactccgaaataagaatttcaaaaccttcctttccctgattacaaagctccccttagatttctaagcgggagtttttagccatcccgaatgggtggcgtcacgacttaaatttgccttaatatttaatttgatatataaatagatttctaaaaagattttaatatatatcgagtaaagattatggaaccaactaaattacaaatgttcgttgctgaggttcagggtactgctgaatctgaatatatgggattgtcaaaacaaatcccgctacgtcttcggattgatctttttgctgaggttatggctcttcatgaaattctttcagctaatcaaaagacaccacgaaataaggttttaaatgaccttttagatattgctattgatcaagttaaaaaagaactagatcaagattcttatgaaaaattgcagactttgtctttccatcattacaaagaactatcttcctttgattcaggtgatttatctgatgacgaggtttcaaaatgactaatctacaaggaactttgataaatgctttccgtgtggatggtggtaaaggtaaagacggcaaagaatatgaagcgcgtgacaaggtgcaaattcttggttcgttggaactcccaaacggggaaattaaacacgaacttgttgacctaacagttgaggattctcgactatttgaaccattcaaaaatcaggttattagtatctcgtgtggtgctatggctatcggtcgtaatgttgttttttatgttcgtaaaggcgcaaaaccgattttagcagaaagtatttgatactcgttaaaaaacagtgagttagcgaagctactcgctgttttttcttttacttgatactattgattaaagtggggacaaaaattgcttaaaaaagacaataaaaaagcccatatttcggacactttggcgggtggagaaatacaggctttgcaatctgct

SEQ ID NO: 162 - protein, 94 aa, Artificial Sequence
  orf
MVVGTMKRKGEVLTSSVRFKVLCIHMRIHSMRKQANGIRIFICLHWLTSGLIIRNFQNIGIALLVTRWLSMFAGREKKKDTAIAKLLPKSVSML

SEQ ID NO: 163 - protein, 74 aa, Artificial Sequence
  orf
MRDIARVLQVLNKVRNGAWRPLVEQRSVLYRSFNVKKPTGESHLLPVFVFGKESEDGTGLCITLIFSHMSNIGY

SEQ ID NO: 164 - protein, 401 aa, Artificial Sequence
  orf
MTRFATLKHRAKNQENYLFTLAQFKENYEKDVKNEESINALKSAQKLNECGNYLLFKNFYTIGEVKLSKLHTCGQHLLCPFCAAIRASRAIQKYVERIDQVLQENRKLKPVLITLTVKNGSDLAERSEHLMKSFRKLLDRRRDYEKKGRGFNEFCKVQGAMYSYENTFNEKTGEWHPHIHMFALVDQWIDHQEFSEYWHSLTGDSMVVDVRRARKEKGHGYSKAAAEVCKYALKFGDLSVEKTWEAFKVLKGKRLTGSFGLLWGVKIPDTMTDDMPEEDLPYLEMLYKFTYGKKSYYDLLITRHVEAQAGERAELGGATTTPDERYSAGASSVKQGSEWSVATPGRTALCSVPKFQRKKAHWRIPPSTRVRVRQRIRRWDGIMYNIDLFPYVEHRLLAFIS

SEQ ID NO: 165 - protein, 153 aa, Artificial Sequence
  orf
MNMTEIEKLKKEIEDLKLIIESKNNQMNMTEIEKLKKEIEDLKLIIESKNNQVESKNKHIKRLERIISNMKKEQSQQMNMTEIEKLKKEIEDLKLIIESKNNQVESKNKHIKRLERIISNMKKEQSQQVESKNKHIKRLERIISNMKKEQSQQ

SEQ ID NO: 166 - protein, 114 aa, Artificial Sequence
  orf
MYRVKIMEPTKLQMFVAEVQGTAESEYMGLSKQIPLRLRIDLFAEVMALHEILSANQKTPRNKVLNDLLDIAIDQVKKELDQDSYEKLQTLSFHHYKELSSFDSGDLSDDEVSK

SEQ ID NO: 167 - protein, 96 aa, Artificial Sequence
  orf
MTNLQGTLINAFRVDGGKGKDGKEYEARDKVQILGSLELPNGEIKHELVDLTVEDSRLFEPFKNQVISISCGAMAIGRNVVFYVRKGAKPILAESI

SEQ ID NO: 168 - protein, 435 aa, Artificial Sequence
  orf
MLKKDNKKAHISDTLAGGEIQALQSAKADQHRDRITRFATLKHRAKNQENYLFTLAQFKENYEKDVKNEESINALKSAQKLNECGNYLLFKNFYTIGEVKLSKLHTCGQHLLCPFCAAIRASRAIQKYVERIDQVLQENRKLKPVLITLTVKNGSDLAERSEHLMKSFRKLLDRRRDYEKKGRGFNEFCKVQGAMYSYENTFNEKTGEWHPHIHMFALVDQWIDHQEFSEYWHSLTGDSMVVDVRRARKEKGHGYSKAAAEVCKYALKFGDLSVEKTWEAFKVLKGKRLTGSFGLLWGVKIPDTMTDDMPEEDLPYLEMLYKFTYGKKSYYDLLITRHVEAQAGERAELGGATTTPDERYSAGASSVKQGSEWSVATPGRTALCSVPKFQRKKAHWRIPPSTRVRVRQRIRRWDGIMYNIDLFPYVEHRLLAFIS

SEQ ID NO: 169 - protein, 100 aa, Artificial Sequence
  orf
MNGSNSRESSTVRSTSSCLISPFGSSNEPRICTLSRASYSLPSLPLPPSTRKAFIKVPCRLVILKPRHQINHLNQRKIVLCNDGKTKSAIFHKNLDLVLF

SEQ ID NO: 170 - protein, 63 aa, Artificial Sequence; identical to SEQ ID NO: 20
  orf

SEQ ID NO: 171 - protein, 54 aa, Artificial Sequence
  orf
MSSVIVSGILTPHRSPKEPVKRLPLRTLKASQVFSTDRSPNFKAYLQTSAAALL

SEQ ID NO: 172 - protein, 50 aa, Artificial Sequence
  orf
MFLQDLINTFNVFLDSTRCTNGSTERTKQMLSAGVELGEFNFAYCVKIFE

SEQ ID NO: 173 - protein, 56 aa, Artificial Sequence
  orf
MIFLILSPMFQSRKTCYSIPVLICLSRLQSLYFSTRQSVRNMGFFIVFFKQFLSPL

SEQ ID NO: 174 - DNA, 282 bp, Artificial Sequence
  orf
atcgtcgtagggactatgaaaagaaagggcgaggttttaacgagttctgtaaggttcaaggtgctatgtattcatatgagaatacattcaatgagaaaacaggcgaatggcatccgcatattcatatgtttgcactggttgaccagtggattgatcatcaggaattttcagaatattggcatagccttactggtgactcgatggttgtcgatgttcgcagggcgagaaaagaaaaaggacacggctatagcaaagctgctgccgaagtctgtaagtatgctt

SEQ ID NO: 175 - DNA, 222 bp, Artificial Sequence
  orf
acgagagatatagcgcgggtgcttcaagtgttaaacaaggttcggaatggagcgtggcgacccctggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctat

SEQ ID NO: 176 - DNA, 1203 bp, Artificial Sequence
  orf
ataacacgttttgcgactttgaaacatagggctaagaatcaggaaaattatttatttacactggctcagtttaaagagaactatgaaaaagacgttaaaaacgaggaatctatcaacgccctaaaatctgctcagaagttgaatgaatgcggaaactatctgctattcaaaaatttttacacaataggcgaagttaaactctccaagctccacacctgcggacagcatttgctttgtcctttctgtgctgccattcgtgcatctcgtgctatccaaaaatacgttgaacgtattgatcaagtcctgcaagaaaatcgcaagcttaaacctgttttaattacgcttaccgttaaaaacggttctgacctagcggaacgctctgaacatcttatgaagtcctttagaaagctcctagatcgtcgtagggactatgaaaagaaagggcgaggttttaacgagttctgtaaggttcaaggtgctatgtattcatatgagaatacattcaatgagaaaacaggcgaatggcatccgcatattcatatgtttgcactggttgaccagtggattgatcatcaggaattttcagaatattggcatagccttactggtgactcgatggttgtcgatgttcgcagggcgagaaaagaaaaaggacacggctatagcaaagctgctgccgaagtctgtaagtatgctttgaagtttggcgatctatccgttgaaaaaacttgggaagctttcaaagttctcaaaggtaaacgcttaacaggctcttttgggcttctgtggggtgtcaaaattcctgacacaatgacagacgatatgccagaagaagatttaccttatcttgaaatgctttacaagtttacatacggcaaaaagtcttactacgacttactgataacaagacacgtagaagcgcaggcgggagagcgcgcggagttaggaggagctacgacgacgcccgacgagagatatagcgcgggtgcttcaagtgttaaacaaggttcggaatggagcgtggcgacccctggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattagcttttattagt

SEQ ID NO: 177 - DNA, 153 bp, Artificial Sequence
  orf
ttgaatatgactgaaattgaaaaattgaagaaagagattgaagacttaaagctgattattgaaagtaaaaataatcaggttgaatctaaaaataagcatattaaaagacttgagagaatcatctcgaatatgaaaaaagaacaatctcagcag

SEQ ID NO: 178 - DNA, 342 bp, Artificial Sequence
  orf
atatatcgagtaaagattatggaaccaactaaattacaaatgttcgttgctgaggttcagggtactgctgaatctgaatatatgggattgtcaaaacaaatcccgctacgtcttcggattgatctttttgctgaggttatggctcttcatgaaattctttcagctaatcaaaagacaccacgaaataaggttttaaatgaccttttagatattgctattgatcaagttaaaaaagaactagatcaagattcttatgaaaaattgcagactttgtctttccatcattacaaagaactatcttcctttgattcaggtgatttatctgatgacgaggtttcaaaa

SEQ ID NO: 179 - DNA, 288 bp, Artificial Sequence
  orf
atgactaatctacaaggaactttgataaatgctttccgtgtggatggtggtaaaggtaaagacggcaaagaatatgaagcgcgtgacaaggtgcaaattcttggttcgttggaactcccaaacggggaaattaaacacgaacttgttgacctaacagttgaggattctcgactatttgaaccattcaaaaatcaggttattagtatctcgtgtggtgctatggctatcggtcgtaatgttgttttttatgttcgtaaaggcgcaaaaccgattttagcagaaagtatt

SEQ ID NO: 180 - DNA, 1305 bp, Artificial Sequence
  orf
ttgcttaaaaaagacaataaaaaagcccatatttcggacactttggcgggtggagaaatacaggctttgcaatctgctaaggcagatcaacacagggatagaataacacgttttgcgactttgaaacatagggctaagaatcaggaaaattatttatttacactggctcagtttaaagagaactatgaaaaagacgttaaaaacgaggaatctatcaacgccctaaaatctgctcagaagttgaatgaatgcggaaactatctgctattcaaaaatttttacacaataggcgaagttaaactctccaagctccacacctgcggacagcatttgctttgtcctttctgtgctgccattcgtgcatctcgtgctatccaaaaatacgttgaacgtattgatcaagtcctgcaagaaaatcgcaagcttaaacctgttttaattacgcttaccgttaaaaacggttctgacctagcggaacgctctgaacatcttatgaagtcctttagaaagctcctagatcgtcgtagggactatgaaaagaaagggcgaggttttaacgagttctgtaaggttcaaggtgctatgtattcatatgagaatacattcaatgagaaaacaggcgaatggcatccgcatattcatatgtttgcactggttgaccagtggattgatcatcaggaattttcagaatattggcatagccttactggtgactcgatggttgtcgatgttcgcagggcgagaaaagaaaaaggacacggctatagcaaagctgctgccgaagtctgtaagtatgctttgaagtttggcgatctatccgttgaaaaaacttgggaagctttcaaagttctcaaaggtaaacgcttaacaggctcttttgggcttctgtggggtgtcaaaattcctgacacaatgacagacgatatgccagaagaagatttaccttatcttgaaatgctttacaagtttacatacggcaaaaagtcttactacgacttactgataacaagacacgtagaagcgcaggcgggagagcgcgcggagttaggaggagctacgacgacgcccgacgagagatatagcgcgggtgcttcaagtgttaaacaaggttcggaatggagcgtggcgacccctggtcgaacagcgctctgttctgtaccgaagtttcaacgtaaaaaagcccactggagaatcccaccttctacccgtgttcgtgttcggcaaagaatccgaagatgggacgggattatgtataacattgatcttttcccatatgtcgaacataggctattagcttttattagt

SEQ ID NO: 181 - DNA, 300 bp, Artificial Sequence
  orf
ttgaatggttcaaatagtcgagaatcctcaactgttaggtcaacaagttcgtgtttaatttccccgtttgggagttccaacgaaccaagaatttgcaccttgtcacgcgcttcatattctttgccgtctttacctttaccaccatccacacggaaagcatttatcaaagttccttgtagattagtcattttgaaacctcgtcatcagataaatcacctgaatcaaaggaagatagttctttgtaatgatggaaagacaaagtctgcaatttttcataagaatcttgatctagttcttttt

SEQ ID NO: 182 - DNA, 189 bp, Artificial Sequence
  orf
atgttcgacatatgggaaaagatcaatgttatacataatcccgtcccatcttcggattctttgccgaacacgaacacgggtagaaggtgggattctccagtgggcttttttacgttgaaacttcggtacagaacagagcgctgttcgaccaggggtcgccacgctccattccgaaccttgtttaacact

SEQ ID NO: 183 - DNA, 162 bp, Artificial Sequence
  orf
atatcgtctgtcattgtgtcaggaattttgacaccccacagaagcccaaaagagcctgttaagcgtttacctttgagaactttgaaagcttcccaagttttttcaacggatagatcgccaaacttcaaagcatacttacagacttcggcagcagctttgcta

SEQ ID NO: 184 - DNA, 150 bp, Artificial Sequence
  orf
attttcttgcaggacttgatcaatacgttcaacgtatttttggatagcacgagatgcacgaatggcagcacagaaaggacaaagcaaatgctgtccgcaggtgtggagcttggagagtttaacttcgcctattgtgtaaaaatttttgaa

SEQ ID NO: 185 - DNA, 168 bp, Artificial Sequence
  orf
ataattttcctgattcttagccctatgtttcaaagtcgcaaaacgtgttattctatccctgtgttgatctgccttagcagattgcaaagcctgtatttctccacccgccaaagtgtccgaaatatgggcttttttattgtcttttttaagcaatttttgtccccactt

SEQ ID NO: 186 - DNA, 2564 bp, Artificial Sequence
  isolate
caacacagggatagaataacacgttttggtattttgaagcacagatcgaagcaacaagaaaaccttttgtggactcttgcaaaattcaaagaaaactacccaatagacacgccaaatgaagaaactgtaaaggctctaaaatctgcacagaagttgaacggctgtggcaattttttgttgttcaaaaatttttacacaatagatcaaaccaagctcgcaaaatttcatgtttgtaatcagcatttgttgtgtccattttgtgcaggtattcgtgcatctaaagcaattcaaaaatatacagaacgtgttgatgaagttttaaagaaaaatcgcaagctcaagcctgttctaattaccctcacagtgaaaaacggtactgacctaagtgaacggtctgcacatctcataaaatcgcttagaacgcttattaagcgtcgtcaggactataataaaaaaggtcgtggctttaacgaattttgcaaaatcaatggtgcaatgtattcctatgaaaatacctacaatgaacaaaccaaagaatggcatccgcatgttcacatgtttgctcttttggatgattggatagaccaggaacaaatgtcggaaacttggcatgaaatcactggtgattcgtttattgtagatattcgaaaagttaaaaaagaaaaagacttaggctatgcaaaggctgctgctgaagtatgtaaatacgctcttaaatttggtgatctaactgttgaaaatacttgggaagcattcaaagttctaaagggtaaacgcttaagcggtgcatttggatcactttatggtgtcaagattcctgaaaatcttgctgacgatatgcccgatgaaaaagaccttccgtatttagagatgctctataaattcgtgttcgggaaacagtcttactatgacctacaaatgacacgtcatgtagagccacatagcaaggacaacgacgacgaggcggagtttcgacaacgagaagaaggcggacgtagcgatttggcgataacggagaatgctcaccgtatcgcgtgtgacgtgaaaacggggagagttctccgtaaaaagcaacactggcaagtaccaccaacaacgagggtacgagtgaagaaacgaatccgaagatgggatggttatttgtgtgttatgcccgtgtaaaaacataaacttatgataaaattctagtcgctgaatagtcggacggcaatccaactacccagctaaccaatcagatctattaaggagatcatcatggctgttgcacatactactgttttgtctttgcttggcaaagatgtttcattttctgttttattggatgagcaaataaaatccttttttcctgaaggaaatataactggcttagtcgaagaagttattatcgctctaaatggtaatcaccaaatcctagttggtgatgagtattaccaaatgtccaaaatccattttatataaaaatctctgtttaattgaatgcgcctattagggcgcatttttgtttttgatagcatttcggcgacttttgcctatcctgcgctagtctgtttcttgagaaacgcgtaagcgtgcattctgaaaaaatcaaccgaacgagtgtctacgagtgacgcatccggattgaaaatcctaaaaccaccttttccctgattacaagctcccctttgccgttctccagtaagaaccgagtcatcccgaatgggtggcggaatagtgatattgatttaatatcatcaatgatatataaatataaaaccaaatatgtacaagacatatcaatttaatatcaggtgaaaaaatggaaccaactaatttacaaatcttcgttgctgaagttaagggtactggcgaatcggaatacatgggtatttataaacaagtgcctttacgtcttcgtgcaggtgtttttgcagaagttgaagcgcttcaagaaatgatggctcgaactcaaaaggtatcaagaaataaggttattaatgacttgcttgaaattgctattgatcaggttaaaggctcattagatgagaaatctttagaacaatttaatatgttcgcctctagtcactataacgatttcactggttctggagatttatcagatgattaaattagaaggaacaattattaacgttttcacccaacagggtggcaaagataaaaaaacaggcgatgcttttgaagatcgcgacaaggttcagctcatgggtgagttagagcttccaaatggtgatattaagcatgaattaatcaatctaagtgttgatgatggcaagcactatgaaacactaaaaaacaagcgtgtcagcattccatgcggtgcgcttgcttctggtcgtaatgtgattttttacgttacaaaaggcggtacgcctaagattcttccaagtatatgaaaaatggaaaaaaacagtgagttagcgaagcggctcgctgtttttttatttacttgatactattaattaagtcggggacaaaatttcatgagaaatgacaataaaaaacccctgttatcggacagtttggcgaccggtgataacaagggtttgcaatctgctaaggcagat

SEQ ID NO: 187 - protein, 381 aa, Artificial Sequence
  orf
MTRFGILKHRSKQQENLLWTLAKFKENYPIDTPNEETVKALKSAQKLNGCGNFLLFKNFYTIDQTKLAKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFALLDDWIDQEQMSETWHEITGDSFIVDIRKVKKEKDLGYAKAAAEVCKYALKFGDLTVENTWEAFKVLKGKRLSGAFGSLYGVKIPENLADDMPDEKDLPYLEMLYKFVFGKQSYYDLQMTRHVEPHSKDNDDEAEFRQREEGGRSDLAITENAHRIACDVKTGRVLRKKQHWQVPPTTRVRVKKRIRRWDGYLCVMPV

SEQ ID NO: 188 - protein, 75 aa, Artificial Sequence
  orf
MRSIKEIIMAVAHTTVLSLLGKDVSFSVLLDEQIKSFFPEGNITGLVEEVIIALNGNHQILVGDEYYQMSKIHFI

SEQ ID NO: 189 - protein, 56 aa, Artificial Sequence
  orf
MRIENPKTTFSLITSSPLPFSSKNRVIPNGWRNSDIDLISSMIYKYKTKYVQDISI

SEQ ID NO: 190 - protein, 115 aa, Artificial Sequence
  orf
MCTRHINLISGEKMEPTNLQIFVAEVKGTGESEYMGIYKQVPLRLRAGVFAEVEALQEMMARTQKVSRNKVINDLLEIAIDQVKGSLDEKSLEQFNMFASSHYNDFTGSGDLSDD

SEQ ID NO: 191 - protein, 115 aa, Artificial Sequence
  orf
MCSPLVTITISLVLEIYQMIKLEGTIINVFTQQGGKDKKTGDAFEDRDKVQLMGELELPNGDIKHELINLSVDDGKHYETLKNKRVSIPCGALASGRNVIFYVTKGGTPKILPSI

SEQ ID NO: 192 - protein, 414 aa, Artificial Sequence
  orf
MRNDNKKPLLSDSLATGDNKGLQSAKADQHRDRITRFGILKHRSKQQENLLWTLAKFKENYPIDTPNEETVKALKSAQKLNGCGNFLLFKNFYTIDQTKLAKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFALLDDWIDQEQMSETWHEITGDSFIVDIRKVKKEKDLGYAKAAAEVCKYALKFGDLTVENTWEAFKVLKGKRLSGAFGSLYGVKIPENLADDMPDEKDLPYLEMLYKFVFGKQSYYDLQMTRHVEPHSKDNDDEAEFRQREEGGRSDLAITENAHRIACDVKTGRVLRKKQHWQVPPTTRVRVKKRIRRWDGYLCVMPV

SEQ ID NO: 193 - protein, 59 aa, Artificial Sequence
  orf
MPMYSDSPVPLTSATKICKLVGSIFSPDIKLICLVHIWFYIYISLMILNQYHYSATHSG

SEQ ID NO: 194 - protein, 59 aa, Artificial Sequence
  orf
MDMSCTYLVLYLYIIDDIKSISLFRHPFGMTRFLLENGKGELVIREKVVLGFSIRMRHS

SEQ ID NO: 195 - protein, 64 aa, Artificial Sequence
  orf
MNITIPPPIRDDSVLTGERQRGACNQGKGGFRIFNPDASLVDTRSVDFFRMHAYAFLKKQTSAG

SEQ ID NO: 196 - protein, 53 aa, Artificial Sequence
  orf
MITSSTKPVIFPSGKKDFICSSNKTENETSLPSKDKTVVCATAMMISLIDLIG

SEQ ID NO: 197 - protein, 100 aa, Artificial Sequence
  orf
MDCRPTIQRLEFYHKFMFLHGHNTQITIPSSDSFLHSYPRCWWYLPVLLFTENSPRFHVTRDTVSILRYRQIATSAFFSLSKLRLVVVVLAMWLYMTCHL

SEQ ID NO: 198 - protein, 106 aa, Artificial Sequence
  orf
MFGFVSSLVPSLLVVLASVAFYGELSPFSRHTRYGEHSPLSPNRYVRLLLVVETPPRRRCPCYVALHDVSFVGHSKTVSRTRIYRASLNTEGLFHRAYRQQDFQES

SEQ ID NO: 199 - protein, 86 aa, Artificial Sequence
  orf
MYNKRITSDFMPSFRHLFLVYPIIQKSKHVNMRMPFFGLFIVGIFIGIHCTIDFAKFVKATTFFIIVLTTLNKRSKRFYEMCRPFT

SEQ ID NO: 200 - protein, 58 aa, Artificial Sequence
  orf
MNTFCIFLNCFRCTNTCTKWTQQMLITNMKFCELGLIYCVKIFEQQKIATAVQLLCRF

SEQ ID NO: 201 - protein, 57 aa, Artificial Sequence
  orf
MWRVYWVVFFEFCKSPQKVFLLLRSVLQNTKTCYSIPVLICLSRLQTLVITGRQTVR

SEQ ID NO: 202 - DNA, 1143 bp, Artificial Sequence
  orf
ataacacgttttggtattttgaagcacagatcgaagcaacaagaaaaccttttgtggactcttgcaaaattcaaagaaaactacccaatagacacgccaaatgaagaaactgtaaaggctctaaaatctgcacagaagttgaacggctgtggcaattttttgttgttcaaaaatttttacacaatagatcaaaccaagctcgcaaaatttcatgtttgtaatcagcatttgttgtgtccattttgtgcaggtattcgtgcatctaaagcaattcaaaaatatacagaacgtgttgatgaagttttaaagaaaaatcgcaagctcaagcctgttctaattaccctcacagtgaaaaacggtactgacctaagtgaacggtctgcacatctcataaaatcgcttagaacgcttattaagcgtcgtcaggactataataaaaaaggtcgtggctttaacgaattttgcaaaatcaatggtgcaatgtattcctatgaaaatacctacaatgaacaaaccaaagaatggcatccgcatgttcacatgtttgctcttttggatgattggatagaccaggaacaaatgtcggaaacttggcatgaaatcactggtgattcgtttattgtagatattcgaaaagttaaaaaagaaaaagacttaggctatgcaaaggctgctgctgaagtatgtaaatacgctcttaaatttggtgatctaactgttgaaaatacttgggaagcattcaaagttctaaagggtaaacgcttaagcggtgcatttggatcactttatggtgtcaagattcctgaaaatcttgctgacgatatgcccgatgaaaaagaccttccgtatttagagatgctctataaattcgtgttcgggaaacagtcttactatgacctacaaatgacacgtcatgtagagccacatagcaaggacaacgacgacgaggcggagtttcgacaacgagaagaaggcggacgtagcgatttggcgataacggagaatgctcaccgtatcgcgtgtgacgtgaaaacggggagagttctccgtaaaaagcaacactggcaagtaccaccaacaacgagggtacgagtgaagaaacgaatccgaagatgggatggttatttgtgtgttatgcccgtg

SEQ ID NO: 203 - DNA, 225 bp, Artificial Sequence
  orf
atcagatctattaaggagatcatcatggctgttgcacatactactgttttgtctttgcttggcaaagatgtttcattttctgttttattggatgagcaaataaaatccttttttcctgaaggaaatataactggcttagtcgaagaagttattatcgctctaaatggtaatcaccaaatcctagttggtgatgagtattaccaaatgtccaaaatccattttata

SEQ ID NO: 204 - DNA, 168 bp, Artificial Sequence
  orf
atccggattgaaaatcctaaaaccaccttttccctgattacaagctcccctttgccgttctccagtaagaaccgagtcatcccgaatgggtggcggaatagtgatattgatttaatatcatcaatgatatataaatataaaaccaaatatgtacaagacatatcaatt

SEQ ID NO: 205 - DNA, 345 bp, Artificial Sequence
  orf
atatgtacaagacatatcaatttaatatcaggtgaaaaaatggaaccaactaatttacaaatcttcgttgctgaagttaagggtactggcgaatcggaatacatgggtatttataaacaagtgcctttacgtcttcgtgcaggtgtttttgcagaagttgaagcgcttcaagaaatgatggctcgaactcaaaaggtatcaagaaataaggttattaatgacttgcttgaaattgctattgatcaggttaaaggctcattagatgagaaatctttagaacaatttaatatgttcgcctctagtcactataacgatttcactggttctggagatttatcagatgat

SEQ ID NO: 206 - DNA, 345 bp, Artificial Sequence
  orf
atatgttcgcctctagtcactataacgatttcactggttctggagatttatcagatgattaaattagaaggaacaattattaacgttttcacccaacagggtggcaaagataaaaaaacaggcgatgcttttgaagatcgcgacaaggttcagctcatgggtgagttagagcttccaaatggtgatattaagcatgaattaatcaatctaagtgttgatgatggcaagcactatgaaacactaaaaaacaagcgtgtcagcattccatgcggtgcgcttgcttctggtcgtaatgtgattttttacgttacaaaaggcggtacgcctaagattcttccaagtata

SEQ ID NO: 207 - DNA, 1242 bp, Artificial Sequence
  orf
atgagaaatgacaataaaaaacccctgttatcggacagtttggcgaccggtgataacaagggtttgcaatctgctaaggcagatcaacacagggatagaataacacgttttggtattttgaagcacagatcgaagcaacaagaaaaccttttgtggactcttgcaaaattcaaagaaaactacccaatagacacgccaaatgaagaaactgtaaaggctctaaaatctgcacagaagttgaacggctgtggcaattttttgttgttcaaaaatttttacacaatagatcaaaccaagctcgcaaaatttcatgtttgtaatcagcatttgttgtgtccattttgtgcaggtattcgtgcatctaaagcaattcaaaaatatacagaacgtgttgatgaagttttaaagaaaaatcgcaagctcaagcctgttctaattaccctcacagtgaaaaacggtactgacctaagtgaacggtctgcacatctcataaaatcgcttagaacgcttattaagcgtcgtcaggactataataaaaaaggtcgtggctttaacgaattttgcaaaatcaatggtgcaatgtattcctatgaaaatacctacaatgaacaaaccaaagaatggcatccgcatgttcacatgtttgctcttttggatgattggatagaccaggaacaaatgtcggaaacttggcatgaaatcactggtgattcgtttattgtagatattcgaaaagttaaaaaagaaaaagacttaggctatgcaaaggctgctgctgaagtatgtaaatacgctcttaaatttggtgatctaactgttgaaaatacttgggaagcattcaaagttctaaagggtaaacgcttaagcggtgcatttggatcactttatggtgtcaagattcctgaaaatcttgctgacgatatgcccgatgaaaaagaccttccgtatttagagatgctctataaattcgtgttcgggaaacagtcttactatgacctacaaatgacacgtcatgtagagccacatagcaaggacaacgacgacgaggcggagtttcgacaacgagaagaaggcggacgtagcgatttggcgataacggagaatgctcaccgtatcgcgtgtgacgtgaaaacggggagagttctccgtaaaaagcaacactggcaagtaccaccaacaacgagggtacgagtgaagaaacgaatccgaagatgggatggttatttgtgtgttatgcccgtg

SEQ ID NO: 208 - DNA, 177 bp, Artificial Sequence
  orf
atacccatgtattccgattcgccagtacccttaacttcagcaacgaagatttgtaaattagttggttccattttttcacctgatattaaattgatatgtcttgtacatatttggttttatatttatatatcattgatgatattaaatcaatatcactattccgccacccattcggga

SEQ ID NO: 209 - DNA, 177 bp, Artificial Sequence
  orf
attgatatgtcttgtacatatttggttttatatttatatatcattgatgatattaaatcaatatcactattccgccacccattcgggatgactcggttcttactggagaacggcaaaggggagcttgtaatcagggaaaaggtggttttaggattttcaatccggatgcgtcactcg

SEQ ID NO: 210 - DNA, 192 bp, Artificial Sequence
  orf
atcaatatcactattccgccacccattcgggatgactcggttcttactggagaacggcaaaggggagcttgtaatcagggaaaaggtggttttaggattttcaatccggatgcgtcactcgtagacactcgttcggttgattttttcagaatgcacgcttacgcgtttctcaagaaacagactagcgcagga

SEQ ID NO: 211 - DNA, 154 bp, Artificial Sequence
  orf
ataataacttcttcgactaagccagttatatttccttcaggaaaaaaggattttatttgctcatccaataaaacagaaaatgaaacatctttgccaagcaaagacaaaacagtagtatgtgcaacagccatgatgatctccttaatagatctga

SEQ ID NO: 212 - DNA, 300 bp, Artificial Sequence
  orf
ttggattgccgtccgactattcagcgactagaattttatcataagtttatgtttttacacgggcataacacacaaataaccatcccatcttcggattcgtttcttcactcgtaccctcgttgttggtggtacttgccagtgttgctttttacggagaactctccccgttttcacgtcacacgcgatacggtgagcattctccgttatcgccaaatcgctacgtccgccttcttctcgttgtcgaaactccgcctcgtcgtcgttgtccttgctatgtggctctacatgacgtgtcatttg

SEQ ID NO: 213 - DNA, 318 bp, Artificial Sequence
  orf
atcttcggattcgtttcttcactcgtaccctcgttgttggtggtacttgccagtgttgctttttacggagaactctccccgttttcacgtcacacgcgatacggtgagcattctccgttatcgccaaatcgctacgtccgccttcttctcgttgtcgaaactccgcctcgtcgtcgttgtccttgctatgtggctctacatgacgtgtcatttgtaggtcatagtaagactgtttcccgaacacgaatttatagagcatctctaaatacggaaggtctttttcatcgggcatatcgtcagcaagattttcaggaatct

SEQ ID NO: 214 - DNA, 258 bp, Artificial Sequence
  orf
atctacaataaacgaatcaccagtgatttcatgccaagtttccgacatttgttcctggtctatccaatcatccaaaagagcaaacatgtgaacatgcggatgccattctttggtttgttcattgtaggtattttcataggaatacattgcaccattgattttgcaaaattcgttaaagccacgaccttttttattatagtcctgacgacgcttaataagcgttctaagcgattttatgagatgtgcagaccgttcact

SEQ ID NO: 215 - DNA, 174 bp, Artificial Sequence
  orf
atcaacacgttctgtatatttttgaattgctttagatgcacgaatacctgcacaaaatggacacaacaaatgctgattacaaacatgaaattttgcgagcttggtttgatctattgtgtaaaaatttttgaacaacaaaaaattgccacagccgttcaacttctgtgcagattt

SEQ ID NO: 216 - DNA, 170 bp, Artificial Sequence
  orf
atttggcgtgtctattgggtagttttctttgaattttgcaagagtccacaaaaggttttcttgttgcttcgatctgtgcttcaaaataccaaaacgtgttattctatccctgtgttgatctgccttagcagattgcaaacccttgttatcaccggtcgccaaactgtccg

SEQ ID NO: 217 - DNA, 2850 bp, Artificial Sequence
  isolate
caacacagggatagaataacacgttttgggattttgaaacatagagcgaagcaacaagaaaattttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactaagctctcaaaatttcatgtatgtgggcagcatttgttatgtcctttctgtgctggtattcgtgcatctaaagcaattcaaaaatatactgaacgtgttgatgaagtgcttaagaaaaatcgcaagcttaagcctgttttaatcacgtttacggtgaagaatggaatcgacttaggggaaagatcatcccacctcataaaatcgtttagaacgcttctgaagcgtcgtagggactatattaaaaaaggtcgtgggtttaatgagttctgtaagatcaatggggctgtgtattcttatgaaaacacgtataacgagcaaacaaaggaatggcatccgcacttacatatgtttgcacttttggatgattggatagaccaggaagagctttcacagtattggcattccatcactggtgattcaatgattgtagatattcgcagggtcaaaaaagaaaaaggcttaggctattcaaaggctgctgcggaagtatgtaaatatgctcttaaattcggtgatttgtctgtccaaaatacttgggaagctttcaaagtcttaaaaggtaagcgtttaagtggtgcatttggttctttatggggtgtcaaaattcctgaaaatcttgctgatgatatgcctgatgaaaaagatcttccttacttagaaatgctctataaattcgtatttggaaaacagtcttactatgacttaactatgacacgacacgtagagccacaatgcaaggacagggacaacgacgaagaggaggtagcgacgactgacggaggcggaaatggacgcagcagtgaggcgatcaaggacgggcgaggacgggcggagcgcaccgagggcacgcagggtgcacggcaccgcccgcagcacggacgtaaaaaacaacactggcaagtacctccagtaacgagggtacgagtgaaaaaacggattaaaagatgggatggttatttgtgtgttatgcacatgtaaaatcataaacccatgataaaattctagtcgctgaatagtcggattgcagtccaactacccagctaaccacaacgatctaagaaggagatcatcatggctgttgcacatactactgttttgtctttgcttggcaaagaagtttcattttctgttttattagatgaccaaaataaaatcttttcccctggcggtattcatataaccggattagttgaaaaagttgtaattgatttaactggtgatcatgcaattttagttggtgatgagttttatcaactttccgaaatagacttacctgtttagttaacgattttaccaatgcccgaaaatacatatttttgggcatttttttgtccgtttcttgagaaatgcgtaagcgtgcattctgaaaaaataaacattgagattggcgaagagattcggggcgctgggcgtggggtcgggatgtcactcgatgtcccgccctgtgactggcgacacgagatattcgacaatctcactttagaaacaatatcagagggcacggagtgtgcccgaactgaagaaataaaaattaaaaactacctttccctgattacaagctcccttttgattttctccgggataaacagagccatccggatggtggcgactgactattacttttgcttttttacttaatatggtttataagttggtttacaggttaaaccatataaaatctaaccttaatttaggaattatttatgacaaattatcatgatgaaccaactaaagtagaaatgcttctttctgaaattaaccgtactggtaaatcttcttattctggtgctcttaaaccattatctattcgtttacctattcaaacttacgcaaaagtcgttgctattgaaaactttattggtgctgaaaaaactagtaaaaataaagttatcaatgatttgttagaaattgcttttgatcagatttatccatctttaaatgaatctcaaaaacatgcatttgatcatttttcacagtctcttttagatggtttggagtcaggaaaattatgattggacctcaagttctttcaaagggaacaatggattccctggaagaagcatctgcttatttcaagcaagttttacaagaacatcttgataacaaacgtgacatcacattatttgcgattgctgttgtaggcggtcgtttgaaagtcgtagagggattaccaaaatgattaaattagaaggcacaattattaatgtttttacccagcaaggcggtaaagacaaaaaaactggtgaagcatttgccgatcgtgacaagattcaattgttaggtgagtttgaactccctaacggtgataaaaaaaatgaattaattgatcttactgttgaaaacggtaaggaatatgaaggtatgaaaaataagcgtgtgagcattccatgtggtgctcttgcttcgggtcgaaatgtgattttttatgtcgcaaaaggcgcaacacccaaacttttaaacacagtttgatactcgttaaaaaacagcaacttacgaagtaagttcgctgttttttcttttacttgatactattaattaaagtggggacaaaaattgactgtaaaagacaataaaaaagcccatatttctgatgctttggcgggcgacgaaatacaggctttgcaatctgctaaggcagat

SEQ ID NO: 218 - protein, 390 aa, Artificial Sequence
  orf
MTRFGILKHRAKQQENFLWTLAKFKENYPKDAPSEESTRAIKSAQKLKECGNFLLFKNFYTIDQTKLSKFHVCGQHLLCPFCAGIRASKAIQKYTERVDEVLKKNRKLKPVLITFTVKNGIDLGERSSHLIKSFRTLLKRRRDYIKKGRGFNEFCKINGAVYSYENTYNEQTKEWHPHLHMFALLDDWIDQEELSQYWHSITGDSMIVDIRRVKKEKGLGYSKAAAEVCKYALKFGDLSVQNTWEAFKVLKGKRLSGAFGSLWGVKIPENLADDMPDEKDLPYLEMLYKFVFGKQSYYDLTMTRHVEPQCKDRDNDEEEVATTDGGGNGRSSEAIKDGRGRAERTEGTQGARHRPQHGRKKQHWQVPPVTRVRVKKRIKRWDGYLCVMHM

SEQ ID NO: 219 - protein, 71 aa, Artificial Sequence
  orf
MIMAVAHTTVLSLLGKEVSFSVLLDDQNKIFSPGGIHITGLVEKVVIDLTGDHAILVGDEFYQLSEIDLPV

SEQ ID NO: 220 - protein, 117 aa, Artificial Sequence
  orf
MILPMPENTYFWAFFCPFLEKCVSVHSEKINIEIGEEIRGAGRGVGMSLDVPPCDWRHEIFDNLTLETISEGTECARTEEIKIKNYLSLITSSLLIFSGINRAIRMVATDYYFCFFT

SEQ ID NO: 221 - protein, 51 aa, Artificial Sequence
  orf
MKLTVLVNLLILVLLNHYLFVYLFKLTQKSLLLKTLLVLKKLVKIKLSMIC

SEQ ID NO: 222 - protein, 102 aa, Artificial Sequence
  orf
MTNYHDEPTKVEMLLSEINRTGKSSYSGALKPLSIRLPIQTYAKVVAIENFIGAEKTSKNKVINDLLEIAFDQIYPSLNESQKHAFDHFSQSLLDGLESGKL

SEQ ID NO: 223 - protein, 57 aa, Artificial Sequence
  orf
MMIGPQVLSKGTMDSLEEASAYFKQVLQEHLDNKRDITLFAIAVVGGRLKVVEGLPK

SEQ ID NO: 224 - protein, 116 aa, Artificial Sequence
  orf
MICDCCCRRSFESRRGITKMIKLEGTIINVFTQQGGKDKKTGEAFADRDKIQLLGEFELPNGDKKNELIDLTVENGKEYEGMKNKRVSIPCGALASGRNVIFYVAKGATPKLLNTV

SEQ ID NO: 225 - protein, 424 aa, Artificial Sequence
  orf
MTVKDNKKAHISDALAGDEIQALQSAKADQHRDRITRFGILKHRAKQQENFLWTLAKFKENYPKDAPSEESTRAIKSAQKLKECGNFLLFKNFYTIDQTKLSKFHVCGQHLLCPFCAGIRASKAIQKYTERVDEVLKKNRKLKPVLITFTVKNGIDLGERSSHLIKSFRTLLKRRRDYIKKGRGFNEFCKINGAVYSYENTYNEQTKEWHPHLHMFALLDDWIDQEELSQYWHSITGDSMIVDIRRVKKEKGLGYSKAAAEVCKYALKFGDLSVQNTWEAFKVLKGKRLSGAFGSLWGVKIPENLADDMPDEKDLPYLEMLYKFVFGKQSYYDLTMTRHVEPQCKDRDNDEEEVATTDGGGNGRSSEAIKDGRGRAERTEGTQGARHRPQHGRKKQHWQVPPVTRVRVKKRIKRWDGYLCVMHM

SEQ ID NO: 226 - protein, 51 aa, Artificial Sequence
  orf
MLSRSANASPVFLSLPPCWVKTLIIVPSNLIILVIPLRLSNDRLQQQSQIM

SEQ ID NO: 227 - protein, 84 aa, Artificial Sequence
  orf
MFISSVRAHSVPSDIVSKVRLSNISCRQSQGGTSSDIPTPRPAPRISSPISMFIFSECTLTHFSRNGQKNAQKYVFSGIGKIVN

SEQ ID NO: 228 - protein, 125 aa, Artificial Sequence
  orf
MSRVASHRAGHRVTSRPHAQRPESLRQSQCLFFQNARLRISQETDKKMPKNMYFRALVKSLTKQVSLFRKVDKTHHQLKLHDHQLNQLQLFQLIRLYEYRQGKRFYFGHLIKQKMKLLCQAKTKQ

SEQ ID NO: 229 - protein, 109 aa, Artificial Sequence
  orf
MDCNPTIQRLEFYHGFMILHVHNTQITIPSFNPFFHSYPRYWRYLPVLFFTSVLRAVPCTLRALGALRPSSPVLDRLTAASISASVSRRYLLFVVVPVLALWLYVSCHS

SEQ ID NO: 230 - protein, 80 aa, Artificial Sequence
  orf
MRFFTRTLVTGGTCQCCFLRPCCGRCRAPCVPSVRSARPRPSLIASLLRPFPPPSVVATSSSSLSLSLHCGSTCRVIVKS

SEQ ID NO: 231 - protein, 54 aa, Artificial Sequence
  orf
MSSARFSGILTPHKEPNAPLKRLPFKTLKASQVFWTDKSPNLRAYLHTSAAAFE

SEQ ID NO: 232 - protein, 50 aa, Artificial Sequence
  orf
MTLRISTIIESPVMECQYCESSSWSIQSSKSANICKCGCHSFVCSLYVFS

SEQ ID NO: 233 - protein, 51 aa, Artificial Sequence
  orf
MRMPFLCLLVIRVFIRIHSPIDLTELIKPTTFFNIVPTTLQKRSKRFYEVG

SEQ ID NO: 234 - protein, 84 aa, Artificial Sequence
  orf
MCRFNCSCAFFTWSIFRIVFFEFCQSPQKIFLLLRSMFQNPKTCYSIPVLICLSRLQSLYFVARQSIRNMGFFIVFYSQFLSPL

SEQ ID NO: 235 - DNA, 1170 bp, Artificial Sequence
  orf
ataacacgttttgggattttgaaacatagagcgaagcaacaagaaaattttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactaagctctcaaaatttcatgtatgtgggcagcatttgttatgtcctttctgtgctggtattcgtgcatctaaagcaattcaaaaatatactgaacgtgttgatgaagtgcttaagaaaaatcgcaagcttaagcctgttttaatcacgtttacggtgaagaatggaatcgacttaggggaaagatcatcccacctcataaaatcgtttagaacgcttctgaagcgtcgtagggactatattaaaaaaggtcgtgggtttaatgagttctgtaagatcaatggggctgtgtattcttatgaaaacacgtataacgagcaaacaaaggaatggcatccgcacttacatatgtttgcacttttggatgattggatagaccaggaagagctttcacagtattggcattccatcactggtgattcaatgattgtagatattcgcagggtcaaaaaagaaaaaggcttaggctattcaaaggctgctgcggaagtatgtaaatatgctcttaaattcggtgatttgtctgtccaaaatacttgggaagctttcaaagtcttaaaaggtaagcgtttaagtggtgcatttggttctttatggggtgtcaaaattcctgaaaatcttgctgatgatatgcctgatgaaaaagatcttccttacttagaaatgctctataaattcgtatttggaaaacagtcttactatgacttaactatgacacgacacgtagagccacaatgcaaggacagggacaacgacgaagaggaggtagcgacgactgacggaggcggaaatggacgcagcagtgaggcgatcaaggacgggcgaggacgggcggagcgcaccgagggcacgcagggtgcacggcaccgcccgcagcacggacgtaaaaaacaacactggcaagtacctccagtaacgagggtacgagtgaaaaaacggattaaaagatgggatggttatttgtgtgttatgcacatg

SEQ ID NO: 236 - DNA, 213 bp, Artificial Sequence
  orf
atcatcatggctgttgcacatactactgttttgtctttgcttggcaaagaagtttcattttctgttttattagatgaccaaaataaaatcttttcccctggcggtattcatataaccggattagttgaaaaagttgtaattgatttaactggtgatcatgcaattttagttggtgatgagttttatcaactttccgaaatagacttacctgtt

SEQ ID NO: 237 - DNA, 351 bp, Artificial Sequence
  orf
acgattttaccaatgcccgaaaatacatatttttgggcatttttttgtccgtttcttgagaaatgcgtaagcgtgcattctgaaaaaataaacattgagattggcgaagagattcggggcgctgggcgtggggtcgggatgtcactcgatgtcccgccctgtgactggcgacacgagatattcgacaatctcactttagaaacaatatcagagggcacggagtgtgcccgaactgaagaaataaaaattaaaaactacctttccctgattacaagctcccttttgattttctccgggataaacagagccatccggatggtggcgactgactattacttttgcttttttact

SEQ ID NO: 238 - DNA, 153 bp, Artificial Sequence
  orf
ctgaaattaaccgtactggtaaatcttcttattctggtgctcttaaaccattatctattcgtttacctattcaaacttacgcaaaagtcgttgctattgaaaactttattggtgctgaaaaaactagtaaaaataaagttatcaatgatttgt

SEQ ID NO: 239 - DNA, 306 bp, Artificial Sequence
  orf
atgacaaattatcatgatgaaccaactaaagtagaaatgcttctttctgaaattaaccgtactggtaaatcttcttattctggtgctcttaaaccattatctattcgtttacctattcaaacttacgcaaaagtcgttgctattgaaaactttattggtgctgaaaaaactagtaaaaataaagttatcaatgatttgttagaaattgcttttgatcagatttatccatctttaaatgaatctcaaaaacatgcatttgatcatttttcacagtctcttttagatggtttggagtcaggaaaatta

SEQ ID NO: 240 - DNA, 171 bp, Artificial Sequence
  orf
attatgattggacctcaagttctttcaaagggaacaatggattccctggaagaagcatctgcttatttcaagcaagttttacaagaacatcttgataacaaacgtgacatcacattatttgcgattgctgttgtaggcggtcgtttgaaagtcgtagagggattaccaaaa

SEQ ID NO: 241 - DNA, 348 bp, Artificial Sequence
  orf
attatttgcgattgctgttgtaggcggtcgtttgaaagtcgtagagggattaccaaaatgattaaattagaaggcacaattattaatgtttttacccagcaaggcggtaaagacaaaaaaactggtgaagcatttgccgatcgtgacaagattcaattgttaggtgagtttgaactccctaacggtgataaaaaaaatgaattaattgatcttactgttgaaaacggtaaggaatatgaaggtatgaaaaataagcgtgtgagcattccatgtggtgctcttgcttcgggtcgaaatgtgattttttatgtcgcaaaaggcgcaacacccaaacttttaaacacagtt

SEQ ID NO: 242 - DNA, 1272 bp, Artificial Sequence
  orf
ttgactgtaaaagacaataaaaaagcccatatttctgatgctttggcgggcgacgaaatacaggctttgcaatctgctaaggcagatcaacacagggatagaataacacgttttgggattttgaaacatagagcgaagcaacaagaaaattttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactaagctctcaaaatttcatgtatgtgggcagcatttgttatgtcctttctgtgctggtattcgtgcatctaaagcaattcaaaaatatactgaacgtgttgatgaagtgcttaagaaaaatcgcaagcttaagcctgttttaatcacgtttacggtgaagaatggaatcgacttaggggaaagatcatcccacctcataaaatcgtttagaacgcttctgaagcgtcgtagggactatattaaaaaaggtcgtgggtttaatgagttctgtaagatcaatggggctgtgtattcttatgaaaacacgtataacgagcaaacaaaggaatggcatccgcacttacatatgtttgcacttttggatgattggatagaccaggaagagctttcacagtattggcattccatcactggtgattcaatgattgtagatattcgcagggtcaaaaaagaaaaaggcttaggctattcaaaggctgctgcggaagtatgtaaatatgctcttaaattcggtgatttgtctgtccaaaatacttgggaagctttcaaagtcttaaaaggtaagcgtttaagtggtgcatttggttctttatggggtgtcaaaattcctgaaaatcttgctgatgatatgcctgatgaaaaagatcttccttacttagaaatgctctataaattcgtatttggaaaacagtcttactatgacttaactatgacacgacacgtagagccacaatgcaaggacagggacaacgacgaagaggaggtagcgacgactgacggaggcggaaatggacgcagcagtgaggcgatcaaggacgggcgaggacgggcggagcgcaccgagggcacgcagggtgcacggcaccgcccgcagcacggacgtaaaaaacaacactggcaagtacctccagtaacgagggtacgagtgaaaaaacggattaaaagatgggatggttatttgtgtgttatgcacatg

SEQ ID NO: 243 - DNA, 152 bp, Artificial Sequence
  orf
tcttgtcacgatcggcaaatgcttcaccagtttttttgtctttaccgccttgctgggtaaaaacattaataattgtgccttctaatttaatcattttggtaatccctctacgactttcaaacgaccgcctacaacagcaatcgcaaataatg

SEQ ID NO: 244 - DNA, 252 bp, Artificial Sequence
  orf
atttttatttcttcagttcgggcacactccgtgccctctgatattgtttctaaagtgagattgtcgaatatctcgtgtcgccagtcacagggcgggacatcgagtgacatcccgaccccacgcccagcgccccgaatctcttcgccaatctcaatgtttattttttcagaatgcacgcttacgcatttctcaagaaacggacaaaaaaatgcccaaaaatatgtattttcgggcattggtaaaatcgttaac

SEQ ID NO: 245 - DNA, 375 bp, Artificial Sequence
  orf
atatctcgtgtcgccagtcacagggcgggacatcgagtgacatcccgaccccacgcccagcgccccgaatctcttcgccaatctcaatgtttattttttcagaatgcacgcttacgcatttctcaagaaacggacaaaaaaatgcccaaaaatatgtattttcgggcattggtaaaatcgttaactaaacaggtaagtctatttcggaaagttgataaaactcatcaccaactaaaattgcatgatcaccagttaaatcaattacaactttttcaactaatccggttatatgaataccgccaggggaaaagattttattttggtcatctaataaaacagaaaatgaaacttctttgccaagcaaagacaaaacag

SEQ ID NO: 246 - DNA, 327 bp, Artificial Sequence
  orf
ttggactgcaatccgactattcagcgactagaattttatcatgggtttatgattttacatgtgcataacacacaaataaccatcccatcttttaatccgttttttcactcgtaccctcgttactggaggtacttgccagtgttgttttttacgtccgtgctgcgggcggtgccgtgcaccctgcgtgccctcggtgcgctccgcccgtcctcgcccgtccttgatcgcctcactgctgcgtccatttccgcctccgtcagtcgtcgctacctcctcttcgtcgttgtccctgtccttgcattgtggctctacgtgtcgtgtcatagt

SEQ ID NO: 247 - DNA, 240 bp, Artificial Sequence
  orf
atccgttttttcactcgtaccctcgttactggaggtacttgccagtgttgttttttacgtccgtgctgcgggcggtgccgtgcaccctgcgtgccctcggtgcgctccgcccgtcctcgcccgtccttgatcgcctcactgctgcgtccatttccgcctccgtcagtcgtcgctacctcctcttcgtcgttgtccctgtccttgcattgtggctctacgtgtcgtgtcatagttaagtca

SEQ ID NO: 248 - DNA, 162 bp, Artificial Sequence
  orf
atatcatcagcaagattttcaggaattttgacaccccataaagaaccaaatgcaccacttaaacgcttaccttttaagactttgaaagcttcccaagtattttggacagacaaatcaccgaatttaagagcatatttacatacttccgcagcagcctttgaa

SEQ ID NO: 249 - DNA, 150 bp, Artificial Sequence
  orf
ttgaccctgcgaatatctacaatcattgaatcaccagtgatggaatgccaatactgtgaaagctcttcctggtctatccaatcatccaaaagtgcaaacatatgtaagtgcggatgccattcctttgtttgctcgttatacgtgttttca

SEQ ID NO: 250 - DNA, 153 bp, Artificial Sequence
  orf
gtgcggatgccattcctttgtttgctcgttatacgtgttttcataagaatacacagccccattgatcttacagaactcattaaacccacgaccttttttaatatagtccctacgacgcttcagaagcgttctaaacgattttatgaggtggga

SEQ ID NO: 251 - DNA, 252 bp, Artificial Sequence
  orf
ctgtgcagatttaattgctcttgtgctttcttcacttggagcatctttaggatagttttctttgaattttgccagagtccacaaaaaattttcttgttgcttcgctctatgtttcaaaatcccaaaacgtgttattctatccctgtgttgatctgccttagcagattgcaaagcctgtatttcgtcgcccgccaaagcatcagaaatatgggcttttttattgtcttttacagtcaatttttgtccccactt

SEQ ID NO: 252 - DNA, 3090 bp, Artificial Sequence
  isolate
caacacagggatagaataacacgttttgcgactttgaaacatagggctaagaatcaggaaaattatttatttaccctggctaagtttaaagagaactatgaaaaagacgttaaaaacgaagaatctatcaaggctttaaaatctgctcataaattgaatgaatgcggaaactatctgctattcaaaaatttttacacaataggtgaagttaaactctccaagctccgcacctgcggacagcatttgctctgccctttctgtgctgctattcgtgcttctcgtgctatccaaaaatacgttgaacgtattgatcaggtgttgcaagaaaatcgcaagctcaagcccgttctaatcactcttaccgttaaaaacggttctgacctagcggaacgctctgaacatcttatgaagtcctttaggacgcttttagatcgtcgtagggactatgaaaagaaagggcgaggttttaacgagttctgtaaggttcaaggggctatgtactcttacgagagtacatacaacgaaaaaacagttgaatggcatccgcatatccatatgtttgctttggttgatcagtggattgatcagcaagaattttcagaatattggcatagccttactggggactcgatggttgtcgatgttcgcagggcaagaaaagaaaaaggacacggctatagcaaagcggctgccgaagtttgtaagtatgctttaaagtttggtgatctatccgttgaaaacacttgggaagcctttaaagttctcaagggcaagcgtttaacaggttcttttggtctgttatggggtgtcaaaattcctgaaaacttagcagatgaaattccagatgatcaggacttgccatatcttgaaatgctgtacaagttcgtttacggcaaaaattcacattatgacttagctatgactcgtatggttgagccacaagcgaagattgagcgaatgagtgaggaggaggcaacgaccgaccgacacgacatgagagaaatagagcgagtggcgataaaggacgatgtgaggacggtggcaggtggggcaccactgcgctgcccgcaacgaggacgcaaaaagcaacattggcaagtatcaccttacgtccgtgttaggacacggactagaatccgtcaatgggacgggttcttatacaacatagatttattcccttatgtagaaaaacgattatatcgctatttagttgttgattaattgccatttagttgttataatggttttatcaactggagttcatgctatgactattcaatcaagattaaacaaagatactgttcagctaattaagattgctcaacgtatagaatttattagaaaacaagatgaaatgggcaaccaaaaagttatagctatgatttatatatcagaagattctaaccctattcgagcgatggctggtgatgttacttgggacgctcatgacattgataaggctaaacgctctattcgtcgtcataataaaacttgtttaattattgatagacgagatcaagttatttaatcgctggatgattgagttacgacctaaaccatccagctaaccacaacgatctatttcggagatcatcatggctgtgccacatactactgttttgtctttgcttggcaaagaagtttcattttctgtattactagatgaagaaataaaaatcttttttcctcaaggcattcacataaatggatgcgtccaagaagtaatcatagctctcaatggtaatcaccaaattttggttgatgacgaattttatgaattatcacgtattgacttattttcttaaatttacccgataggggagaccctgaatcctttattcataaaggtttcagggtttttttatcctttttacataatctgtcttataggaactctcctactaaacattacttatataatctttagttactggagacattctctccaggaacagagggcacggagtgtgcccgaactgacgcaataaaaatctaaaaacctccctctctgattacaagctcccctctgcattctcacagtaagtgccgagccatcccgaatgggtggcgactcactattgatttgatataaaatcggtgtataatttattcttaaattatgagtaaatcatttattgaaaggtacatagtatgccaaatgatagaatttcacctttagaggttgttttaggaaatattcttggtgaggaatcaccaagtagaccagaccaaaaagcagtttctttccgaattgattttgataactacgtcaatctttgcgctctgcaaagcattactccgaaaggagttagtcggaatcagcttttaaatgatcttctagctgttgctattgatcaggtttcatctgctttacctgatggtgtttctgatgcttatgaagaatctgtcgttcaccatgaacaagggttgaacgaacttttagcaattgagggccatttatgattggccctcaagttattagcaagggaaccatggattcccttgaagaagcatcagcttatttcaaacaggtcttacaagtgcacttggataacaagcgcgatatcacattgtttgcgattaccgttgttggcggtcgccttaaggtagtagagggtttaccaaaatgatcaaactagaaggcacaattatcaatgtattcacccagcagggtggcacgagcaagaaaacaggcgaagcgtttgcggatcgtgacaagattcagctaatgggagaactggaactcccaaacggttccataaaacatgaattgattgaccttaccgttgaaaatggcaaggaatatgaaaccctaaaaggcaagcgtgtcagcatcccatgtggtgctctcgcttcaggtcgtaaagtgattttttatgttgcgaaaggtggaacacctaaacttttgacagcagtatgatactcgttaaaaaacagtgagttagcgaagcggctcgctgttttttcttttacttgatactattgattaaagtggggacaaaattgcttaaaaaagacaataaaaaagcccatatttcgaacactttggcgggtggagaaatacaggctttgcaatctgctaaggcagat

SEQ ID NO: 253 - protein, 94 aa, Artificial Sequence
  orf
MVVGTMKRKGEVLTSSVRFKGLCTLTRVHTTKKQLNGIRISICLLWLISGLISKNFQNIGIALLGTRWLSMFAGQEKKKDTAIAKRLPKFVSML

SEQ ID NO: 254 - protein, 401 aa, Artificial Sequence
  orf
MTRFATLKHRAKNQENYLFTLAKFKENYEKDVKNEESIKALKSAHKLNECGNYLLFKNFYTIGEVKLSKLRTCGQHLLCPFCAAIRASRAIQKYVERIDQVLQENRKLKPVLITLTVKNGSDLAERSEHLMKSFRTLLDRRRDYEKKGRGFNEFCKVQGAMYSYESTYNEKTVEWHPHIHMFALVDQWIDQQEFSEYWHSLTGDSMVVDVRRARKEKGHGYSKAAAEVCKYALKFGDLSVENTWEAFKVLKGKRLTGSFGLLWGVKIPENLADEIPDDQDLPYLEMLYKFVYGKNSHYDLAMTRMVEPQAKIERMSEEEATTDRHDMREIERVAIKDDVRTVAGGAPLRCPQRGRKKQHWQVSPYVRVRTRTRIRQWDGFLYNIDLFPYVEKRLYRYLVVD

SEQ ID NO: 255 - protein, 105 aa, Artificial Sequence
  orf
MLINCHLVVIMVLSTGVHAMTIQSRLNKDTVQLIKIAQRIEFIRKQDEMGNQKVIAMIYISEDSNPIRAMAGDVTWDAHDIDKAKRSIRRHNKTCLIIDRRDQVI

SEQ ID NO: 256 - protein, 74 aa, Artificial Sequence
  orf
MSEIIMAVPHTTVLSLLGKEVSFSVLLDEEIKIFFPQGIHINGCVQEVIIALNGNHQILVDDEFYELSRIDLFS

SEQ ID NO: 257 - protein, 60 aa, Artificial Sequence
  orf
MYPIGETLNPLFIKVSGFFYPFYIICLIGTLLLNITYIIFSYWRHSLQEQRARSVPELTQ

SEQ ID NO: 258 - protein, 103 aa, Artificial Sequence
  orf
MPNDRISPLEVVLGNILGEESPSRPDQKAVSFRIDFDNYVNLCALQSITPKGVSRNQLLNDLLAVAIDQVSSALPDGVSDAYEESVVHHEQGLNELLAIEGHL

SEQ ID NO: 259 - protein, 56 aa, Artificial Sequence
  orf
MIGPQVISKGTMDSLEEASAYFKQVLQVHLDNKRDITLFAITVVGGRLKVVEGLPK

SEQ ID NO: 260 - protein, 97 aa, Artificial Sequence
  orf
MIKLEGTIINVFTQQGGTSKKTGEAFADRDKIQLMGELELPNGSIKHELIDLTVENGKEYETLKGKRVSIPCGALASGRKVIFYVAKGGTPKLLTAV

SEQ ID NO: 261 - protein, 60 aa, Artificial Sequence
  orf
MNYTPILYQINSESPPIRDGSALTVRMQRGACNQRGRFLDFYCVSSGTLRALCSWRECLQ

SEQ ID NO: 262 - protein, 62 aa, Artificial Sequence
  orf
MILANVAFCVLVAGSAVVPHLPPSSHRPLSPLALFLSCRVGRSLPPPHSFAQSSLVAQPYES

SEQ ID NO: 263 - protein, 61 aa, Artificial Sequence
  orf
MPMLLFASSLRAAQWCPTCHRPHIVLYRHSLYFSHVVSVGRCLLLTHSLNLRLWLNHTSHS

SEQ ID NO: 264 - protein, 58 aa, Artificial Sequence
  orf
MRPRCGQRSGAPPATVLTSSFIATRSISLMSCRSVVASSSLIRSIFACGSTIRVIAKS

SEQ ID NO: 265 - protein, 54 aa, Artificial Sequence
  orf
MSSAKFSGILTPHNRPKEPVKRLPLRTLKASQVFSTDRSPNFKAYLQTSAAALL

SEQ ID NO: 266 - protein, 54 aa, Artificial Sequence
  orf
MLINPLINQSKHMDMRMPFNCFFVVCTLVRVHSPLNLTELVKTSPFLFIVPTTI

SEQ ID NO: 267 - protein, 50 aa, Artificial Sequence
  orf
MFLQHLINTFNVFLDSTRSTNSSTERAEQMLSAGAELGEFNFTYCVKIFE

SEQ ID NO: 268 - protein, 58 aa, Artificial Sequence
  orf
MIFLILSPMFQSRKTCYSIPVLICLSRLQSLYFSTRQSVRNMGFFIVFFKQFCPHFNQ

SEQ ID NO: 269 - DNA, 282 bp, Artificial Sequence
  orf
atcgtcgtagggactatgaaaagaaagggcgaggttttaacgagttctgtaaggttcaaggggctatgtactcttacgagagtacatacaacgaaaaaacagttgaatggcatccgcatatccatatgtttgctttggttgatcagtggattgatcagcaagaattttcagaatattggcatagccttactggggactcgatggttgtcgatgttcgcagggcaagaaaagaaaaaggacacggctatagcaaagcggctgccgaagtttgtaagtatgctt

SEQ ID NO: 270 - DNA, 1203 bp, Artificial Sequence
  orf
ataacacgttttgcgactttgaaacatagggctaagaatcaggaaaattatttatttaccctggctaagtttaaagagaactatgaaaaagacgttaaaaacgaagaatctatcaaggctttaaaatctgctcataaattgaatgaatgcggaaactatctgctattcaaaaatttttacacaataggtgaagttaaactctccaagctccgcacctgcggacagcatttgctctgccctttctgtgctgctattcgtgcttctcgtgctatccaaaaatacgttgaacgtattgatcaggtgttgcaagaaaatcgcaagctcaagcccgttctaatcactcttaccgttaaaaacggttctgacctagcggaacgctctgaacatcttatgaagtcctttaggacgcttttagatcgtcgtagggactatgaaaagaaagggcgaggttttaacgagttctgtaaggttcaaggggctatgtactcttacgagagtacatacaacgaaaaaacagttgaatggcatccgcatatccatatgtttgctttggttgatcagtggattgatcagcaagaattttcagaatattggcatagccttactggggactcgatggttgtcgatgttcgcagggcaagaaaagaaaaaggacacggctatagcaaagcggctgccgaagtttgtaagtatgctttaaagtttggtgatctatccgttgaaaacacttgggaagcctttaaagttctcaagggcaagcgtttaacaggttcttttggtctgttatggggtgtcaaaattcctgaaaacttagcagatgaaattccagatgatcaggacttgccatatcttgaaatgctgtacaagttcgtttacggcaaaaattcacattatgacttagctatgactcgtatggttgagccacaagcgaagattgagcgaatgagtgaggaggaggcaacgaccgaccgacacgacatgagagaaatagagcgagtggcgataaaggacgatgtgaggacggtggcaggtggggcaccactgcgctgcccgcaacgaggacgcaaaaagcaacattggcaagtatcaccttacgtccgtgttaggacacggactagaatccgtcaatgggacgggttcttatacaacatagatttattcccttatgtagaaaaacgattatatcgctatttagttgttgat

SEQ ID NO: 271 - DNA, 315 bp, Artificial Sequence
  orf
ttgttgattaattgccatttagttgttataatggttttatcaactggagttcatgctatgactattcaatcaagattaaacaaagatactgttcagctaattaagattgctcaacgtatagaatttattagaaaacaagatgaaatgggcaaccaaaaagttatagctatgatttatatatcagaagattctaaccctattcgagcgatggctggtgatgttacttgggacgctcatgacattgataaggctaaacgctctattcgtcgtcataataaaacttgtttaattattgatagacgagatcaagttatt

SEQ ID NO: 272 - DNA, 222 bp, Artificial Sequence
  orf
atttcggagatcatcatggctgtgccacatactactgttttgtctttgcttggcaaagaagtttcattttctgtattactagatgaagaaataaaaatcttttttcctcaaggcattcacataaatggatgcgtccaagaagtaatcatagctctcaatggtaatcaccaaattttggttgatgacgaattttatgaattatcacgtattgacttattttct

SEQ ID NO: 273 - DNA, 180 bp, Artificial Sequence
  orf
atttacccgataggggagaccctgaatcctttattcataaaggtttcagggtttttttatcctttttacataatctgtcttataggaactctcctactaaacattacttatataatctttagttactggagacattctctccaggaacagagggcacggagtgtgcccgaactgacgcaa

SEQ ID NO: 274 - DNA, 309 bp, Artificial Sequence
  orf
atgccaaatgatagaatttcacctttagaggttgttttaggaaatattcttggtgaggaatcaccaagtagaccagaccaaaaagcagtttctttccgaattgattttgataactacgtcaatctttgcgctctgcaaagcattactccgaaaggagttagtcggaatcagcttttaaatgatcttctagctgttgctattgatcaggtttcatctgctttacctgatggtgtttctgatgcttatgaagaatctgtcgttcaccatgaacaagggttgaacgaacttttagcaattgagggccattta

SEQ ID NO: 275 - DNA, 168 bp, Artificial Sequence
  orf
atgattggccctcaagttattagcaagggaaccatggattcccttgaagaagcatcagcttatttcaaacaggtcttacaagtgcacttggataacaagcgcgatatcacattgtttgcgattaccgttgttggcggtcgccttaaggtagtagagggtttaccaaaa

SEQ ID NO: 276 - DNA, 291 bp, Artificial Sequence
  orf
atgatcaaactagaaggcacaattatcaatgtattcacccagcagggtggcacgagcaagaaaacaggcgaagcgtttgcggatcgtgacaagattcagctaatgggagaactggaactcccaaacggttccataaaacatgaattgattgaccttaccgttgaaaatggcaaggaatatgaaaccctaaaaggcaagcgtgtcagcatcccatgtggtgctctcgcttcaggtcgtaaagtgattttttatgttgcgaaaggtggaacacctaaacttttgacagcagta

SEQ ID NO: 277 - DNA, 1335 bp, Artificial Sequence
  orf
ttgatactattgattaaagtggggacaaaattgcttaaaaaagacaataaaaaagcccatatttcgaacactttggcgggtggagaaatacaggctttgcaatctgctaaggcagatcaacacagggatagaataacacgttttgcgactttgaaacatagggctaagaatcaggaaaattatttatttaccctggctaagtttaaagagaactatgaaaaagacgttaaaaacgaagaatctatcaaggctttaaaatctgctcataaattgaatgaatgcggaaactatctgctattcaaaaatttttacacaataggtgaagttaaactctccaagctccgcacctgcggacagcatttgctctgccctttctgtgctgctattcgtgcttctcgtgctatccaaaaatacgttgaacgtattgatcaggtgttgcaagaaaatcgcaagctcaagcccgttctaatcactcttaccgttaaaaacggttctgacctagcggaacgctctgaacatcttatgaagtcctttaggacgcttttagatcgtcgtagggactatgaaaagaaagggcgaggttttaacgagttctgtaaggttcaaggggctatgtactcttacgagagtacatacaacgaaaaaacagttgaatggcatccgcatatccatatgtttgctttggttgatcagtggattgatcagcaagaattttcagaatattggcatagccttactggggactcgatggttgtcgatgttcgcagggcaagaaaagaaaaaggacacggctatagcaaagcggctgccgaagtttgtaagtatgctttaaagtttggtgatctatccgttgaaaacacttgggaagcctttaaagttctcaagggcaagcgtttaacaggttcttttggtctgttatggggtgtcaaaattcctgaaaacttagcagatgaaattccagatgatcaggacttgccatatcttgaaatgctgtacaagttcgtttacggcaaaaattcacattatgacttagctatgactcgtatggttgagccacaagcgaagattgagcgaatgagtgaggaggaggcaacgaccgaccgacacgacatgagagaaatagagcgagtggcgataaaggacgatgtgaggacggtggcaggtggggcaccactgcgctgcccgcaacgaggacgcaaaaagcaacattggcaagtatcaccttacgtccgtgttaggacacggactagaatccgtcaatgggacgggttcttatacaacatagatttattcccttatgtagaaaaacgattatatcgctatttagttgttgat

SEQ ID NO: 278 - DNA, 180 bp, Artificial Sequence
  orf
ataaattatacaccgattttatatcaaatcaatagtgagtcgccacccattcgggatggctcggcacttactgtgagaatgcagaggggagcttgtaatcagagagggaggtttttagatttttattgcgtcagttcgggcacactccgtgccctctgttcctggagagaatgtctccag

SEQ ID NO: 279 - DNA, 186 bp, Artificial Sequence
  orf
gtgatacttgccaatgttgctttttgcgtcctcgttgcgggcagcgcagtggtgccccacctgccaccgtcctcacatcgtcctttatcgccactcgctctatttctctcatgtcgtgtcggtcggtcgttgcctcctcctcactcattcgctcaatcttcgcttgtggctcaaccatacgagtca

SEQ ID NO: 280 - DNA, 183 bp, Artificial Sequence
  orf
ttgccaatgttgctttttgcgtcctcgttgcgggcagcgcagtggtgccccacctgccaccgtcctcacatcgtcctttatcgccactcgctctatttctctcatgtcgtgtcggtcggtcgttgcctcctcctcactcattcgctcaatcttcgcttgtggctcaaccatacgagtcatagc

SEQ ID NO: 281 - DNA, 174 bp, Artificial Sequence
  orf
ttgcgtcctcgttgcgggcagcgcagtggtgccccacctgccaccgtcctcacatcgtcctttatcgccactcgctctatttctctcatgtcgtgtcggtcggtcgttgcctcctcctcactcattcgctcaatcttcgcttgtggctcaaccatacgagtcatagctaagtca

SEQ ID NO: 282 - DNA, 162 bp, Artificial Sequence
  orf
atttcatctgctaagttttcaggaattttgacaccccataacagaccaaaagaacctgttaaacgcttgcccttgagaactttaaaggcttcccaagtgttttcaacggatagatcaccaaactttaaagcatacttacaaacttcggcagccgctttgcta

SEQ ID NO: 283 - DNA, 162 bp, Artificial Sequence
  orf
ttgctgatcaatccactgatcaaccaaagcaaacatatggatatgcggatgccattcaactgttttttcgttgtatgtactctcgtaagagtacatagccccttgaaccttacagaactcgttaaaacctcgccctttcttttcatagtccctacgacgatc

SEQ ID NO: 284 - DNA, 150 bp, Artificial Sequence
  orf
attttcttgcaacacctgatcaatacgttcaacgtatttttggatagcacgagaagcacgaatagcagcacagaaagggcagagcaaatgctgtccgcaggtgcggagcttggagagtttaacttcacctattgtgtaaaaatttttgaa

SEQ ID NO: 285 - DNA, 174 bp, Artificial Sequence
  orf
ataattttcctgattcttagccctatgtttcaaagtcgcaaaacgtgttattctatccctgtgttgatctgccttagcagattgcaaagcctgtatttctccacccgccaaagtgttcgaaatatgggcttttttattgtcttttttaagcaattttgtccccactttaatcaa

SEQ ID NO: 286 - DNA, 2736 bp, Artificial Sequence
  isolate
caacacagggatagaataacacgttttggtattttgaaacacagatcgaagcaacaagaaaactttttgtggactcttgcaaaattcaaagaaaactacccaatagacacgccaaatgaagaaactgtaaaggctctaaaatctgcacagaagttgaacggctgtggcaattttttgttgttcaaaaatttttacacaatagatcaaaccaagctcgcaaaatttcatgtttgtaatcagcatttgttgtgtccattttgtgcaggtattcgtgcatctaaagcaattcaaaaatatacagaacgtgttgatgaggtcttaaagaaaaatcgcaagctcaagcctgttctaatcacgctcacagtaaaaaacggtactgacctaagtgaacggtctgcacatctcataaaatcgcttagaacgcttattaagcgtcgtcaggactataataaaaaaggtcgtggattcaacgagttttgtaaaatcaacggtgcaatgtactcctatgaaaatacttataacgaacaaaccgaagaatggcatccgcatgttcacatgtttgcccttttggatgattggatagaccaggaacaaatgtctgaaacttggcatgaaatcactggtgattcattcattgtagatattcgaaaagttaaaaaagaaaaagacttaggctatgcaaaggctgctgctgaggtatgcaaatacgctcttaaatttggtgatctaactgttgaaaatacttgggaagcattcaaggttctaaagggcaaacgcttaagcggtgcatttggatcactttatggtgtcaaaattcctgaaaatcttgctgatgatatgccagatgaaaaagaccttccgtatttagaaatgctctataaattcgtgtttagaaaacagtcttactacgatctacaaatgacacgtcatgtagagccacaggcgaatgatcgacagagggagaaggaggaacgaccgtccgacccgactggagagaatgagaaggtggtgattggggacgaggcgaggacggtggcaggtggagggctcgcaccactgcaccgcccgcagcaaggacgcaaaaaacaacactggcaagtaccaccagtaacgagggtacgagtgaaaaaacgtatcaaaagatgggatggttatttgtgtgttatacacatgtaaaatcataaacctatgttaaaattctagtcgctgaatagtcggacggcaattcaactacccagctagccaatcagatctatcgaggagatcatcatggctgttgcacatactactgttttgtctttgcttggcaaagatgtttcattttctgttttattggatgagcaaataaaatccttttttcctgaaggaataaatataactggcttattcgaagaagttattatcgctctaaatggtaatcaccaaatcctagttggtgatgagttttaccaactttcaaaaattgacctaaatctttaaataaccccgagaggggatacctcgaacccttttaatataaaggtttcggggttttttttatctattaacataatgatgactatgggaaattagctactaaagattactgctataatctttagcgagtgtctacgagtgacgctcccacaataaaaatttcaaagcctttcccctctgattacaagctcccttttgcctttctccggtaagaaccgagccatcccgaatgggtggcgactcccctattgatttgtttaagattcggtgtatgatttattgataaattacgaacgaatcattaatgattaggtaaaaatatgccaaatgatagaatttcccctctagatgttgttctaggaaatattcttggtgcagatgcatcaagtagacccgaccaaaaagcagtttcctttcgaattgattttaataattacgtgaacctttgcgccttgcagagcatcacacctaagggagttagccgaaatcaacttttgaatgatcttttagctgttgctattgatcgggttgtatcgtccttacctgatggcgtttcagacgcttatgaagaagctgttgcccataatgaagaaggtttaacaatgatcttagaaagtgagggtcatttatgattaggcccgaagttattagtagtaccccaatggaatctcttgaagaagcatgtgcttatttcaagcaagtattacaagaacatttagataaaaaacgtgacatcacattatttgcgattgctgttgtaggcggtcgtttgaaagtagtagagggattaccaaaatgattaaattagaaggcacaattattaatgtttttacccaacaaggtggtaaagataaaaaaactggtgatgcttttgaagatcgtgacaaggttcaacttatgggtgagttagaactcccaaacggtgatattaagcatgaattaatcaacctaagtgttgatgatggtaagcattacgaaacactaaaaaacaagcgtgtgagcattccttgcggtgctctagcttctggtcgaaatgtgattttttatgtcacaaaaggcggtacacctaaaattcttccaagtatctaaaatttgaaaaaaaacagtgagttagcgaagcggctcgctgtttttttatttacttgatactattcattaagtcggggacaaaatttcatgagaaatgacaataaaaaacccctgttatcagaagctttggcgggcgtagataacaagggttcgcaatctgctaaggcagat

SEQ ID NO: 287 - protein, 386 aa, Artificial Sequence
  orf
MTRFGILKHRSKQQENFLWTLAKFKENYPIDTPNEETVKALKSAQKLNGCGNFLLFKNFYTIDQTKLAKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTEEWHPHVHMFALLDDWIDQEQMSETWHEITGDSFIVDIRKVKKEKDLGYAKAAAEVCKYALKFGDLTVENTWEAFKVLKGKRLSGAFGSLYGVKIPENLADDMPDEKDLPYLEMLYKFVFRKQSYYDLQMTRHVEPQANDRQREKEERPSDPTGENEKVVIGDEARTVAGGGLAPLHRPQQGRKKQHWQVPPVTRVRVKKRIKRWDGYLCVIHM

SEQ ID NO: 288 - protein, 77 aa, Artificial Sequence
  orf
MRSIEEIIMAVAHTTVLSLLGKDVSFSVLLDEQIKSFFPEGINITGLFEEVIIALNGNHQILVGDEFYQLSKIDLNL

SEQ ID NO: 289 - protein, 103 aa, Artificial Sequence
  orf
MPNDRISPLDVVLGNILGADASSRPDQKAVSFRIDFNNYVNLCALQSITPKGVSRNQLLNDLLAVAIDRVVSSLPDGVSDAYEEAVAHNEEGLTMILESEGHL

SEQ ID NO: 290 - protein, 56 aa, Artificial Sequence
  orf
MIRPEVISSTPMESLEEACAYFKQVLQEHLDKKRDITLFAIAVVGGRLKVVEGLPK

SEQ ID NO: 291 - protein, 116 aa, Artificial Sequence
  orf
MICDCCCRRSFESSRGITKMIKLEGTIINVFTQQGGKDKKTGDAFEDRDKVQLMGELELPNGDIKHELINLSVDDGKHYETLKNKRVSIPCGALASGRNVIFYVTKGGTPKILPSI

SEQ ID NO: 292 - protein, 419 aa, Artificial Sequence
  orf
MRNDNKKPLLSEALAGVDNKGSQSAKADQHRDRITRFGILKHRSKQQENFLWTLAKFKENYPIDTPNEETVKALKSAQKLNGCGNFLLFKNFYTIDQTKLAKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTEEWHPHVHMFALLDDWIDQEQMSETWHEITGDSFIVDIRKVKKEKDLGYAKAAAEVCKYALKFGDLTVENTWEAFKVLKGKRLSGAFGSLYGVKIPENLADDMPDEKDLPYLEMLYKFVFRKQSYYDLQMTRHVEPQANDRQREKEERPSDPTGENEKVVIGDEARTVAGGGLAPLHRPQQGRKKQHWQVPPVTRVRVKKRIKRWDGYLCVIHM

SEQ ID NO: 293 - protein, 50 aa, Artificial Sequence
  orf
MSRSSKASPVFLSLPPCWVKTLIIVPSNLIILVIPLLLSNDRLQQQSQIM

SEQ ID NO: 294 - protein, 54 aa, Artificial Sequence
  orf
MITSSNKPVIFIPSGKKDFICSSNKTENETSLPSKDKTVVCATAMMISSIDLIG

SEQ ID NO: 295 - protein, 70 aa, Artificial Sequence
  orf
MIRFFTRTLVTGGTCQCCFLRPCCGRCSGASPPPATVLASSPITTFSFSPVGSDGRSSFSLCRSFACGST

SEQ ID NO: 296 - protein, 89 aa, Artificial Sequence
  orf
MILHVYNTQITIPSFDTFFHSYPRYWWYLPVLFFASLLRAVQWCEPSTCHRPRLVPNHHLLILSSRVGRSFLLLPLSIIRLWLYMTCHL

SEQ ID NO: 297 - protein, 72 aa, Artificial Sequence
  orf
MVVLASVVFCVLAAGGAVVRALHLPPSSPRPQSPPSHSLQSGRTVVPPSPSVDHSPVALHDVSFVDRSKTVF

SEQ ID NO: 298 - protein, 81 aa, Artificial Sequence
  orf
MTSDFMPSFRHLFLVYPIIQKGKHVNMRMPFFGLFVISIFIGVHCTVDFTKLVESTTFFIIVLTTLNKRSKRFYEMCRPFT

SEQ ID NO: 299 - protein, 58 aa, Artificial Sequence; identical to SEQ ID NO: 200
  orf

SEQ ID NO: 300 - protein, 56 aa, Artificial Sequence
  orf
MWRVYWVVFFEFCKSPQKVFLLLRSVFQNTKTCYSIPVLICLSRLRTLVIYARQSF

SEQ ID NO: 301 - DNA, 1158 bp, Artificial Sequence
  orf
ataacacgttttggtattttgaaacacagatcgaagcaacaagaaaactttttgtggactcttgcaaaattcaaagaaaactacccaatagacacgccaaatgaagaaactgtaaaggctctaaaatctgcacagaagttgaacggctgtggcaattttttgttgttcaaaaatttttacacaatagatcaaaccaagctcgcaaaatttcatgtttgtaatcagcatttgttgtgtccattttgtgcaggtattcgtgcatctaaagcaattcaaaaatatacagaacgtgttgatgaggtcttaaagaaaaatcgcaagctcaagcctgttctaatcacgctcacagtaaaaaacggtactgacctaagtgaacggtctgcacatctcataaaatcgcttagaacgcttattaagcgtcgtcaggactataataaaaaaggtcgtggattcaacgagttttgtaaaatcaacggtgcaatgtactcctatgaaaatacttataacgaacaaaccgaagaatggcatccgcatgttcacatgtttgcccttttggatgattggatagaccaggaacaaatgtctgaaacttggcatgaaatcactggtgattcattcattgtagatattcgaaaagttaaaaaagaaaaagacttaggctatgcaaaggctgctgctgaggtatgcaaatacgctcttaaatttggtgatctaactgttgaaaatacttgggaagcattcaaggttctaaagggcaaacgcttaagcggtgcatttggatcactttatggtgtcaaaattcctgaaaatcttgctgatgatatgccagatgaaaaagaccttccgtatttagaaatgctctataaattcgtgtttagaaaacagtcttactacgatctacaaatgacacgtcatgtagagccacaggcgaatgatcgacagagggagaaggaggaacgaccgtccgacccgactggagagaatgagaaggtggtgattggggacgaggcgaggacggtggcaggtggagggctcgcaccactgcaccgcccgcagcaaggacgcaaaaaacaacactggcaagtaccaccagtaacgagggtacgagtgaaaaaacgtatcaaaagatgggatggttatttgtgtgttatacacatg

SEQ ID NO: 302 - DNA, 231 bp, Artificial Sequence
  orf
atcagatctatcgaggagatcatcatggctgttgcacatactactgttttgtctttgcttggcaaagatgtttcattttctgttttattggatgagcaaataaaatccttttttcctgaaggaataaatataactggcttattcgaagaagttattatcgctctaaatggtaatcaccaaatcctagttggtgatgagttttaccaactttcaaaaattgacctaaatctt

SEQ ID NO: 303 - DNA, 309 bp, Artificial Sequence
  orf
atgccaaatgatagaatttcccctctagatgttgttctaggaaatattcttggtgcagatgcatcaagtagacccgaccaaaaagcagtttcctttcgaattgattttaataattacgtgaacctttgcgccttgcagagcatcacacctaagggagttagccgaaatcaacttttgaatgatcttttagctgttgctattgatcgggttgtatcgtccttacctgatggcgtttcagacgcttatgaagaagctgttgcccataatgaagaaggtttaacaatgatcttagaaagtgagggtcattta

SEQ ID NO: 304 - DNA, 168 bp, Artificial Sequence
  orf
atgattaggcccgaagttattagtagtaccccaatggaatctcttgaagaagcatgtgcttatttcaagcaagtattacaagaacatttagataaaaaacgtgacatcacattatttgcgattgctgttgtaggcggtcgtttgaaagtagtagagggattaccaaaa

SEQ ID NO: 305 - DNA, 348 bp, Artificial Sequence
  orf
attatttgcgattgctgttgtaggcggtcgtttgaaagtagtagagggattaccaaaatgattaaattagaaggcacaattattaatgtttttacccaacaaggtggtaaagataaaaaaactggtgatgcttttgaagatcgtgacaaggttcaacttatgggtgagttagaactcccaaacggtgatattaagcatgaattaatcaacctaagtgttgatgatggtaagcattacgaaacactaaaaaacaagcgtgtgagcattccttgcggtgctctagcttctggtcgaaatgtgattttttatgtcacaaaaggcggtacacctaaaattcttccaagtatc

SEQ ID NO: 306 - DNA, 1257 bp, Artificial Sequence
  orf
atgagaaatgacaataaaaaacccctgttatcagaagctttggcgggcgtagataacaagggttcgcaatctgctaaggcagatcaacacagggatagaataacacgttttggtattttgaaacacagatcgaagcaacaagaaaactttttgtggactcttgcaaaattcaaagaaaactacccaatagacacgccaaatgaagaaactgtaaaggctctaaaatctgcacagaagttgaacggctgtggcaattttttgttgttcaaaaatttttacacaatagatcaaaccaagctcgcaaaatttcatgtttgtaatcagcatttgttgtgtccattttgtgcaggtattcgtgcatctaaagcaattcaaaaatatacagaacgtgttgatgaggtcttaaagaaaaatcgcaagctcaagcctgttctaatcacgctcacagtaaaaaacggtactgacctaagtgaacggtctgcacatctcataaaatcgcttagaacgcttattaagcgtcgtcaggactataataaaaaaggtcgtggattcaacgagttttgtaaaatcaacggtgcaatgtactcctatgaaaatacttataacgaacaaaccgaagaatggcatccgcatgttcacatgtttgcccttttggatgattggatagaccaggaacaaatgtctgaaacttggcatgaaatcactggtgattcattcattgtagatattcgaaaagttaaaaaagaaaaagacttaggctatgcaaaggctgctgctgaggtatgcaaatacgctcttaaatttggtgatctaactgttgaaaatacttgggaagcattcaaggttctaaagggcaaacgcttaagcggtgcatttggatcactttatggtgtcaaaattcctgaaaatcttgctgatgatatgccagatgaaaaagaccttccgtatttagaaatgctctataaattcgtgtttagaaaacagtcttactacgatctacaaatgacacgtcatgtagagccacaggcgaatgatcgacagagggagaaggaggaacgaccgtccgacccgactggagagaatgagaaggtggtgattggggacgaggcgaggacggtggcaggtggagggctcgcaccactgcaccgcccgcagcaaggacgcaaaaaacaacactggcaagtaccaccagtaacgagggtacgagtgaaaaaacgtatcaaaagatgggatggttatttgtgtgttatacacatg

SEQ ID NO: 307 - DNA, 150 bp, Artificial Sequence
  orf
ttgtcacgatcttcaaaagcatcaccagtttttttatctttaccaccttgttgggtaaaaacattaataattgtgccttctaatttaatcattttggtaatccctctactactttcaaacgaccgcctacaacagcaatcgcaaataatg

SEQ ID NO: 308 - DNA, 162 bp, Artificial Sequence
  orf
ataataacttcttcgaataagccagttatatttattccttcaggaaaaaaggattttatttgctcatccaataaaacagaaaatgaaacatctttgccaagcaaagacaaaacagtagtatgtgcaacagccatgatgatctcctcgatagatctgattggc

SEQ ID NO: 309 - DNA, 210 bp, Artificial Sequence
  orf
ttgatacgttttttcactcgtaccctcgttactggtggtacttgccagtgttgttttttgcgtccttgctgcgggcggtgcagtggtgcgagccctccacctgccaccgtcctcgcctcgtccccaatcaccaccttctcattctctccagtcgggtcggacggtcgttcctccttctccctctgtcgatcattcgcctgtggctctaca

SEQ ID NO: 310 - DNA, 267 bp, Artificial Sequence
  orf
atgattttacatgtgtataacacacaaataaccatcccatcttttgatacgttttttcactcgtaccctcgttactggtggtacttgccagtgttgttttttgcgtccttgctgcgggcggtgcagtggtgcgagccctccacctgccaccgtcctcgcctcgtccccaatcaccaccttctcattctctccagtcgggtcggacggtcgttcctccttctccctctgtcgatcattcgcctgtggctctacatgacgtgtcatttg

SEQ ID NO: 311 - DNA, 216 bp, Artificial Sequence
  orf
ctggtggtacttgccagtgttgttttttgcgtccttgctgcgggcggtgcagtggtgcgagccctccacctgccaccgtcctcgcctcgtccccaatcaccaccttctcattctctccagtcgggtcggacggtcgttcctccttctccctctgtcgatcattcgcctgtggctctacatgacgtgtcatttgtagatcgtagtaagactgttttc

SEQ ID NO: 312 - DNA, 243 bp, Artificial Sequence
  orf
atcaccagtgatttcatgccaagtttcagacatttgttcctggtctatccaatcatccaaaagggcaaacatgtgaacatgcggatgccattcttcggtttgttcgttataagtattttcataggagtacattgcaccgttgattttacaaaactcgttgaatccacgaccttttttattatagtcctgacgacgcttaataagcgttctaagcgattttatgagatgtgcagaccgttcact

SEQ ID NO: 313 - DNA, 174 bp, Artificial Sequence; identical to SEQ ID NO: 215
  orf

SEQ ID NO: 314 - DNA, 168 bp, Artificial Sequence
  orf
atttggcgtgtctattgggtagttttctttgaattttgcaagagtccacaaaaagttttcttgttgcttcgatctgtgtttcaaaataccaaaacgtgttattctatccctgtgttgatctgccttagcagattgcgaacccttgttatctacgcccgccaaagcttc

SEQ ID NO: 315 - DNA, 2405 bp, Artificial Sequence
  isolate
aaggcagatcaacacagggatagaatatcacgttttggacttttgaaacatagagcgaaactacaagaacaatatttgtggacgcaagttgatttcaaatctgaaggcgaagacgatctatcctataaggctcttaaggctgcaacaaaattaaaaggttgtggtcagtttcttatattcagaaattactacaccatcgaccaagttaagctcgaaaaattccacgtatgcggacagcatttgctatgccccatgtgtgcaggtattcgcgctgcacgttcaatgaaacgctatttagaccgtatccatgaattaatgcgtcagaatccgcgtttaaagcccgtcctgatcactttaacggtcaagaatggggaagacctagaagaacgctataatcaccttacaagctcatttagaacgcttctatcgcgttatagggattacaagaaaaagggttgggggtttaatcaattctgtaagattgatggcgctttctataccactgaatacacttacaacgataaaactaagcaatggcatccgcatattcatattttcgctttactcaatgaatggatagaccaagaagaacttgcagaaacttggcacgatatcacccttgattcttatattgtcgatattcgtagagttaaaaaaaccaaagaacacggctatagcaaagctgttgctgaggtatgtaaatacgctcttaagtttggcgatctatcagttgaaaacacttgggacgcatttttaacacttaagggtaaacgccttacaggttctttcggttctatgcacggtgtcaaaattcctgaaaaattcactgatgatatgccactggaggagctgccttacttagagatgttctaccgttttgtatttggtccgaaatcatattacaacttagaaataacaaaagacgttaagccgaacaaaaggaatgaggagtgaggaggaggcaccgaccgaccgacacgaccgaatgacttgtgaaggcggtacgtcaatcggttgtctgtgatgtgacgttccgtgaacgtgcatcatcatcagacaatcgaataagcaagcctacctgtatacacaggacttgtagggggttcgaccccttagaccccaaagggcgcactttaccaaacctttcaggttcggcagtgctccccataataggattatagatatgaacgaattactagaattgaaacaccgtattgataactgtactgatgatctccaatctttcgaacttcgaaagatttatgacaagctcattattgatctatggcaatctgaaaaaattactttgtcagaaatgcagtctatggctctccctctccagtagcagagggcgcggagtgcgcccgaactgacatcatctaaatttcaaaaccttccttccctgattacaagctccccttggatttccaagcgggagcttttagcaatcccgctgggttgcgtcacattgaaacattgaaacattgaaacattgaaacattgaaacattgaaacattgaaacattgaaacattgaaacattgaaacattgaaacattgtttcaatgacacatttatttgcatatttttcaataatgatttataagtggttcacagatgaattttttataaaactatccatgaataggggctgatatgccaaatccacttgatgtagtccttggaaatatccttggttcagaacaaacccgacctgatcaaaaggctgtttcctttcgtattgattttaaaaactatgtgaagctgtctgctttacaaagcattacacctaaaggtgttagccgtaatcagcttttaaatgatcttttagctgttgctcttgatcaagttgaatcgtccttgcctgatggtgcttcggatgcttatgaacatgcacttgtagaaaatgaagaaggtttaactatgcttttagaaacggaaggtcatttatgattaagttagaaggaattgttttaaacgtatttacccagcaaggcggtaaagataaaaaaacaggtgatcaatttgctgaccgtgacaaggtgcaaattttaggtgctatggatctgcctaatggtgacgtaaaaaacgaactatttacgttatctgtagaaaactatcgggactttaaagacttcctaaataagaaaatctgtattgctgttggtgcaatggcaagcggtcgaaatgttattttttacgttgctaaaggcgcacagcctgttttagcagaaacaatgtgaaacatgaaaaaaaacagtgagttagccttggcttctcgctgtttttttctttctcttgatactattaactagagtggggatgaaattgactaaaaaagacaataaaaaagcccataattctgacactttggcgggtgatgaattacaggctttgcaatctgct

SEQ ID NO: 316 - protein, 62 aa, Artificial Sequence
  orf
MITLQAHLERFYRVIGITRKRVGGLINSVRLMALSIPLNTLTTIKLSNGIRIFIFSLYSMNG

SEQ ID NO: 317 - protein, 54 aa, Artificial Sequence
  orf
MQKLGTISPLILILSIFVELKKPKNTAIAKLLLRYVNTLLSLAIYQLKTLGTHF

SEQ ID NO: 318 - protein, 309 aa, Artificial Sequence
  orf
MSRFGLLKHRAKLQEQYLWTQVDFKSEGEDDLSYKALKAATKLKGCGQFLIFRNYYTIDQVKLEKFHVCGQHLLCPMCAGIRAARSMKRYLDRIHELMRQNPRLKPVLITLTVKNGEDLEERYNHLTSSFRTLLSRYRDYKKKGWGFNQFCKIDGAFYTTEYTYNDKTKQWHPHIHIFALLNEWIDQEELAETWHDITLDSYIVDIRRVKKTKEHGYSKAVAEVCKYALKFGDLSVENTWDAFLTLKGKRLTGSFGSMHGVKIPEKFTDDMPLEELPYLEMFYRFVFGPKSYYNLEITKDVKPNKRNEE

SEQ ID NO: 319 - protein, 62 aa, Artificial Sequence
  orf
MKAVRQSVVCDVTFRERASSSDNRISKPTCIHRTCRGFDPLDPKGRTLPNLSGSAVLPIIGL

SEQ ID NO: 320 - protein, 56 aa, Artificial Sequence
  orf
MIDMNELLELKHRIDNCTDDLQSFELRKIYDKLIIDLWQSEKITLSEMQSMALPLQ

SEQ ID NO: 321 - protein, 52 aa, Artificial Sequence
  orf
MITVLMISNLSNFERFMTSSLLIYGNLKKLLCQKCSLWLSLSSSRGRGVRPN

SEQ ID NO: 322 - protein, 102 aa, Artificial Sequence
  orf
MGADMPNPLDVVLGNILGSEQTRPDQKAVSFRIDFKNYVKLSALQSITPKGVSRNQLLNDLLAVALDQVESSLPDGASDAYEHALVENEEGLTMLLETEGHL

SEQ ID NO: 323 - protein, 102 aa, Artificial Sequence; identical to SEQ ID NO: 322
  orf

SEQ ID NO: 324 - protein, 97 aa, Artificial Sequence
  orf
MIKLEGIVLNVFTQQGGKDKKTGDQFADRDKVQILGAMDLPNGDVKNELFTLSVENYRDFKDFLNKKICIAVGAMASGRNVIFYVAKGAQPVLAETM

SEQ ID NO: 325 - protein, 353 aa, Artificial Sequence
  orf
MILLTRVGMKLTKKDNKKAHNSDTLAGDELQALQSAKADQHRDRISRFGLLKHRAKLQEQYLWTQVDFKSEGEDDLSYKALKAATKLKGCGQFLIFRNYYTIDQVKLEKFHVCGQHLLCPMCAGIRAARSMKRYLDRIHELMRQNPRLKPVLITLTVKNGEDLEERYNHLTSSFRTLLSRYRDYKKKGWGFNQFCKIDGAFYTTEYTYNDKTKQWHPHIHIFALLNEWIDQEELAETWHDITLDSYIVDIRRVKKTKEHGYSKAVAEVCKYALKFGDLSVENTWDAFLTLKGKRLTGSFGSMHGVKIPEKFTDDMPLEELPYLEMFYRFVFGPKSYYNLEITKDVKPNKRNEE

SEQ ID NO: 326 - protein, 70 aa, Artificial Sequence
  orf
MIIEKYANKCVIETMFQCFNVSMFQCFNVSMFQCFNVSMFQCFNVTQPSGIAKSSRLEIQGELVIREGRF

SEQ ID NO: 327 - protein, 61 aa, Artificial Sequence
  orf
MFRSSKDWRSSVQLSIRCFNSSNSFISIILLWGALPNLKGLVKCALWGLRGRTPYKSCVYR

SEQ ID NO: 328 - protein, 58 aa, Artificial Sequence
  orf
MYNPIMGSTAEPERFGKVRPLGSKGSNPLQVLCIQVGLLIRLSDDDARSRNVTSQTTD

SEQ ID NO: 329 - protein, 54 aa, Artificial Sequence
  orf
MSSVNFSGILTPCIEPKEPVRRLPLSVKNASQVFSTDRSPNLRAYLHTSATALL

SEQ ID NO: 330 - protein, 53 aa, Artificial Sequence
  orf
MAKLKSVFTYLSNSFAIAVFFGFFNSTNIDNIRIKGDIVPSFCKFFLVYPFIE

SEQ ID NO: 331 - DNA, 186 bp, Artificial Sequence
  orf
ataatcaccttacaagctcatttagaacgcttctatcgcgttatagggattacaagaaaaagggttgggggtttaatcaattctgtaagattgatggcgctttctataccactgaatacacttacaacgataaaactaagcaatggcatccgcatattcatattttcgctttactcaatgaatgga

SEQ ID NO: 332 - DNA, 162 bp, Artificial Sequence
  orf
ttgcagaaacttggcacgatatcacccttgattcttatattgtcgatattcgtagagttaaaaaaaccaaagaacacggctatagcaaagctgttgctgaggtatgtaaatacgctcttaagtttggcgatctatcagttgaaaacacttgggacgcatttt

SEQ ID NO: 333 - DNA, 927 bp, Artificial Sequence
  orf
atatcacgttttggacttttgaaacatagagcgaaactacaagaacaatatttgtggacgcaagttgatttcaaatctgaaggcgaagacgatctatcctataaggctcttaaggctgcaacaaaattaaaaggttgtggtcagtttcttatattcagaaattactacaccatcgaccaagttaagctcgaaaaattccacgtatgcggacagcatttgctatgccccatgtgtgcaggtattcgcgctgcacgttcaatgaaacgctatttagaccgtatccatgaattaatgcgtcagaatccgcgtttaaagcccgtcctgatcactttaacggtcaagaatggggaagacctagaagaacgctataatcaccttacaagctcatttagaacgcttctatcgcgttatagggattacaagaaaaagggttgggggtttaatcaattctgtaagattgatggcgctttctataccactgaatacacttacaacgataaaactaagcaatggcatccgcatattcatattttcgctttactcaatgaatggatagaccaagaagaacttgcagaaacttggcacgatatcacccttgattcttatattgtcgatattcgtagagttaaaaaaaccaaagaacacggctatagcaaagctgttgctgaggtatgtaaatacgctcttaagtttggcgatctatcagttgaaaacacttgggacgcatttttaacacttaagggtaaacgccttacaggttctttcggttctatgcacggtgtcaaaattcctgaaaaattcactgatgatatgccactggaggagctgccttacttagagatgttctaccgttttgtatttggtccgaaatcatattacaacttagaaataacaaaagacgttaagccgaacaaaaggaatgaggag

SEQ ID NO: 334 - DNA, 168 bp, Artificial Sequence
  orf
attatagatatgaacgaattactagaattgaaacaccgtattgataactgtactgatgatctccaatctttcgaacttcgaaagatttatgacaagctcattattgatctatggcaatctgaaaaaattactttgtcagaaatgcagtctatggctctccctctccag

SEQ ID NO: 335 - DNA, 168 bp, Artificial Sequence; identical to SEQ ID NO: 334
  orf

SEQ ID NO: 336 - DNA, 156 bp, Artificial Sequence
  orf
ttgataactgtactgatgatctccaatctttcgaacttcgaaagatttatgacaagctcattattgatctatggcaatctgaaaaaattactttgtcagaaatgcagtctatggctctccctctccagtagcagagggcgcggagtgcgcccgaac

SEQ ID NO: 337 - DNA, 306 bp, Artificial Sequence
  orf
ataggggctgatatgccaaatccacttgatgtagtccttggaaatatccttggttcagaacaaacccgacctgatcaaaaggctgtttcctttcgtattgattttaaaaactatgtgaagctgtctgctttacaaagcattacacctaaaggtgttagccgtaatcagcttttaaatgatcttttagctgttgctcttgatcaagttgaatcgtccttgcctgatggtgcttcggatgcttatgaacatgcacttgtagaaaatgaagaaggtttaactatgcttttagaaacggaaggtcattta

SEQ ID NO: 338 - DNA, 291 bp, Artificial Sequence
  orf
atgattaagttagaaggaattgttttaaacgtatttacccagcaaggcggtaaagataaaaaaacaggtgatcaatttgctgaccgtgacaaggtgcaaattttaggtgctatggatctgcctaatggtgacgtaaaaaacgaactatttacgttatctgtagaaaactatcgggactttaaagacttcctaaataagaaaatctgtattgctgttggtgcaatggcaagcggtcgaaatgttattttttacgttgctaaaggcgcacagcctgttttagcagaaacaatg

SEQ ID NO: 339 - DNA, 1059 bp, Artificial Sequence
  orf
ttgatactattaactagagtggggatgaaattgactaaaaaagacaataaaaaagcccataattctgacactttggcgggtgatgaattacaggctttgcaatctgctaaggcagatcaacacagggatagaatatcacgttttggacttttgaaacatagagcgaaactacaagaacaatatttgtggacgcaagttgatttcaaatctgaaggcgaagacgatctatcctataaggctcttaaggctgcaacaaaattaaaaggttgtggtcagtttcttatattcagaaattactacaccatcgaccaagttaagctcgaaaaattccacgtatgcggacagcatttgctatgccccatgtgtgcaggtattcgcgctgcacgttcaatgaaacgctatttagaccgtatccatgaattaatgcgtcagaatccgcgtttaaagcccgtcctgatcactttaacggtcaagaatggggaagacctagaagaacgctataatcaccttacaagctcatttagaacgcttctatcgcgttatagggattacaagaaaaagggttgggggtttaatcaattctgtaagattgatggcgctttctataccactgaatacacttacaacgataaaactaagcaatggcatccgcatattcatattttcgctttactcaatgaatggatagaccaagaagaacttgcagaaacttggcacgatatcacccttgattcttatattgtcgatattcgtagagttaaaaaaaccaaagaacacggctatagcaaagctgttgctgaggtatgtaaatacgctcttaagtttggcgatctatcagttgaaaacacttgggacgcatttttaacacttaagggtaaacgccttacaggttctttcggttctatgcacggtgtcaaaattcctgaaaaattcactgatgatatgccactggaggagctgccttacttagagatgttctaccgttttgtatttggtccgaaatcatattacaacttagaaataacaaaagacgttaagccgaacaaaaggaatgaggag

SEQ ID NO: 340 - DNA, 210 bp, Artificial Sequence
  orf
atcattattgaaaaatatgcaaataaatgtgtcattgaaacaatgtttcaatgtttcaatgtttcaatgtttcaatgtttcaatgtttcaatgtttcaatgtttcaatgtttcaatgtttcaatgtttcaatgtgacgcaacccagcgggattgctaaaagctcccgcttggaaatccaaggggagcttgtaatcagggaaggaaggttt

SEQ ID NO: 341 - DNA, 183 bp, Artificial Sequence
  orf
atctttcgaagttcgaaagattggagatcatcagtacagttatcaatacggtgtttcaattctagtaattcgttcatatctataatcctattatggggagcactgccgaacctgaaaggtttggtaaagtgcgccctttggggtctaaggggtcgaaccccctacaagtcctgtgtatacagg

SEQ ID NO: 342 - DNA, 174 bp, Artificial Sequence
  orf
atctataatcctattatggggagcactgccgaacctgaaaggtttggtaaagtgcgccctttggggtctaaggggtcgaaccccctacaagtcctgtgtatacaggtaggcttgcttattcgattgtctgatgatgatgcacgttcacggaacgtcacatcacagacaaccgat

SEQ ID NO: 343 - DNA, 162 bp, Artificial Sequence
  orf
atatcatcagtgaatttttcaggaattttgacaccgtgcatagaaccgaaagaacctgtaaggcgtttacccttaagtgttaaaaatgcgtcccaagtgttttcaactgatagatcgccaaacttaagagcgtatttacatacctcagcaacagctttgcta

SEQ ID NO: 344 - DNA, 159 bp, Artificial Sequence
  orf
atcgccaaacttaagagcgtatttacatacctcagcaacagctttgctatagccgtgttctttggtttttttaactctacgaatatcgacaatataagaatcaagggtgatatcgtgccaagtttctgcaagttcttcttggtctatccattcattgag

SEQ ID NO: 345 - DNA, 2365 bp, Artificial Sequence
  isolate
caacacagggatagaataacacgttttgcgactttgaaacatagggctaagaatcaggaaaattatttatttaccctggctaagtttaaagagaactatgagaaagacgttaaaaacgaagaatctatcaaggctctaaaatctgctcagaaattgaatgaatgcggaaactatctgctattcaaaaatttttacacaataggcgaagttaaactctccaagctccgcacctgcggacagcatttgctttgccctttctgtgctgccattcgtgcttctcgtgctattcaaaaatacgttgaacgtattgatcaagtcctgcaagaaaatcgcaagctcaagcccgttctaatcacgctcaccgttaaaaacggttctaacctagcggaacgctctgaacaccttatgaagtcctttaggacgcttctagagcgtcgtagggactatgaaaagaaaggtcgaggttttaatgagttctgtaaggttcaaggggctatgtactcctatgaaaatacattcaatgaaaaaacaggcgaatggcatccgcatattcatatgttcgctttggttggtcaatggattgatcagcaagagttttcggaatattggcatagccttactggggactcgatggttgtcgatgtccgcagggcaagaaaagaaaaaggtcacggttatagcaaagcggctgccgaagtctgtaagtatgctttgaagtttggtgatctgtccgttgaaaagacttgggaagcatttaaggttcttaagggaaagcgtttaacaggttcttttggtctgctatggggtgtcaaaatccctgacacaatgacagacgatatgccatcagaagacttgccatatctcgaaatgctgtacaagtttgcctacagtaaaaagtcttactacgacttactaatcacaaggcacgtagagccaaaagaaactgaccgaatgaggagtgaggaggagggaccgaccgaccgacacgaccgaatgagggatgttgatttggcgatgacggacgaagcgaggacgttgccggtgcgagggcacgcagtcacggaacgaccacagcgaggacgtaaaaaagcccactggagaatcccgcctaaaacccgtgtaagggttcgggaacgaatccgaagatgggacggaattatgtataacattgatcttttcccatatgttgaacgtaggctattggctttcatcgggtaacttacgaaccttgagtgaaacgaaaggtgtagtaagtacaccttctccagtagccgagcgagtgtctacgagcgacgactccgaaattagaatttcaaaaccttcctttccctgattacaagctcccatctgattttagatcgggagcttttagccatcccgaatgggtggcgacgcattgacattctcataagaatctataatattctaataacaatctaatagaatctaaatggaatctaaaaatgttattcaaatctcttggtattgctaccgctacaaatacagcttctaattctgaggactctaggcctgtgactattcgtttaactgatagtgaagttcaactttacgatgctgttgctaagtctatgggcttaactcgacaagacttcttagctcacttaatccgcaataacttcaagcaggctttaactgatttcttaaccggttatacccaatcatctccgtcagtttctttgtgtgaattaattaattcaaatactgacagtgacgaagttaaacagaaggtttcttggttactacgttctatctcacaagaattaatggaagaagacgagaaagaaattcacgaacacattgagaacgagcgtttctatcaattcgctactcctcgtaaaggcatttttgctgaaacgcctaagccagaggagaaataacaatgattaagctggaaggaacggtattgaatgtctttactcagcaaggcggtaaagacaaaaaaacaggtgatcaattttctgatcgtgacaaggtacaaattttaggtgctatggatctgccaaatggtgacgtaaaaaatgaactttttacgttatcggtagagaattatcgggactttaaagactacctaaatcagaaaatttcaattgctgttggtgcaatggcaagtggtcgtaatgtgattttttatgttgctaaaggtgcaaaacctgttttagcggaaacaatgtgaaacgtgaaaaaaaacagtgagttagcgaagctactcgctgttttttcttttacttgatactattgattaaagtggggacaaaaattgcttaaaaaagacaataaaaaagcccatatttcggacactttggctggtgatgaaatacaggctttgcaatctgctaaggcagat

SEQ ID NO: 346 - protein, 89 aa, Artificial Sequence
  orf
MKRKVEVLMSSVRFKGLCTPMKIHSMKKQANGIRIFICSLWLVNGLISKSFRNIGIALLGTRWLSMSAGQEKKKVTVIAKRLPKSVSML

SEQ ID NO: 347 - protein, 400 aa, Artificial Sequence
  orf
MTRFATLKHRAKNQENYLFTLAKFKENYEKDVKNEESIKALKSAQKLNECGNYLLFKNFYTIGEVKLSKLRTCGQHLLCPFCAAIRASRAIQKYVERIDQVLQENRKLKPVLITLTVKNGSNLAERSEHLMKSFRTLLERRRDYEKKGRGFNEFCKVQGAMYSYENTFNEKTGEWHPHIHMFALVGQWIDQQEFSEYWHSLTGDSMVVDVRRARKEKGHGYSKAAAEVCKYALKFGDLSVEKTWEAFKVLKGKRLTGSFGLLWGVKIPDTMTDDMPSEDLPYLEMLYKFAYSKKSYYDLLITRHVEPKETDRMRSEEEGPTDRHDRMRDVDLAMTDEARTLPVRGHAVTERPQRGRKKAHWRIPPKTRVRVRERIRRWDGIMYNIDLFPYVERRLLAFIG

SEQ ID NO: 348 - protein, 60 aa, Artificial Sequence
  orf
MELCITLIFSHMLNVGYWLSSGNLRTLSETKGVVSTPSPVAERVSTSDDSEIRISKPSFP

SEQ ID NO: 349 - protein, 152 aa, Artificial Sequence
  orf
MESKWNLKMLFKSLGIATATNTASNSEDSRPVTIRLTDSEVQLYDAVAKSMGLTRQDFLAHLIRNNFKQALTDFLTGYTQSSPSVSLCELINSNTDSDEVKQKVSWLLRSISQELMEEDEKEIHEHIENERFYQFATPRKGIFAETPKPEEK

SEQ ID NO: 350 - protein, 99 aa, Artificial Sequence
  orf
MTMIKLEGTVLNVFTQQGGKDKKTGDQFSDRDKVQILGAMDLPNGDVKNELFTLSVENYRDFKDYLNQKISIAVGAMASGRNVIFYVAKGAKPVLAETM

SEQ ID NO: 351 - protein, 434 aa, Artificial Sequence
  orf
MLKKDNKKAHISDTLAGDEIQALQSAKADQHRDRITRFATLKHRAKNQENYLFTLAKFKENYEKDVKNEESIKALKSAQKLNECGNYLLFKNFYTIGEVKLSKLRTCGQHLLCPFCAAIRASRAIQKYVERIDQVLQENRKLKPVLITLTVKNGSNLAERSEHLMKSFRTLLERRRDYEKKGRGFNEFCKVQGAMYSYENTFNEKTGEWHPHIHMFALVGQWIDQQEFSEYWHSLTGDSMVVDVRRARKEKGHGYSKAAAEVCKYALKFGDLSVEKTWEAFKVLKGKRLTGSFGLLWGVKIPDTMTDDMPSEDLPYLEMLYKFAYSKKSYYDLLITRHVEPKETDRMRSEEEGPTDRHDRMRDVDLAMTDEARTLPVRGHAVTERPQRGRKKAHWRIPPKTRVRVRERIRRWDGIMYNIDLFPYVERRLLAFIG

SEQ ID NO: 352 - protein, 53 aa, Artificial Sequence
  orf
MYLVTIRKLITCFFVFTALLSKDIQYRSFQLNHCYFSSGLGVSAKMPLRGVAN

SEQ ID NO: 353 - protein, 93 aa, Artificial Sequence
  orf
MFNIWEKINVIHNSVPSSDSFPNPYTGFRRDSPVGFFTSSLWSFRDCVPSHRQRPRFVRHRQINIPHSVVSVGRSLLLTPHSVSFFWLYVPCD

SEQ ID NO: 354 - protein, 121 aa, Artificial Sequence
  orf
MEKVYLLHLSFHSRFVSYPMKANSLRSTYGKRSMLYIIPSHLRIRSRTLTRVLGGILQWAFLRPRCGRSVTACPRTGNVLASSVIAKSTSLIRSCRSVGPSSSLLIRSVSFGSTCLVISKS

SEQ ID NO: 355 - protein, 67 aa, Artificial Sequence
  orf
MYSISRYGKSSDGISSVIVSGILTPHSRPKEPVKRFPLRTLNASQVFSTDRSPNFKAYLQTSAAALL

SEQ ID NO: 356 - protein, 116 aa, Artificial Sequence
  orf
MLSLKNLKCFPSLFNGQITKLQSILTDFGSRFAITVTFFFSCPADIDNHRVPSKAMPIFRKLLLINPLTNQSEHMNMRMPFACFFIECIFIGVHSPLNLTELIKTSTFLFIVPTTL

SEQ ID NO: 357 - protein, 50 aa, Artificial Sequence
  orf
MFLQDLINTFNVFLNSTRSTNGSTERAKQMLSAGAELGEFNFAYCVKIFE

SEQ ID NO: 358 - protein, 56 aa, Artificial Sequence
  orf
MIFLILSPMFQSRKTCYSIPVLICLSRLQSLYFITSQSVRNMGFFIVFFKQFLSPL

SEQ ID NO: 359 - DNA, 267 bp, Artificial Sequence
  orf
atgaaaagaaaggtcgaggttttaatgagttctgtaaggttcaaggggctatgtactcctatgaaaatacattcaatgaaaaaacaggcgaatggcatccgcatattcatatgttcgctttggttggtcaatggattgatcagcaagagttttcggaatattggcatagccttactggggactcgatggttgtcgatgtccgcagggcaagaaaagaaaaaggtcacggttatagcaaagcggctgccgaagtctgtaagtatgctt

SEQ ID NO: 360 - DNA, 1200 bp, Artificial Sequence
  orf
ataacacgttttgcgactttgaaacatagggctaagaatcaggaaaattatttatttaccctggctaagtttaaagagaactatgagaaagacgttaaaaacgaagaatctatcaaggctctaaaatctgctcagaaattgaatgaatgcggaaactatctgctattcaaaaatttttacacaataggcgaagttaaactctccaagctccgcacctgcggacagcatttgctttgccctttctgtgctgccattcgtgcttctcgtgctattcaaaaatacgttgaacgtattgatcaagtcctgcaagaaaatcgcaagctcaagcccgttctaatcacgctcaccgttaaaaacggttctaacctagcggaacgctctgaacaccttatgaagtcctttaggacgcttctagagcgtcgtagggactatgaaaagaaaggtcgaggttttaatgagttctgtaaggttcaaggggctatgtactcctatgaaaatacattcaatgaaaaaacaggcgaatggcatccgcatattcatatgttcgctttggttggtcaatggattgatcagcaagagttttcggaatattggcatagccttactggggactcgatggttgtcgatgtccgcagggcaagaaaagaaaaaggtcacggttatagcaaagcggctgccgaagtctgtaagtatgctttgaagtttggtgatctgtccgttgaaaagacttgggaagcatttaaggttcttaagggaaagcgtttaacaggttcttttggtctgctatggggtgtcaaaatccctgacacaatgacagacgatatgccatcagaagacttgccatatctcgaaatgctgtacaagtttgcctacagtaaaaagtcttactacgacttactaatcacaaggcacgtagagccaaaagaaactgaccgaatgaggagtgaggaggagggaccgaccgaccgacacgaccgaatgagggatgttgatttggcgatgacggacgaagcgaggacgttgccggtgcgagggcacgcagtcacggaacgaccacagcgaggacgtaaaaaagcccactggagaatcccgcctaaaacccgtgtaagggttcgggaacgaatccgaagatgggacggaattatgtataacattgatcttttcccatatgttgaacgtaggctattggctttcatcggg

SEQ ID NO: 361 - DNA, 180 bp, Artificial Sequence
  orf
acggaattatgtataacattgatcttttcccatatgttgaacgtaggctattggctttcatcgggtaacttacgaaccttgagtgaaacgaaaggtgtagtaagtacaccttctccagtagccgagcgagtgtctacgagcgacgactccgaaattagaatttcaaaaccttcctttccc

SEQ ID NO: 362 - DNA, 456 bp, Artificial Sequence
  orf
atagaatctaaatggaatctaaaaatgttattcaaatctcttggtattgctaccgctacaaatacagcttctaattctgaggactctaggcctgtgactattcgtttaactgatagtgaagttcaactttacgatgctgttgctaagtctatgggcttaactcgacaagacttcttagctcacttaatccgcaataacttcaagcaggctttaactgatttcttaaccggttatacccaatcatctccgtcagtttctttgtgtgaattaattaattcaaatactgacagtgacgaagttaaacagaaggtttcttggttactacgttctatctcacaagaattaatggaagaagacgagaaagaaattcacgaacacattgagaacgagcgtttctatcaattcgctactcctcgtaaaggcatttttgctgaaacgcctaagccagaggagaaa

SEQ ID NO: 363 - DNA, 297 bp, Artificial Sequence
  orf
ataacaatgattaagctggaaggaacggtattgaatgtctttactcagcaaggcggtaaagacaaaaaaacaggtgatcaattttctgatcgtgacaaggtacaaattttaggtgctatggatctgccaaatggtgacgtaaaaaatgaactttttacgttatcggtagagaattatcgggactttaaagactacctaaatcagaaaatttcaattgctgttggtgcaatggcaagtggtcgtaatgtgattttttatgttgctaaaggtgcaaaacctgttttagcggaaacaatg

SEQ ID NO: 364 - DNA, 1302 bp, Artificial Sequence
  orf
ttgcttaaaaaagacaataaaaaagcccatatttcggacactttggctggtgatgaaatacaggctttgcaatctgctaaggcagatcaacacagggatagaataacacgttttgcgactttgaaacatagggctaagaatcaggaaaattatttatttaccctggctaagtttaaagagaactatgagaaagacgttaaaaacgaagaatctatcaaggctctaaaatctgctcagaaattgaatgaatgcggaaactatctgctattcaaaaatttttacacaataggcgaagttaaactctccaagctccgcacctgcggacagcatttgctttgccctttctgtgctgccattcgtgcttctcgtgctattcaaaaatacgttgaacgtattgatcaagtcctgcaagaaaatcgcaagctcaagcccgttctaatcacgctcaccgttaaaaacggttctaacctagcggaacgctctgaacaccttatgaagtcctttaggacgcttctagagcgtcgtagggactatgaaaagaaaggtcgaggttttaatgagttctgtaaggttcaaggggctatgtactcctatgaaaatacattcaatgaaaaaacaggcgaatggcatccgcatattcatatgttcgctttggttggtcaatggattgatcagcaagagttttcggaatattggcatagccttactggggactcgatggttgtcgatgtccgcagggcaagaaaagaaaaaggtcacggttatagcaaagcggctgccgaagtctgtaagtatgctttgaagtttggtgatctgtccgttgaaaagacttgggaagcatttaaggttcttaagggaaagcgtttaacaggttcttttggtctgctatggggtgtcaaaatccctgacacaatgacagacgatatgccatcagaagacttgccatatctcgaaatgctgtacaagtttgcctacagtaaaaagtcttactacgacttactaatcacaaggcacgtagagccaaaagaaactgaccgaatgaggagtgaggaggagggaccgaccgaccgacacgaccgaatgagggatgttgatttggcgatgacggacgaagcgaggacgttgccggtgcgagggcacgcagtcacggaacgaccacagcgaggacgtaaaaaagcccactggagaatcccgcctaaaacccgtgtaagggttcgggaacgaatccgaagatgggacggaattatgtataacattgatcttttcccatatgttgaacgtaggctattggctttcatcggg

SEQ ID NO: 365 - DNA, 159 bp, Artificial Sequence
  orf
ttgtaccttgtcacgatcagaaaattgatcacctgtttttttgtctttaccgccttgctgagtaaagacattcaataccgttccttccagcttaatcattgttatttctcctctggcttaggcgtttcagcaaaaatgcctttacgaggagtagcgaat

SEQ ID NO: 366 - DNA, 279 bp, Artificial Sequence
  orf
acgttcaacatatgggaaaagatcaatgttatacataattccgtcccatcttcggattcgttcccgaacccttacacgggttttaggcgggattctccagtgggcttttttacgtcctcgctgtggtcgttccgtgactgcgtgccctcgcaccggcaacgtcctcgcttcgtccgtcatcgccaaatcaacatccctcattcggtcgtgtcggtcggtcggtccctcctcctcactcctcattcggtcagtttcttttggctctacgtgccttgtgat

SEQ ID NO: 367 - DNA, 363 bp, Artificial Sequence
  orf
ctggagaaggtgtacttactacacctttcgtttcactcaaggttcgtaagttacccgatgaaagccaatagcctacgttcaacatatgggaaaagatcaatgttatacataattccgtcccatcttcggattcgttcccgaacccttacacgggttttaggcgggattctccagtgggcttttttacgtcctcgctgtggtcgttccgtgactgcgtgccctcgcaccggcaacgtcctcgcttcgtccgtcatcgccaaatcaacatccctcattcggtcgtgtcggtcggtcggtccctcctcctcactcctcattcggtcagtttcttttggctctacgtgccttgtgattagtaagtcg

SEQ ID NO: 368 - DNA, 201 bp, Artificial Sequence
  orf
ttgtacagcatttcgagatatggcaagtcttctgatggcatatcgtctgtcattgtgtcagggattttgacaccccatagcagaccaaaagaacctgttaaacgctttcccttaagaaccttaaatgcttcccaagtcttttcaacggacagatcaccaaacttcaaagcatacttacagacttcggcagccgctttgcta

SEQ ID NO: 369 - DNA, 348 bp, Artificial Sequence
  orf
acgctttcccttaagaaccttaaatgcttcccaagtcttttcaacggacagatcaccaaacttcaaagcatacttacagacttcggcagccgctttgctataaccgtgacctttttcttttcttgccctgcggacatcgacaaccatcgagtccccagtaaggctatgccaatattccgaaaactcttgctgatcaatccattgaccaaccaaagcgaacatatgaatatgcggatgccattcgcctgttttttcattgaatgtattttcataggagtacatagccccttgaaccttacagaactcattaaaacctcgacctttcttttcatagtccctacgacgctc

SEQ ID NO: 370 - DNA, 150 bp, Artificial Sequence
  orf
attttcttgcaggacttgatcaatacgttcaacgtatttttgaatagcacgagaagcacgaatggcagcacagaaagggcaaagcaaatgctgtccgcaggtgcggagcttggagagtttaacttcgcctattgtgtaaaaatttttgaa

SEQ ID NO: 371 - DNA, 168 bp, Artificial Sequence
  orf
ataattttcctgattcttagccctatgtttcaaagtcgcaaaacgtgttattctatccctgtgttgatctgccttagcagattgcaaagcctgtatttcatcaccagccaaagtgtccgaaatatgggcttttttattgtcttttttaagcaatttttgtccccactt

SEQ ID NO: 372 - DNA, 2278 bp, Artificial Sequence
  isolate
caacacagggatagaataacacgttttggtattttgaaacacagatcgaagcaacaagaaaactttttgtggactcttgcaaaattcaaagaaaactaccctcaagacctgccaagtgaggaaacaacaaaagctttaaagtctgctcaaaagttgcaaggctgtggcaattttttgctcttcaaaaatttttacacaatagatcaaactaagttagccaaatttcatgtctgtggtcagcatttgttatgtcctttttgtgctggtatccgtgcttcaaagtctattcagaaatattcagaacgtattgatgaagttctcaagaaaaaccgtaagctaaaagtcgctcttttgacgctgacggttaaaaacggttctgacctaggggaacgtgctaatcatctcttaacgtcgtttagaacgcttatcaagcgcagacaggactatttaaaaagggtcgtgggtttaatgagttctgtaaaattgatggtgcgatgtattcctatgaaaatacctacaatgaaaaaacaggtgaatggcatcctcatgttcacatgcttgtacttctcaatgattggatagatcaggaagccttgggcgaaacttggcatgaaattactggtgattcttatatcgttgatgttcgaaaagttaaaaaatctaaagaattagggtatgccaaagcagcagcagaggtttgtaaatatgctcttaagtttagtgatctgactgttgaaaaaacttgggaggcatttaaaaccctgaagggtaaaaaccttagtggttcatttggttccctgcgtggtgtcaaaatccctaaaaaccttgctgacgatatgcctgaagatcaggatttaccttatctcgaaatgctgtacaagttcgtatttggcaagcattcgcactatgacttagttatgactagacacgtagaagcgcaggcgggagagcgcgcggagttaggaggagctgcgacgacgcccgacgagagatatagcgcgggtgcttcaagtgtagacgatttgagtacggaggcaggtggagggctcgcaccactgcgacgtacgaaaagaggacgcaaaaaacagcactggagaatcccaccttctacccgtgttcgtgttcggcaaagaatccgaagatgggacgggattatgtataacattgatcttttcccatatgtcgaacataggctattggctttcattggttagtctgtttcttgagaaacgcggaagcgtgcattctgaaaaactcaccacttttgaaaaaagtgtagtgagtacacttctccagtagccgaacgagtgtctacgagtgacgactcctaaataagaatttcaaaaccttccttccttgattacaagctccccttggatttccaagcgggagtttttagtcatcccgaatgggtgacgacgcaatactatttgattttttcatgattcaaggtatgattcattgtgaatcacattcctatatatgaaaaaatcaggtgagaatatgcaaattgatgaaccaacaaacttccaagtattcatggcagaagtaaacaagacagcaaagacagaaagtattggtgtttaccatcaagtacctttccgtatggctcgatggaactttgcaagattagaagggcttcgtaaccacatgggagaacctcgaaacaaagttcttaattcactaatcgaaattgctttagatcaggtattcgaacaacttgagcatggcagtaaagagatccgtcgctctgttcttgaggaagtttccaaagttctcgaatcaattgaacatgatggttctgggagcttagacaatgattaatctacaaggaactctaatcaatgctttccgtgtggatggtggtaaaggtaaagacggcaaagaatatgaagcgcgtgacaaggtgcaaattcttggttcgttggaactcccaaacggggaaattaaacacgaacttgttgacctaacagttgaggattctcgactgtttgaaccattcaaaaatcaggttattagcatctcttgcggtgctatggctatcggtcgtaatgttgttttttatgttcgtaaaagcgcaaaaccgatcttagcacagagcatttgatattcgttaaaaaacagtgagttagcgaagctactcgctgttttttctttctcttgatactattaattaattcggggacaaaatttcatgaaaaatgacaataaaaaacccctgttatcggaagctttggcgggcgtagataacaagggtttgcaatctgctaaggcagat

SEQ ID NO: 373 - protein, 188 aa, Artificial Sequence
  orf
MTRFGILKHRSKQQENFLWTLAKFKENYPQDLPSEETTKALKSAQKLQGCGNFLLFKNFYTIDQTKLAKFHVCGQHLLCPFCAGIRASKSIQKYSERIDEVLKKNRKLKVALLTLTVKNGSDLGERANHLLTSFRTLIKRRQDYLKRVVGLMSSVKLMVRCIPMKIPTMKKQVNGILMFTCLYFSMIG

SEQ ID NO: 374 - protein, 243 aa, Artificial Sequence
  orf
MDGAMYSYENTYNEKTGEWHPHVHMLVLLNDWIDQEALGETWHEITGDSYIVDVRKVKKSKELGYAKAAAEVCKYALKFSDLTVEKTWEAFKTLKGKNLSGSFGSLRGVKIPKNLADDMPEDQDLPYLEMLYKFVFGKHSHYDLVMTRHVEAQAGERAELGGAATTPDERYSAGASSVDDLSTEAGGGLAPLRRTKRGRKKQHWRIPPSTRVRVRQRIRRWDGIMYNIDLFPYVEHRLLAFIG

SEQ ID NO: 375 - protein, 86 aa, Artificial Sequence
  orf
MEGSHHCDVRKEDAKNSTGESHLLPVFVFGKESEDGTGLCITLIFSHMSNIGYWLSLVSLFLEKRGSVHSEKLTTFEKSVVSTLLQ

SEQ ID NO: 376 - protein, 118 aa, Artificial Sequence
  orf
MTFLYMKKSGENMQIDEPTNFQVFMAEVNKTAKTESIGVYHQVPFRMARWNFARLEGLRNHMGEPRNKVLNSLIEIALDQVFEQLEHGSKEIRRSVLEEVSKVLESIEHDGSGSLDND

SEQ ID NO: 377 - protein, 96 aa, Artificial Sequence
  orf
MINLQGTLINAFRVDGGKGKDGKEYEARDKVQILGSLELPNGEIKHELVDLTVEDSRLFEPFKNQVISISCGAMAIGRNVVFYVRKSAKPILAQSI

SEQ ID NO: 378 - protein, 227 aa, Artificial Sequence
  orf
MRGQNFMKNDNKKPLLSEALAGVDNKGLQSAKADQHRDRITRFGILKHRSKQQENFLWTLAKFKENYPQDLPSEETTKALKSAQKLQGCGNFLLFKNFYTIDQTKLAKFHVCGQHLLCPFCAGIRASKSIQKYSERIDEVLKKNRKLKVALLTLTVKNGSDLGERANHLLTSFRTLIKRRQDYLKRVVGLMSSVKLMVRCIPMKIPTMKKQVNGILMFTCLYFSMIG

SEQ ID NO: 379 - protein, 64 aa, Artificial Sequence
  orf
MNGSNSRESSTVRSTSSCLISPFGSSNEPRICTLSRASYSLPSLPLPPSTRKALIRVPCRLIIV

SEQ ID NO: 380 - protein, 60 aa, Artificial Sequence
  orf
MFDIWEKINVIHNPVPSSDSLPNTNTGRRWDSPVLFFASSFRTSQWCEPSTCLRTQIVYT

SEQ ID NO: 381 - protein, 60 aa, Artificial Sequence
  orf
MGFSSAVFCVLFSYVAVVRALHLPPYSNRLHLKHPRYISRRASSQLLLTPRALPPALLRV

SEQ ID NO: 382 - protein, 100 aa, Artificial Sequence
  orf
MKANSLCSTYGKRSMLYIIPSHLRILCRTRTRVEGGILQCCFLRPLFVRRSGASPPPASVLKSSTLEAPALYLSSGVVAAPPNSARSPACASTCLVITKS

SEQ ID NO: 383 - protein, 69 aa, Artificial Sequence
  orf
MSSARFLGILTPRREPNEPLRFLPFRVLNASQVFSTVRSLNLRAYLQTSAAALAYPNSLDFLTFRTSTI

SEQ ID NO: 384 - protein, 65 aa, Artificial Sequence
  orf
MNDIRITSNFMPSFAQGFLIYPIIEKYKHVNMRMPFTCFFIVGIFIGIHRTINFTELIKPTTLFK

SEQ ID NO: 385 - protein, 53 aa, Artificial Sequence
  orf
MRVVFFEFCKSPQKVFLLLRSVFQNTKTCYSIPVLICLSRLQTLVIYARQSFR

SEQ ID NO: 386 - DNA, 564 bp, Artificial Sequence
  orf
ataacacgttttggtattttgaaacacagatcgaagcaacaagaaaactttttgtggactcttgcaaaattcaaagaaaactaccctcaagacctgccaagtgaggaaacaacaaaagctttaaagtctgctcaaaagttgcaaggctgtggcaattttttgctcttcaaaaatttttacacaatagatcaaactaagttagccaaatttcatgtctgtggtcagcatttgttatgtcctttttgtgctggtatccgtgcttcaaagtctattcagaaatattcagaacgtattgatgaagttctcaagaaaaaccgtaagctaaaagtcgctcttttgacgctgacggttaaaaacggttctgacctaggggaacgtgctaatcatctcttaacgtcgtttagaacgcttatcaagcgcagacaggactatttaaaaagggtcgtgggtttaatgagttctgtaaaattgatggtgcgatgtattcctatgaaaatacctacaatgaaaaaacaggtgaatggcatcctcatgttcacatgcttgtacttctcaatgattgga

SEQ ID NO: 387 - DNA, 729 bp, Artificial Sequence
  orf
attgatggtgcgatgtattcctatgaaaatacctacaatgaaaaaacaggtgaatggcatcctcatgttcacatgcttgtacttctcaatgattggatagatcaggaagccttgggcgaaacttggcatgaaattactggtgattcttatatcgttgatgttcgaaaagttaaaaaatctaaagaattagggtatgccaaagcagcagcagaggtttgtaaatatgctcttaagtttagtgatctgactgttgaaaaaacttgggaggcatttaaaaccctgaagggtaaaaaccttagtggttcatttggttccctgcgtggtgtcaaaatccctaaaaaccttgctgacgatatgcctgaagatcaggatttaccttatctcgaaatgctgtacaagttcgtatttggcaagcattcgcactatgacttagttatgactagacacgtagaagcgcaggcgggagagcgcgcggagttaggaggagctgcgacgacgcccgacgagagatatagcgcgggtgcttcaagtgtagacgatttgagtacggaggcaggtggagggctcgcaccactgcgacgtacgaaaagaggacgcaaaaaacagcactggagaatcccaccttctacccgtgttcgtgttcggcaaagaatccgaagatgggacgggattatgtataacattgatcttttcccatatgtcgaacataggctattggctttcattggt

SEQ ID NO: 388 - DNA, 258 bp, Artificial Sequence
  orf
gtggagggctcgcaccactgcgacgtacgaaaagaggacgcaaaaaacagcactggagaatcccaccttctacccgtgttcgtgttcggcaaagaatccgaagatgggacgggattatgtataacattgatcttttcccatatgtcgaacataggctattggctttcattggttagtctgtttcttgagaaacgcggaagcgtgcattctgaaaaactcaccacttttgaaaaaagtgtagtgagtacacttctccag

SEQ ID NO: 389 - DNA, 354 bp, Artificial Sequence
  orf
atcacattcctatatatgaaaaaatcaggtgagaatatgcaaattgatgaaccaacaaacttccaagtattcatggcagaagtaaacaagacagcaaagacagaaagtattggtgtttaccatcaagtacctttccgtatggctcgatggaactttgcaagattagaagggcttcgtaaccacatgggagaacctcgaaacaaagttcttaattcactaatcgaaattgctttagatcaggtattcgaacaacttgagcatggcagtaaagagatccgtcgctctgttcttgaggaagtttccaaagttctcgaatcaattgaacatgatggttctgggagcttagacaatgat

SEQ ID NO: 390 - DNA, 288 bp, Artificial Sequence
  orf
atgattaatctacaaggaactctaatcaatgctttccgtgtggatggtggtaaaggtaaagacggcaaagaatatgaagcgcgtgacaaggtgcaaattcttggttcgttggaactcccaaacggggaaattaaacacgaacttgttgacctaacagttgaggattctcgactgtttgaaccattcaaaaatcaggttattagcatctcttgcggtgctatggctatcggtcgtaatgttgttttttatgttcgtaaaagcgcaaaaccgatcttagcacagagcatt

SEQ ID NO: 391 - DNA, 681 bp, Artificial Sequence
  orf
attcggggacaaaatttcatgaaaaatgacaataaaaaacccctgttatcggaagctttggcgggcgtagataacaagggtttgcaatctgctaaggcagatcaacacagggatagaataacacgttttggtattttgaaacacagatcgaagcaacaagaaaactttttgtggactcttgcaaaattcaaagaaaactaccctcaagacctgccaagtgaggaaacaacaaaagctttaaagtctgctcaaaagttgcaaggctgtggcaattttttgctcttcaaaaatttttacacaatagatcaaactaagttagccaaatttcatgtctgtggtcagcatttgttatgtcctttttgtgctggtatccgtgcttcaaagtctattcagaaatattcagaacgtattgatgaagttctcaagaaaaaccgtaagctaaaagtcgctcttttgacgctgacggttaaaaacggttctgacctaggggaacgtgctaatcatctcttaacgtcgtttagaacgcttatcaagcgcagacaggactatttaaaaagggtcgtgggtttaatgagttctgtaaaattgatggtgcgatgtattcctatgaaaatacctacaatgaaaaaacaggtgaatggcatcctcatgttcacatgcttgtacttctcaatgattgga

SEQ ID NO: 392 - DNA, 192 bp, Artificial Sequence
  orf
ttgaatggttcaaacagtcgagaatcctcaactgttaggtcaacaagttcgtgtttaatttccccgtttgggagttccaacgaaccaagaatttgcaccttgtcacgcgcttcatattctttgccgtctttacctttaccaccatccacacggaaagcattgattagagttccttgtagattaatcattgtc

SEQ ID NO: 393 - DNA, 180 bp, Artificial Sequence
  orf
atgttcgacatatgggaaaagatcaatgttatacataatcccgtcccatcttcggattctttgccgaacacgaacacgggtagaaggtgggattctccagtgctgttttttgcgtcctcttttcgtacgtcgcagtggtgcgagccctccacctgcctccgtactcaaatcgtctacact

SEQ ID NO: 394 - DNA, 180 bp, Artificial Sequence
  orf
gtgggattctccagtgctgttttttgcgtcctcttttcgtacgtcgcagtggtgcgagccctccacctgcctccgtactcaaatcgtctacacttgaagcacccgcgctatatctctcgtcgggcgtcgtcgcagctcctcctaactccgcgcgctctcccgcctgcgcttctacgtgtc

SEQ ID NO: 395 - DNA, 300 bp, Artificial Sequence
  orf
atgaaagccaatagcctatgttcgacatatgggaaaagatcaatgttatacataatcccgtcccatcttcggattctttgccgaacacgaacacgggtagaaggtgggattctccagtgctgttttttgcgtcctcttttcgtacgtcgcagtggtgcgagccctccacctgcctccgtactcaaatcgtctacacttgaagcacccgcgctatatctctcgtcgggcgtcgtcgcagctcctcctaactccgcgcgctctcccgcctgcgcttctacgtgtctagtcataactaagtca

SEQ ID NO: 396 - DNA, 207 bp, Artificial Sequence
  orf
atatcgtcagcaaggtttttagggattttgacaccacgcagggaaccaaatgaaccactaaggtttttacccttcagggttttaaatgcctcccaagttttttcaacagtcagatcactaaacttaagagcatatttacaaacctctgctgctgctttggcataccctaattctttagattttttaacttttcgaacatcaacgata

SEQ ID NO: 397 - DNA, 195 bp, Artificial Sequence
  orf
atcaacgatataagaatcaccagtaatttcatgccaagtttcgcccaaggcttcctgatctatccaatcattgagaagtacaagcatgtgaacatgaggatgccattcacctgttttttcattgtaggtattttcataggaatacatcgcaccatcaattttacagaactcattaaacccacgaccctttttaaa

SEQ ID NO: 398 - DNA, 159 bp, Artificial Sequence
  orf
ttgagggtagttttctttgaattttgcaagagtccacaaaaagttttcttgttgcttcgatctgtgtttcaaaataccaaaacgtgttattctatccctgtgttgatctgccttagcagattgcaaacccttgttatctacgcccgccaaagcttccga

SEQ ID NO: 399 - DNA, 2832 bp, Artificial Sequence
  isolate
caacacagggatagaataacacgttttggtattttgaaacatagatcgaagcaacaagaaaactttttgtggactctagcaaaattcaaagaaaactatccaaatgacctaccaagcgaagagactataaaggctctgaaatctgcacagaaattgcaagggtgcggaaactttttgcttttcaaaaatttttacacaatagatcaaactaagctctcaaaatttcatgtatgtaatcaacatttgttatgtccattttgtgcaggtatccgtgcatctaaggcaattcaaaaatatactgaacgtgttgatgaggtcttaaagaaaaatcgtaagcttaagcccgttttaatcactctcacggtaaaaaacggtactgacctaagtgaacggtctgcgcatcttataaaatcgcttagaacgcttattaagcgtcgtcaggactataataaaaaaggtcgtggatttaatgagttttgtaagatcaatggggctatgtattcttatgaaaacacgtacaacgaacaaactaaagagtggcatccccatgttcacatgtttgcccttttggatgattggatagaccaagaacacatgtcagaaacctggcatgaagtcactggtgactcgttcattgtagatattcgaaaagtaaaaaaaagaaaaagacttaggctattcaaaagctgctgcggaagtttgtaaatatgcacttaaatttggcgatctttcagtagaaaacacatgggaagcattcaagattttgaaaggtaagcgtttaagcggtgcatttggctctctttatggtgtaaaaattcctgaaaatcttactgatgaaatgcccgatgaaaacgatcttccttatttagaaatgctctataaatttgtcttttcgaagcagtcttactatgacttaacaatgacacgacacgtagagccacaatgcaaggacagggacaacgacgaagaggaggtagcgacgactgacggaggcggaaatggacgcagcagtgaggcgattaaggacgggcgaggacgggcggagcgcaccgagggcacgcagggtgcacggcaccgcccacagcacggacgtaaaaaacaacactggcaagtacctccagtaacgagggtacgagtgaaaaaacgcatcaaaagatgggatggttatttgtgtgttatgcacatgtaaaatcataaacctatgataaaattctagtcgctgaatagtcggattgcagtccaactacccagctaaccaaaacgatctaataaggagatcatcatggctgttgcacatactactgttttgtctttgcttggcaaagaagtttcattttctgttttattggatgaccaaataaaacccttttttcctgacggtgttcatgtatctggctctgttgaagctgtatatattgctttaaatggcaatcatcatattttggtttctgataatttatatgaactctctgaaatagatttaattatttgatatctgattttaccaatgcccgaaaatacatatttttgggcttttttttgtctgtttcttgagaaacgcggaagcgtgcattctgaaaaaaatatacattgagattggcggagagatcggagcgctgggcgtgggattgggatgtcgctcgatgtcctagcctgcgactggcgatacgagatatacgccaatttaacttcataaaataatatcagagggcacggagtgtgcccgaactgaagaaataaaaaatcaaaaactcctttccctgattacaagctcccatttgtttctactcgataagaacagagccatccggatggtggcgactgactattgatttgatattaaatttgatatataaatatctaaccataaatatcaaatccaatatgagaaccaatatcatggaattatcaaatttatctcaatttatgatcgaacttaaacactctggtaaatctgaatatcacggtgcttataagcaaattcctttgcgtcttcgttttgtttcaaatgctcgtgtaactgcattaacttcaatgtttggaggtgcaagtaaaaataaaattttgaatgacttgatcgaaattgctttagatcaagtctatgaaaaaatgactgatgaacaaaaagctgtatatgaagaaattcagtctttcactttggctggcacaattaaaagcgttgaatcgggtgacgaaaaagatgattaaacctgaagtcatcagcaacactccaatggaatcgcttgaagaagcatgttcttacttcaagcaagtgttgcaagaacacttagacaaaaaacgtgacatcacattatttgccattgccgttgtaggtgggcgcttaaaagttgtagaaggattaccaaaatgattaatttagaaggcacaattatcaatgtttttacccaacagggtggtaaagacaaaaaaacaggtgaagcttttgcagatcgggacaaaatacaattattgggtgagttagaactccccaatggtgacaaaaagaatgaattaattgatcttaccgttgaaaacggaaaggcttatgaaaacctaaaaaacaggcgtgtgagcattccttgtggtgctcttgcttcaggtagaaatgtgattttttatgtcgcaaaaggtgcaacacctaaacttttgagcacagtatgacactcgttaaaaaacagcaacttacgaagtaagttcgctgttttttcttttacttgatactattcattaagtcggggacaaaatttcatgagaaatgacataaaaaaacccctgttatctagcactttggttggtggcgataacaagggtttgcaatctgctaaggcagat

SEQ ID NO: 400 - protein, 229 aa, Artificial Sequence
  orf
MTRFGILKHRSKQQENFLWTLAKFKENYPNDLPSEETIKALKSAQKLQGCGNFLLFKNFYTIDQTKLSKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFALLDDWIDQEHMSETWHEVTGDSFIVDIRKVKKRKRLRLFKSCCGSL

SEQ ID NO: 401 - protein, 142 aa, Artificial Sequence
  orf
MLKGKRLSGAFGSLYGVKIPENLTDEMPDENDLPYLEMLYKFVFSKQSYYDLTMTRHVEPQCKDRDNDEEEVATTDGGGNGRSSEAIKDGRGRAERTEGTQGARHRPQHGRKKQHWQVPPVTRVRVKKRIKRWDGYLCVMHM

SEQ ID NO: 402 - protein, 71 aa, Artificial Sequence
  orf
MIMAVAHTTVLSLLGKEVSFSVLLDDQIKPFFPDGVHVSGSVEAVYIALNGNHHILVSDNLYELSEIDLII

SEQ ID NO: 403 - protein, 143 aa, Artificial Sequence
  orf
MCFYSIRTEPSGWWRLTIDLILNLIYKYLTINIKSNMRTNIMELSNLSQFMIELKHSGKSEYHGAYKQIPLRLRFVSNARVTALTSMFGGASKNKILNDLIEIALDQVYEKMTDEQKAVYEEIQSFTLAGTIKSVESGDEKDD

SEQ ID NO: 404 - protein, 85 aa, Artificial Sequence
  orf
MMNKKLYMKKFSLSLWLAQLKALNRVTKKMIKPEVISNTPMESLEEACSYFKQVLQEHLDKKRDITLFAIAVVGGRLKVVEGLPK

SEQ ID NO: 405 - protein, 116 aa, Artificial Sequence
  orf
MICHCRCRWALKSCRRITKMINLEGTIINVFTQQGGKDKKTGEAFADRDKIQLLGELELPNGDKKNELIDLTVENGKAYENLKNRRVSIPCGALASGRNVIFYVAKGATPKLLSTV

SEQ ID NO: 406 - protein, 262 aa, Artificial Sequence
  orf
MRNDIKKPLLSSTLVGGDNKGLQSAKADQHRDRITRFGILKHRSKQQENFLWTLAKFKENYPNDLPSEETIKALKSAQKLQGCGNFLLFKNFYTIDQTKLSKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFALLDDWIDQEHMSETWHEVTGDSFIVDIRKVKKRKRLRLFKSCCGSL

SEQ ID NO: 407 - protein, 73 aa, Artificial Sequence
  orf
MVRSINSFFLSPLGSSNSPNNCILSRSAKASPVFLSLPPCWVKTLIIVPSKLIILVILLQLLSAHLQRQWQIM

SEQ ID NO: 408 - protein, 65 aa, Artificial Sequence
  orf
MQLFVHQSFFHRLDLKQFRSSHSKFYFYLHLQTLKLMQLHEHLKQNEDAKEFAYKHRDIQIYQSV

SEQ ID NO: 409 - protein, 69 aa, Artificial Sequence
  orf
MKLNWRISRIASRRLGHRATSQSHAQRSDLSANLNVYFFQNARFRVSQETDKKKPKNMYFRALVKSDIK

SEQ ID NO: 410 - protein, 90 aa, Artificial Sequence
  orf
MILFYEVKLAYISYRQSQARTSSDIPIPRPALRSLRQSQCIFFSECTLPRFSRNRQKKAQKYVFSGIGKIRYQIIKSISESSYKLSETKI

SEQ ID NO: 411 - protein, 53 aa, Artificial Sequence
  orf
MNYQKPKYDDCHLKQYIQLQQSQIHEHRQEKRVLFGHPIKQKMKLLCQAKTKQ

SEQ ID NO: 412 - protein, 109 aa, Artificial Sequence
  orf
MDCNPTIQRLEFYHRFMILHVHNTQITIPSFDAFFHSYPRYWRYLPVLFFTSVLWAVPCTLRALGALRPSSPVLNRLTAASISASVSRRYLLFVVVPVLALWLYVSCHC

SEQ ID NO: 413 - protein, 81 aa, Artificial Sequence
  orf
MMRFFTRTLVTGGTCQCCFLRPCCGRCRAPCVPSVRSARPRPSLIASLLRPFPPPSVVATSSSSLSLSLHCGSTCRVIVKS

SEQ ID NO: 414 - protein, 53 aa, Artificial Sequence
  orf
MHIYKLPQQLLNSLSLFLFFTFRISTMNESPVTSCQVSDMCSWSIQSSKRANM

SEQ ID NO: 415 - protein, 70 aa, Artificial Sequence
  orf
MFLVYPIIQKGKHVNMGMPLFSLFVVRVFIRIHSPIDLTKLIKSTTFFIIVLTTLNKRSKRFYKMRRPFT

SEQ ID NO: 416 - DNA, 687 bp, Artificial Sequence
  orf
ataacacgttttggtattttgaaacatagatcgaagcaacaagaaaactttttgtggactctagcaaaattcaaagaaaactatccaaatgacctaccaagcgaagagactataaaggctctgaaatctgcacagaaattgcaagggtgcggaaactttttgcttttcaaaaatttttacacaatagatcaaactaagctctcaaaatttcatgtatgtaatcaacatttgttatgtccattttgtgcaggtatccgtgcatctaaggcaattcaaaaatatactgaacgtgttgatgaggtcttaaagaaaaatcgtaagcttaagcccgttttaatcactctcacggtaaaaaacggtactgacctaagtgaacggtctgcgcatcttataaaatcgcttagaacgcttattaagcgtcgtcaggactataataaaaaaggtcgtggatttaatgagttttgtaagatcaatggggctatgtattcttatgaaaacacgtacaacgaacaaactaaagagtggcatccccatgttcacatgtttgcccttttggatgattggatagaccaagaacacatgtcagaaacctggcatgaagtcactggtgactcgttcattgtagatattcgaaaagtaaaaaaaagaaaaagacttaggctattcaaaagctgctgcggaagtttg

SEQ ID NO: 417 - DNA, 426 bp, Artificial Sequence
  orf
attttgaaaggtaagcgtttaagcggtgcatttggctctctttatggtgtaaaaattcctgaaaatcttactgatgaaatgcccgatgaaaacgatcttccttatttagaaatgctctataaatttgtcttttcgaagcagtcttactatgacttaacaatgacacgacacgtagagccacaatgcaaggacagggacaacgacgaagaggaggtagcgacgactgacggaggcggaaatggacgcagcagtgaggcgattaaggacgggcgaggacgggcggagcgcaccgagggcacgcagggtgcacggcaccgcccacagcacggacgtaaaaaacaacactggcaagtacctccagtaacgagggtacgagtgaaaaaacgcatcaaaagatgggatggttatttgtgtgttatgcacatg

SEQ ID NO: 418 - DNA, 213 bp, Artificial Sequence
  orf
atcatcatggctgttgcacatactactgttttgtctttgcttggcaaagaagtttcattttctgttttattggatgaccaaataaaacccttttttcctgacggtgttcatgtatctggctctgttgaagctgtatatattgctttaaatggcaatcatcatattttggtttctgataatttatatgaactctctgaaatagatttaattatt

SEQ ID NO: 419 - DNA, 429 bp, Artificial Sequence
  orf
atttgtttctactcgataagaacagagccatccggatggtggcgactgactattgatttgatattaaatttgatatataaatatctaaccataaatatcaaatccaatatgagaaccaatatcatggaattatcaaatttatctcaatttatgatcgaacttaaacactctggtaaatctgaatatcacggtgcttataagcaaattcctttgcgtcttcgttttgtttcaaatgctcgtgtaactgcattaacttcaatgtttggaggtgcaagtaaaaataaaattttgaatgacttgatcgaaattgctttagatcaagtctatgaaaaaatgactgatgaacaaaaagctgtatatgaagaaattcagtctttcactttggctggcacaattaaaagcgttgaatcgggtgacgaaaaagatgat

SEQ ID NO: 420 - DNA, 255 bp, Artificial Sequence
  orf
ctgatgaacaaaaagctgtatatgaagaaattcagtctttcactttggctggcacaattaaaagcgttgaatcgggtgacgaaaaagatgattaaacctgaagtcatcagcaacactccaatggaatcgcttgaagaagcatgttcttacttcaagcaagtgttgcaagaacacttagacaaaaaacgtgacatcacattatttgccattgccgttgtaggtgggcgcttaaaagttgtagaaggattaccaaaa

SEQ ID NO: 421 - DNA, 348 bp, Artificial Sequence
  orf
attatttgccattgccgttgtaggtgggcgcttaaaagttgtagaaggattaccaaaatgattaatttagaaggcacaattatcaatgtttttacccaacagggtggtaaagacaaaaaaacaggtgaagcttttgcagatcgggacaaaatacaattattgggtgagttagaactccccaatggtgacaaaaagaatgaattaattgatcttaccgttgaaaacggaaaggcttatgaaaacctaaaaaacaggcgtgtgagcattccttgtggtgctcttgcttcaggtagaaatgtgattttttatgtcgcaaaaggtgcaacacctaaacttttgagcacagta

SEQ ID NO: 422 - DNA, 786 bp, Artificial Sequence
  orf
atgagaaatgacataaaaaaacccctgttatctagcactttggttggtggcgataacaagggtttgcaatctgctaaggcagatcaacacagggatagaataacacgttttggtattttgaaacatagatcgaagcaacaagaaaactttttgtggactctagcaaaattcaaagaaaactatccaaatgacctaccaagcgaagagactataaaggctctgaaatctgcacagaaattgcaagggtgcggaaactttttgcttttcaaaaatttttacacaatagatcaaactaagctctcaaaatttcatgtatgtaatcaacatttgttatgtccattttgtgcaggtatccgtgcatctaaggcaattcaaaaatatactgaacgtgttgatgaggtcttaaagaaaaatcgtaagcttaagcccgttttaatcactctcacggtaaaaaacggtactgacctaagtgaacggtctgcgcatcttataaaatcgcttagaacgcttattaagcgtcgtcaggactataataaaaaaggtcgtggatttaatgagttttgtaagatcaatggggctatgtattcttatgaaaacacgtacaacgaacaaactaaagagtggcatccccatgttcacatgtttgcccttttggatgattggatagaccaagaacacatgtcagaaacctggcatgaagtcactggtgactcgttcattgtagatattcgaaaagtaaaaaaaagaaaaagacttaggctattcaaaagctgctgcggaagtttg

SEQ ID NO: 423 - DNA, 219 bp, Artificial Sequence
  orf
acggtaagatcaattaattcattctttttgtcaccattggggagttctaactcacccaataattgtattttgtcccgatctgcaaaagcttcacctgtttttttgtctttaccaccctgttgggtaaaaacattgataattgtgccttctaaattaatcattttggtaatccttctacaacttttaagcgcccacctacaacggcaatggcaaataatg

SEQ ID NO: 424 - DNA, 195 bp, Artificial Sequence
  orf
atacagctttttgttcatcagtcattttttcatagacttgatctaaagcaatttcgatcaagtcattcaaaattttatttttacttgcacctccaaacattgaagttaatgcagttacacgagcatttgaaacaaaacgaagacgcaaaggaatttgcttataagcaccgtgatattcagatttaccagagtgtt

SEQ ID NO: 425 - DNA, 207 bp, Artificial Sequence
  orf
atgaagttaaattggcgtatatctcgtatcgccagtcgcaggctaggacatcgagcgacatcccaatcccacgcccagcgctccgatctctccgccaatctcaatgtatatttttttcagaatgcacgcttccgcgtttctcaagaaacagacaaaaaaaagcccaaaaatatgtattttcgggcattggtaaaatcagatatcaaa

SEQ ID NO: 426 - DNA, 270 bp, Artificial Sequence
  orf
ctgatattattttatgaagttaaattggcgtatatctcgtatcgccagtcgcaggctaggacatcgagcgacatcccaatcccacgcccagcgctccgatctctccgccaatctcaatgtatatttttttcagaatgcacgcttccgcgtttctcaagaaacagacaaaaaaaagcccaaaaatatgtattttcgggcattggtaaaatcagatatcaaataattaaatctatttcagagagttcatataaattatcagaaaccaaaata

SEQ ID NO: 427 - DNA, 159 bp, Artificial Sequence
  orf
ataaattatcagaaaccaaaatatgatgattgccatttaaagcaatatatacagcttcaacagagccagatacatgaacaccgtcaggaaaaaagggttttatttggtcatccaataaaacagaaaatgaaacttctttgccaagcaaagacaaaacag

SEQ ID NO: 428 - DNA, 327 bp, Artificial Sequence
  orf
ttggactgcaatccgactattcagcgactagaattttatcataggtttatgattttacatgtgcataacacacaaataaccatcccatcttttgatgcgttttttcactcgtaccctcgttactggaggtacttgccagtgttgttttttacgtccgtgctgtgggcggtgccgtgcaccctgcgtgccctcggtgcgctccgcccgtcctcgcccgtccttaatcgcctcactgctgcgtccatttccgcctccgtcagtcgtcgctacctcctcttcgtcgttgtccctgtccttgcattgtggctctacgtgtcgtgtcattgt

SEQ ID NO: 429 - DNA, 243 bp, Artificial Sequence
  orf
ttgatgcgttttttcactcgtaccctcgttactggaggtacttgccagtgttgttttttacgtccgtgctgtgggcggtgccgtgcaccctgcgtgccctcggtgcgctccgcccgtcctcgcccgtccttaatcgcctcactgctgcgtccatttccgcctccgtcagtcgtcgctacctcctcttcgtcgttgtccctgtccttgcattgtggctctacgtgtcgtgtcattgttaagtca

SEQ ID NO: 430 - DNA, 159 bp, Artificial Sequence
  orf
gtgcatatttacaaacttccgcagcagcttttgaatagcctaagtctttttcttttttttacttttcgaatatctacaatgaacgagtcaccagtgacttcatgccaggtttctgacatgtgttcttggtctatccaatcatccaaaagggcaaacatg

SEQ ID NO: 431 - DNA, 210 bp, Artificial Sequence
  orf
gtgttcttggtctatccaatcatccaaaagggcaaacatgtgaacatggggatgccactctttagtttgttcgttgtacgtgttttcataagaatacatagccccattgatcttacaaaactcattaaatccacgaccttttttattatagtcctgacgacgcttaataagcgttctaagcgattttataagatgcgcagaccgttcact

SEQ ID NO: 432 - DNA, 2537 bp, Artificial Sequence
  isolate
caacacagggatagaataacacgttttgggattttgaaacatagagcgaagcaacaagaaaattttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactaagctctcaaaatttcatgtatgtgggcagcatttgttatgtcctttctgtgctggtattcgtgcatctaaagcaattcaaaaatatactgaacgtgttgatgaagtgcttaagaaaaatcgcaagcttaagcctgttttaatcacgtttacggtgaagaatggaatcgacttaggggaaagatcatcccacctcataaaatcgtttagaacgcttctgaagcgtcgtagggactatattaaaaaaggtcgtgggtttaatgagttctgtaagatcaatggggctatgtattcttatgaaaacacgtataacgagcaaacaaaggaatggcatccgcacttacatatgtttgcacttttggatgattggatagaccaggaagagctttcacagtattggcattccatcactggtgattcaatgattgtagatattcgcagggtcaaaaaagaaaaaggcttaggctattcaaaggctgctgcggaagtatgtaaatatgctcttaaattcggtgttttgtctgtccaaaatacttgggaagctttcaaagtcttaaaaggtaagcgtttaagtggtgcatttggttctttatggggtgtcaaaattcctgaagatcttgctgatgatatgcctgatgaaaaagatcttccttacttagaaatgctctataaattcgtatttggaaaacagtcttactatgacttaactatgacacgacatgtagagccacaaaataaggacgacgccgacgacgaggagaaggaacgtcgacgagaaggagaaggcggacgcaagttagtggcgaatgaggacgatgtgaggagtgacgaggagcgtgccgaccgacggacacgaacgacacgaggacgaaaaaaacaacactggcaagtacctccagtaacgagggtacgagtgaaaaaacggattaaaagatgggatggttatttgtgtgttatgcacatgtaaaatcataaacctatgataaaattctagtcgctgaatagtcggattgcagtccaactacccagctaaccacaacgatctaagaaggagatcataatggctgttgcacatactactgttttgtctttgcttggcaaagaagtttcattttctgttttattagatgaccaaataaaacccttttttcctgacggtgttcatgtatctggctctgttgaagctgtatatattgctttaaatggcaatcatcatattttggtttctgataatttatatgaactctctgaaatagatttagttatttgatatctgattttaccaatgcccgaaaatacatatttttgggcttttttttgtccgtttcttaagaaacgcgtaagcgtacattctgaaaaaatcaaccgaacgagtgtctacgagtgacgactccggtataaaaatttcaaaacctcttttccctgattacaagctcccctttgccgttctccagtaagaaccgagccatcccgaatgggtggcgactctcctattgatttgttatggattcagtgtatgattcttttgtgaatccaataatctctttaatgaatcaggtatgaatatggaacctacaaatttagatattttcttaagtgaagttaaaaaaacaggtgaaacgaaattaactggtgttttccaccaagttccatttcgtatgttctctccacactatcaacgtgtcgaaggtcttcgaaattatatgaatgaacctcgtaataaggttcttaactctctaatagaaattgctttagatcaggtttttgaaaacctatctaaagatcctgaaatctataaagctgttatggctgaggtttcaaaaattgacattggtggctttgatggttctggagacttagatcgtgattaaccttgaaggaacaattattaatgtttttactcagcaaggcggtaaagacaaaaaaacaggtgaagcttttgctgatcgtgacaaaattcagctattgggagaattagaacttcccaatggtgataaaaaaaacgaattaattgatcttaccgttgaaaacggaaaggcatatgaaaacctaaaaaataagcgtgtgagcatcccttgcggtgctctagcttctggtcgaaatgttattttttatgttgcaaaaggcgcaactccaaaactcttaaaccaagtatgatactcgttaaaaaacagcaacttacgaagtaagttcgctgttttttcttttacttgatactattaattaaagtggggacaaaaattgactgtaaaagacaataaaaaagcccatatttctgatgctttggcgggcgacgaaatacaggctttgcaatctgctaaggcagat

SEQ ID NO: 433 - protein, 386 aa, Artificial Sequence
  orf
MTRFGILKHRAKQQENFLWTLAKFKENYPKDAPSEESTRAIKSAQKLKECGNFLLFKNFYTIDQTKLSKFHVCGQHLLCPFCAGIRASKAIQKYTERVDEVLKKNRKLKPVLITFTVKNGIDLGERSSHLIKSFRTLLKRRRDYIKKGRGFNEFCKINGAMYSYENTYNEQTKEWHPHLHMFALLDDWIDQEELSQYWHSITGDSMIVDIRRVKKEKGLGYSKAAAEVCKYALKFGVLSVQNTWEAFKVLKGKRLSGAFGSLWGVKIPEDLADDMPDEKDLPYLEMLYKFVFGKQSYYDLTMTRHVEPQNKDDADDEEKERRREGEGGRKLVANEDDVRSDEERADRRTRTTRGRKKQHWQVPPVTRVRVKKRIKRWDGYLCVMHM

SEQ ID NO: 434 - protein, 71 aa, Artificial Sequence
  orf
MIMAVAHTTVLSLLGKEVSFSVLLDDQIKPFFPDGVHVSGSVEAVYIALNGNHHILVSDNLYELSEIDLVI

SEQ ID NO: 435 - protein, 103 aa, Artificial Sequence
  orf
MNMEPTNLDIFLSEVKKTGETKLTGVFHQVPFRMFSPHYQRVEGLRNYMNEPRNKVLNSLIEIALDQVFENLSKDPEIYKAVMAEVSKIDIGGFDGSGDLDRD

SEQ ID NO: 436 - protein, 99 aa, Artificial Sequence
  orf
VMVINLEGTIINVFTQQGGKDKKTGEAFADRDKIQLLGELELPNGDKKNELIDLTVENGKAYENLKNKRVSIPCGALASGRNVIFYVAKGATPKLLNQV

SEQ ID NO: 437 - protein, 420 aa, Artificial Sequence
  orf
MTVKDNKKAHISDALAGDEIQALQSAKADQHRDRITRFGILKHRAKQQENFLWTLAKFKENYPKDAPSEESTRAIKSAQKLKECGNFLLFKNFYTIDQTKLSKFHVCGQHLLCPFCAGIRASKAIQKYTERVDEVLKKNRKLKPVLITFTVKNGIDLGERSSHLIKSFRTLLKRRRDYIKKGRGFNEFCKINGAMYSYENTYNEQTKEWHPHLHMFALLDDWIDQEELSQYWHSITGDSMIVDIRRVKKEKGLGYSKAAAEVCKYALKFGVLSVQNTWEAFKVLKGKRLSGAFGSLWGVKIPEDLADDMPDEKDLPYLEMLYKFVFGKQSYYDLTMTRHVEPQNKDDADDEEKERRREGEGGRKLVANEDDVRSDEERADRRTRTTRGRKKQHWQVPPVTRVRVKKRIKRWDGYLCVMHM

SEQ ID NO: 438 - protein, 52 aa, Artificial Sequence
  orf
MNFVTISKSFTCFFVFTALLSKNINNCSFKVNHDLSLQNHQSHQCQFLKPQP

SEQ ID NO: 439 - protein, 52 aa, Artificial Sequence
  orf
MRNGTWWKTPVNFVSPVFLTSLKKISKFVGSIFIPDSLKRLLDSQKNHTLNP

SEQ ID NO: 440 - protein, 50 aa, Artificial Sequence
  orf
MIFSECTLTRFLRNGQKKAQKYVFSGIGKIRYQITKSISESSYKLSETKI

SEQ ID NO: 441 - protein, 53 aa, Artificial Sequence
  orf
MNYQKPKYDDCHLKQYIQLQQSQIHEHRQEKRVLFGHLIKQKMKLLCQAKTKQ

SEQ ID NO: 442 - protein, 74 aa, Artificial Sequence
  orf
MDCNPTIQRLEFYHRFMILHVHNTQITIPSFNPFFHSYPRYWRYLPVLFFSSSCRSCPSVGTLLVTPHIVLIRH

SEQ ID NO: 443 - protein, 63 aa, Artificial Sequence
  orf
MEVLASVVFFVLVSFVSVGRHAPRHSSHRPHSPLTCVRLLLLVDVPSPRRRRRPYFVALHVVS

SEQ ID NO: 444 - protein, 76 aa, Artificial Sequence
  orf
MRFFTRTLVTGGTCQCCFFRPRVVRVRRSARSSSLLTSSSFATNLRPPSPSRRRSFSSSSASSLFCGSTCRVIVKS

SEQ ID NO: 445 - protein, 54 aa, Artificial Sequence
  orf
MSSARSSGILTPHKEPNAPLKRLPFKTLKASQVFWTDKTPNLRAYLHTSAAAFE

SEQ ID NO: 446 - protein, 50 aa, Artificial Sequence; identical to SEQ ID NO: 232
  orf

SEQ ID NO: 447 - protein, 51 aa, Artificial Sequence; identical to SEQ ID NO: 233
  orf

SEQ ID NO: 448 - protein, 84 aa, Artificial Sequence; identical to SEQ ID NO: 234
  orf

SEQ ID NO: 449 - DNA, 1158 bp, Artificial Sequence
  orf
ataacacgttttgggattttgaaacatagagcgaagcaacaagaaaattttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactaagctctcaaaatttcatgtatgtgggcagcatttgttatgtcctttctgtgctggtattcgtgcatctaaagcaattcaaaaatatactgaacgtgttgatgaagtgcttaagaaaaatcgcaagcttaagcctgttttaatcacgtttacggtgaagaatggaatcgacttaggggaaagatcatcccacctcataaaatcgtttagaacgcttctgaagcgtcgtagggactatattaaaaaaggtcgtgggtttaatgagttctgtaagatcaatggggctatgtattcttatgaaaacacgtataacgagcaaacaaaggaatggcatccgcacttacatatgtttgcacttttggatgattggatagaccaggaagagctttcacagtattggcattccatcactggtgattcaatgattgtagatattcgcagggtcaaaaaagaaaaaggcttaggctattcaaaggctgctgcggaagtatgtaaatatgctcttaaattcggtgttttgtctgtccaaaatacttgggaagctttcaaagtcttaaaaggtaagcgtttaagtggtgcatttggttctttatggggtgtcaaaattcctgaagatcttgctgatgatatgcctgatgaaaaagatcttccttacttagaaatgctctataaattcgtatttggaaaacagtcttactatgacttaactatgacacgacatgtagagccacaaaataaggacgacgccgacgacgaggagaaggaacgtcgacgagaaggagaaggcggacgcaagttagtggcgaatgaggacgatgtgaggagtgacgaggagcgtgccgaccgacggacacgaacgacacgaggacgaaaaaaacaacactggcaagtacctccagtaacgagggtacgagtgaaaaaacggattaaaagatgggatggttatttgtgtgttatgcacatg

SEQ ID NO: 450 - DNA, 213 bp, Artificial Sequence
  orf
atcataatggctgttgcacatactactgttttgtctttgcttggcaaagaagtttcattttctgttttattagatgaccaaataaaacccttttttcctgacggtgttcatgtatctggctctgttgaagctgtatatattgctttaaatggcaatcatcatattttggtttctgataatttatatgaactctctgaaatagatttagttatt

SEQ ID NO: 451 - DNA, 309 bp, Artificial Sequence
  orf
atgaatatggaacctacaaatttagatattttcttaagtgaagttaaaaaaacaggtgaaacgaaattaactggtgttttccaccaagttccatttcgtatgttctctccacactatcaacgtgtcgaaggtcttcgaaattatatgaatgaacctcgtaataaggttcttaactctctaatagaaattgctttagatcaggtttttgaaaacctatctaaagatcctgaaatctataaagctgttatggctgaggtttcaaaaattgacattggtggctttgatggttctggagacttagatcgtgat

SEQ ID NO: 452 - DNA, 294 bp, Artificial Sequence
  orf
atcgtgattaaccttgaaggaacaattattaatgtttttactcagcaaggcggtaaagacaaaaaaacaggtgaagcttttgctgatcgtgacaaaattcagctattgggagaattagaacttcccaatggtgataaaaaaaacgaattaattgatcttaccgttgaaaacggaaaggcatatgaaaacctaaaaaataagcgtgtgagcatcccttgcggtgctctagcttctggtcgaaatgttattttttatgttgcaaaaggcgcaactccaaaactcttaaaccaagta

SEQ ID NO: 453 - DNA, 1260 bp, Artificial Sequence
  orf
ttgactgtaaaagacaataaaaaagcccatatttctgatgctttggcgggcgacgaaatacaggctttgcaatctgctaaggcagatcaacacagggatagaataacacgttttgggattttgaaacatagagcgaagcaacaagaaaattttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactaagctctcaaaatttcatgtatgtgggcagcatttgttatgtcctttctgtgctggtattcgtgcatctaaagcaattcaaaaatatactgaacgtgttgatgaagtgcttaagaaaaatcgcaagcttaagcctgttttaatcacgtttacggtgaagaatggaatcgacttaggggaaagatcatcccacctcataaaatcgtttagaacgcttctgaagcgtcgtagggactatattaaaaaaggtcgtgggtttaatgagttctgtaagatcaatggggctatgtattcttatgaaaacacgtataacgagcaaacaaaggaatggcatccgcacttacatatgtttgcacttttggatgattggatagaccaggaagagctttcacagtattggcattccatcactggtgattcaatgattgtagatattcgcagggtcaaaaaagaaaaaggcttaggctattcaaaggctgctgcggaagtatgtaaatatgctcttaaattcggtgttttgtctgtccaaaatacttgggaagctttcaaagtcttaaaaggtaagcgtttaagtggtgcatttggttctttatggggtgtcaaaattcctgaagatcttgctgatgatatgcctgatgaaaaagatcttccttacttagaaatgctctataaattcgtatttggaaaacagtcttactatgacttaactatgacacgacatgtagagccacaaaataaggacgacgccgacgacgaggagaaggaacgtcgacgagaaggagaaggcggacgcaagttagtggcgaatgaggacgatgtgaggagtgacgaggagcgtgccgaccgacggacacgaacgacacgaggacgaaaaaaacaacactggcaagtacctccagtaacgagggtacgagtgaaaaaacggattaaaagatgggatggttatttgtgtgttatgcacatg

SEQ ID NO: 454 - DNA, 156 bp, Artificial Sequence
  orf
ctgaattttgtcacgatcagcaaaagcttcacctgtttttttgtctttaccgccttgctgagtaaaaacattaataattgttccttcaaggttaatcacgatctaagtctccagaaccatcaaagccaccaatgtcaatttttgaaacctcagcca

SEQ ID NO: 455 - DNA, 156 bp, Artificial Sequence
  orf
atacgaaatggaacttggtggaaaacaccagttaatttcgtttcacctgtttttttaacttcacttaagaaaatatctaaatttgtaggttccatattcatacctgattcattaaagagattattggattcacaaaagaatcatacactgaatcca

SEQ ID NO: 456 - DNA, 150 bp, Artificial Sequence
  orf
ttgattttttcagaatgtacgcttacgcgtttcttaagaaacggacaaaaaaaagcccaaaaatatgtattttcgggcattggtaaaatcagatatcaaataactaaatctatttcagagagttcatataaattatcagaaaccaaaata

SEQ ID NO: 457 - DNA, 159 bp, Artificial Sequence
  orf
ataaattatcagaaaccaaaatatgatgattgccatttaaagcaatatatacagcttcaacagagccagatacatgaacaccgtcaggaaaaaagggttttatttggtcatctaataaaacagaaaatgaaacttctttgccaagcaaagacaaaacag

SEQ ID NO: 458 - DNA, 222 bp, Artificial Sequence
  orf
ttggactgcaatccgactattcagcgactagaattttatcataggtttatgattttacatgtgcataacacacaaataaccatcccatcttttaatccgttttttcactcgtaccctcgttactggaggtacttgccagtgttgtttttttcgtcctcgtgtcgttcgtgtccgtcggtcggcacgctcctcgtcactcctcacatcgtcctcattcgccac

SEQ ID NO: 459 - DNA, 189 bp, Artificial Sequence
  orf
ctggaggtacttgccagtgttgtttttttcgtcctcgtgtcgttcgtgtccgtcggtcggcacgctcctcgtcactcctcacatcgtcctcattcgccactaacttgcgtccgccttctccttctcgtcgacgttccttctcctcgtcgtcggcgtcgtccttattttgtggctctacatgtcgtgtca

SEQ ID NO: 460 - DNA, 228 bp, Artificial Sequence
  orf
atccgttttttcactcgtaccctcgttactggaggtacttgccagtgttgtttttttcgtcctcgtgtcgttcgtgtccgtcggtcggcacgctcctcgtcactcctcacatcgtcctcattcgccactaacttgcgtccgccttctccttctcgtcgacgttccttctcctcgtcgtcggcgtcgtccttattttgtggctctacatgtcgtgtcatagttaagtca

SEQ ID NO: 461 - DNA, 162 bp, Artificial Sequence
  orf
atatcatcagcaagatcttcaggaattttgacaccccataaagaaccaaatgcaccacttaaacgcttaccttttaagactttgaaagcttcccaagtattttggacagacaaaacaccgaatttaagagcatatttacatacttccgcagcagcctttgaa

SEQ ID NO: 462 - DNA, 150 bp, Artificial Sequence; identical to SEQ ID NO: 249
  orf

SEQ ID NO: 463 - DNA, 153 bp, Artificial Sequence
  orf
gtgcggatgccattcctttgtttgctcgttatacgtgttttcataagaatacatagccccattgatcttacagaactcattaaacccacgaccttttttaatatagtccctacgacgcttcagaagcgttctaaacgattttatgaggtggga

SEQ ID NO: 464 - DNA, 252 bp, Artificial Sequence; identical to SEQ ID NO: 251
  orf

SEQ ID NO: 465 - DNA, 2593 bp, Artificial Sequence
  isolate
caacacagggatagaataacacgttttggtattttgaaacacaggtcgaagcaacaagaaaactttttgtggacgctagcaaaattcaaagaaaactacccaatagactcgccaaatgaagaaactgtaaaggccctaaaatctgcacagaagttaaacggctgcggtaattttttgctgttcaaaaatttttacacaatagatcaaactaagctcacaaagttccatgtctgtaatcaacatttgctttgcccattttgtgcaggtattcgtgcatctaaagcaattcaaaaatatacagagcgtgttgatgaagtattaaagaaaaatcgtaagctcaagcccgttctaatcacgctcactgtgaaaaacggtactgacctaagtgaacggtctgcacacctcataaaatcgcttagaacgcttattaagcgccgtcaggactataataaaaaaggtcgtggttttaacgaattttgtaaaattaatggtgcgatgtattcgtacgaaaatacttacaatgaacaaaccaaagaatggcatccgcacgttcatatgtttgctcttttggatgattggatagaccaggaactactttctgaaacatggcatgaaatcactggtgattcatttgttgtggatattcgaaaagttaaaaaacaaaaagacttaggctatgcaaaagctgctgctgaggtgtgtaaatacgctcttaagtttggggatctaacggtcgaaaatacatgggaagctttcaaagtcctaaaaggtaagcgcttaagcggtgcatttggctctctttatggtgtcaaaattccggaaaaccttgctgacgatatgccagaagaaaaagatcttccgtatttagagatgctctataagttcgtctttggaaaacactcttactacaatcttgaaattactagacacgtagaaccgcaggcgggagagcgcgcggagttaggaggagttgcgacgacgcccgacgagagatatagcgcgggcggttctagtgtagacgatttgagtacggaggcaggtggagggctcgcaccactgcgacgtacgaaaagaggacgcaaaaaacagcactggcaactaccaccagtaacgagagttagagtcaaaaaacgaatcaaaagatgggatggttatttgtgtgttatacacatgtaaaatcatgaacctatgataaaattctagccgctgaatagtaggacggcaatcctactacccagctaaccaaaacgatctaagaaggagatcatcatggctgtttcgaatattactgttttgtctttgcttggcaaagaagtttcattttctgtttcgttagatgaccaaataaaatccgttttccctaatggtgttcagattgctggagctgtcgaacaggtcattattgcattaaatggaaatcaccaaattcttgttggtgatgagttttatccagtttctgatattcaaaaaatttatgtcaaaacttgtatagaattttcctaattaccccgagaggggagacctcgaagcctttattcataaaggtttcggggtttttttatgtctttaacataatgcgtactattgaaaataaggcactaaagagtacttatataatctttagttcccgaacgagtgtctacgagtgacgactccagaataaaaatcacaaattcccctttctctgattacaagctcccctttgattttctccggtagaaacagagccatccggatggtggcgactcccctgttgaattatttgcgattcagtgtatgattcataatgtgaatcacaaaaacttttatcaaaattcaggtgagaatatggaacctacaaatttacaagtattcatgggtgaagtgaataaaacagcaaaaaccgaaactattggtgtttatcaccaagtaccatttcgaatgtctacatggaattttgagcgccttgaggggttgcgaaattacatgtcagagcctcgtaacaaggtcttaaattctcttattgaaattgctcttgatcaggttttttcagaactcgaaaagggtgatgaaaacattaaaaatgccattctttcagaatgtgcaaaggttaatgcaattcaaaaacatgatggttctggagacttagacaatgattaagttagagggaaccgtgattaatacatttcatcttgaaggtggaaaaaataaaaagggggaggaatacgaagctagtgacaaggtgcagttacttggttcgttggagctacccaatgggcaaactaaaaacgaatttattgatcttaaagtcgaagatgcaagcatctacgatgcattcaaaaataagcttattagcattagttgtggcgcatttcccgcagggaaaaacgtagtgttttatgttcgaaaaggtgcaaagcctgttttagccgacggtctttgatttatgttaaaaaacagtgagttagcgaagctactcgctgttttttattttacttgatactattcattaagtcggggacaaaatttcatgataaatgacaataaaaaacccctgttatcggacagtttggcgaccggtgataacaagggtttgcaatctgctaaggcagat

SEQ ID NO: 466 - protein, 386 aa, Artificial Sequence
  orf
MTRFGILKHRSKQQENFLWTLAKFKENYPIDSPNEETVKALKSAQKLNGCGNFLLFKNFYTIDQTKLTKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFALLDDWIDQELLSETWHEITGDSFVVDIRKVKKQKDLGYAKAAAEVCKYALKFGDLTVENTWEAFKVLKGKRLSGAFGSLYGVKIPENLADDMPEEKDLPYLEMLYKFVFGKHSYYNLEITRHVEPQAGERAELGGVATTPDERYSAGGSSVDDLSTEAGGGLAPLRRTKRGRKKQHWQLPPVTRVRVKKRIKRWDGYLCVIHM

SEQ ID NO: 467 - protein, 79 aa, Artificial Sequence
  orf
MIMAVSNITVLSLLGKEVSFSVSLDDQIKSVFPNGVQIAGAVEQVIIALNGNHQILVGDEFYPVSDIQKIYVKTCIEFS

SEQ ID NO: 468 - protein, 54 aa, Artificial Sequence
  orf
MYRIFLITPRGETSKPLFIKVSGFFYVFNIMRTIENKALKSTYIIFSSRTSVYE

SEQ ID NO: 469 - protein, 123 aa, Artificial Sequence
  orf
MQCMIHNVNHKNFYQNSGENMEPTNLQVFMGEVNKTAKTETIGVYHQVPFRMSTWNFERLEGLRNYMSEPRNKVLNSLIEIALDQVFSELEKGDENIKNAILSECAKVNAIQKHDGSGDLDND

SEQ ID NO: 470 - protein, 70 aa, Artificial Sequence
  orf
MHFILKVEKIKRGRNTKLVTRCSYLVRWSYPMGKLKTNLLILKSKMQASTMHSKISLLALVVAHFPQGKT

SEQ ID NO: 471 - protein, 96 aa, Artificial Sequence
  orf
MIKLEGTVINTFHLEGGKNKKGEEYEASDKVQLLGSLELPNGQTKNEFIDLKVEDASIYDAFKNKLISISCGAFPAGKNVVFYVRKGAKPVLADGL

SEQ ID NO: 472 - protein, 419 aa, Artificial Sequence
  orf
MINDNKKPLLSDSLATGDNKGLQSAKADQHRDRITRFGILKHRSKQQENFLWTLAKFKENYPIDSPNEETVKALKSAQKLNGCGNFLLFKNFYTIDQTKLTKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFALLDDWIDQELLSETWHEITGDSFVVDIRKVKKQKDLGYAKAAAEVCKYALKFGDLTVENTWEAFKVLKGKRLSGAFGSLYGVKIPENLADDMPEEKDLPYLEMLYKFVFGKHSYYNLEITRHVEPQAGERAELGGVATTPDERYSAGGSSVDDLSTEAGGGLAPLRRTKRGRKKQHWQLPPVTRVRVKKRIKRWDGYLCVIHM

SEQ ID NO: 473 - protein, 50 aa, Artificial Sequence
  orf
MNFDKSFCDSHYESYTESQIIQQGSRHHPDGSVSTGENQRGACNQRKGNL

SEQ ID NO: 474 - protein, 77 aa, Artificial Sequence
  orf
MIRFLTLTLVTGGSCQCCFLRPLFVRRSGASPPPASVLKSSTLEPPALYLSSGVVATPPNSARSPACGSTCLVISRL

SEQ ID NO: 475 - protein, 68 aa, Artificial Sequence
  orf
MVYPIIQKSKHMNVRMPFFGLFIVSIFVRIHRTINFTKFVKTTTFFIIVLTALNKRSKRFYEVCRPFT

SEQ ID NO: 476 - protein, 52 aa, Artificial Sequence
  orf
MNTLCIFLNCFRCTNTCTKWAKQMLITDMELCELSLIYCVKIFEQQKITAAV

SEQ ID NO: 477 - DNA, 1158 bp, Artificial Sequence
  orf
ataacacgttttggtattttgaaacacaggtcgaagcaacaagaaaactttttgtggacgctagcaaaattcaaagaaaactacccaatagactcgccaaatgaagaaactgtaaaggccctaaaatctgcacagaagttaaacggctgcggtaattttttgctgttcaaaaatttttacacaatagatcaaactaagctcacaaagttccatgtctgtaatcaacatttgctttgcccattttgtgcaggtattcgtgcatctaaagcaattcaaaaatatacagagcgtgttgatgaagtattaaagaaaaatcgtaagctcaagcccgttctaatcacgctcactgtgaaaaacggtactgacctaagtgaacggtctgcacacctcataaaatcgcttagaacgcttattaagcgccgtcaggactataataaaaaaggtcgtggttttaacgaattttgtaaaattaatggtgcgatgtattcgtacgaaaatacttacaatgaacaaaccaaagaatggcatccgcacgttcatatgtttgctcttttggatgattggatagaccaggaactactttctgaaacatggcatgaaatcactggtgattcatttgttgtggatattcgaaaagttaaaaaacaaaaagacttaggctatgcaaaagctgctgctgaggtgtgtaaatacgctcttaagtttggggatctaacggtcgaaaatacatgggaagctttcaaagtcctaaaaggtaagcgcttaagcggtgcatttggctctctttatggtgtcaaaattccggaaaaccttgctgacgatatgccagaagaaaaagatcttccgtatttagagatgctctataagttcgtctttggaaaacactcttactacaatcttgaaattactagacacgtagaaccgcaggcgggagagcgcgcggagttaggaggagttgcgacgacgcccgacgagagatatagcgcgggcggttctagtgtagacgatttgagtacggaggcaggtggagggctcgcaccactgcgacgtacgaaaagaggacgcaaaaaacagcactggcaactaccaccagtaacgagagttagagtcaaaaaacgaatcaaaagatgggatggttatttgtgtgttatacacatg

SEQ ID NO: 478 - DNA, 237 bp, Artificial Sequence
  orf
atcatcatggctgtttcgaatattactgttttgtctttgcttggcaaagaagtttcattttctgtttcgttagatgaccaaataaaatccgttttccctaatggtgttcagattgctggagctgtcgaacaggtcattattgcattaaatggaaatcaccaaattcttgttggtgatgagttttatccagtttctgatattcaaaaaatttatgtcaaaacttgtatagaattttcc

SEQ ID NO: 479 - DNA, 162 bp, Artificial Sequence
  orf
ttgtatagaattttcctaattaccccgagaggggagacctcgaagcctttattcataaaggtttcggggtttttttatgtctttaacataatgcgtactattgaaaataaggcactaaagagtacttatataatctttagttcccgaacgagtgtctacgag

SEQ ID NO: 480 - DNA, 369 bp, Artificial Sequence
  orf
attcagtgtatgattcataatgtgaatcacaaaaacttttatcaaaattcaggtgagaatatggaacctacaaatttacaagtattcatgggtgaagtgaataaaacagcaaaaaccgaaactattggtgtttatcaccaagtaccatttcgaatgtctacatggaattttgagcgccttgaggggttgcgaaattacatgtcagagcctcgtaacaaggtcttaaattctcttattgaaattgctcttgatcaggttttttcagaactcgaaaagggtgatgaaaacattaaaaatgccattctttcagaatgtgcaaaggttaatgcaattcaaaaacatgatggttctggagacttagacaatgat

SEQ ID NO: 481 - DNA, 210 bp, Artificial Sequence
  orf
atacatttcatcttgaaggtggaaaaaataaaaagggggaggaatacgaagctagtgacaaggtgcagttacttggttcgttggagctacccaatgggcaaactaaaaacgaatttattgatcttaaagtcgaagatgcaagcatctacgatgcattcaaaaataagcttattagcattagttgtggcgcatttcccgcagggaaaaacg

SEQ ID NO: 482 - DNA, 288 bp, Artificial Sequence
  orf
atgattaagttagagggaaccgtgattaatacatttcatcttgaaggtggaaaaaataaaaagggggaggaatacgaagctagtgacaaggtgcagttacttggttcgttggagctacccaatgggcaaactaaaaacgaatttattgatcttaaagtcgaagatgcaagcatctacgatgcattcaaaaataagcttattagcattagttgtggcgcatttcccgcagggaaaaacgtagtgttttatgttcgaaaaggtgcaaagcctgttttagccgacggtctt

SEQ ID NO: 483 - DNA, 1257 bp, Artificial Sequence
  orf
atgataaatgacaataaaaaacccctgttatcggacagtttggcgaccggtgataacaagggtttgcaatctgctaaggcagatcaacacagggatagaataacacgttttggtattttgaaacacaggtcgaagcaacaagaaaactttttgtggacgctagcaaaattcaaagaaaactacccaatagactcgccaaatgaagaaactgtaaaggccctaaaatctgcacagaagttaaacggctgcggtaattttttgctgttcaaaaatttttacacaatagatcaaactaagctcacaaagttccatgtctgtaatcaacatttgctttgcccattttgtgcaggtattcgtgcatctaaagcaattcaaaaatatacagagcgtgttgatgaagtattaaagaaaaatcgtaagctcaagcccgttctaatcacgctcactgtgaaaaacggtactgacctaagtgaacggtctgcacacctcataaaatcgcttagaacgcttattaagcgccgtcaggactataataaaaaaggtcgtggttttaacgaattttgtaaaattaatggtgcgatgtattcgtacgaaaatacttacaatgaacaaaccaaagaatggcatccgcacgttcatatgtttgctcttttggatgattggatagaccaggaactactttctgaaacatggcatgaaatcactggtgattcatttgttgtggatattcgaaaagttaaaaaacaaaaagacttaggctatgcaaaagctgctgctgaggtgtgtaaatacgctcttaagtttggggatctaacggtcgaaaatacatgggaagctttcaaagtcctaaaaggtaagcgcttaagcggtgcatttggctctctttatggtgtcaaaattccggaaaaccttgctgacgatatgccagaagaaaaagatcttccgtatttagagatgctctataagttcgtctttggaaaacactcttactacaatcttgaaattactagacacgtagaaccgcaggcgggagagcgcgcggagttaggaggagttgcgacgacgcccgacgagagatatagcgcgggcggttctagtgtagacgatttgagtacggaggcaggtggagggctcgcaccactgcgacgtacgaaaagaggacgcaaaaaacagcactggcaactaccaccagtaacgagagttagagtcaaaaaacgaatcaaaagatgggatggttatttgtgtgttatacacatg

SEQ ID NO: 484 - DNA, 150 bp, Artificial Sequence
  orf
ctgaattttgataaaagtttttgtgattcacattatgaatcatacactgaatcgcaaataattcaacaggggagtcgccaccatccggatggctctgtttctaccggagaaaatcaaaggggagcttgtaatcagagaaaggggaatttg

SEQ ID NO: 485 - DNA, 231 bp, Artificial Sequence
  orf
ttgattcgttttttgactctaactctcgttactggtggtagttgccagtgctgttttttgcgtcctcttttcgtacgtcgcagtggtgcgagccctccacctgcctccgtactcaaatcgtctacactagaaccgcccgcgctatatctctcgtcgggcgtcgtcgcaactcctcctaactccgcgcgctctcccgcctgcggttctacgtgtctagtaatttcaagattg

SEQ ID NO: 486 - DNA, 204 bp, Artificial Sequence
  orf
ctggtctatccaatcatccaaaagagcaaacatatgaacgtgcggatgccattctttggtttgttcattgtaagtattttcgtacgaatacatcgcaccattaattttacaaaattcgttaaaaccacgaccttttttattatagtcctgacggcgcttaataagcgttctaagcgattttatgaggtgtgcagaccgttcact

SEQ ID NO: 487 - DNA, 156 bp, Artificial Sequence
  orf
atcaacacgctctgtatatttttgaattgctttagatgcacgaatacctgcacaaaatgggcaaagcaaatgttgattacagacatggaactttgtgagcttagtttgatctattgtgtaaaaatttttgaacagcaaaaaattaccgcagccgtt

SEQ ID NO: 488 - DNA, 2554 bp, Artificial Sequence
  isolate
caacacagggatagaataacacgttttggtattttgaaacacagatcgaagcaacaagaaaactttttgtggactcttgcaaaattcaaagaaaactacccaatagactcgccaaatgaagaaactgtaaaggctctaaaatctgcacagaagttgagcggctgtggcaattttttgttgttcaaaaatttttacacaatagatcaaaccaagctctcaaaatttcatgtttgtaatcagcatttgttgtgtccgttttgtacaggtattcgtgcttctaaagcaattcaaaaatattctgaacgtgttgatgaagtacttaaacaaaatcgcaagctcaagcctgttttaatcaccctgacagtgaaaatggtactgacctaagggaaaggtctgaacatctcataaagtcgcttagaacgctaattaagcgtcgtcaggactacaataaaaagaatcgtggttttaacgagttttgcaaaatcaacggtgcaatgtattcatacgaaaacacctacaatgaaaaaacaggcgaatggcatccgcacgttcatatgtttgcactgctcgatacttggatcgatcaagaacaactttctgagacttggcacgagatcacaggtgattcttttgttgttgatattcgaaaggttaaaaaagacaaagactttggttattcaaaggcagcagctgaagtatgcaaatatgctttaaagtttggtgatctgtcagttgaacatacctgggaagcgttcaaggttctcaaaggcaaacgcctaagcggtgcattcggttgtctttatggggttaaaattcctgaaaatcttgcagacgacatgcccgatgaaaaagaccttccgtatttagaaatgctttatagattcgtgtttggaaaacagccttactatgatctacaaatgacacgtcatgtagagccacaggcgaatgaacctgagaacgtgaggagctttgacgaatgtgatcgggtttgtgagccattggcgatgacggacgagacgaggacgttgccggtgcgagggcacgcagtcacggaacgaccgcagcgagtacgtaaaaagcaacactggcaagtaccaccagtaacgagggtacgagtgaaaaaacgtattaaaagatgggatggttatttgtgtgttatgcacatgtaaaatcataaacatatgttaaaattctagtcgctgaatagtcggatggccgtccaactacccagctaaccaatcagatctaagaaggagatcatcatggctgttgcacatactactgttttggaaatgctaggcaaagaagtctcattttctgttttgctagatgaccaaataaaatgctttttccctgaaggtattcagattactggagacgttcaggaagtgattattgcccttgatggcaatcatcaaatcttagttctagatgaatattatcaactctcaagaattgctctgaaatcttaattagccccgataggggagacctcaaacccattgtttacaatggttttggggtttttttatgccctttacataatgtgtattatcttaaccgccttactaaagattactcatgtaatctttagcgagtgtctacgagcgtctctccagtaataaaaatttcaaacctttttccctctgattacaagctcccttttgcctttctccgggataacccgagccaacccgaatgggtggcgactcccctattgatttgttatttattcagtgtatgattcttttgtgaatcatataaattaatcaacgaatcaggtatgaatatggaacctacaaatttagatatttttttaagcgaagttaagaagactggtgaatcaaaacttgttggtatttatcatcaagtcccattccgtatgttctctccacattatcaacgtgttgaaggccttcgaaattatatgaatgagcctcggaacaaagttcttaattctttgatcgaaattgctttagatcaggtttttgaaaacctgtctaaggatccagaagtttataaagcagtaatgactgaagtttataaaatcaatattggtgaatctttcgatggttctggagatttatctaatgattaaattagaaggcacagtcttgaacgttctcgttcaacaaggcggaaaagataagaaaacaggcgatcagtttgaggatcgtgacaaggtacaaattttgggtgctatggagcttccaaacggtggcgttaaaaatgagctttttacgttatcagtagaaaattatcgtgattttaaagacttcataaatcaaaaaatcacaattgctgttggtgcaatggccagtggtagaaacgtaattttttacgttgcaaagggtgctttacctgttttagtacaggattgatatttgttagaaaaaacagtgagttagcgaagcaactcgctgtttttttatttacttgatactattcattaagtcggggacaaaatttcatgagaaatgacaataaaaaacccctgttatcagaagctttggcgggcgtagataacaagggttcgcaatctgctaaggcagat

SEQ ID NO: 489 - protein, 122 aa, Artificial Sequence
  orf
MTRFGILKHRSKQQENFLWTLAKFKENYPIDSPNEETVKALKSAQKLSGCGNFLLFKNFYTIDQTKLSKFHVCNQHLLCPFCTGIRASKAIQKYSERVDEVLKQNRKLKPVLITLTVKMVLT

SEQ ID NO: 490 - protein, 88 aa, Artificial Sequence
  orf
MKRIVVLTSFAKSTVQCIHTKTPTMKKQANGIRTFICLHCSILGSIKNNFLRLGTRSQVILLLLIFERLKKTKTLVIQRQQLKYANML

SEQ ID NO: 491 - protein, 254 aa, Artificial Sequence
  orf
MKSLRTLIKRRQDYNKKNRGFNEFCKINGAMYSYENTYNEKTGEWHPHVHMFALLDTWIDQEQLSETWHEITGDSFVVDIRKVKKDKDFGYSKAAAEVCKYALKFGDLSVEHTWEAFKVLKGKRLSGAFGCLYGVKIPENLADDMPDEKDLPYLEMLYRFVFGKQPYYDLQMTRHVEPQANEPENVRSFDECDRVCEPLAMTDETRTLPVRGHAVTERPQRVRKKQHWQVPPVTRVRVKKRIKRWDGYLCVMHM

SEQ ID NO: 492 - protein, 77 aa, Artificial Sequence
  orf
MRSKKEIIMAVAHTTVLEMLGKEVSFSVLLDDQIKCFFPEGIQITGDVQEVIIALDGNHQILVLDEYYQLSRIALKS

SEQ ID NO: 493 - protein, 52 aa, Artificial Sequence
  orf
MLSTLKNCSEILISPDRGDLKPIVYNGFGVFLCPLHNVYYLNRLTKDYSCNL

SEQ ID NO: 494 - protein, 109 aa, Artificial Sequence
  orf
MNESGMNMEPTNLDIFLSEVKKTGESKLVGIYHQVPFRMFSPHYQRVEGLRNYMNEPRNKVLNSLIEIALDQVFENLSKDPEVYKAVMTEVYKINIGESFDGSGDLSND

SEQ ID NO: 495 - protein, 115 aa, Artificial Sequence
  orf
MKFIKSILVNLSMVLEIYLMIKLEGTVLNVLVQQGGKDKKTGDQFEDRDKVQILGAMELPNGGVKNELFTLSVENYRDFKDFINQKITIAVGAMASGRNVIFYVAKGALPVLVQD

SEQ ID NO: 496 - protein, 155 aa, Artificial Sequence
  orf
MRNDNKKPLLSEALAGVDNKGSQSAKADQHRDRITRFGILKHRSKQQENFLWTLAKFKENYPIDSPNEETVKALKSAQKLSGCGNFLLFKNFYTIDQTKLSKFHVCNQHLLCPFCTGIRASKAIQKYSERVDEVLKQNRKLKPVLITLTVKMVLT

SEQ ID NO: 497 - protein, 67 aa, Artificial Sequence
  orf
MRFFTRTLVTGGTCQCCFLRTRCGRSVTACPRTGNVLVSSVIANGSQTRSHSSKLLTFSGSFACGST

SEQ ID NO: 498 - protein, 89 aa, Artificial Sequence
  orf
MFMILHVHNTQITIPSFNTFFHSYPRYWWYLPVLLFTYSLRSFRDCVPSHRQRPRLVRHRQWLTNPITFVKAPHVLRFIRLWLYMTCHL

SEQ ID NO: 499 - protein, 79 aa, Artificial Sequence
  orf
MRALAPATSSSRPSSPMAHKPDHIRQSSSRSQVHSPVALHDVSFVDHSKAVFQTRIYKAFLNTEGLFHRACRLQDFQEF

SEQ ID NO: 500 - protein, 58 aa, Artificial Sequence
  orf
MTKVFVFFNLSNINNKRITCDLVPSLRKLFLIDPSIEQCKHMNVRMPFACFFIVGVFV

SEQ ID NO: 501 - protein, 56 aa, Artificial Sequence; identical to SEQ ID NO: 300
  orf

SEQ ID NO: 502 - DNA, 366 bp, Artificial Sequence
  orf
ataacacgttttggtattttgaaacacagatcgaagcaacaagaaaactttttgtggactcttgcaaaattcaaagaaaactacccaatagactcgccaaatgaagaaactgtaaaggctctaaaatctgcacagaagttgagcggctgtggcaattttttgttgttcaaaaatttttacacaatagatcaaaccaagctctcaaaatttcatgtttgtaatcagcatttgttgtgtccgttttgtacaggtattcgtgcttctaaagcaattcaaaaatattctgaacgtgttgatgaagtacttaaacaaaatcgcaagctcaagcctgttttaatcaccctgacagtgaaaatggtactgacc

SEQ ID NO: 503 - DNA, 264 bp, Artificial Sequence
  orf
ataaaaagaatcgtggttttaacgagttttgcaaaatcaacggtgcaatgtattcatacgaaaacacctacaatgaaaaaacaggcgaatggcatccgcacgttcatatgtttgcactgctcgatacttggatcgatcaagaacaactttctgagacttggcacgagatcacaggtgattcttttgttgttgatattcgaaaggttaaaaaagacaaagactttggttattcaaaggcagcagctgaagtatgcaaatatgctt

SEQ ID NO: 504 - DNA, 762 bp, Artificial Sequence
  orf
ataaagtcgcttagaacgctaattaagcgtcgtcaggactacaataaaaagaatcgtggttttaacgagttttgcaaaatcaacggtgcaatgtattcatacgaaaacacctacaatgaaaaaacaggcgaatggcatccgcacgttcatatgtttgcactgctcgatacttggatcgatcaagaacaactttctgagacttggcacgagatcacaggtgattcttttgttgttgatattcgaaaggttaaaaaagacaaagactttggttattcaaaggcagcagctgaagtatgcaaatatgctttaaagtttggtgatctgtcagttgaacatacctgggaagcgttcaaggttctcaaaggcaaacgcctaagcggtgcattcggttgtctttatggggttaaaattcctgaaaatcttgcagacgacatgcccgatgaaaaagaccttccgtatttagaaatgctttatagattcgtgtttggaaaacagccttactatgatctacaaatgacacgtcatgtagagccacaggcgaatgaacctgagaacgtgaggagctttgacgaatgtgatcgggtttgtgagccattggcgatgacggacgagacgaggacgttgccggtgcgagggcacgcagtcacggaacgaccgcagcgagtacgtaaaaagcaacactggcaagtaccaccagtaacgagggtacgagtgaaaaaacgtattaaaagatgggatggttatttgtgtgttatgcacatg

SEQ ID NO: 505 - DNA, 231 bp, Artificial Sequence
  orf
atcagatctaagaaggagatcatcatggctgttgcacatactactgttttggaaatgctaggcaaagaagtctcattttctgttttgctagatgaccaaataaaatgctttttccctgaaggtattcagattactggagacgttcaggaagtgattattgcccttgatggcaatcatcaaatcttagttctagatgaatattatcaactctcaagaattgctctgaaatct

SEQ ID NO: 506 - DNA, 156 bp, Artificial Sequence
  orf
atattatcaactctcaagaattgctctgaaatcttaattagccccgataggggagacctcaaacccattgtttacaatggttttggggtttttttatgccctttacataatgtgtattatcttaaccgccttactaaagattactcatgtaatctt

SEQ ID NO: 507 - DNA, 327 bp, Artificial Sequence
  orf
atcaacgaatcaggtatgaatatggaacctacaaatttagatatttttttaagcgaagttaagaagactggtgaatcaaaacttgttggtatttatcatcaagtcccattccgtatgttctctccacattatcaacgtgttgaaggccttcgaaattatatgaatgagcctcggaacaaagttcttaattctttgatcgaaattgctttagatcaggtttttgaaaacctgtctaaggatccagaagtttataaagcagtaatgactgaagtttataaaatcaatattggtgaatctttcgatggttctggagatttatctaatgat

SEQ ID NO: 508 - DNA, 345 bp, Artificial Sequence
  orf
ctgaagtttataaaatcaatattggtgaatctttcgatggttctggagatttatctaatgattaaattagaaggcacagtcttgaacgttctcgttcaacaaggcggaaaagataagaaaacaggcgatcagtttgaggatcgtgacaaggtacaaattttgggtgctatggagcttccaaacggtggcgttaaaaatgagctttttacgttatcagtagaaaattatcgtgattttaaagacttcataaatcaaaaaatcacaattgctgttggtgcaatggccagtggtagaaacgtaattttttacgttgcaaagggtgctttacctgttttagtacaggat

SEQ ID NO: 509 - DNA, 465 bp, Artificial Sequence
  orf
atgagaaatgacaataaaaaacccctgttatcagaagctttggcgggcgtagataacaagggttcgcaatctgctaaggcagatcaacacagggatagaataacacgttttggtattttgaaacacagatcgaagcaacaagaaaactttttgtggactcttgcaaaattcaaagaaaactacccaatagactcgccaaatgaagaaactgtaaaggctctaaaatctgcacagaagttgagcggctgtggcaattttttgttgttcaaaaatttttacacaatagatcaaaccaagctctcaaaatttcatgtttgtaatcagcatttgttgtgtccgttttgtacaggtattcgtgcttctaaagcaattcaaaaatattctgaacgtgttgatgaagtacttaaacaaaatcgcaagctcaagcctgttttaatcaccctgacagtgaaaatggtactgacc

SEQ ID NO: 510 - DNA, 201 bp, Artificial Sequence
  orf
atacgttttttcactcgtaccctcgttactggtggtacttgccagtgttgctttttacgtactcgctgcggtcgttccgtgactgcgtgccctcgcaccggcaacgtcctcgtctcgtccgtcatcgccaatggctcacaaacccgatcacattcgtcaaagctcctcacgttctcaggttcattcgcctgtggctctaca

SEQ ID NO: 511 - DNA, 267 bp, Artificial Sequence
  orf
atgtttatgattttacatgtgcataacacacaaataaccatcccatcttttaatacgttttttcactcgtaccctcgttactggtggtacttgccagtgttgctttttacgtactcgctgcggtcgttccgtgactgcgtgccctcgcaccggcaacgtcctcgtctcgtccgtcatcgccaatggctcacaaacccgatcacattcgtcaaagctcctcacgttctcaggttcattcgcctgtggctctacatgacgtgtcatttg

SEQ ID NO: 512 - DNA, 237 bp, Artificial Sequence
  orf
ctgcgtgccctcgcaccggcaacgtcctcgtctcgtccgtcatcgccaatggctcacaaacccgatcacattcgtcaaagctcctcacgttctcaggttcattcgcctgtggctctacatgacgtgtcatttgtagatcatagtaaggctgttttccaaacacgaatctataaagcatttctaaatacggaaggtctttttcatcgggcatgtcgtctgcaagattttcaggaattt

SEQ ID NO: 513 - DNA, 174 bp, Artificial Sequence
  orf
ataaccaaagtctttgtcttttttaacctttcgaatatcaacaacaaaagaatcacctgtgatctcgtgccaagtctcagaaagttgttcttgatcgatccaagtatcgagcagtgcaaacatatgaacgtgcggatgccattcgcctgttttttcattgtaggtgttttcgta

SEQ ID NO: 514 - DNA, 168 bp, Artificial Sequence
  orf
atttggcgagtctattgggtagttttctttgaattttgcaagagtccacaaaaagttttcttgttgcttcgatctgtgtttcaaaataccaaaacgtgttattctatccctgtgttgatctgccttagcagattgcgaacccttgttatctacgcccgccaaagcttc

SEQ ID NO: 515 - DNA, 2505 bp, Artificial Sequence
  orf
aaggcagatcaacacagggatagaataacacgttttggaattttgaaacatagatctaaactgcaagaacaatatttgtggacgcaggttgatttcaaatccgaaggcgaagacgacctatctaataaggctcttaaggctgcaacaaaattaaaaggttgtggtcaatttctgctgttcagaaattactacacgatagaccaaatcaagctcgaaaaattccatgtatgcggtcaacatttgctatgcccaatgtgtgctggtattcgtgcagctcgttctatgagtcgttatattcagcgcattgaagaattgatgcgtcagaatcgccatttaaagcccgtattgatcactttaacggtaaagaatggtgaagacctgcaagaacgctttgatcaccttacacgctcatttaagacccttttagctcgttatcgggactataagaaaaaaggcaggggttttaatcaattctgtaagattgatggtgcgttttataccactgaatacacttacaacgataagactaagcaatggcatccgcacattcatatttttgctttactcaatgaatggattgaccaggaagaattggcagaaacttggcacgacattaccctggattcttatatcgttgatattcgcagagttaaaaaaaaaccaaagaacacggttatagcaaagctgttgcagaggtttgtaagtacgctcttaagtttagcgatctgtcactggagaacacatgggaagcctttttgactcttaaagataaacgccttacaggttctttcggttctatgcacggtgtaaaagtccctgaaaaagctacagacgaaatgccaaaagaagaactcccatatctagagctgcagtaccgtttcgtatttggtccgaaatcttattacaacctagaaataacaaaagacgtaaggccacaaactaaggaatgaggggtgaggaggagctgcgacgaccgacacgaccgaatgactttgtgagtggcgataaggacgaagcaatcgcatccgacatcccggcttgcgggtgcggtgcgaagcgaggacgttaagcaagcctacctgtatatacagaacttgtagggggttcgaccccttagaccccaaagggcgcactttaccaaacccttcaggttcggcagtgctcccaaaaattcccattggagaacattatgaaattaatctatagaccgtatccatttaagcctaactacgtttttcttgaagatcgcttaagttcttatgttctttgcccttggtggtggaattaattaacaaagctactggagaacattctctccagtagtaaccgagggggcggagtgcccccgaactgacatcatctaaattctcaaaccttccttctctgattacaagctccccttagattctaagcgggagtttttagccatcccgaatgggtggcggcacaaagaaaattgacaaaataataattagtattactatggtattaccaaccacacaaaatatatggtacatgaaatgggtaatacatttaaaaatcttggtcatttaaaagctgtatcagaattagaagaggaaatggagggtaatacctctcaaattcattcacctctaaaacctttttctcttaaagttccacaacacgttcttgagtctttggactttttggctgaaacaatgggtcttagccgtagtgctttaatcgttaatatttttgatcaatatcttggtcaagttacttatgaatatttagatggctataacggtttagtaactccagaagcacctcctgaagttgttgtttctgaagatttagaacaacgtttaaaaaattcagaattatcagaatcagcaaaacaatatttaacagatggtgtctatcacgttgcttttggaatgtgacatgacacaaatgcctaaagaacatttagaacaagcgaaagacttttttgcttggattatccaagaacacatgaaagacaaccgaaccttaacaaccatcaaaatctttaaacaagatggccaattaaaaattttggagttatcaaatgattaaattagaagggacattgttaaacacctttactcaacaaggcggacaaaataagaaaactggtgatcagtttgaagaccgtgacaaagtacaaattctaggcgctatggatctgccaaacggtgacgttaaaaatgaactttttacgttatctgtagagaattatcgggactttaaagacttcctaaatcacaaaatttctgttgcagttggtgcaatggcaagtggacgtaatgtgattttttatgttgctaaaggcgcaaagcctattttagccgaacaagcgtgaaacctaaaaaaaacagtgagttagcgaagcggctcgctgttttttctttctcttgatactattaactaaagtggggacaagaattgcttaaaaaagacaatgaaaaagcccatatttctgatgctttggcgggcgacgaaatacaggctttgcaatctgct

SEQ ID NO: 516 - protein, 52 aa, Artificial Sequence
  orf
MKRLWSISAVQKLLHDRPNQARKIPCMRSTFAMPNVCWYSCSSFYESLYSAH

SEQ ID NO: 517 - protein, 81 aa, Artificial Sequence
  orf
MGTIRKKAGVLINSVRLMVRFIPLNTLTTIRLSNGIRTFIFLLYSMNGLTRKNWQKLGTTLPWILISLIFAELKKNQRTRL

SEQ ID NO: 518 - protein, 244 aa, Artificial Sequence
  orf
MTRFGILKHRSKLQEQYLWTQVDFKSEGEDDLSNKALKAATKLKGCGQFLLFRNYYTIDQIKLEKFHVCGQHLLCPMCAGIRAARSMSRYIQRIEELMRQNRHLKPVLITLTVKNGEDLQERFDHLTRSFKTLLARYRDYKKKGRGFNQFCKIDGAFYTTEYTYNDKTKQWHPHIHIFALLNEWIDQEELAETWHDITLDSYIVDIRRVKKKPKNTVIAKLLQRFVSTLLSLAICHWRTHGKPF

SEQ ID NO: 519 - protein, 74 aa, Artificial Sequence
  orf
MSLENTWEAFLTLKDKRLTGSFGSMHGVKVPEKATDEMPKEELPYLELQYRFVFGPKSYYNLEITKDVRPQTKE

SEQ ID NO: 520 - protein, 60 aa, Artificial Sequence
  orf
MRHPGLRVRCEARTLSKPTCIYRTCRGFDPLDPKGRTLPNPSGSAVLPKIPIGEHYEINL

SEQ ID NO: 521 - protein, 138 aa, Artificial Sequence
  orf
MTNHTKYMVHEMGNTFKNLGHLKAVSELEEEMEGNTSQIHSPLKPFSLKVPQHVLESLDFLAETMGLSRSALIVNIFDQYLGQVTYEYLDGYNGLVTPEAPPEVVVSEDLEQRLKNSELSESAKQYLTDGVYHVAFGM

SEQ ID NO: 522 - protein, 61 aa, Artificial Sequence
  orf
MVSITLLLECDMTQMPKEHLEQAKDFFAWIIQEHMKDNRTLTTIKIFKQDGQLKILELSND

SEQ ID NO: 523 - protein, 97 aa, Artificial Sequence
  orf
MIKLEGTLLNTFTQQGGQNKKTGDQFEDRDKVQILGAMDLPNGDVKNELFTLSVENYRDFKDFLNHKISVAVGAMASGRNVIFYVAKGAKPILAEQA

SEQ ID NO: 524 - protein, 278 aa, Artificial Sequence
  orf
MLKKDNEKAHISDALAGDEIQALQSAKADQHRDRITRFGILKHRSKLQEQYLWTQVDFKSEGEDDLSNKALKAATKLKGCGQFLLFRNYYTIDQIKLEKFHVCGQHLLCPMCAGIRAARSMSRYIQRIEELMRQNRHLKPVLITLTVKNGEDLQERFDHLTRSFKTLLARYRDYKKKGRGFNQFCKIDGAFYTTEYTYNDKTKQWHPHIHIFALLNEWIDQEELAETWHDITLDSYIVDIRRVKKKPKNTVIAKLLQRFVSTLLSLAICHWRTHGKPF

SEQ ID NO: 525 - protein, 56 aa, Artificial Sequence
  orf
MFNWPSCLKILMVVKVRLSFMCSWIIQAKKSFACSKCSLGICVMSHSKSNVIDTIC

SEQ ID NO: 526 - protein, 63 aa, Artificial Sequence
  orf
MFFRHLCHVTFQKQRDRHHLLNIVLLILIILNFLNVVLNLQKQQLQEVLLELLNRYSHLNIHK

SEQ ID NO: 527 - protein, 54 aa, Artificial Sequence
  orf
MREGRFENLDDVSSGALRPLGYYWRECSPVALLINSTTKGKEHKNLSDLQEKRS

SEQ ID NO: 528 - protein, 89 aa, Artificial Sequence
  orf
MGIFGSTAEPEGFGKVRPLGSKGSNPLQVLYIQVGLLNVLASHRTRKPGCRMRLLRPYRHSQSHSVVSVVAAPPHPSFLSLWPYVFCYF

SEQ ID NO: 529 - protein, 54 aa, Artificial Sequence
  orf
MSSLRTAPASRDVGCDCFVLIATHKVIRSCRSSQLLLTPHSLVCGLTSFVISRL

SEQ ID NO: 530 - protein, 69 aa, Artificial Sequence
  orf
MKRYCSSRYGSSSFGISSVAFSGTFTPCIEPKEPVRRLSLRVKKASHVFSSDRSLNLRAYLQTSATALL

SEQ ID NO: 531 - protein, 62 aa, Artificial Sequence
  orf
MNLRPQILFLQFRSMFQNSKTCYSIPVLICLSRLQSLYFVARQSIRNMGFFIVFFKQFLSPL

SEQ ID NO: 532 - DNA, 156 bp, Artificial Sequence
  orf
attaaaaggttgtggtcaatttctgctgttcagaaattactacacgatagaccaaatcaagctcgaaaaattccatgtatgcggtcaacatttgctatgcccaatgtgtgctggtattcgtgcagctcgttctatgagtcgttatattcagcgcat

SEQ ID NO: 533 - DNA, 243 bp, Artificial Sequence
  orf
atcgggactataagaaaaaaggcaggggttttaatcaattctgtaagattgatggtgcgttttataccactgaatacacttacaacgataagactaagcaatggcatccgcacattcatatttttgctttactcaatgaatggattgaccaggaagaattggcagaaacttggcacgacattaccctggattcttatatcgttgatattcgcagagttaaaaaaaaaccaaagaacacggtta

SEQ ID NO: 534 - DNA, 732 bp, Artificial Sequence
  orf
ataacacgttttggaattttgaaacatagatctaaactgcaagaacaatatttgtggacgcaggttgatttcaaatccgaaggcgaagacgacctatctaataaggctcttaaggctgcaacaaaattaaaaggttgtggtcaatttctgctgttcagaaattactacacgatagaccaaatcaagctcgaaaaattccatgtatgcggtcaacatttgctatgcccaatgtgtgctggtattcgtgcagctcgttctatgagtcgttatattcagcgcattgaagaattgatgcgtcagaatcgccatttaaagcccgtattgatcactttaacggtaaagaatggtgaagacctgcaagaacgctttgatcaccttacacgctcatttaagacccttttagctcgttatcgggactataagaaaaaaggcaggggttttaatcaattctgtaagattgatggtgcgttttataccactgaatacacttacaacgataagactaagcaatggcatccgcacattcatatttttgctttactcaatgaatggattgaccaggaagaattggcagaaacttggcacgacattaccctggattcttatatcgttgatattcgcagagttaaaaaaaaaccaaagaacacggttatagcaaagctgttgcagaggtttgtaagtacgctcttaagtttagcgatctgtcactggagaacacatgggaagcctttt

SEQ ID NO: 535 - DNA, 222 bp, Artificial Sequence
  orf
ctgtcactggagaacacatgggaagcctttttgactcttaaagataaacgccttacaggttctttcggttctatgcacggtgtaaaagtccctgaaaaagctacagacgaaatgccaaaagaagaactcccatatctagagctgcagtaccgtttcgtatttggtccgaaatcttattacaacctagaaataacaaaagacgtaaggccacaaactaaggaa

SEQ ID NO: 536 - DNA, 180 bp, Artificial Sequence
  orf
atccgacatcccggcttgcgggtgcggtgcgaagcgaggacgttaagcaagcctacctgtatatacagaacttgtagggggttcgaccccttagaccccaaagggcgcactttaccaaacccttcaggttcggcagtgctcccaaaaattcccattggagaacattatgaaattaatcta

SEQ ID NO: 537 - DNA, 414 bp, Artificial Sequence
  orf
attaccaaccacacaaaatatatggtacatgaaatgggtaatacatttaaaaatcttggtcatttaaaagctgtatcagaattagaagaggaaatggagggtaatacctctcaaattcattcacctctaaaacctttttctcttaaagttccacaacacgttcttgagtctttggactttttggctgaaacaatgggtcttagccgtagtgctttaatcgttaatatttttgatcaatatcttggtcaagttacttatgaatatttagatggctataacggtttagtaactccagaagcacctcctgaagttgttgtttctgaagatttagaacaacgtttaaaaaattcagaattatcagaatcagcaaaacaatatttaacagatggtgtctatcacgttgcttttggaatg

SEQ ID NO: 538 - DNA, 183 bp, Artificial Sequence
  orf
atggtgtctatcacgttgcttttggaatgtgacatgacacaaatgcctaaagaacatttagaacaagcgaaagacttttttgcttggattatccaagaacacatgaaagacaaccgaaccttaacaaccatcaaaatctttaaacaagatggccaattaaaaattttggagttatcaaatgat

SEQ ID NO: 539 - DNA, 291 bp, Artificial Sequence
  orf
atgattaaattagaagggacattgttaaacacctttactcaacaaggcggacaaaataagaaaactggtgatcagtttgaagaccgtgacaaagtacaaattctaggcgctatggatctgccaaacggtgacgttaaaaatgaactttttacgttatctgtagagaattatcgggactttaaagacttcctaaatcacaaaatttctgttgcagttggtgcaatggcaagtggacgtaatgtgattttttatgttgctaaaggcgcaaagcctattttagccgaacaagcg

SEQ ID NO: 540 - DNA, 834 bp, Artificial Sequence
  orf
ttgcttaaaaaagacaatgaaaaagcccatatttctgatgctttggcgggcgacgaaatacaggctttgcaatctgctaaggcagatcaacacagggatagaataacacgttttggaattttgaaacatagatctaaactgcaagaacaatatttgtggacgcaggttgatttcaaatccgaaggcgaagacgacctatctaataaggctcttaaggctgcaacaaaattaaaaggttgtggtcaatttctgctgttcagaaattactacacgatagaccaaatcaagctcgaaaaattccatgtatgcggtcaacatttgctatgcccaatgtgtgctggtattcgtgcagctcgttctatgagtcgttatattcagcgcattgaagaattgatgcgtcagaatcgccatttaaagcccgtattgatcactttaacggtaaagaatggtgaagacctgcaagaacgctttgatcaccttacacgctcatttaagacccttttagctcgttatcgggactataagaaaaaaggcaggggttttaatcaattctgtaagattgatggtgcgttttataccactgaatacacttacaacgataagactaagcaatggcatccgcacattcatatttttgctttactcaatgaatggattgaccaggaagaattggcagaaacttggcacgacattaccctggattcttatatcgttgatattcgcagagttaaaaaaaaaccaaagaacacggttatagcaaagctgttgcagaggtttgtaagtacgctcttaagtttagcgatctgtcactggagaacacatgggaagcctttt

SEQ ID NO: 541 - DNA, 168 bp, Artificial Sequence
  orf
atttttaattggccatcttgtttaaagattttgatggttgttaaggttcggttgtctttcatgtgttcttggataatccaagcaaaaaagtctttcgcttgttctaaatgttctttaggcatttgtgtcatgtcacattccaaaagcaacgtgatagacaccatctgt

SEQ ID NO: 542 - DNA, 189 bp, Artificial Sequence
  orf
atgttctttaggcatttgtgtcatgtcacattccaaaagcaacgtgatagacaccatctgttaaatattgttttgctgattctgataattctgaattttttaaacgttgttctaaatcttcagaaacaacaacttcaggaggtgcttctggagttactaaaccgttatagccatctaaatattcataag

SEQ ID NO: 543 - DNA, 162 bp, Artificial Sequence
  orf
atcagagaaggaaggtttgagaatttagatgatgtcagttcgggggcactccgccccctcggttactactggagagaatgttctccagtagctttgttaattaattccaccaccaagggcaaagaacataagaacttaagcgatcttcaagaaaaacgtagt

SEQ ID NO: 544 - DNA, 267 bp, Artificial Sequence
  orf
atgggaatttttgggagcactgccgaacctgaagggtttggtaaagtgcgccctttggggtctaaggggtcgaaccccctacaagttctgtatatacaggtaggcttgcttaacgtcctcgcttcgcaccgcacccgcaagccgggatgtcggatgcgattgcttcgtccttatcgccactcacaaagtcattcggtcgtgtcggtcgtcgcagctcctcctcacccctcattccttagtttgtggccttacgtcttttgttatttc

SEQ ID NO: 545 - DNA, 162 bp, Artificial Sequence
  orf
acgtcctcgcttcgcaccgcacccgcaagccgggatgtcggatgcgattgcttcgtccttatcgccactcacaaagtcattcggtcgtgtcggtcgtcgcagctcctcctcacccctcattccttagtttgtggccttacgtcttttgttatttctaggttg

SEQ ID NO: 546 - DNA, 207 bp, Artificial Sequence
  orf
acgaaacggtactgcagctctagatatgggagttcttcttttggcatttcgtctgtagctttttcagggacttttacaccgtgcatagaaccgaaagaacctgtaaggcgtttatctttaagagtcaaaaaggcttcccatgtgttctccagtgacagatcgctaaacttaagagcgtacttacaaacctctgcaacagctttgcta

SEQ ID NO: 547 - DNA, 186 bp, Artificial Sequence
  orf
atcaacctgcgtccacaaatattgttcttgcagtttagatctatgtttcaaaattccaaaacgtgttattctatccctgtgttgatctgccttagcagattgcaaagcctgtatttcgtcgcccgccaaagcatcagaaatatgggctttttcattgtcttttttaagcaattcttgtccccactt

SEQ ID NO: 548 - DNA, 2590 bp, Artificial Sequence
  isolate
caacacagggatagaataacacgttttggtattttgaaacacaggtcgaagcaacaagaaaactttttgtggacgctagcaaaattcaaagaaaactacccaatagactcgccaaatgaagaaactgtaaaggccctaaaatctgcacagaagttaaacggctgcggtaattttttgctgttcaaaaatctttacacaatagatcaaactaagctcacaaaatttcatgtctgtaatcaacatttgctttgcccattttgtgcaggtattcgtgcatctaaagcaattcaaaaatatacagaacgtgttgatgaagtattaaagaaaaatcgtaagctcaagcccgttctaatcacgctcactgtgaaaaacggtactgacctaagtgaacggtctgcacacctcataaaatcgcttagaacgcttattaagcgccgtcaggactataataaaaaaggtcgtggttttaacgaattttgtaaaattaatggtgcgatgtactcgtacgaaaatacttacaatgaacaaaccaaagaatggcatccgcacgttcatatgtttgctcctttggatgattggatagaccaggaactactttctgaaacatggcatgaaatcactggtgattcatttgttgtggacattcgaaaagttaaaaaacaaaaagacttaggctatgcaaaagctgctgctgaggtgtgtaaatacgctcttaagtttggggatctaacggtcgaaaatacatgggaagcttttaaagtcctaaaaggtaagcgcttaagcggtgcatttggctctctttatggtgtcaaaattcctgaaaaccttgctgacgatatgccagaagaaaaagatcttccgtatttagagatgctctataagttcgtctttggaaaacactcttactacaatctcgaaattactagacacgtaggaccgcaggcgggagagcgcgcggagttaggaggagttgcgacgacgcccgacgagagatatagcgcgggcggttctagtgtagacgatttgagtacggaggcaggtggagggctcgcaccactgcgacgtacgaaaagaggacgcaaaaaacagcactggcaattaccaccagtaacgagagttagagtcaaaaaacgaatcaaaagatgggatggttatttgtgtgttatacacatgtaaaatcatgaacctatgataaaattctagtcgctgaatagtaggacggcaatcctactacccagctaaccaaaacgatctattgaggagatcatcatggctgtgccacatactacattgcttggctatctaggcaaacacatcacattcaaagttccttgtccaattgagttcaataactcaggttttgatcagctttcagggaaagtgatcgctgttaatattgaattaaatgaaaaacattctttgtgtgttatgcatgacgaatcaacagattcggctgattattacgaatttgaaaaaatgctaatacagccctaatttttagatttgtacccgagaggggagacctcgaagtctttatttataaaggtttcggggtttttttatgtctttaacataatgcgtactattgaaaataaggcactaaagagtacttatataatctttagttcccgaacgagtgtctacgagtgacgactccggaataaaaattacaaatccccctttctctgattacaagctcccctttttttcgttctccggtaagaacagagccatcccgaatgggtggcgacccccctattgatttgttcttgattcagtgtatgattcatttgtgaatcacataatcttgttaatgaatcaggtatgaatatggaacctacaaatttagatatttttttaagtgaagttaaaaaaacaggtgaaacgaaattaactggtgttttccaccaagttccatttcgaatgttctctccacactatcgacgtgttgaaggtcttcgcaattatatgaatgagcctcgtaataaagttcttaattctctaattgaaattgctttagatcaggtttttgaaaatctgtctaaggatccagaaatttatgaagctgtaatggccgaagtttcaaaaattgatattggtacgtttgatggctctggagacttagatcgtgattaaactagagggaaccgtgataaatacatttcatcttgaaggtggaaaaaataaaaagggggaagaatacgaagccagtgacaaggtgcagttacttggttcgttggagctacccaatgggcaaattaaaaacgaattgattgatcttaaagtcgaagatgcaagcatctacgatgcattcaaaaataagcttattagcattagttgtggcgcatttcccgcagggaaaaacgtcgtgttttatgttcgaaaaggtgcaaagcctgttttagctgacggtctttgatttatgttaaaaaacagtgagttagcgaagctactcgctgttttttattttacttgatactattcattaagtcggggacaaaatttcatgataaatgacaataaaaaacccctgttatcggacagtttggcgaccggtgataacaagggtttgcaatctgctaaggcagat

SEQ ID NO: 549 - protein, 386 aa, Artificial Sequence
  orf
MTRFGILKHRSKQQENFLWTLAKFKENYPIDSPNEETVKALKSAQKLNGCGNFLLFKNLYTIDQTKLTKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFAPLDDWIDQELLSETWHEITGDSFVVDIRKVKKQKDLGYAKAAAEVCKYALKFGDLTVENTWEAFKVLKGKRLSGAFGSLYGVKIPENLADDMPEEKDLPYLEMLYKFVFGKHSYYNLEITRHVGPQAGERAELGGVATTPDERYSAGGSSVDDLSTEAGGGLAPLRRTKRGRKKQHWQLPPVTRVRVKKRIKRWDGYLCVIHM

SEQ ID NO: 550 - protein, 79 aa, Artificial Sequence
  orf
MEEIIMAVPHTTLLGYLGKHITFKVPCPIEFNNSGFDQLSGKVIAVNIELNEKHSLCVMHDESTDSADYYEFEKMLIQP

SEQ ID NO: 551 - protein, 115 aa, Artificial Sequence
  orf
MCESHNLVNESGMNMEPTNLDIFLSEVKKTGETKLTGVFHQVPFRMFSPHYRRVEGLRNYMNEPRNKVLNSLIEIALDQVFENLSKDPEIYEAVMAEVSKIDIGTFDGSGDLDRD

SEQ ID NO: 552 - protein, 97 aa, Artificial Sequence
  orf
MVIKLEGTVINTFHLEGGKNKKGEEYEASDKVQLLGSLELPNGQIKNELIDLKVEDASIYDAFKNKLISISCGAFPAGKNVVFYVRKGAKPVLADGL

SEQ ID NO: 553 - protein, 419 aa, Artificial Sequence
  orf
MINDNKKPLLSDSLATGDNKGLQSAKADQHRDRITRFGILKHRSKQQENFLWTLAKFKENYPIDSPNEETVKALKSAQKLNGCGNFLLFKNLYTIDQTKLTKFHVCNQHLLCPFCAGIRASKAIQKYTERVDEVLKKNRKLKPVLITLTVKNGTDLSERSAHLIKSLRTLIKRRQDYNKKGRGFNEFCKINGAMYSYENTYNEQTKEWHPHVHMFAPLDDWIDQELLSETWHEITGDSFVVDIRKVKKQKDLGYAKAAAEVCKYALKFGDLTVENTWEAFKVLKGKRLSGAFGSLYGVKIPENLADDMPEEKDLPYLEMLYKFVFGKHSYYNLEITRHVGPQAGERAELGGVATTPDERYSAGGSSVDDLSTEAGGGLAPLRRTKRGRKKQHWQLPPVTRVRVKKRIKRWDGYLCVIHM

SEQ ID NO: 554 - protein, 85 aa, Artificial Sequence
  orf
MRNGTWWKTPVNFVSPVFLTSLKKISKFVGSIFIPDSLTRLCDSQMNHTLNQEQINRGVATHSGWLCSYRRTKKRGACNQRKGDL

SEQ ID NO: 555 - protein, 50 aa, Artificial Sequence
  orf
MKNKSIGGSPPIRDGSVLTGERKKGELVIRERGICNFYSGVVTRRHSFGN

SEQ ID NO: 556 - protein, 77 aa, Artificial Sequence
  orf
MIRFLTLTLVTGGNCQCCFLRPLFVRRSGASPPPASVLKSSTLEPPALYLSSGVVATPPNSARSPACGPTCLVISRL

SEQ ID NO: 557 - protein, 68 aa, Artificial Sequence
  orf
MVYPIIQRSKHMNVRMPFFGLFIVSIFVRVHRTINFTKFVKTTTFFIIVLTALNKRSKRFYEVCRPFT

SEQ ID NO: 558 - protein, 52 aa, Artificial Sequence
  orf
MNTFCIFLNCFRCTNTCTKWAKQMLITDMKFCELSLIYCVKIFEQQKITAAV

SEQ ID NO: 559 - DNA, 1158 bp, Artificial Sequence
  orf
ataacacgttttggtattttgaaacacaggtcgaagcaacaagaaaactttttgtggacgctagcaaaattcaaagaaaactacccaatagactcgccaaatgaagaaactgtaaaggccctaaaatctgcacagaagttaaacggctgcggtaattttttgctgttcaaaaatctttacacaatagatcaaactaagctcacaaaatttcatgtctgtaatcaacatttgctttgcccattttgtgcaggtattcgtgcatctaaagcaattcaaaaatatacagaacgtgttgatgaagtattaaagaaaaatcgtaagctcaagcccgttctaatcacgctcactgtgaaaaacggtactgacctaagtgaacggtctgcacacctcataaaatcgcttagaacgcttattaagcgccgtcaggactataataaaaaaggtcgtggttttaacgaattttgtaaaattaatggtgcgatgtactcgtacgaaaatacttacaatgaacaaaccaaagaatggcatccgcacgttcatatgtttgctcctttggatgattggatagaccaggaactactttctgaaacatggcatgaaatcactggtgattcatttgttgtggacattcgaaaagttaaaaaacaaaaagacttaggctatgcaaaagctgctgctgaggtgtgtaaatacgctcttaagtttggggatctaacggtcgaaaatacatgggaagcttttaaagtcctaaaaggtaagcgcttaagcggtgcatttggctctctttatggtgtcaaaattcctgaaaaccttgctgacgatatgccagaagaaaaagatcttccgtatttagagatgctctataagttcgtctttggaaaacactcttactacaatctcgaaattactagacacgtaggaccgcaggcgggagagcgcgcggagttaggaggagttgcgacgacgcccgacgagagatatagcgcgggcggttctagtgtagacgatttgagtacggaggcaggtggagggctcgcaccactgcgacgtacgaaaagaggacgcaaaaaacagcactggcaattaccaccagtaacgagagttagagtcaaaaaacgaatcaaaagatgggatggttatttgtgtgttatacacatg

SEQ ID NO: 560 - DNA, 237 bp, Artificial Sequence
  orf
attgaggagatcatcatggctgtgccacatactacattgcttggctatctaggcaaacacatcacattcaaagttccttgtccaattgagttcaataactcaggttttgatcagctttcagggaaagtgatcgctgttaatattgaattaaatgaaaaacattctttgtgtgttatgcatgacgaatcaacagattcggctgattattacgaatttgaaaaaatgctaatacagccc

SEQ ID NO: 561 - DNA, 345 bp, Artificial Sequence
  orf
atttgtgaatcacataatcttgttaatgaatcaggtatgaatatggaacctacaaatttagatatttttttaagtgaagttaaaaaaacaggtgaaacgaaattaactggtgttttccaccaagttccatttcgaatgttctctccacactatcgacgtgttgaaggtcttcgcaattatatgaatgagcctcgtaataaagttcttaattctctaattgaaattgctttagatcaggtttttgaaaatctgtctaaggatccagaaatttatgaagctgtaatggccgaagtttcaaaaattgatattggtacgtttgatggctctggagacttagatcgtgat

SEQ ID NO: 562 - DNA, 291 bp, Artificial Sequence
  orf
atcgtgattaaactagagggaaccgtgataaatacatttcatcttgaaggtggaaaaaataaaaagggggaagaatacgaagccagtgacaaggtgcagttacttggttcgttggagctacccaatgggcaaattaaaaacgaattgattgatcttaaagtcgaagatgcaagcatctacgatgcattcaaaaataagcttattagcattagttgtggcgcatttcccgcagggaaaaacgtcgtgttttatgttcgaaaaggtgcaaagcctgttttagctgacggtctt

SEQ ID NO: 563 - DNA, 1257 bp, Artificial Sequence
  orf
atgataaatgacaataaaaaacccctgttatcggacagtttggcgaccggtgataacaagggtttgcaatctgctaaggcagatcaacacagggatagaataacacgttttggtattttgaaacacaggtcgaagcaacaagaaaactttttgtggacgctagcaaaattcaaagaaaactacccaatagactcgccaaatgaagaaactgtaaaggccctaaaatctgcacagaagttaaacggctgcggtaattttttgctgttcaaaaatctttacacaatagatcaaactaagctcacaaaatttcatgtctgtaatcaacatttgctttgcccattttgtgcaggtattcgtgcatctaaagcaattcaaaaatatacagaacgtgttgatgaagtattaaagaaaaatcgtaagctcaagcccgttctaatcacgctcactgtgaaaaacggtactgacctaagtgaacggtctgcacacctcataaaatcgcttagaacgcttattaagcgccgtcaggactataataaaaaaggtcgtggttttaacgaattttgtaaaattaatggtgcgatgtactcgtacgaaaatacttacaatgaacaaaccaaagaatggcatccgcacgttcatatgtttgctcctttggatgattggatagaccaggaactactttctgaaacatggcatgaaatcactggtgattcatttgttgtggacattcgaaaagttaaaaaacaaaaagacttaggctatgcaaaagctgctgctgaggtgtgtaaatacgctcttaagtttggggatctaacggtcgaaaatacatgggaagcttttaaagtcctaaaaggtaagcgcttaagcggtgcatttggctctctttatggtgtcaaaattcctgaaaaccttgctgacgatatgccagaagaaaaagatcttccgtatttagagatgctctataagttcgtctttggaaaacactcttactacaatctcgaaattactagacacgtaggaccgcaggcgggagagcgcgcggagttaggaggagttgcgacgacgcccgacgagagatatagcgcgggcggttctagtgtagacgatttgagtacggaggcaggtggagggctcgcaccactgcgacgtacgaaaagaggacgcaaaaaacagcactggcaattaccaccagtaacgagagttagagtcaaaaaacgaatcaaaagatgggatggttatttgtgtgttatacacatg

SEQ ID NO: 564 - DNA, 255 bp, Artificial Sequence
  orf
attcgaaatggaacttggtggaaaacaccagttaatttcgtttcacctgtttttttaacttcacttaaaaaaatatctaaatttgtaggttccatattcatacctgattcattaacaagattatgtgattcacaaatgaatcatacactgaatcaagaacaaatcaataggggggtcgccacccattcgggatggctctgttcttaccggagaacgaaaaaaaggggagcttgtaatcagagaaagggggatttg

SEQ ID NO: 565 - DNA, 150 bp, Artificial Sequence
  orf
atcaagaacaaatcaataggggggtcgccacccattcgggatggctctgttcttaccggagaacgaaaaaaaggggagcttgtaatcagagaaagggggatttgtaatttttattccggagtcgtcactcgtagacactcgttcgggaac

SEQ ID NO: 566 - DNA, 231 bp, Artificial Sequence
  orf
ttgattcgttttttgactctaactctcgttactggtggtaattgccagtgctgttttttgcgtcctcttttcgtacgtcgcagtggtgcgagccctccacctgcctccgtactcaaatcgtctacactagaaccgcccgcgctatatctctcgtcgggcgtcgtcgcaactcctcctaactccgcgcgctctcccgcctgcggtcctacgtgtctagtaatttcgagattg

SEQ ID NO: 567 - DNA, 204 bp, Artificial Sequence
  orf
ctggtctatccaatcatccaaaggagcaaacatatgaacgtgcggatgccattctttggtttgttcattgtaagtattttcgtacgagtacatcgcaccattaattttacaaaattcgttaaaaccacgaccttttttattatagtcctgacggcgcttaataagcgttctaagcgattttatgaggtgtgcagaccgttcact

SEQ ID NO: 568 - DNA, 156 bp, Artificial Sequence
  orf
atcaacacgttctgtatatttttgaattgctttagatgcacgaatacctgcacaaaatgggcaaagcaaatgttgattacagacatgaaattttgtgagcttagtttgatctattgtgtaaagatttttgaacagcaaaaaattaccgcagccgtt

SEQ ID NO: 569 - DNA, 2952 bp, Artificial Sequence
  isolate
caacacagggatagaataacacgttttgggattttgaaacatagagcgaagcaacaagaaaactttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactatgctctcaaaatttcatgtatgtggacagcatttgttatgtcctttttgtgctggtattcgtgcatctaaagcaattcaaaaatatactgaacgtgttgatgaagtgcttaagaaaaatcgcaagctcaagcctgttttagtcacgtttacggtgaagaatggaaccgacttaggggaaagatcatcccacctcataaaatcgtttagaacgcttctgaagcgtcgtagggactatattaaaaaaggccgtgggtttaatgagttctgtaagatcaatggggctatgtattcttatgaaaacacgtataacgagcaaacaaatgaatggcatccacacttacatatgtttgcacttttggatgattggatagaccaggaagagctttcacagtattggcattccatcactggtgattcaatgattgtagatattcgccgggtcaaaaaagaaaaaggcttaggctattcaaaggctgctgcggaagtatgtaaatatgctcttaaattcggtgatttgtctgtccaaaatacttgggaagctttcaaagtcttaaaaggtaagcgtttaagtggtgcatttggttgtttatggggcgtaaaaattcctgaaaatcttgctgatgatatgcctgatgaaaaagatcttccttacttagaaatgctctataaattcgtatttggaaaaccgtcttactatgacttaactatgacacgacatgtagagccacaaaataaggacaacgccgacgacgaggagcaggaacgtcgacgagaaggagaaggcggacgcaaattagtggcgaatgaggacgatgtgaggagtgacgaggagcgtgccgaccgacggacacgaacgacacgaggacgaaaaaaacaacactggcaagtacctccagtaacgagggtacgagtgaaaaaacggattaaaagatgggatggttatttgtgtgttatgcacatgtaaaatcataaacctatgataaaattctagtcgctgaatagtcggattgccgtccaactacccagctaaccacaacgatctaagaaggagatcatcatgactgtagcgaatactaatgctttaaattatttaggcaagtatgtttcatttaatcacgaagatatttttgaaatttacggagttatctccaatgtaattttcaacatggacgggaccattgaactgtccattggatgggatgatttttataatttctctgaaatgactaatttaaaagttttaggagaaattagtctttattgactttagatttgaatttaccaatgcccgaaaatacatatttttgggcatttttttgtctgtttcttgagaaacgcgtaagcgtgcattctgaaaaaataaacattgagattggcgaagagattcggggcgctgggcgtggggtcgggatgtcactcgatgtcccgccctgtgactggcgacacgagatattcgacaatctcactttagaaacaatatcagagggcacggagtgtgcccgaactgaagaaataaaaattaaaaactacctttccctgattacaagctcccttttgattttctccgggataaacagagccatccgtatggtggcgatagattgacattctaattaaaatctataatattctataaacaatctagtagaatctaatcggaatctaaaatatgttatttaaatctcttggaattgcttccgctactaataatgcttctaactctgaggatgctaagcctgtgactattcgtttaactgatggtgaggttcaactttatgatgctgtagccaaggctatggggttaactcgtcaagattttcttgcccatttaattcgttctaattttaaacaagcattaaccgattttttaataggttacacggaatcatcaccatctatctctttaaaagaattaatttttttgggaactgacaatgaagttgttcgaaataaagttgattcaatgcttcgatctatttctgcacaactaatggaagatgatgaaaaggcaattcaagaacatcttcaaaatgaaggtaattggtatgaatatgctacccaacgtaaaggtattttccctgaacctgttaaaccagttgaggtaaaaaatgattaaacctcaagtcattagtaacactccaatggaatctcttgaggaagcttgcgattactttaagcaagttcttcaggaacatctcgataaaaaacgtgacatcacattatttgccatcgctgttgtaggtggtcgtttaaaagtcgtagagggattaccaaaatgatcaaattagaaggcacaattattaatgtttttacccaacaaggtggtaaagataaaaaaacaggcgaagcattcgcagatcgggacaagattcaattattaggtgagtttgaactccccaatggtgataaaaaaaatgaattaatagaccttactgttgaaaatggtaaggaatatgagggtatgaaaaacaagcgtgtgagcattccctgtggtgctcttgcttcaggtcgaaatgtgattttttatgtcgcaaaaggcgcaacacctaaacttttaaacacagtttgatactcgttaaaaaacagcgacttacgaagtaagttagctgttttttcttttacttgatactattaattaaagtggggacaaaaattgactgtaaaagacaataaaaaagcccatatttctgatgctttggcgggcgacgaaatacaggctttgcaatctgctaaggcagat

SEQ ID NO: 570 - protein, 386 aa, Artificial Sequence
  orf
MTRFGILKHRAKQQENFLWTLAKFKENYPKDAPSEESTRAIKSAQKLKECGNFLLFKNFYTIDQTMLSKFHVCGQHLLCPFCAGIRASKAIQKYTERVDEVLKKNRKLKPVLVTFTVKNGTDLGERSSHLIKSFRTLLKRRRDYIKKGRGFNEFCKINGAMYSYENTYNEQTNEWHPHLHMFALLDDWIDQEELSQYWHSITGDSMIVDIRRVKKEKGLGYSKAAAEVCKYALKFGDLSVQNTWEAFKVLKGKRLSGAFGCLWGVKIPENLADDMPDEKDLPYLEMLYKFVFGKPSYYDLTMTRHVEPQNKDNADDEEQERRREGEGGRKLVANEDDVRSDEERADRRTRTTRGRKKQHWQVPPVTRVRVKKRIKRWDGYLCVMHM

SEQ ID NO: 571 - protein, 70 aa, Artificial Sequence
  orf
MIMTVANTNALNYLGKYVSFNHEDIFEIYGVISNVIFNMDGTIELSIGWDDFYNFSEMTNLKVLGEISLY

SEQ ID NO: 572 - protein, 114 aa, Artificial Sequence
  orf
MTLDLNLPMPENTYFWAFFCLFLEKRVSVHSEKINIEIGEEIRGAGRGVGMSLDVPPCDWRHEIFDNLTLETISEGTECARTEEIKIKNYLSLITSSLLIFSGINRAIRMVAID

SEQ ID NO: 573 - protein, 148 aa, Artificial Sequence
  orf
MLFKSLGIASATNNASNSEDAKPVTIRLTDGEVQLYDAVAKAMGLTRQDFLAHLIRSNFKQALTDFLIGYTESSPSISLKELIFLGTDNEVVRNKVDSMLRSISAQLMEDDEKAIQEHLQNEGNWYEYATQRKGIFPEPVKPVEVKND

SEQ ID NO: 574 - protein, 56 aa, Artificial Sequence
  orf
MIKPQVISNTPMESLEEACDYFKQVLQEHLDKKRDITLFAIAVVGGRLKVVEGLPK

SEQ ID NO: 575 - protein, 116 aa, Artificial Sequence
  orf
MICHRCCRWSFKSRRGITKMIKLEGTIINVFTQQGGKDKKTGEAFADRDKIQLLGEFELPNGDKKNELIDLTVENGKEYEGMKNKRVSIPCGALASGRNVIFYVAKGATPKLLNTV

SEQ ID NO: 576 - protein, 420 aa, Artificial Sequence
  orf
MTVKDNKKAHISDALAGDEIQALQSAKADQHRDRITRFGILKHRAKQQENFLWTLAKFKENYPKDAPSEESTRAIKSAQKLKECGNFLLFKNFYTIDQTMLSKFHVCGQHLLCPFCAGIRASKAIQKYTERVDEVLKKNRKLKPVLVTFTVKNGTDLGERSSHLIKSFRTLLKRRRDYIKKGRGFNEFCKINGAMYSYENTYNEQTNEWHPHLHMFALLDDWIDQEELSQYWHSITGDSMIVDIRRVKKEKGLGYSKAAAEVCKYALKFGDLSVQNTWEAFKVLKGKRLSGAFGCLWGVKIPENLADDMPDEKDLPYLEMLYKFVFGKPSYYDLTMTRHVEPQNKDNADDEEQERRREGEGGRKLVANEDDVRSDEERADRRTRTTRGRKKQHWQVPPVTRVRVKKRIKRWDGYLCVMHM

SEQ ID NO: 577 - protein, 51 aa, Artificial Sequence
  orf
MLSRSANASPVFLSLPPCWVKTLIIVPSNLIILVIPLRLLNDHLQQRWQIM

SEQ ID NO: 578 - protein, 89 aa, Artificial Sequence
  orf
MFISSVRAHSVPSDIVSKVRLSNISCRQSQGGTSSDIPTPRPAPRISSPISMFIFSECTLTRFSRNRQKNAQKYVFSGIGKFKSKVNKD

SEQ ID NO: 579 - protein, 70 aa, Artificial Sequence
  orf
MSRVASHRAGHRVTSRPHAQRPESLRQSQCLFFQNARLRVSQETDKKMPKNMYFRALVNSNLKSIKTNFS

SEQ ID NO: 580 - protein, 74 aa, Artificial Sequence
  orf
MDGNPTIQRLEFYHRFMILHVHNTQITIPSFNPFFHSYPRYWRYLPVLFFSSSCRSCPSVGTLLVTPHIVLIRH

SEQ ID NO: 581 - protein, 63 aa, Artificial Sequence
  orf
MEVLASVVFFVLVSFVSVGRHAPRHSSHRPHSPLICVRLLLLVDVPAPRRRRCPYFVALHVVS

SEQ ID NO: 582 - protein, 76 aa, Artificial Sequence
  orf
MRFFTRTLVTGGTCQCCFFRPRVVRVRRSARSSSLLTSSSFATNLRPPSPSRRRSCSSSSALSLFCGSTCRVIVKS

SEQ ID NO: 583 - protein, 54 aa, Artificial Sequence
  orf
MSSARFSGIFTPHKQPNAPLKRLPFKTLKASQVFWTDKSPNLRAYLHTSAAAFE

SEQ ID NO: 584 - protein, 50 aa, Artificial Sequence
  orf
MTRRISTIIESPVMECQYCESSSWSIQSSKSANICKCGCHSFVCSLYVFS

SEQ ID NO: 585 - protein, 51 aa, Artificial Sequence
  orf
MWMPFICLLVIRVFIRIHSPIDLTELIKPTAFFNIVPTTLQKRSKRFYEVG

SEQ ID NO: 586 - protein, 84 aa, Artificial Sequence
  orf
MCRFNCSCAFFTWSIFRIVFFEFCQSPQKVFLLLRSMFQNPKTCYSIPVLICLSRLQSLYFVARQSIRNMGFFIVFYSQFLSPL

SEQ ID NO: 587 - DNA, 1158 bp, Artificial Sequence
  orf
ataacacgttttgggattttgaaacatagagcgaagcaacaagaaaactttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactatgctctcaaaatttcatgtatgtggacagcatttgttatgtcctttttgtgctggtattcgtgcatctaaagcaattcaaaaatatactgaacgtgttgatgaagtgcttaagaaaaatcgcaagctcaagcctgttttagtcacgtttacggtgaagaatggaaccgacttaggggaaagatcatcccacctcataaaatcgtttagaacgcttctgaagcgtcgtagggactatattaaaaaaggccgtgggtttaatgagttctgtaagatcaatggggctatgtattcttatgaaaacacgtataacgagcaaacaaatgaatggcatccacacttacatatgtttgcacttttggatgattggatagaccaggaagagctttcacagtattggcattccatcactggtgattcaatgattgtagatattcgccgggtcaaaaaagaaaaaggcttaggctattcaaaggctgctgcggaagtatgtaaatatgctcttaaattcggtgatttgtctgtccaaaatacttgggaagctttcaaagtcttaaaaggtaagcgtttaagtggtgcatttggttgtttatggggcgtaaaaattcctgaaaatcttgctgatgatatgcctgatgaaaaagatcttccttacttagaaatgctctataaattcgtatttggaaaaccgtcttactatgacttaactatgacacgacatgtagagccacaaaataaggacaacgccgacgacgaggagcaggaacgtcgacgagaaggagaaggcggacgcaaattagtggcgaatgaggacgatgtgaggagtgacgaggagcgtgccgaccgacggacacgaacgacacgaggacgaaaaaaacaacactggcaagtacctccagtaacgagggtacgagtgaaaaaacggattaaaagatgggatggttatttgtgtgttatgcacatg

SEQ ID NO: 588 - DNA, 210 bp, Artificial Sequence
  orf
atcatcatgactgtagcgaatactaatgctttaaattatttaggcaagtatgtttcatttaatcacgaagatatttttgaaatttacggagttatctccaatgtaattttcaacatggacgggaccattgaactgtccattggatgggatgatttttataatttctctgaaatgactaatttaaaagttttaggagaaattagtctttat

SEQ ID NO: 589 - DNA, 342 bp, Artificial Sequence
  orf
ttgactttagatttgaatttaccaatgcccgaaaatacatatttttgggcatttttttgtctgtttcttgagaaacgcgtaagcgtgcattctgaaaaaataaacattgagattggcgaagagattcggggcgctgggcgtggggtcgggatgtcactcgatgtcccgccctgtgactggcgacacgagatattcgacaatctcactttagaaacaatatcagagggcacggagtgtgcccgaactgaagaaataaaaattaaaaactacctttccctgattacaagctcccttttgattttctccgggataaacagagccatccgtatggtggcgatagat

SEQ ID NO: 590 - DNA, 444 bp, Artificial Sequence
  orf
atgttatttaaatctcttggaattgcttccgctactaataatgcttctaactctgaggatgctaagcctgtgactattcgtttaactgatggtgaggttcaactttatgatgctgtagccaaggctatggggttaactcgtcaagattttcttgcccatttaattcgttctaattttaaacaagcattaaccgattttttaataggttacacggaatcatcaccatctatctctttaaaagaattaatttttttgggaactgacaatgaagttgttcgaaataaagttgattcaatgcttcgatctatttctgcacaactaatggaagatgatgaaaaggcaattcaagaacatcttcaaaatgaaggtaattggtatgaatatgctacccaacgtaaaggtattttccctgaacctgttaaaccagttgaggtaaaaaatgat

SEQ ID NO: 591 - DNA, 168 bp, Artificial Sequence
  orf
atgattaaacctcaagtcattagtaacactccaatggaatctcttgaggaagcttgcgattactttaagcaagttcttcaggaacatctcgataaaaaacgtgacatcacattatttgccatcgctgttgtaggtggtcgtttaaaagtcgtagagggattaccaaaa

SEQ ID NO: 592 - DNA, 348 bp, Artificial Sequence
  orf
attatttgccatcgctgttgtaggtggtcgtttaaaagtcgtagagggattaccaaaatgatcaaattagaaggcacaattattaatgtttttacccaacaaggtggtaaagataaaaaaacaggcgaagcattcgcagatcgggacaagattcaattattaggtgagtttgaactccccaatggtgataaaaaaaatgaattaatagaccttactgttgaaaatggtaaggaatatgagggtatgaaaaacaagcgtgtgagcattccctgtggtgctcttgcttcaggtcgaaatgtgattttttatgtcgcaaaaggcgcaacacctaaacttttaaacacagtt

SEQ ID NO: 593 - DNA, 1260 bp, Artificial Sequence
  orf
ttgactgtaaaagacaataaaaaagcccatatttctgatgctttggcgggcgacgaaatacaggctttgcaatctgctaaggcagatcaacacagggatagaataacacgttttgggattttgaaacatagagcgaagcaacaagaaaactttttgtggactctggcaaaattcaaagaaaactatcctaaagatgctccaagtgaagaaagcacaagagcaattaaatctgcacagaaattaaaagagtgcggaaatttcttactattcaaaaatttttacacaatagaccaaactatgctctcaaaatttcatgtatgtggacagcatttgttatgtcctttttgtgctggtattcgtgcatctaaagcaattcaaaaatatactgaacgtgttgatgaagtgcttaagaaaaatcgcaagctcaagcctgttttagtcacgtttacggtgaagaatggaaccgacttaggggaaagatcatcccacctcataaaatcgtttagaacgcttctgaagcgtcgtagggactatattaaaaaaggccgtgggtttaatgagttctgtaagatcaatggggctatgtattcttatgaaaacacgtataacgagcaaacaaatgaatggcatccacacttacatatgtttgcacttttggatgattggatagaccaggaagagctttcacagtattggcattccatcactggtgattcaatgattgtagatattcgccgggtcaaaaaagaaaaaggcttaggctattcaaaggctgctgcggaagtatgtaaatatgctcttaaattcggtgatttgtctgtccaaaatacttgggaagctttcaaagtcttaaaaggtaagcgtttaagtggtgcatttggttgtttatggggcgtaaaaattcctgaaaatcttgctgatgatatgcctgatgaaaaagatcttccttacttagaaatgctctataaattcgtatttggaaaaccgtcttactatgacttaactatgacacgacatgtagagccacaaaataaggacaacgccgacgacgaggagcaggaacgtcgacgagaaggagaaggcggacgcaaattagtggcgaatgaggacgatgtgaggagtgacgaggagcgtgccgaccgacggacacgaacgacacgaggacgaaaaaaacaacactggcaagtacctccagtaacgagggtacgagtgaaaaaacggattaaaagatgggatggttatttgtgtgttatgcacatg

SEQ ID NO: 594 - DNA, 153 bp, Artificial Sequence
  orf
atcttgtcccgatctgcgaatgcttcgcctgtttttttatctttaccaccttgttgggtaaaaacattaataattgtgccttctaatttgatcattttggtaatccctctacgacttttaaacgaccacctacaacagcgatggcaaataatg

SEQ ID NO: 595 - DNA, 267 bp, Artificial Sequence
  orf
atttttatttcttcagttcgggcacactccgtgccctctgatattgtttctaaagtgagattgtcgaatatctcgtgtcgccagtcacagggcgggacatcgagtgacatcccgaccccacgcccagcgccccgaatctcttcgccaatctcaatgtttattttttcagaatgcacgcttacgcgtttctcaagaaacagacaaaaaaatgcccaaaaatatgtattttcgggcattggtaaattcaaatctaaagtcaataaagac

SEQ ID NO: 596 - DNA, 210 bp, Artificial Sequence
  orf
atatctcgtgtcgccagtcacagggcgggacatcgagtgacatcccgaccccacgcccagcgccccgaatctcttcgccaatctcaatgtttattttttcagaatgcacgcttacgcgtttctcaagaaacagacaaaaaaatgcccaaaaatatgtattttcgggcattggtaaattcaaatctaaagtcaataaagactaatttctcc

SEQ ID NO: 597 - DNA, 222 bp, Artificial Sequence
  orf
ttggacggcaatccgactattcagcgactagaattttatcataggtttatgattttacatgtgcataacacacaaataaccatcccatcttttaatccgttttttcactcgtaccctcgttactggaggtacttgccagtgttgtttttttcgtcctcgtgtcgttcgtgtccgtcggtcggcacgctcctcgtcactcctcacatcgtcctcattcgccac

SEQ ID NO: 598 - DNA, 189 bp, Artificial Sequence
  orf
ctggaggtacttgccagtgttgtttttttcgtcctcgtgtcgttcgtgtccgtcggtcggcacgctcctcgtcactcctcacatcgtcctcattcgccactaatttgcgtccgccttctccttctcgtcgacgttcctgctcctcgtcgtcggcgttgtccttattttgtggctctacatgtcgtgtca

SEQ ID NO: 599 - DNA, 228 bp, Artificial Sequence
  orf
atccgttttttcactcgtaccctcgttactggaggtacttgccagtgttgtttttttcgtcctcgtgtcgttcgtgtccgtcggtcggcacgctcctcgtcactcctcacatcgtcctcattcgccactaatttgcgtccgccttctccttctcgtcgacgttcctgctcctcgtcgtcggcgttgtccttattttgtggctctacatgtcgtgtcatagttaagtca

SEQ ID NO: 600 - DNA, 162 bp, Artificial Sequence
  orf
atatcatcagcaagattttcaggaatttttacgccccataaacaaccaaatgcaccacttaaacgcttaccttttaagactttgaaagcttcccaagtattttggacagacaaatcaccgaatttaagagcatatttacatacttccgcagcagcctttgaa

SEQ ID NO: 601 - DNA, 150 bp, Artificial Sequence
  orf
ttgacccggcgaatatctacaatcattgaatcaccagtgatggaatgccaatactgtgaaagctcttcctggtctatccaatcatccaaaagtgcaaacatatgtaagtgtggatgccattcatttgtttgctcgttatacgtgttttca

SEQ ID NO: 602 - DNA, 153 bp, Artificial Sequence
  orf
gtgtggatgccattcatttgtttgctcgttatacgtgttttcataagaatacatagccccattgatcttacagaactcattaaacccacggccttttttaatatagtccctacgacgcttcagaagcgttctaaacgattttatgaggtggga

SEQ ID NO: 603 - DNA, 252 bp, Artificial Sequence
  orf
ctgtgcagatttaattgctcttgtgctttcttcacttggagcatctttaggatagttttctttgaattttgccagagtccacaaaaagttttcttgttgcttcgctctatgtttcaaaatcccaaaacgtgttattctatccctgtgttgatctgccttagcagattgcaaagcctgtatttcgtcgcccgccaaagcatcagaaatatgggcttttttattgtcttttacagtcaatttttgtccccactt

SEQ ID NO: 604 - DNA, 2234 bp, Artificial Sequence
  isolate
caacacagggatagaataacacgttttgggattttgaaacatagagcgaagctacaagaacaatacttgtggacgcaggttgatttcaaatcagaaggtgaaaatgatctatccaataaggctcttaaggctgcaaccaaattaaaggggtgcggtcaatttctgctattccataattactacacaatagatcaagttaagctggctaaagctcattattgttctcagcatttactatgccctatgtgtgctggtgtaagggcagctaagtcaatgagtcgatatatccaacgtattgaagaattgatgcgtcagaatcgccatttaaagcccgtattgatcactttgacggttaagaacggtgaagacctacaggaacgctttaagcatcttagaagctcatttagaacgcttttagaccgctataacgattataagaaaaaaggccgtggctttaatcagttttgtaaaattgatagtgctttctattccactgaatatacctacaacccaaaaactaaagaatggcatcctcatattcatatttttgctttactcaatgaatggatagaccaggaagaattagccgaaacttggcacgacattaccctggattcttatatcgtcgatattcgaagagttaagaaaactaaagaacacggctatagcaaagctgttgcagaggtatgtaagtatgcccttaagtttagtgatctctcactggagaatacttgggaagcatatctttctttgaaaggtaataggctgactggctgttttggttctatgtatggtgtcaagctacctgaaaaacttactgatgatctacctcttgatgatcttccatatttggagctgctctatcgtttcgtatttgataaaaaatcctactacaacctagaaataacaaaagacgtaaagccaaacaaaaggaatgaggaaggatgaacgaggagcttcgacgaggcccgacgcccgaatgacttgtgaaggcgacacgtcaaatattagcaaggatacacaaaggtacctttatgccttgtaaggggttcgaccccttaaaccccaaagggcgcactttaccattgagcaaagctcaacggaaattgctcccaaaaataagaggagctaaaaatgaattatttcactttgtatattgttaattgcaataggtcacatccgaatagggataatgcttttcattgttttcatgtggctcaatattatagatatcttgagtatctccattattaacagagggggcggagtgcccccgaactgactcactcgaatttcaaaacttcctttccctgattacaaagctccccttagatttctaagcgggagcttttagccatcccgaatgggtggcgtcacaaaactatttgattttttcatgattcaaggtacgattcattgtgaatcacattcctatatatgaaaaaatcaggtgagaatatgcaaattgatgaaccaacaaacttccaagtattcatggcagaagtaaacaagacagcaaagacagaaagtattggtgtttatcatcaagtacctttccgtatggctcgatggaactttgcacgactggaaggcctccgtaatcacatgggagaacctcgaaacaaagttcttaactcactaatcgaaattgctttagatcaagtatttgaacaacttgagcatggcagtaaagagatccgtcgttctgttctggaggaagtttccaaagttctcgaatcaattgaacatgatggttctgggagcttagacaatgattaatctacaaggaactttgataaatgctttccgtgtggatggtggtaaaggtaaagacggcaaagaatatgaagcgcgtggcaaggtacaaattcttggttcgttggaactcccaaacggggaaattaaacacgaacttgttgacctaacagttgaggattctcgactgtttgaaccattcaaaaatcaggttattagcatctcatgtggtgctatggctatcggtcgtaatgttgttttttatgttcgcaaaggcgcgaaaccagttttagcagaacaaatgtgaaacacgaaaaaaaacagtgagttagccttggcttctcgctgttttttctttctcttgatactattgattaactaggggataaaaattgactaaaaaagacaataaaaaagcccataattcggatgctttggcgggcgatgaattacaggctttgcaatctgctaaggcagat

SEQ ID NO: 605 - protein, 51 aa, Artificial Sequence
  orf
MTLPWILISSIFEELRKLKNTAIAKLLQRYVSMPLSLVISHWRILGKHIFL

SEQ ID NO: 606 - protein, 310 aa, Artificial Sequence
  orf
MTRFGILKHRAKLQEQYLWTQVDFKSEGENDLSNKALKAATKLKGCGQFLLFHNYYTIDQVKLAKAHYCSQHLLCPMCAGVRAAKSMSRYIQRIEELMRQNRHLKPVLITLTVKNGEDLQERFKHLRSSFRTLLDRYNDYKKKGRGFNQFCKIDSAFYSTEYTYNPKTKEWHPHIHIFALLNEWIDQEELAETWHDITLDSYIVDIRRVKKTKEHGYSKAVAEVCKYALKFSDLSLENTWEAYLSLKGNRLTGCFGSMYGVKLPEKLTDDLPLDDLPYLELLYRFVFDKKSYYNLEITKDVKPNKRNEEG

SEQ ID NO: 607 - protein, 58 aa, Artificial Sequence
  orf
MKATRQILARIHKGTFMPCKGFDPLNPKGRTLPLSKAQRKLLPKIRGAKNELFHFVYC

SEQ ID NO: 608 - protein, 118 aa, Artificial Sequence; identical to SEQ ID NO: 376
  orf

SEQ ID NO: 609 - protein, 96 aa, Artificial Sequence
  orf
MINLQGTLINAFRVDGGKGKDGKEYEARGKVQILGSLELPNGEIKHELVDLTVEDSRLFEPFKNQVISISCGAMAIGRNVVFYVRKGAKPVLAEQM

SEQ ID NO: 610 - protein, 344 aa, Artificial Sequence
  orf
MTKKDNKKAHNSDALAGDELQALQSAKADQHRDRITRFGILKHRAKLQEQYLWTQVDFKSEGENDLSNKALKAATKLKGCGQFLLFHNYYTIDQVKLAKAHYCSQHLLCPMCAGVRAAKSMSRYIQRIEELMRQNRHLKPVLITLTVKNGEDLQERFKHLRSSFRTLLDRYNDYKKKGRGFNQFCKIDSAFYSTEYTYNPKTKEWHPHIHIFALLNEWIDQEELAETWHDITLDSYIVDIRRVKKTKEHGYSKAVAEVCKYALKFSDLSLENTWEAYLSLKGNRLTGCFGSMYGVKLPEKLTDDLPLDDLPYLELLYRFVFDKKSYYNLEITKDVKPNKRNEEG

SEQ ID NO: 611 - protein, 64 aa, Artificial Sequence
  orf
MNGSNSRESSTVRSTSSCLISPFGSSNEPRICTLPRASYSLPSLPLPPSTRKAFIKVPCRLIIV

SEQ ID NO: 612 - protein, 52 aa, Artificial Sequence
  orf
MITEAFQSCKVPSSHTERYLMINTNTFCLCCLVYFCHEYLEVCWFINLHILT

SEQ ID NO: 613 - DNA, 153 bp, Artificial Sequence
  orf
acgacattaccctggattcttatatcgtcgatattcgaagagttaagaaaactaaagaacacggctatagcaaagctgttgcagaggtatgtaagtatgcccttaagtttagtgatctctcactggagaatacttgggaagcatatctttctt

SEQ ID NO: 614 - DNA, 930 bp, Artificial Sequence
  orf
ataacacgttttgggattttgaaacatagagcgaagctacaagaacaatacttgtggacgcaggttgatttcaaatcagaaggtgaaaatgatctatccaataaggctcttaaggctgcaaccaaattaaaggggtgcggtcaatttctgctattccataattactacacaatagatcaagttaagctggctaaagctcattattgttctcagcatttactatgccctatgtgtgctggtgtaagggcagctaagtcaatgagtcgatatatccaacgtattgaagaattgatgcgtcagaatcgccatttaaagcccgtattgatcactttgacggttaagaacggtgaagacctacaggaacgctttaagcatcttagaagctcatttagaacgcttttagaccgctataacgattataagaaaaaaggccgtggctttaatcagttttgtaaaattgatagtgctttctattccactgaatatacctacaacccaaaaactaaagaatggcatcctcatattcatatttttgctttactcaatgaatggatagaccaggaagaattagccgaaacttggcacgacattaccctggattcttatatcgtcgatattcgaagagttaagaaaactaaagaacacggctatagcaaagctgttgcagaggtatgtaagtatgcccttaagtttagtgatctctcactggagaatacttgggaagcatatctttctttgaaaggtaataggctgactggctgttttggttctatgtatggtgtcaagctacctgaaaaacttactgatgatctacctcttgatgatcttccatatttggagctgctctatcgtttcgtatttgataaaaaatcctactacaacctagaaataacaaaagacgtaaagccaaacaaaaggaatgaggaagga

SEQ ID NO: 615 - DNA, 174 bp, Artificial Sequence
  orf
gtgaaggcgacacgtcaaatattagcaaggatacacaaaggtacctttatgccttgtaaggggttcgaccccttaaaccccaaagggcgcactttaccattgagcaaagctcaacggaaattgctcccaaaaataagaggagctaaaaatgaattatttcactttgtatattgt

SEQ ID NO: 616 - DNA, 354 bp, Artificial Sequence
  orf
atcacattcctatatatgaaaaaatcaggtgagaatatgcaaattgatgaaccaacaaacttccaagtattcatggcagaagtaaacaagacagcaaagacagaaagtattggtgtttatcatcaagtacctttccgtatggctcgatggaactttgcacgactggaaggcctccgtaatcacatgggagaacctcgaaacaaagttcttaactcactaatcgaaattgctttagatcaagtatttgaacaacttgagcatggcagtaaagagatccgtcgttctgttctggaggaagtttccaaagttctcgaatcaattgaacatgatggttctgggagcttagacaatgat

SEQ ID NO: 617 - DNA, 288 bp, Artificial Sequence
  orf
atgattaatctacaaggaactttgataaatgctttccgtgtggatggtggtaaaggtaaagacggcaaagaatatgaagcgcgtggcaaggtacaaattcttggttcgttggaactcccaaacggggaaattaaacacgaacttgttgacctaacagttgaggattctcgactgtttgaaccattcaaaaatcaggttattagcatctcatgtggtgctatggctatcggtcgtaatgttgttttttatgttcgcaaaggcgcgaaaccagttttagcagaacaaatg

SEQ ID NO: 618 - DNA, 1032 bp, Artificial Sequence
  orf
ttgactaaaaaagacaataaaaaagcccataattcggatgctttggcgggcgatgaattacaggctttgcaatctgctaaggcagatcaacacagggatagaataacacgttttgggattttgaaacatagagcgaagctacaagaacaatacttgtggacgcaggttgatttcaaatcagaaggtgaaaatgatctatccaataaggctcttaaggctgcaaccaaattaaaggggtgcggtcaatttctgctattccataattactacacaatagatcaagttaagctggctaaagctcattattgttctcagcatttactatgccctatgtgtgctggtgtaagggcagctaagtcaatgagtcgatatatccaacgtattgaagaattgatgcgtcagaatcgccatttaaagcccgtattgatcactttgacggttaagaacggtgaagacctacaggaacgctttaagcatcttagaagctcatttagaacgcttttagaccgctataacgattataagaaaaaaggccgtggctttaatcagttttgtaaaattgatagtgctttctattccactgaatatacctacaacccaaaaactaaagaatggcatcctcatattcatatttttgctttactcaatgaatggatagaccaggaagaattagccgaaacttggcacgacattaccctggattcttatatcgtcgatattcgaagagttaagaaaactaaagaacacggctatagcaaagctgttgcagaggtatgtaagtatgcccttaagtttagtgatctctcactggagaatacttgggaagcatatctttctttgaaaggtaataggctgactggctgttttggttctatgtatggtgtcaagctacctgaaaaacttactgatgatctacctcttgatgatcttccatatttggagctgctctatcgtttcgtatttgataaaaaatcctactacaacctagaaataacaaaagacgtaaagccaaacaaaaggaatgaggaagga

SEQ ID NO: 619 - DNA, 192 bp, Artificial Sequence
  orf
ttgaatggttcaaacagtcgagaatcctcaactgttaggtcaacaagttcgtgtttaatttccccgtttgggagttccaacgaaccaagaatttgtaccttgccacgcgcttcatattctttgccgtctttacctttaccaccatccacacggaaagcatttatcaaagttccttgtagattaatcattgtc

SEQ ID NO: 620 - DNA, 156 bp, Artificial Sequence
  orf
gtgattacggaggccttccagtcgtgcaaagttccatcgagccatacggaaaggtacttgatgataaacaccaatactttctgtctttgctgtcttgtttacttctgccatgaatacttggaagtttgttggttcatcaatttgcatattctcacc

(19) United States
(12) Patent Application Publication
(10) Pub. No.: US 2022/0025371 A1
(43) Pub. Date: Jan. 27, 2022

(54) CMI SEQUENCES AS AN EARLY MARKER FOR THE FUTURE DEVELOPMENT OF CANCER, ATHEROSCLEROSIS, DIABETES AND DISEASES OF THE CNS AND AS A TARGET FOR THE TREATMENT AND PREVENTION OF THESE DISEASES

(71) Applicant: DEUTSCHES KREBS-FORSCHUNGSZENTRUM STIFTUNG DES OFFENTLICHEN RECHTS, Heidelberg (DE)

(72) Inventors: Ethel-Michele DE VILLIERS-ZUR HAUSEN, Wald-Michelbach (DE); Harald ZUR HAUSEN, Wald-Michelbach (DE); Karin GUNST, Hirschberg (DE); Timo BUND, Dossenheim (DE)

(21) Appl. No.: 17/371,357

(22) Filed: Jul. 9, 2021

Related U.S. Application Data
(63) Continuation-in-part of application No. PCT/EP2020/051766, filed on Jan. 24, 2020.

(30) Foreign Application Priority Data
Jan. 24, 2019 (EP) 19153535.0

(51) Int. Cl. C12N 15/113; C12Q 1/6813; C12N 15/10; A61K 39/00

(52) U.S. Cl. CPC C12N 15/113; A61K 39/0011; C12N 15/1003; C12Q 1/6813

(57) ABSTRACT

Described are CMI (Cow Milk Isolate) nucleotide sequences as well as probes and primers comprising part of said nucleotide sequences and antibodies against polypeptides encoded by said nucleotide sequences. Said compounds are useful as early markers for the future development of cancer, diabetes, atherosclerosis and diseases of the CNS (Multiple sclerosis MS, Prion-linked diseases, amyotrophic lateral sclerosis, transmissible spongiforme encephalitis, Parkinson's disease, Alzheimer disease) and should represent targets for treatment and prevention.

### RELATED APPLICATIONS AND INCORPORATION BY REFERENCE

This application is a continuation-in-part application of international patent application Serial No. PCT/EP2020/0 517 66 filed 24 Jan. 2020, which published as PCT Publication No. WO 2020/152334 on 30 Jul. 2020, which claims benefit of European patent application Serial No. 19153535.0 filed 24 Jan. 2019.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said ASCII copy is named Y8005_00020SL.txt and is 552 kbytes in size.

### FIELD OF THE INVENTION

The present invention relates to CMI (Cow Milk Isolates) nucleotide sequences as well as probes and primers which may comprise part of said nucleotide sequences and antibodies against polypeptides encoded by said nucleotide sequences. Finally, the present invention relates to the use of said compounds as an early marker for the future development of diseases such as cancer, atherosclerosis, diabetes and diseases of the CNS and as a target for treatment and prevention of these diseases.

### BACKGROUND OF THE INVENTION

A large set of novel viral and phage genomes has been identified during the past several years. These were identified through analyses of the human microbiome (Kowarsky et al., 2017; Fernandes et al., 2018) or of samples from other sources. Metagenomic analyses are commonly applied (Rosario et al., 2012; Gronenborn et al., 2018; Wang et al., 2018; Kazlauskas et al., 2018; Kraberger et al., 2015; Varsani et al., 2018), although the application of more conservative techniques also led to the identification of a number of new infectious agents (de Villiers et al., 2009; Peretti et al., 2015; zur Hausen and de Villiers, 2015). Analyses mostly relied on comparisons to other known agents or the identification of a replication gene in the assembled genome. Unfortunately, several studies concentrated only on the presence of known infectious entities, while paying less attention to identifying yet unknown or putative novel infectious agents (Moustafa et al., 2017; Sadeghi et al., 2017; Legoff et al., 2017). The discovery of chimeric genomes has weakened the strict demarcation between certain types or groups of infectious agents resulting in studies investigating their phylogenetic origin (Kurpovic, 2013; Krupovic et al., 2015; Roux et al., 2013; Wawrzyniak et al., 2017; Kazlauskas et al., 2017).

Published data on the epidemiology of colon and breast cancer suggested milk and meat factors (BMMFs) derived from dairy cattle as presumably species-specific risk for these cancers after consumption of products from these animals (zur Hausen, 2012; 2015; zur Hausen and de Villiers, 2015; zur Hausen et al., 2017; 2018). Putative Rep genes were identified as part of the BMMF's DNA sequences obtained by in silico comparisons to available sequences. Amplification using abutting primers in the rep gene led to the isolation of full and partial circular DNA genomes from bovine sera (Funk et al., 2014). This was extended to samples from commercially available milk products for the presence of specific circular single-stranded DNA genomes. Full-length circular single-stranded DNA molecules of 14 different isolates of (˜1100 to 3000 nucleotides) were cloned and sequenced (Whitley et al., 2014; Gunst et al., 2014; Funk et al., 2014; Lamberto et al., 2014). Four additional isolates were obtained from human brain and serum (all from patients with multiple sclerosis) (Whitley et al., 2014; Gunst et al., 2014; Lamberto et al., 2014). The 18 isolates were divided into four different groups BMMF1 through BMMF4, according to their molecular characteristics (zur Hausen et al., 2017). Three of these groups revealed a remarkable degree of similarity to Acinetobacter baumannii and Psychrobacter plasmids. The fourth group comprised 3 isolates being representatives of Gemycirularviridae. Representatives of all four BMMF groups have been analyzed for expression of genetic activity and replication in human cells (zur Hausen et al., 2017; Eilebrecht et al., 2018).

Citation or identification of any document in this application is not an admission that such document is available as prior art to the present invention.

### BRIEF DESCRIPTION OF THE PRESENT INVENTION

Thus, the technical problem underlying the present invention is to identify specific nucleotide sequences that might be associated with diseases such as cancer, atherosclerosis, diabetes or diseases of the CNS and, thus, to provide means for diagnosis and therapy.

The solution to said technical problem is achieved by providing the embodiments characterized in the claims.

Recently, circular DNA genomes (Healthy Cattle Blood Isolate: HCBI1, HCBI2 and HCBI7) were isolated from serum from healthy cattle (Funk et al., 2014). These isolates were grouped as BMMF2 based on their nucleotide identity to the previously described Sphinx2.36 (acc. no HQ444405, Manuelidis et al., 2011, zur Hausen et al., 2017). The DNA sequences and open reading frames of several of these components showed a recognizable relationship to two sequences which were already described in transmissible spongiform encephalopathies (TSE) for TSE-diseases of sheep and humans.

The TSE isolates have also been hypothesized to play a role in cancer induction (Manuelidis, 2011). The inventors of the present invention postulated a role of infections in specifically colon, breast and prostate cancers but also Hodgkin's disease and others, and diseases of the CNS, e.g. Multiple sclerosis MS, amyotrophic lateral sclerosis, Parkinson's disease, Alzheimer disease (zur Hausen, 2012, zur Hausen et al., 2017, 2018).

The inventors subsequently used two sets of abutting PCR primers overlapping in the highly conserved region of their Rep genes to amplify full-length circular DNA molecules. The inventors analyzed 16 cow milk products, mainly milk samples for the presence of additional members of this group. The full-length circular genomes of 97 isolates (16 cow milk products, 81 cow milk) belonging to the BMMF2 group were generated ranging from 2102 to 3090 nucleotides in size and the 23 sequences (all from cow milk samples and all belonging to the BMMF2 group; C2MI) claimed in the present application are representative members thereof. According to the present invention the term “cow milk sample” embraces cow milk as such and cow milk products (e.g. yoghurt, cheese, kefir, cream cheese, cream, curd cheese, milk powder, sour cream, etc.) BLASTN comparisons to all available DNA databanks, revealed similarity mainly to plasmids of Acinetobacter baumanii, (Acinetobacter baumannii str. AYE plasmid p4ABAYE, acc. no CU459139, Acinetobacter baumannii strain A85 plasmid pA85-1, acc. no CP021783 and Acinetobacter baumannii strain DS002 plasmid pTS236 acc. no JN872565, Longkumar et al., 2013), but also to ssDNA isolates from Rat gut (pRGRH0103 acc. no LN852793 and pRGRH0636 acc. no. LN853262). The nucleotide sequence of p4ABAYE differs from pA85-1 in 66 positions (Hamidian et al., 2014).

In silico analyses of putative open reading frames (ORF, >50aa) were performed on each genome by using alternative start codons (Kearse and Wilusz, 2017). This resulted in the identification of a replication gene in each isolate with mainly 2 additional major ORFs on the positive DNA strand. The encoded putative proteins ranged from 101 to 138 amino acids and 96 to 148 amino acids in size, respectively. Additional smaller ORFs were randomly present. Multiple ORFs (average number 2 to 10, up to 125 amino acids in size) were identified on the reverse strand. BLASTP analyses of the latter putative proteins revealed no or very weak similarity to known proteins.

Bacterial plasmids are classified based on nucleotide homology of their replicase genes (Bertini et al., 2010). The inventors compared the putative Rep proteins of all BMMF2, as well as BMMF1 isolates. A core conserved region (329aa) was used in the phylogenetic analyses. BMMF2 rep proteins were grouped into 2 clades (A and B), whereas BMMF1 rep proteins formed a third clade (C). Clades A and B shared 60 to 65% similarity. Rep proteins in clade A shared 69-90% similarity within the clade and 70-82% to the p4ABAYE rep. Similarly clade B rep proteins shared 80-90% within the clade and 59-62% to p4ABAYE rep. The rep proteins of BMMF1 (clade C) are more diverse and shared almost no similarity (10-12%) to clades A and B.

The putative rep proteins of several BMMF2 isolates did not result only from one ORF, but were compiled of more than one truncated ORE. In order to ascertain whether potential introns were present in these interrupted BMMF2 genomes, the inventors performed GENSCAN analyses (Burge and Karlin, 1997). This tool has been developed to identify genes in prokaryotes, eukaryotes and plants. The inventors aligned these genes to the respective full-length genome. Very notable in the GENSCAN products are the presence of a polyadenosine tails (polyA) in almost every isolate, including the plasmid p4ABAYE. PolyA plays an important role in the maturation of RNA transcripts of almost all eukaryotes by stabilizing the mRNA for transport to the cytoplasm and subsequent translation (Curinha et al., 2014).

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

### Abbreviations

Rep=replication protein;

CMI: cattle milk isolate

C2MI cattle milk isolate belonging to the BMMF2 group

HCBI: healthy cattle blood isolate

Sphinx: slow progressive hidden infection of variable (X) latency

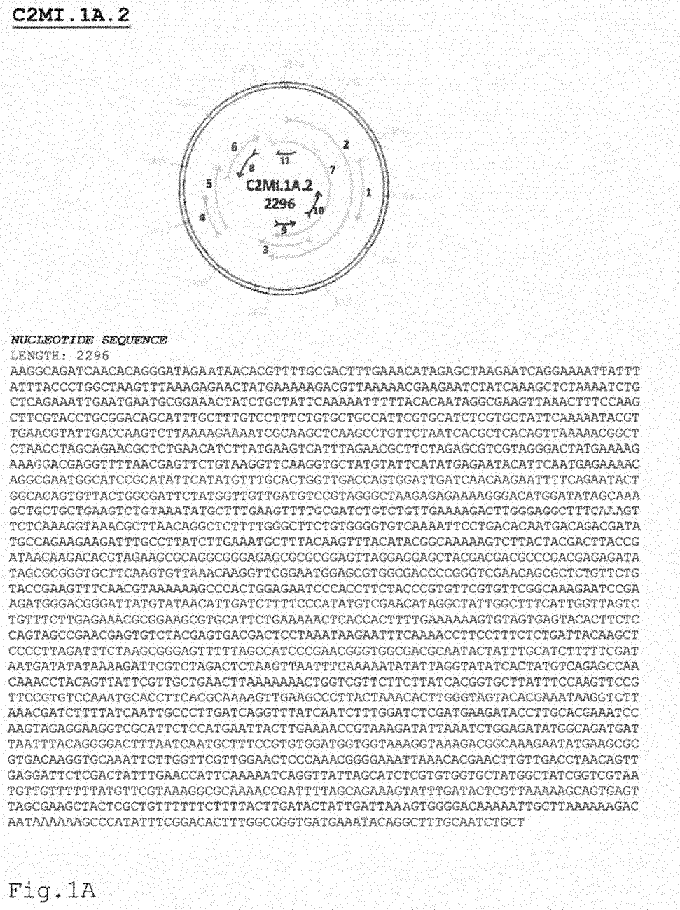

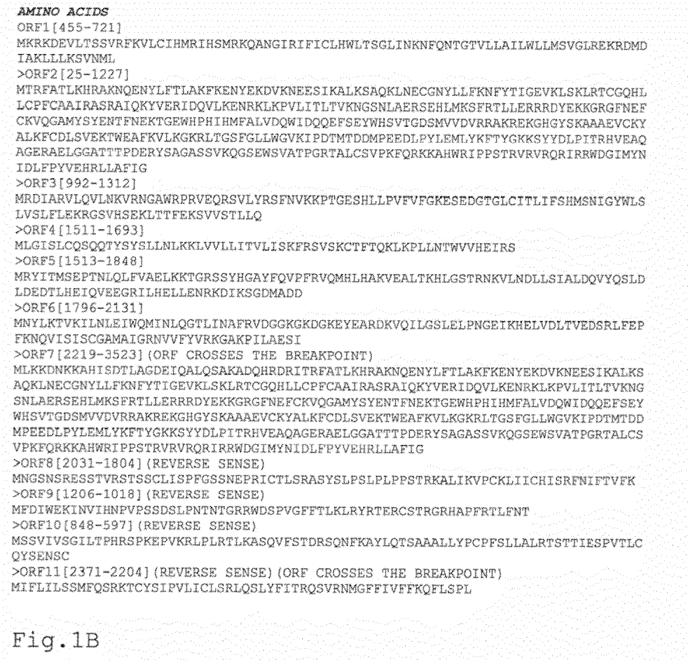

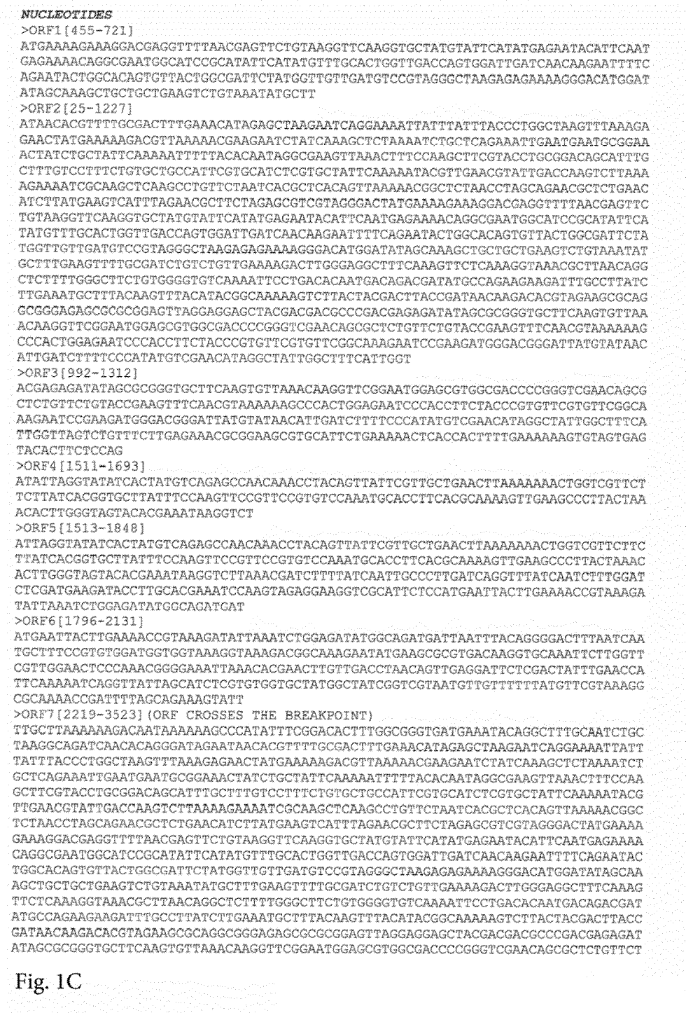

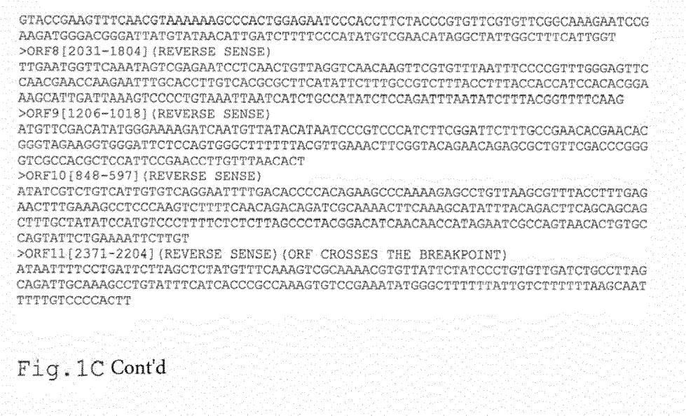

FIGS. 1-22: Nucleotide and amino acid sequences of

C2MI.1A.2 (FIG. 1A-1C)

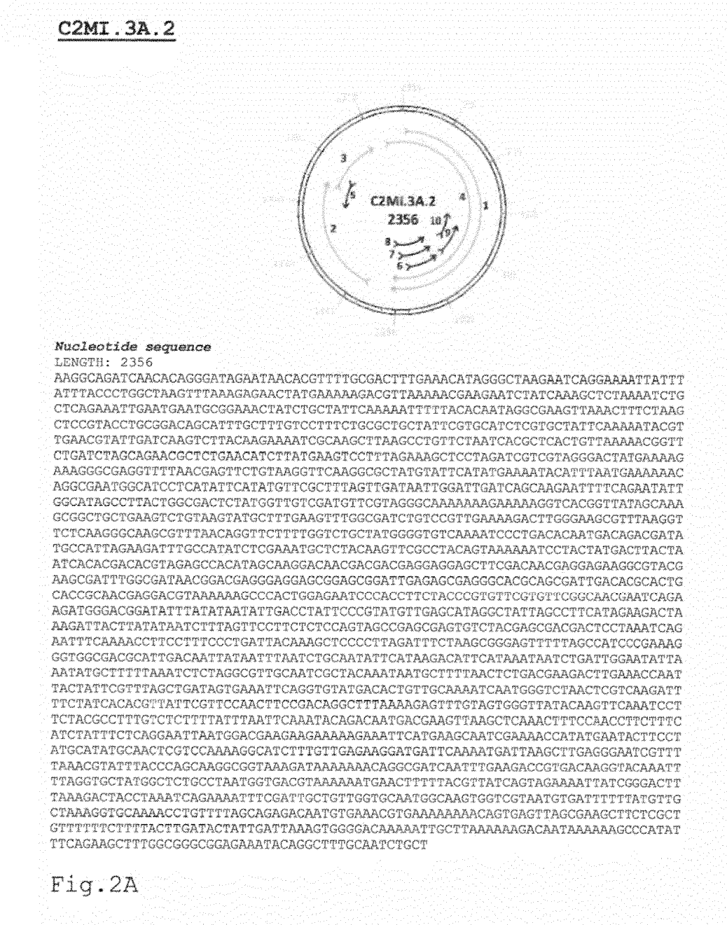

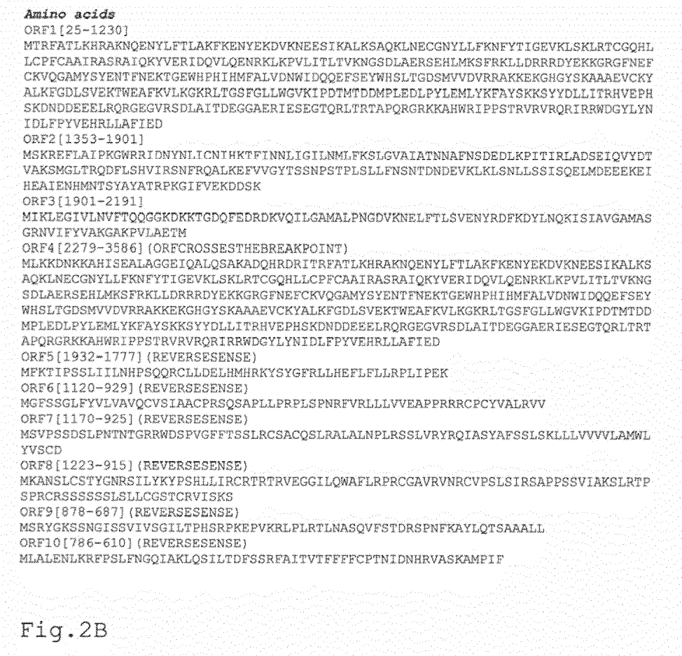

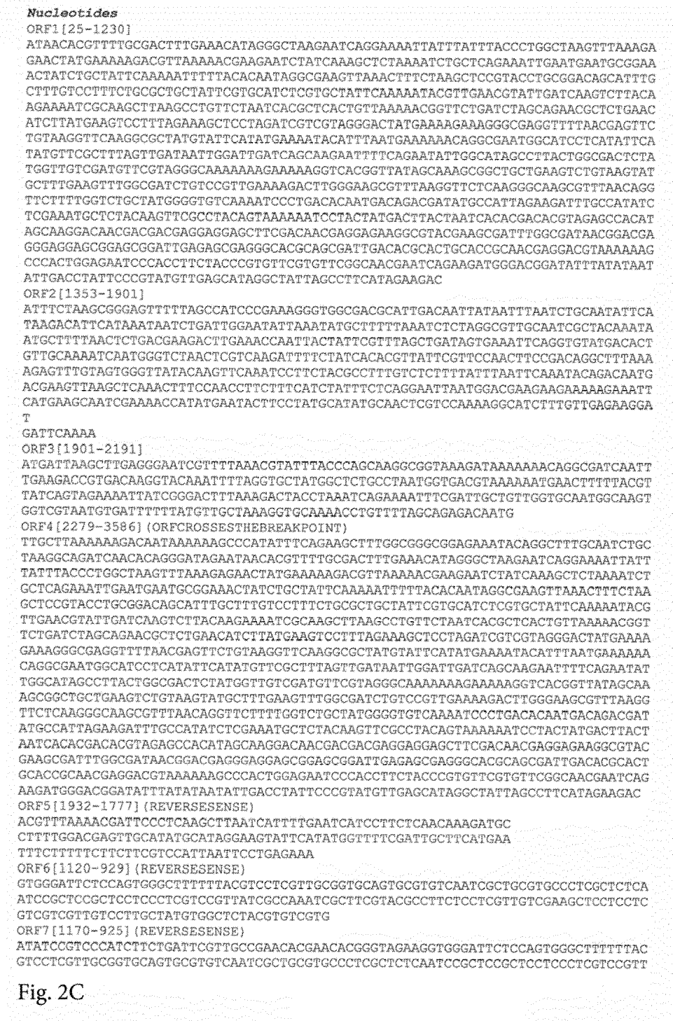

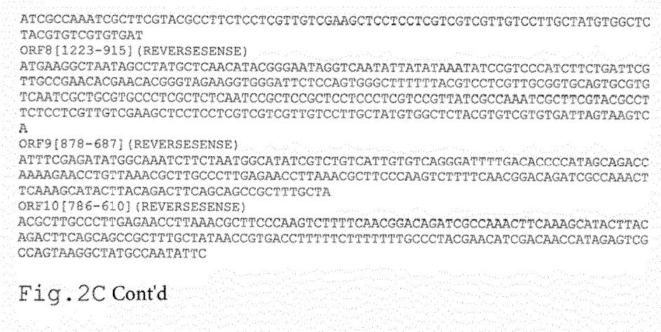

C2MI.3A.2 (FIG. 2A-2C)

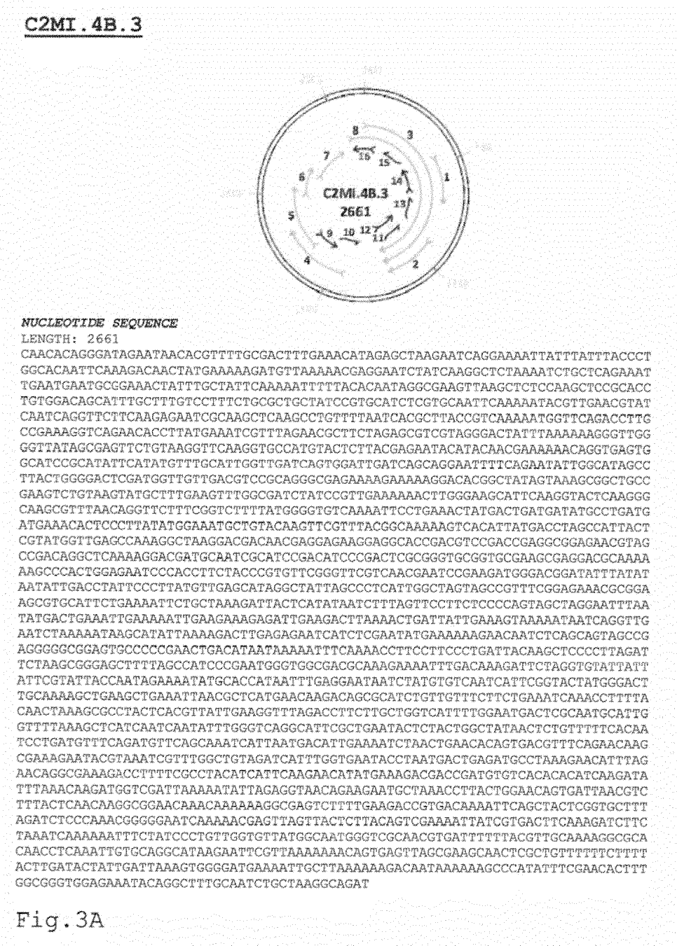

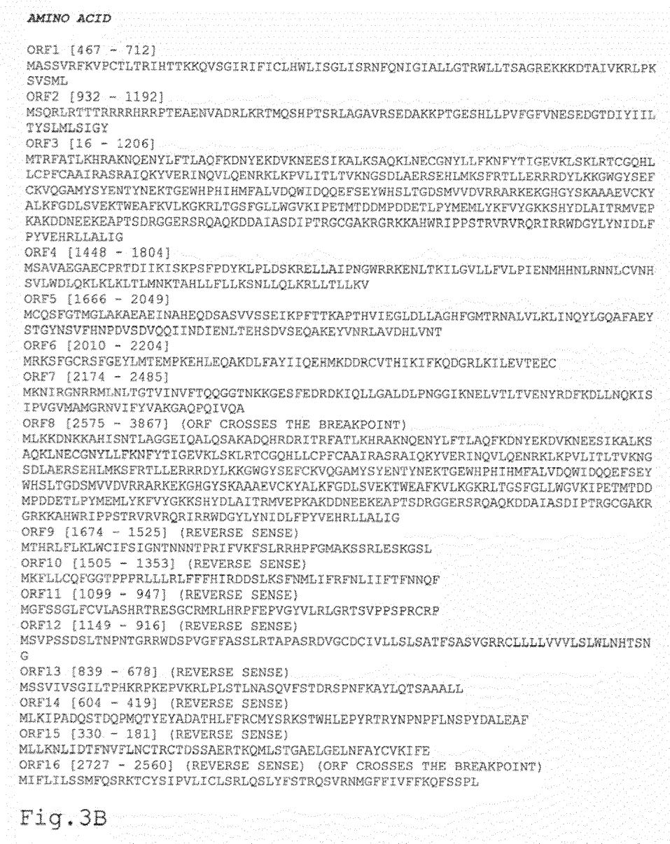

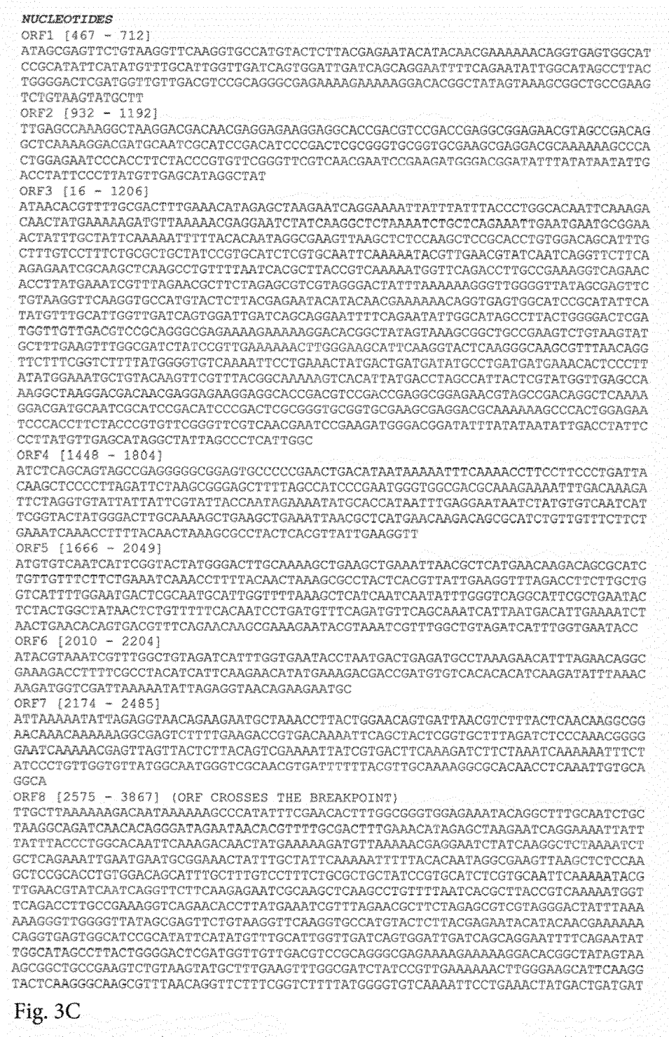

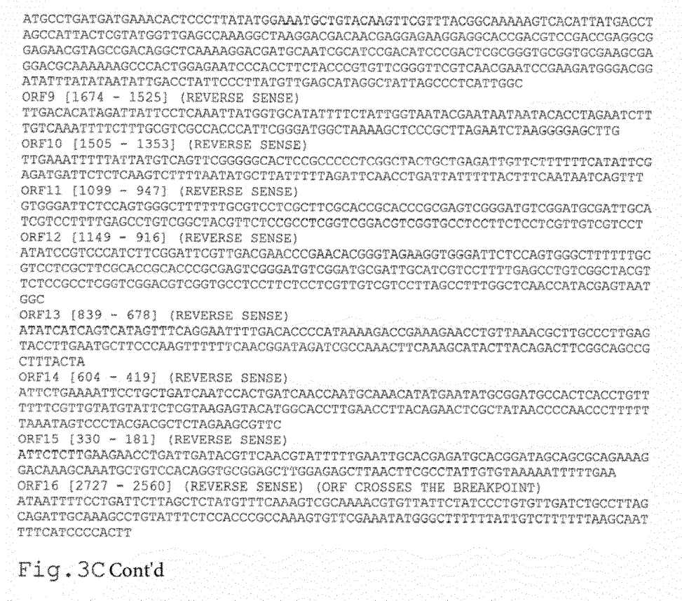

C2MI.4B.3 (FIG. 3A-3C)

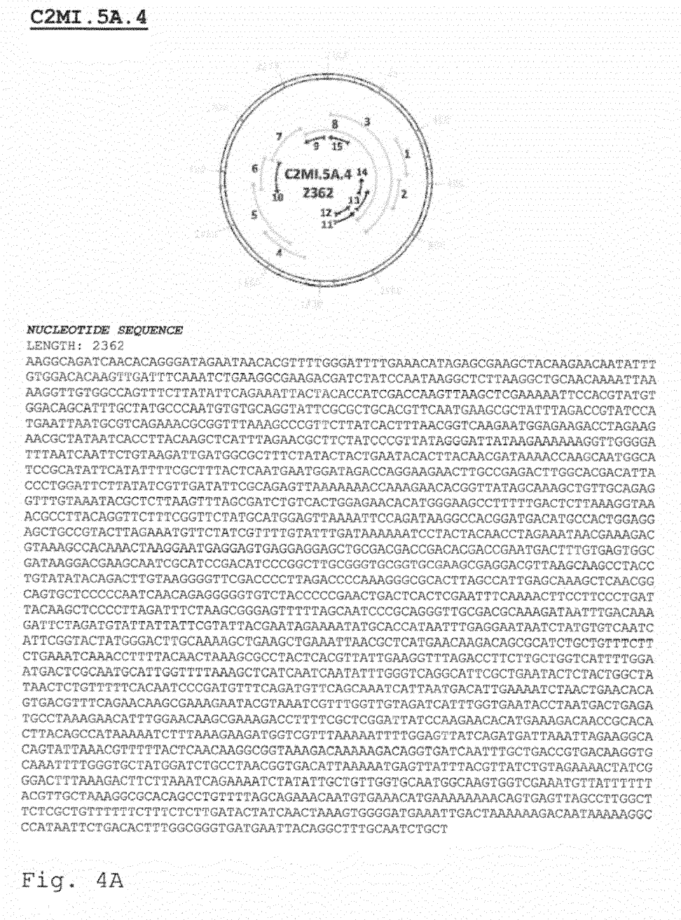

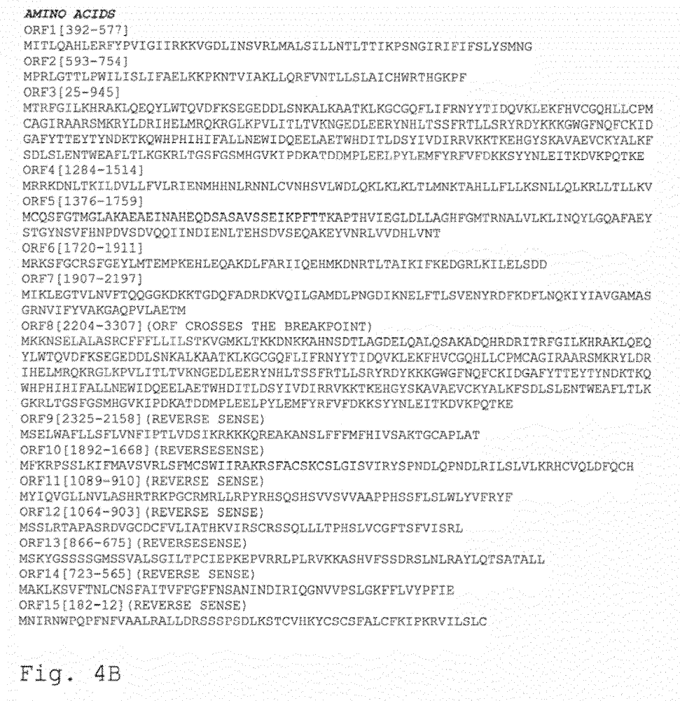

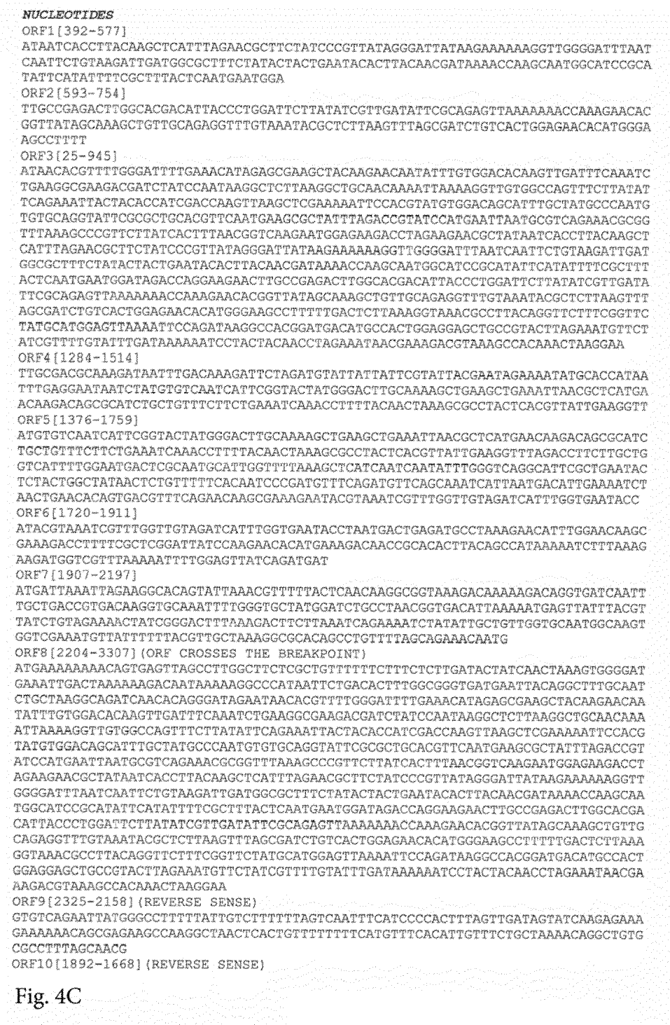

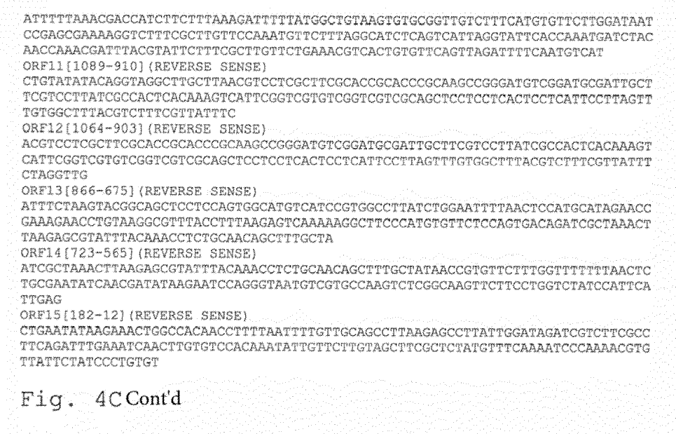

C2MI.5A.4 (FIG. 4A-4C)

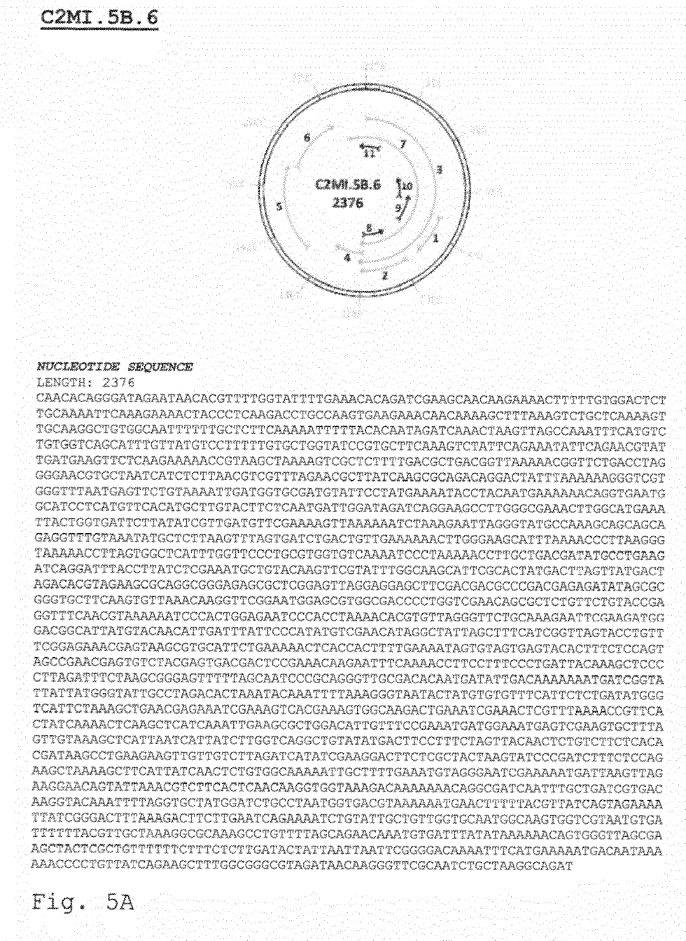

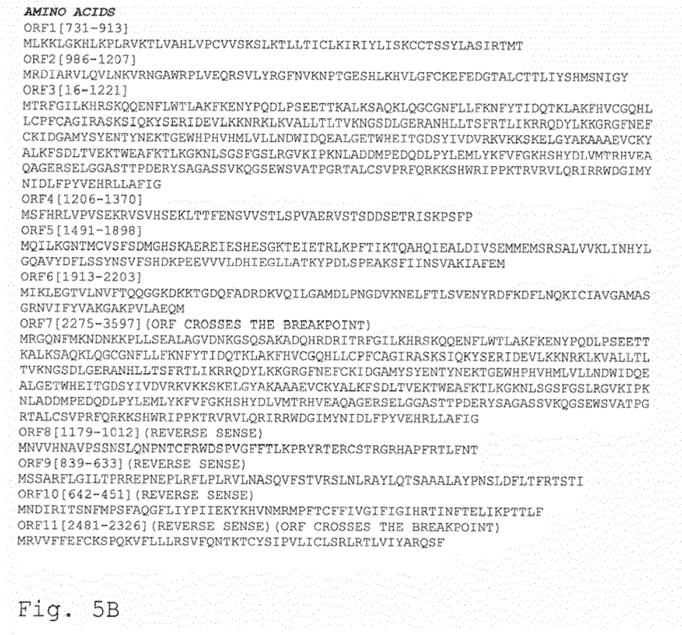

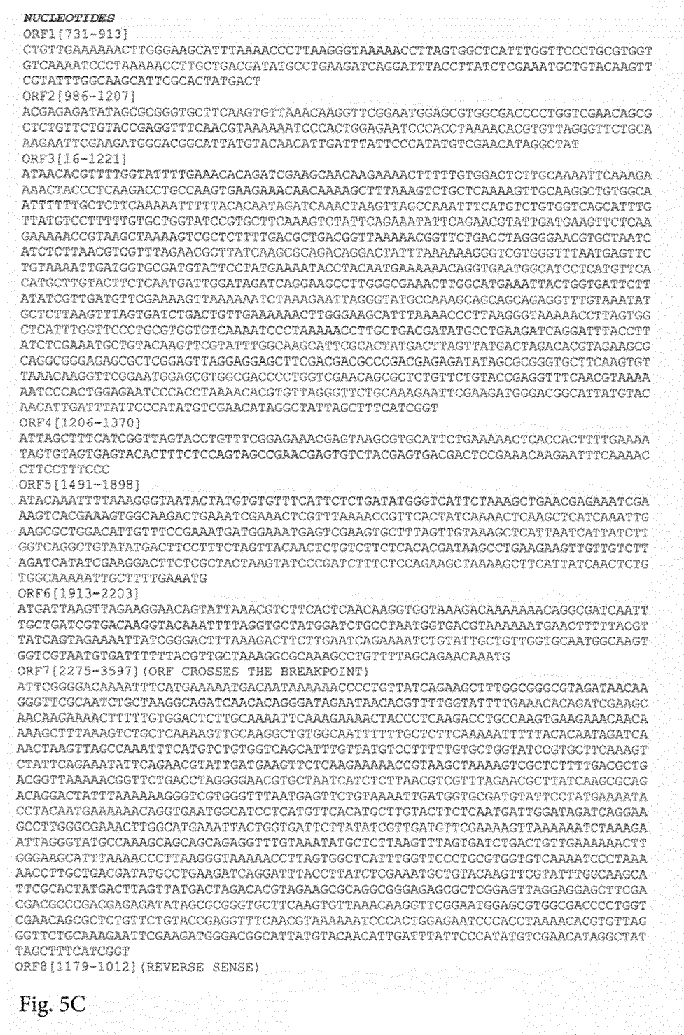

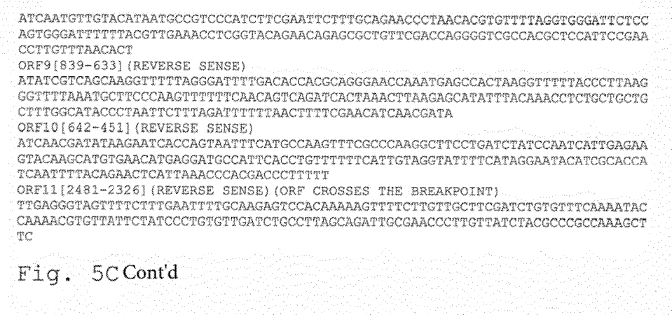

C2MI.5B.6 (FIG. 5A-5C)

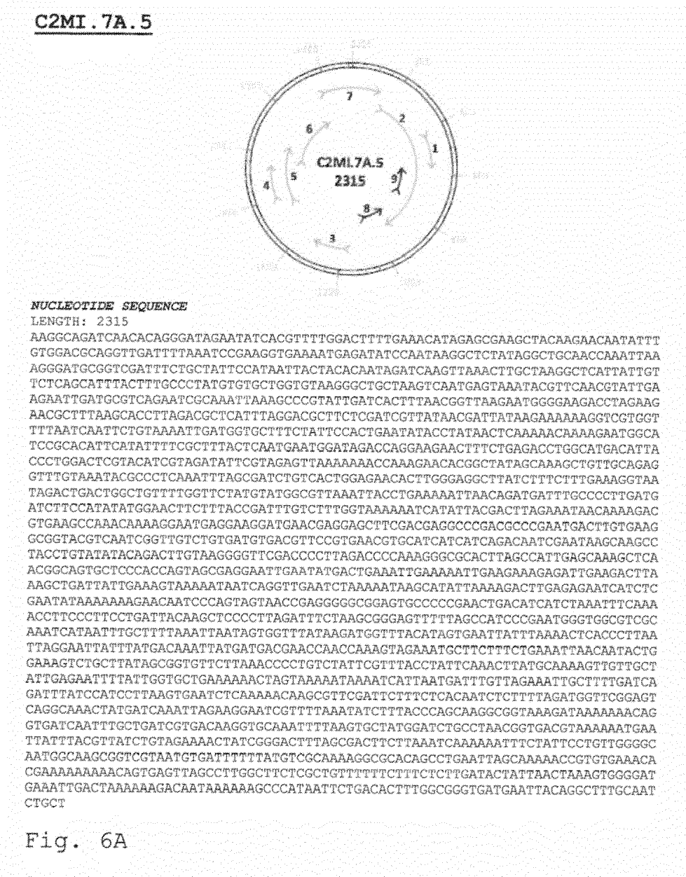

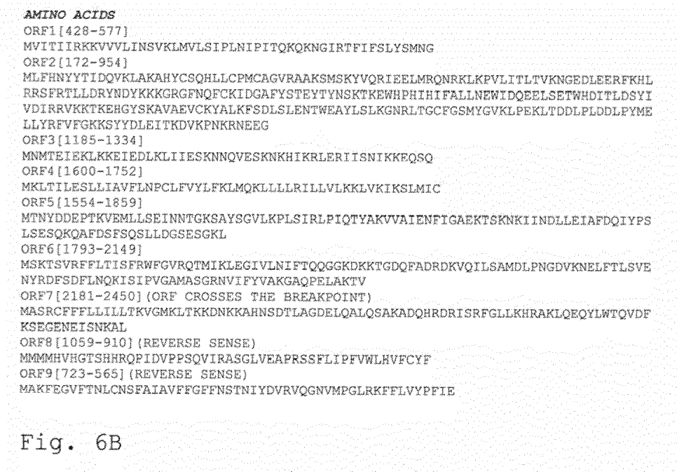

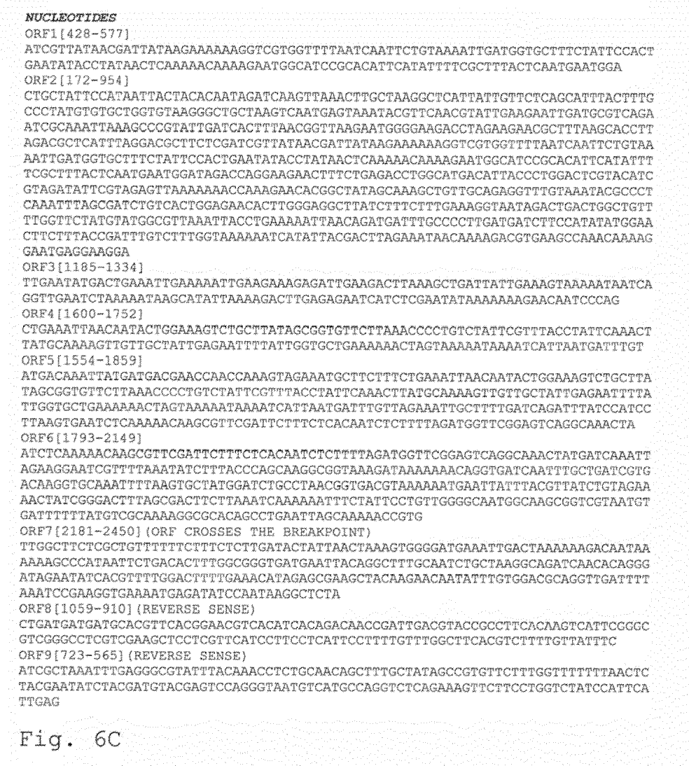

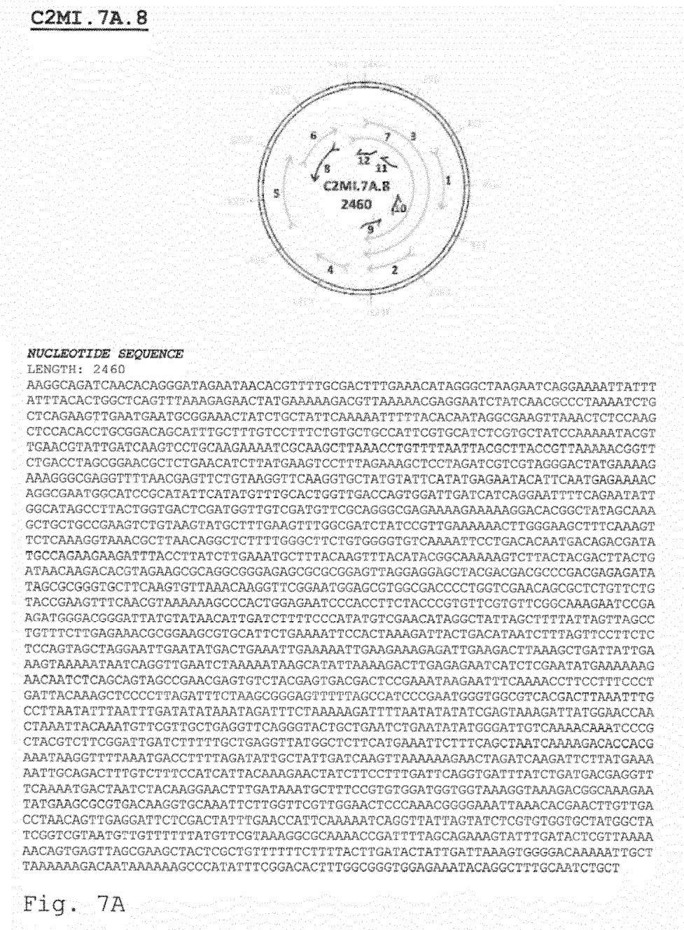

C2MI.7A.5 (FIG. 6A-6C)

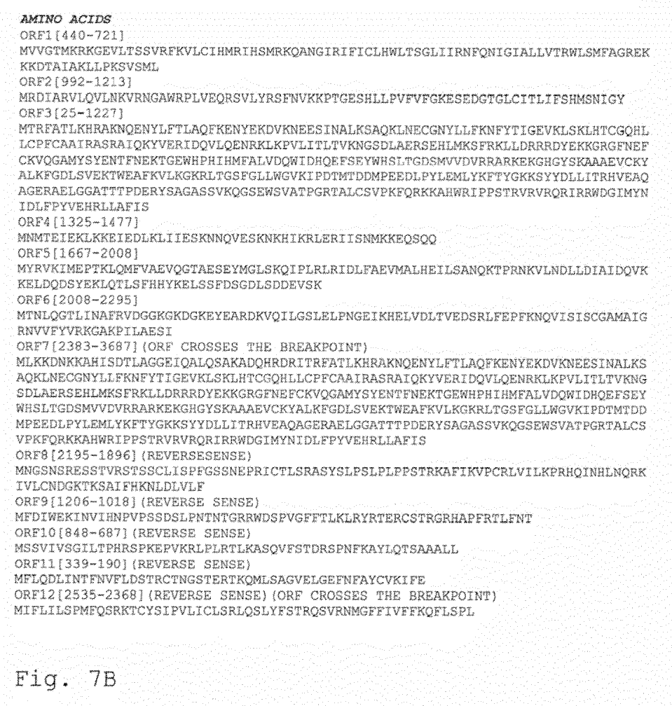

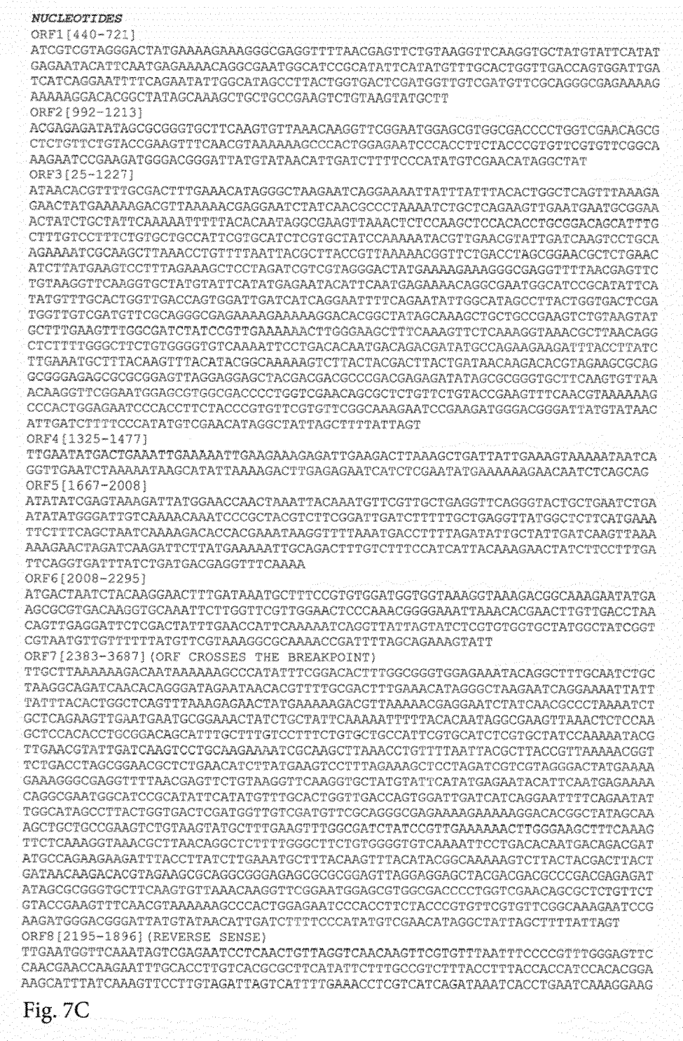

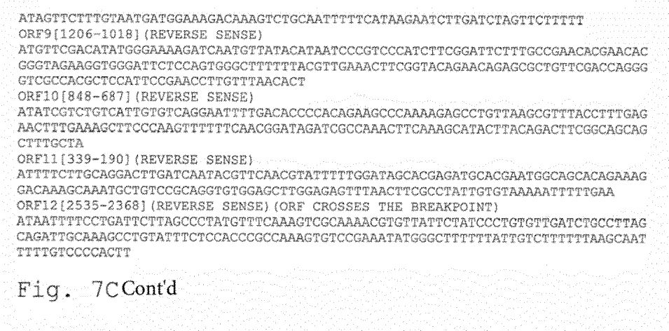

C2MI.7A.8 (FIG. 7A-7C)

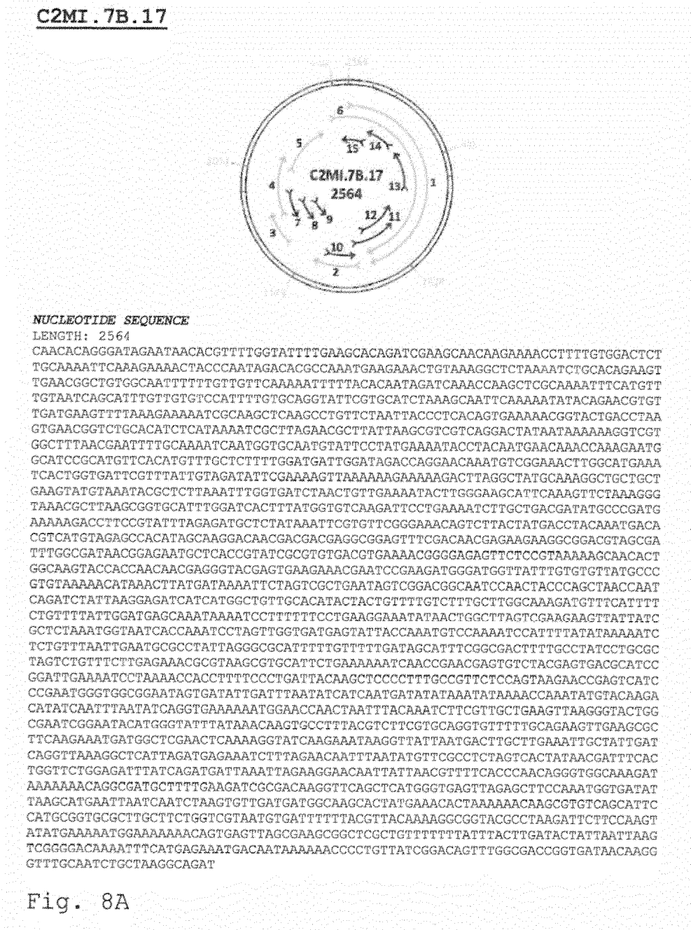

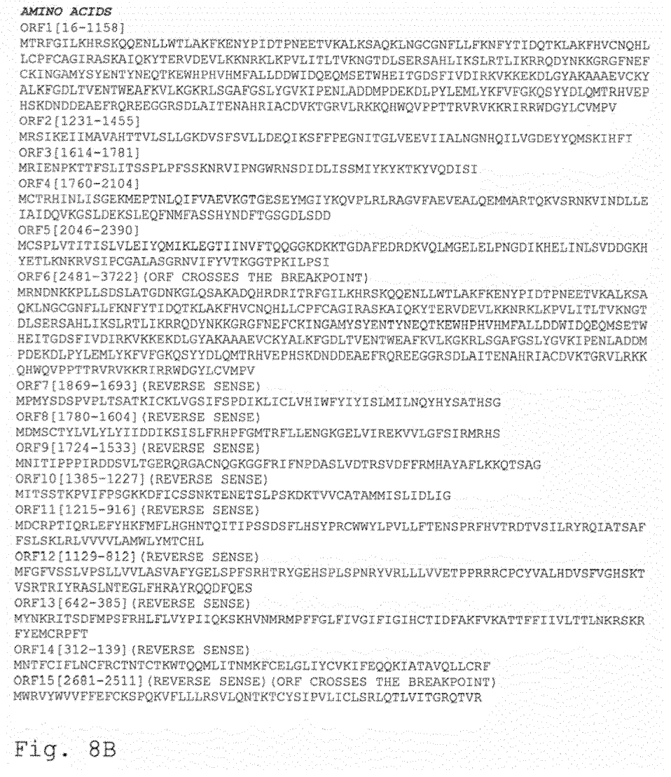

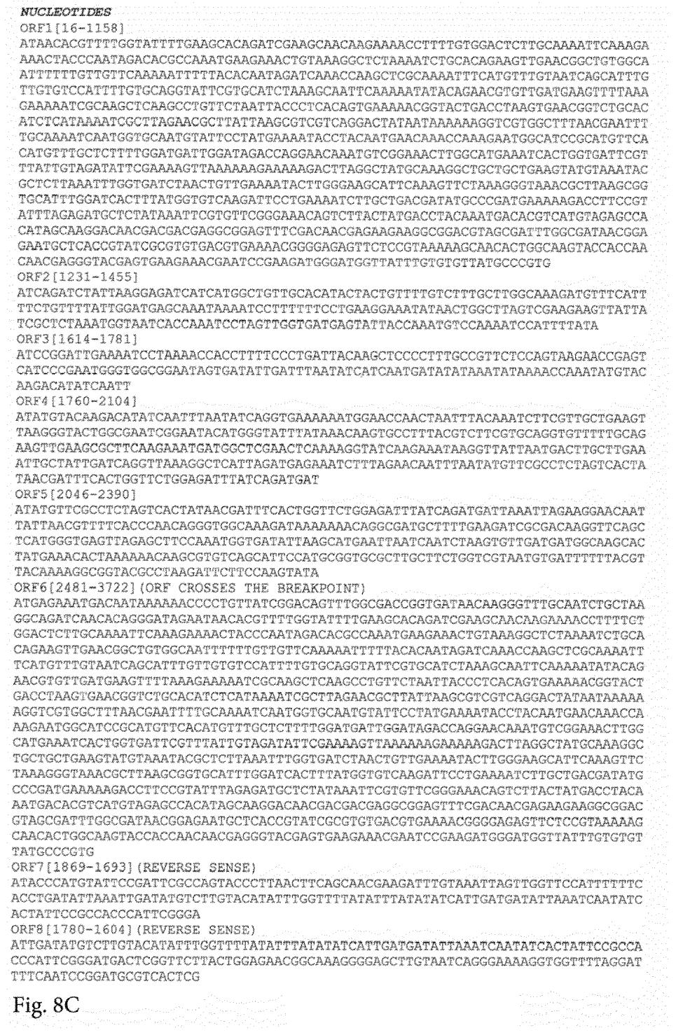

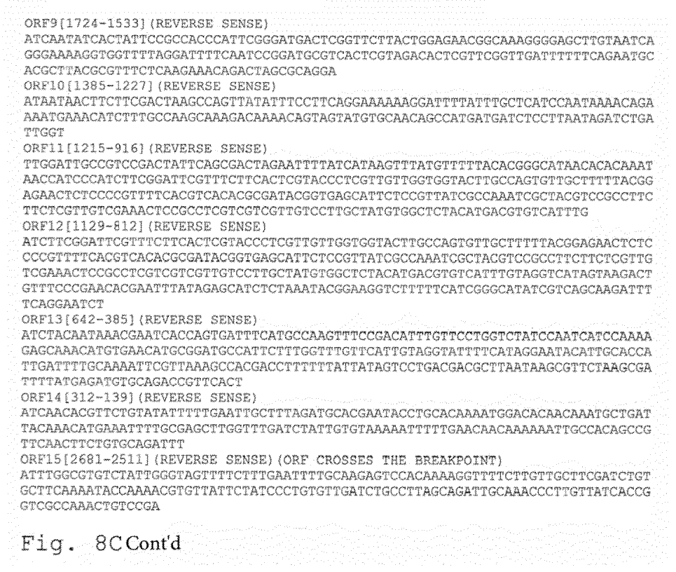

C2MI.7B.17 (FIG. 8A-8C)

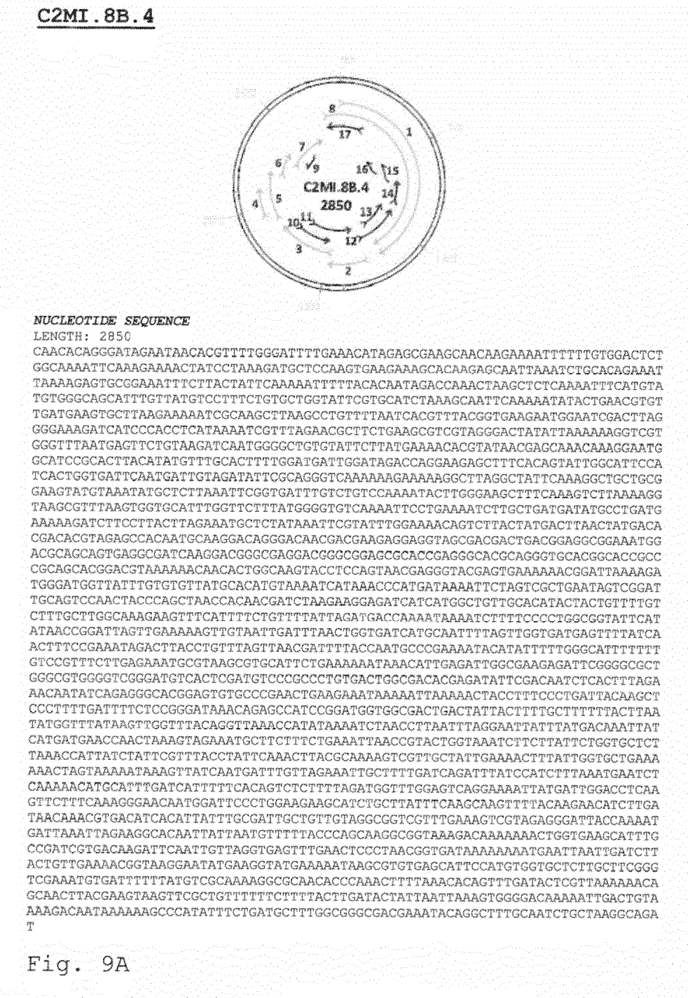

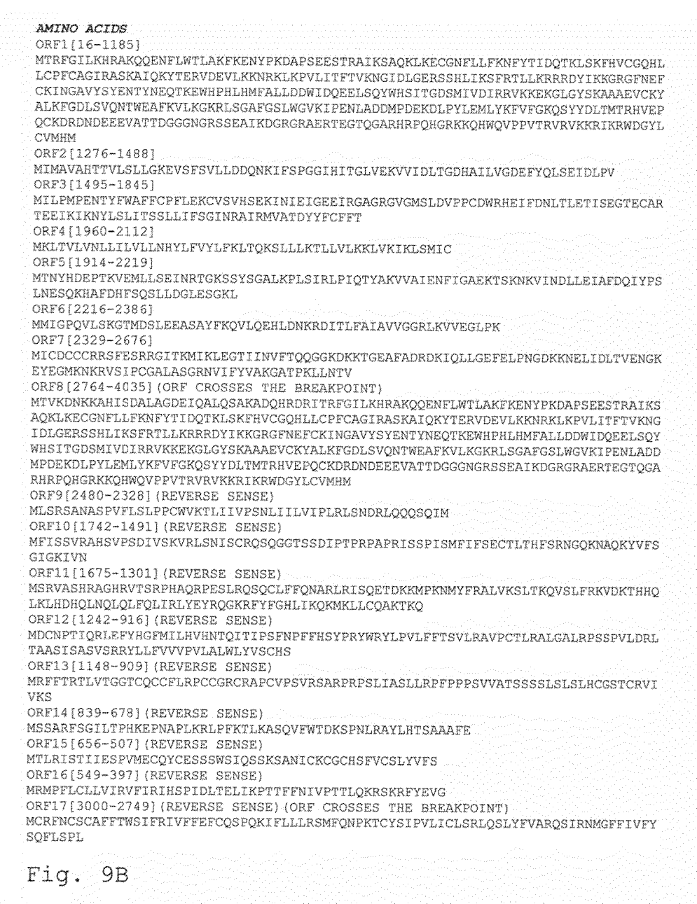

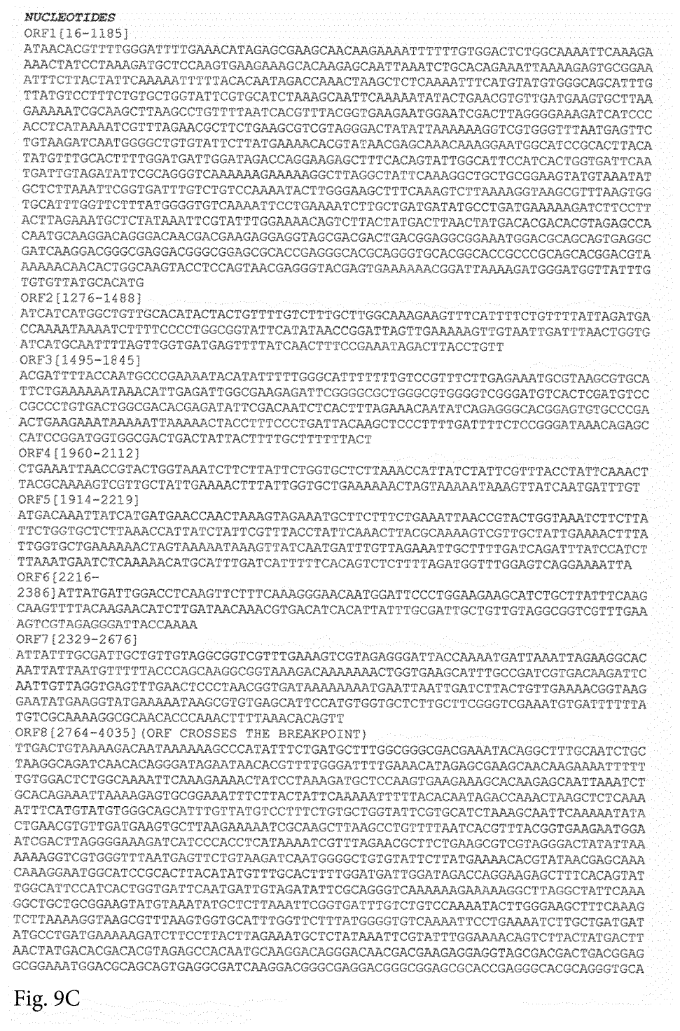

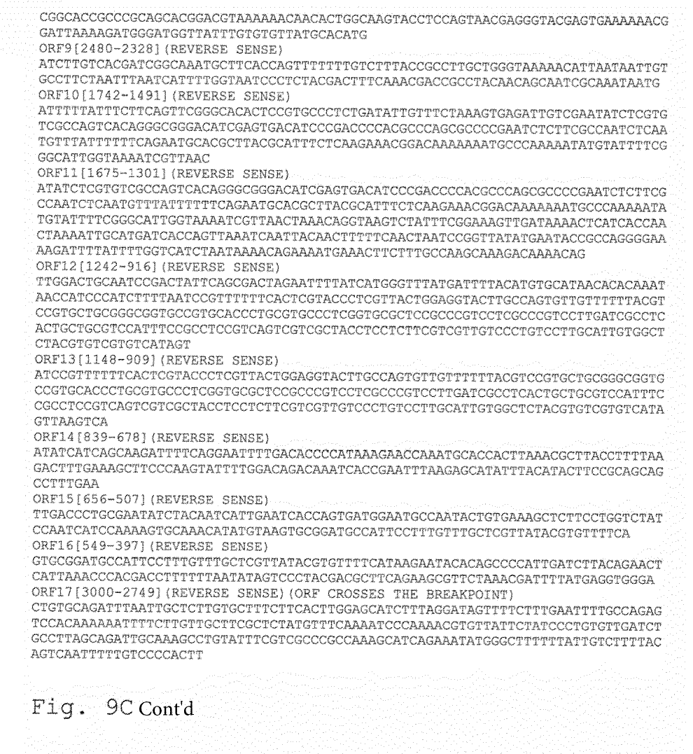

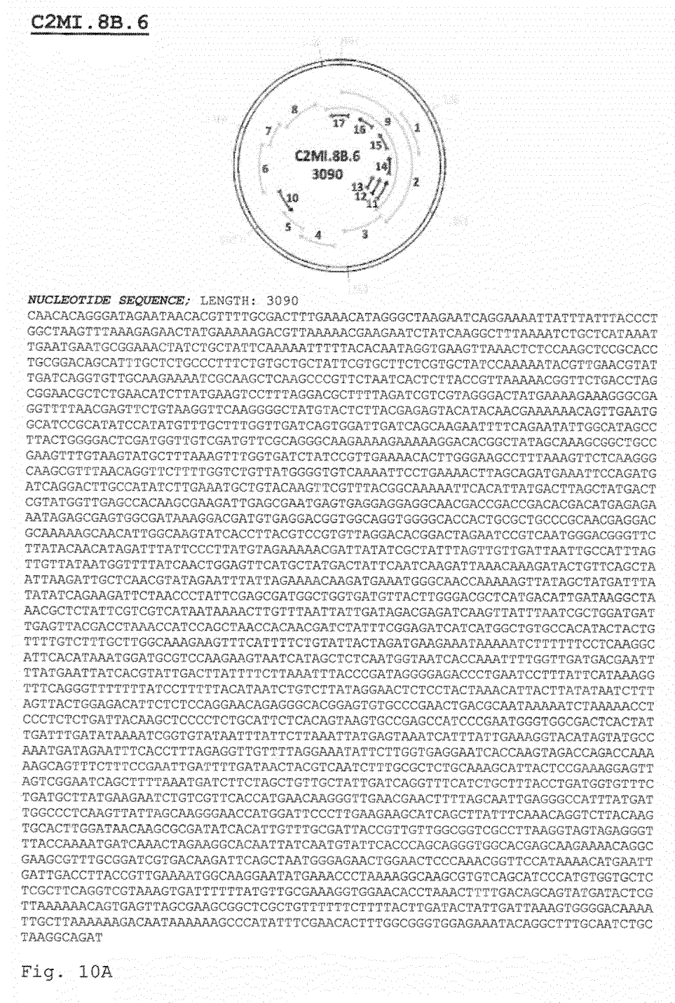

C2MI.8B.4 (FIG. 9A-9C)

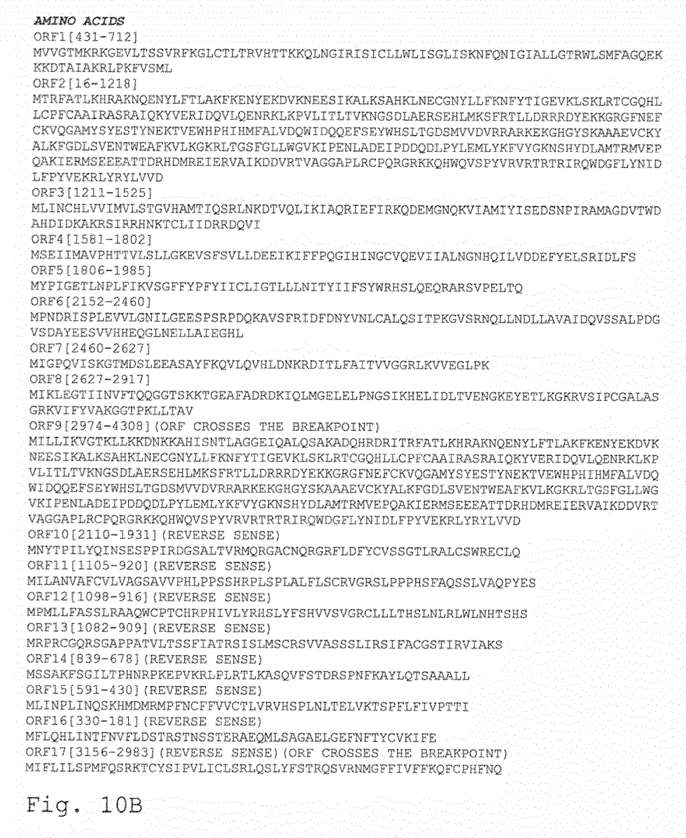

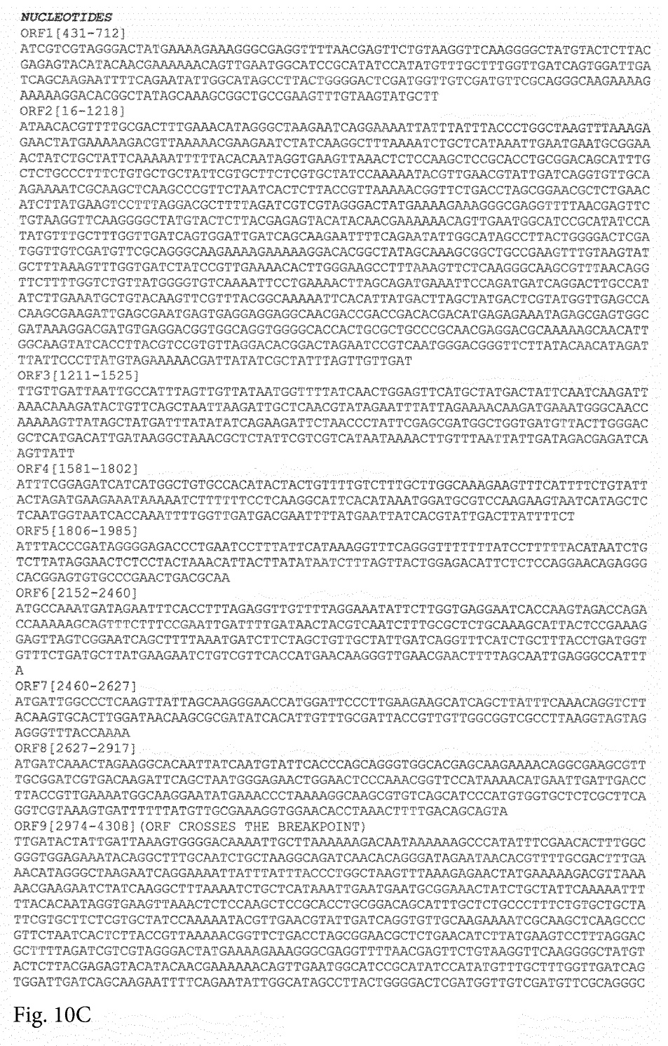

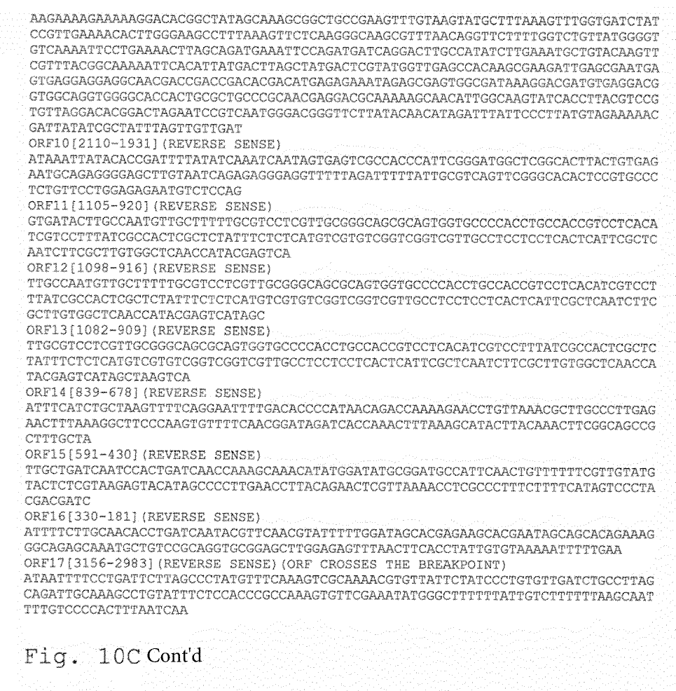

C2MI.8B.6 (FIG. 10A-10C)

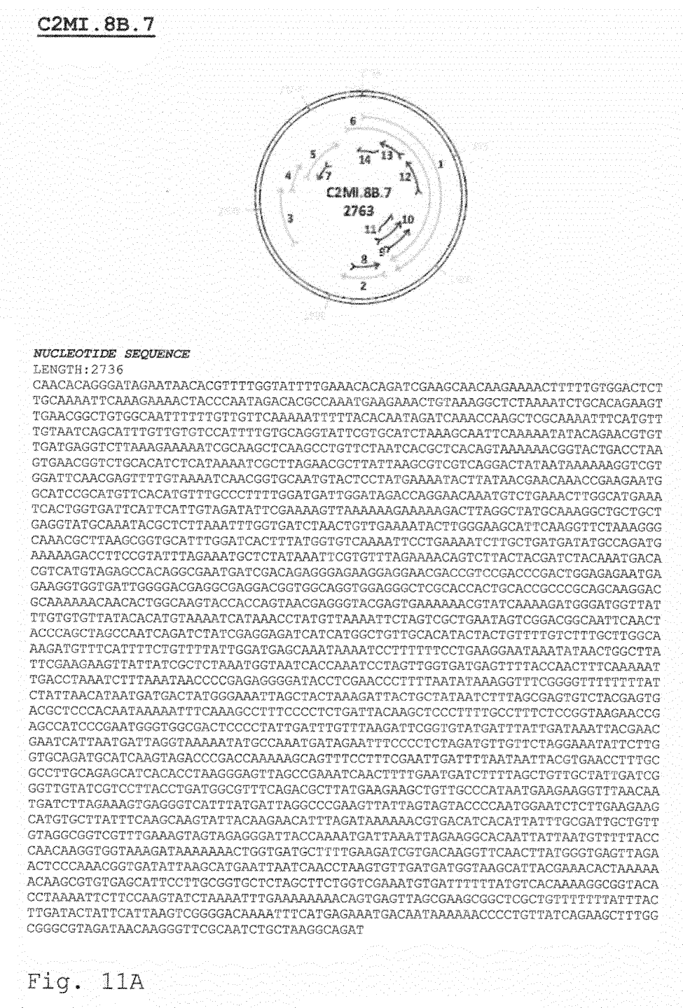

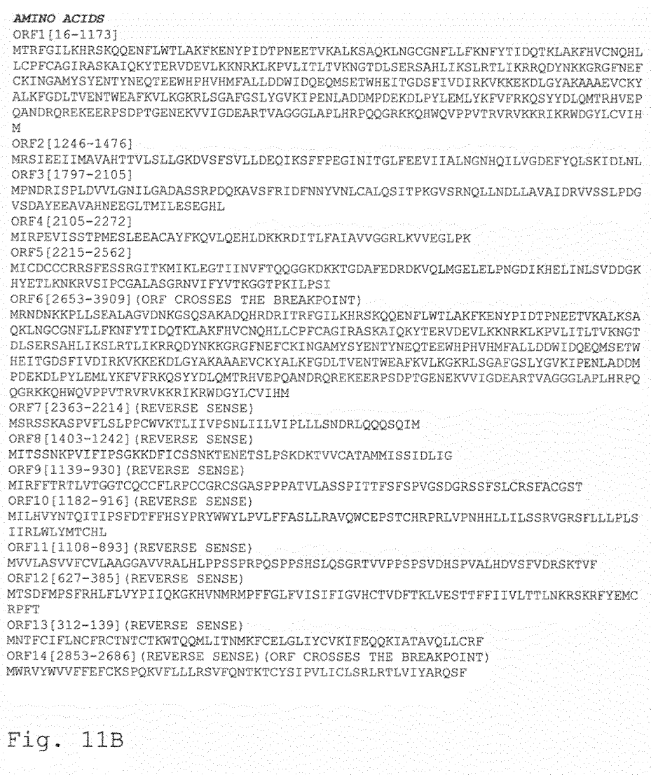

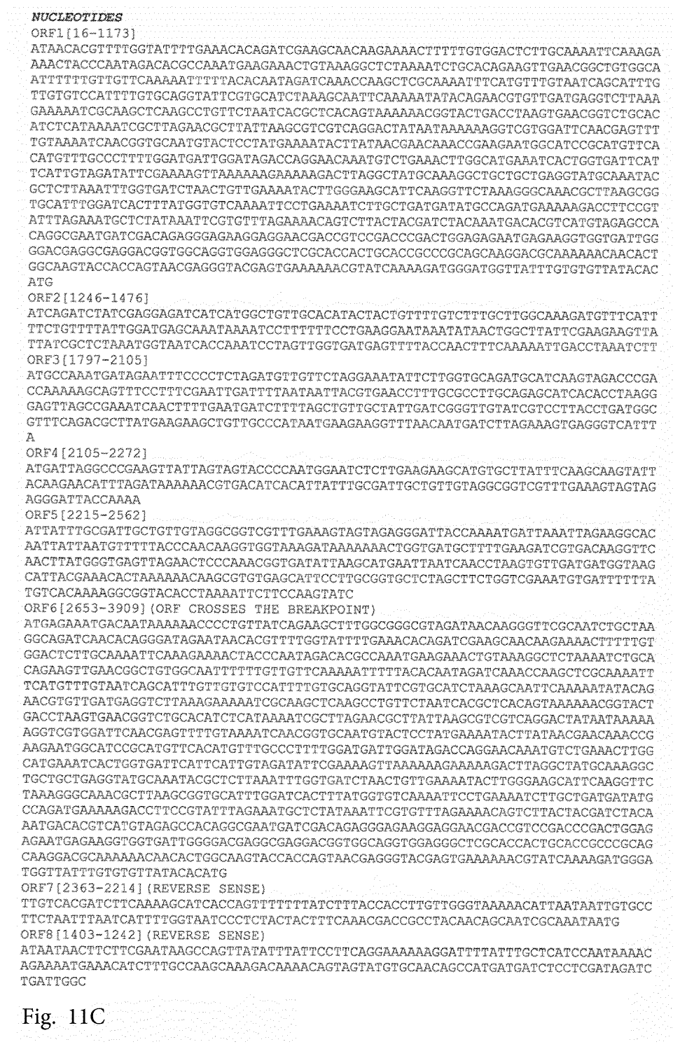

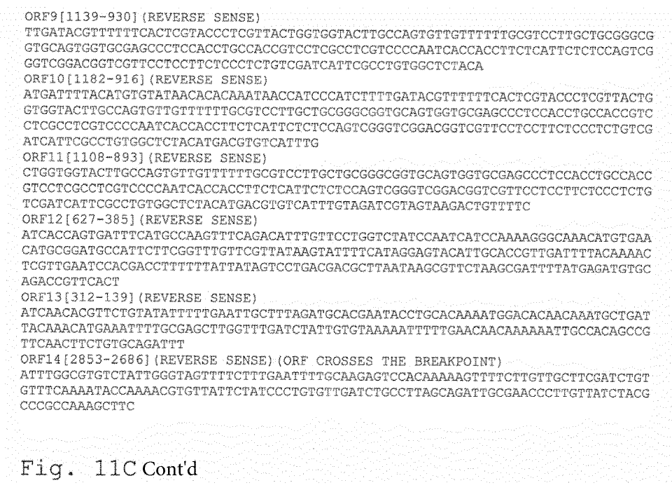

C2MI.8B.7 (FIG. 11A-11C)

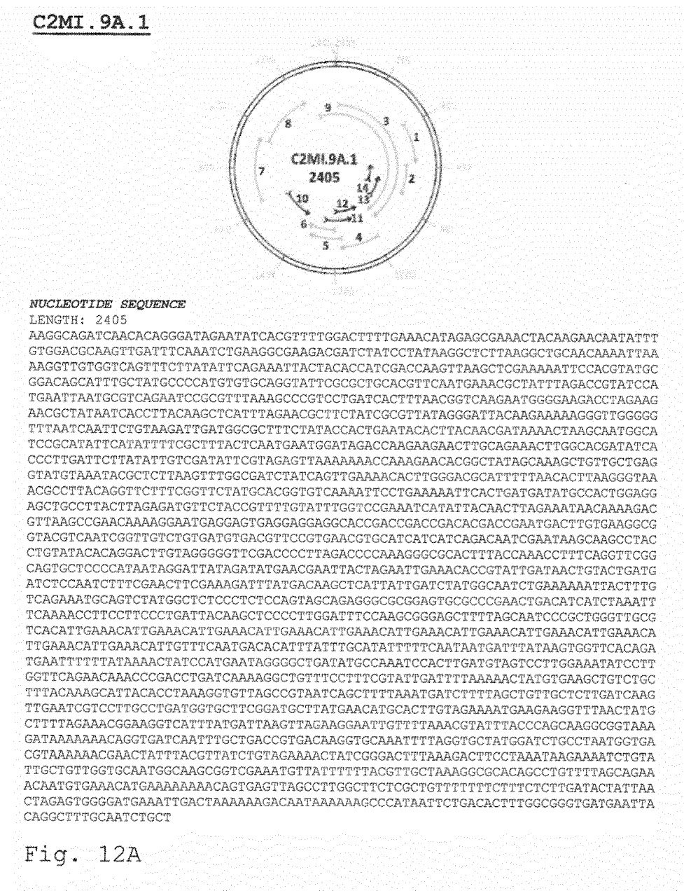

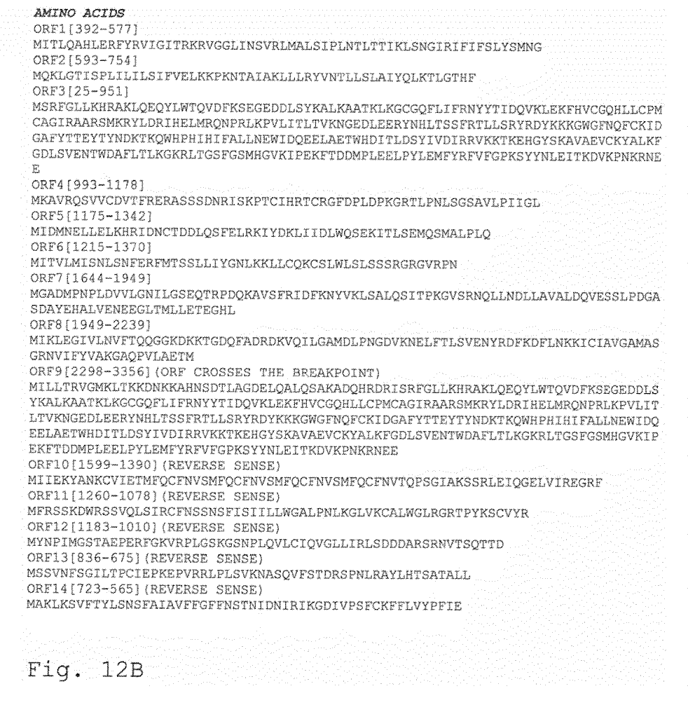

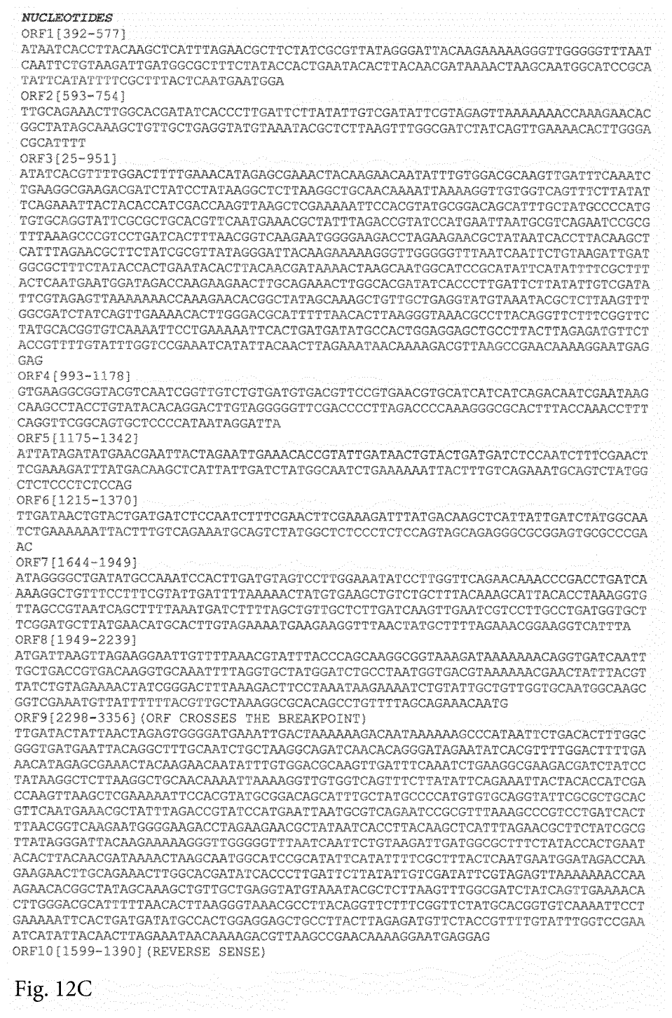

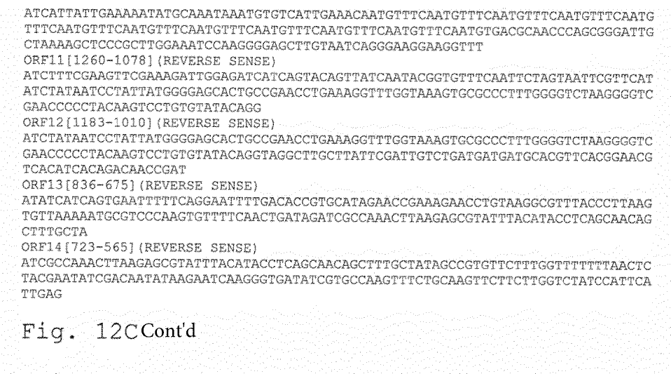

C2MI.9A.1 (FIG. 12A-12C)

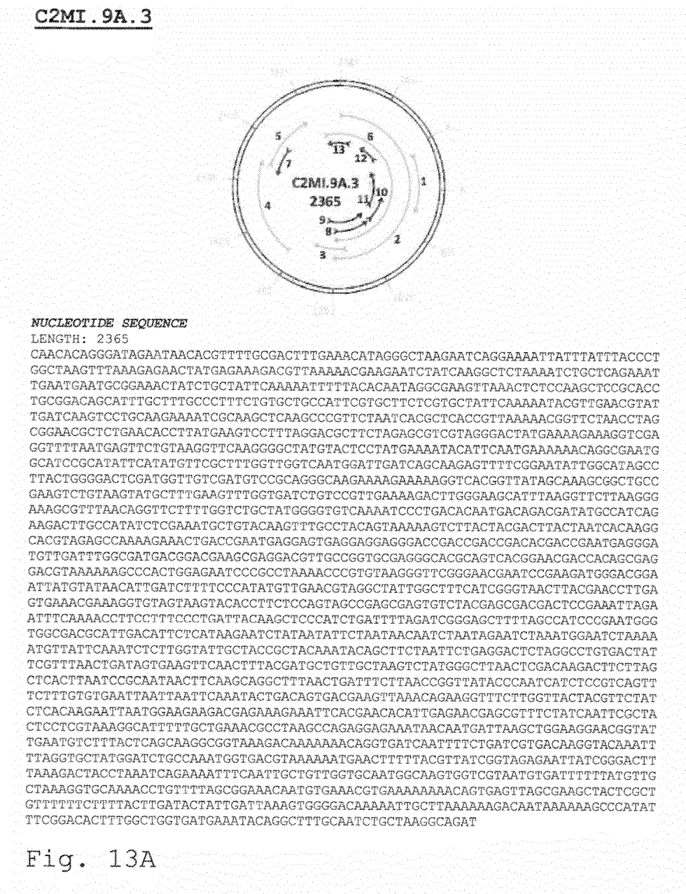

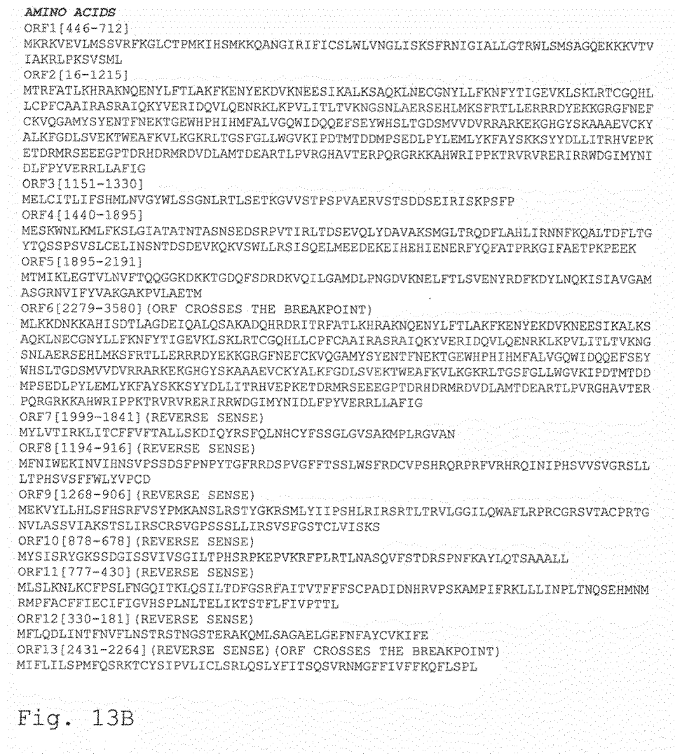

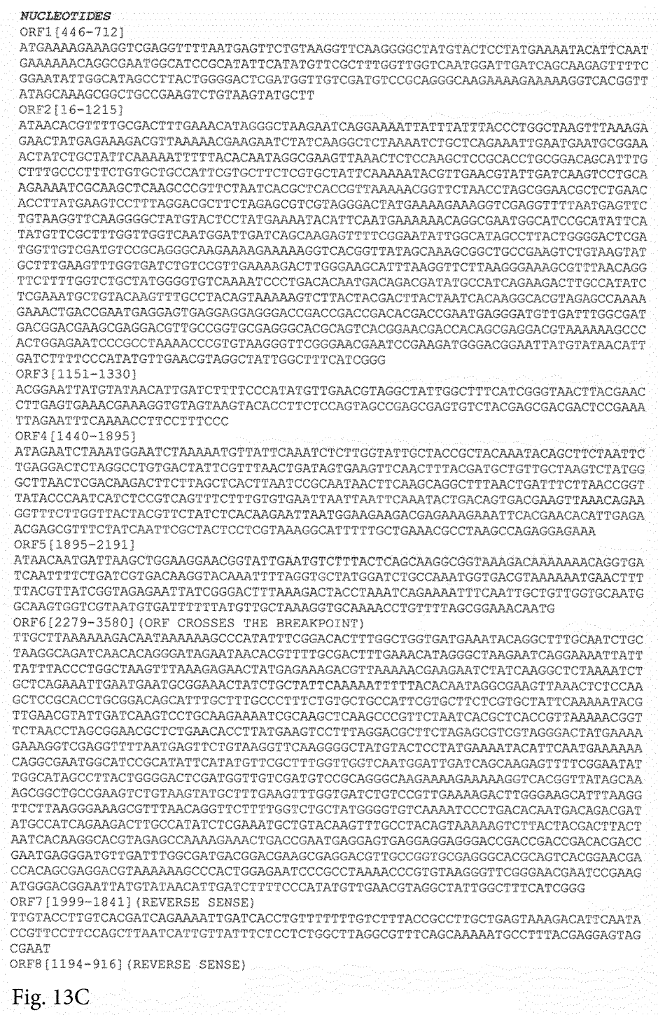

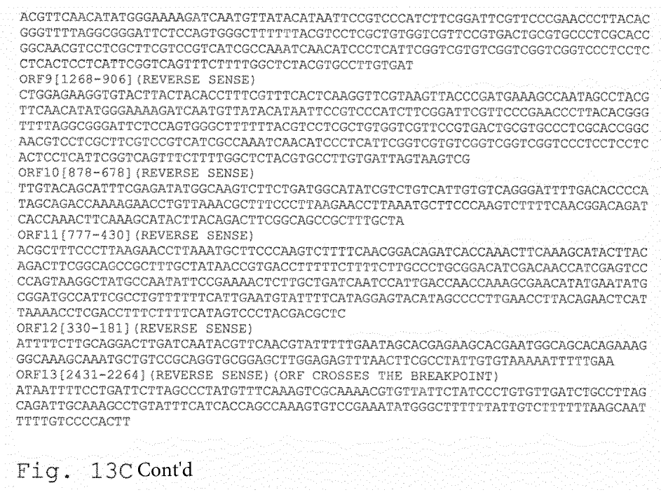

C2MI.9A.3 (FIG. 13A-13C)

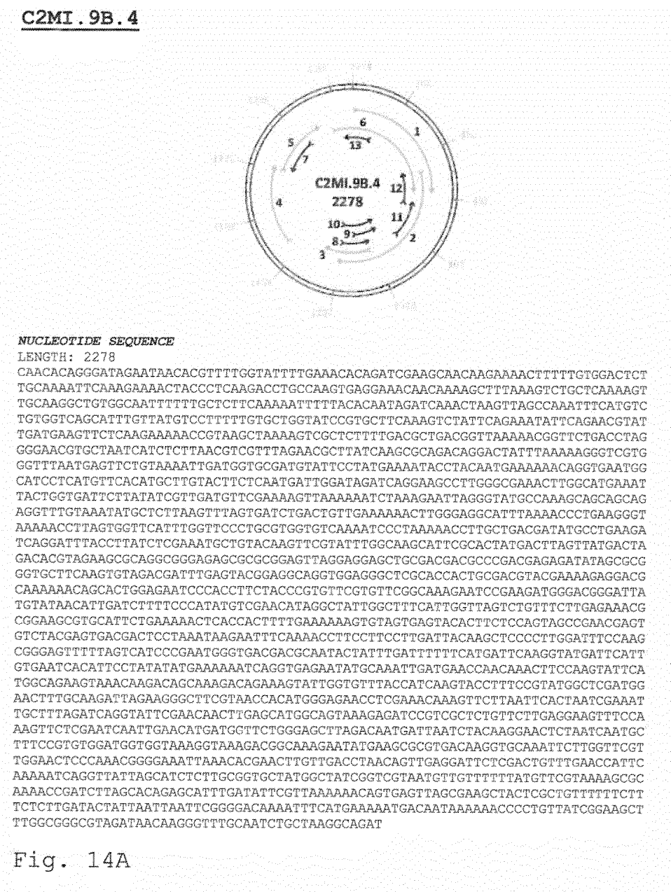

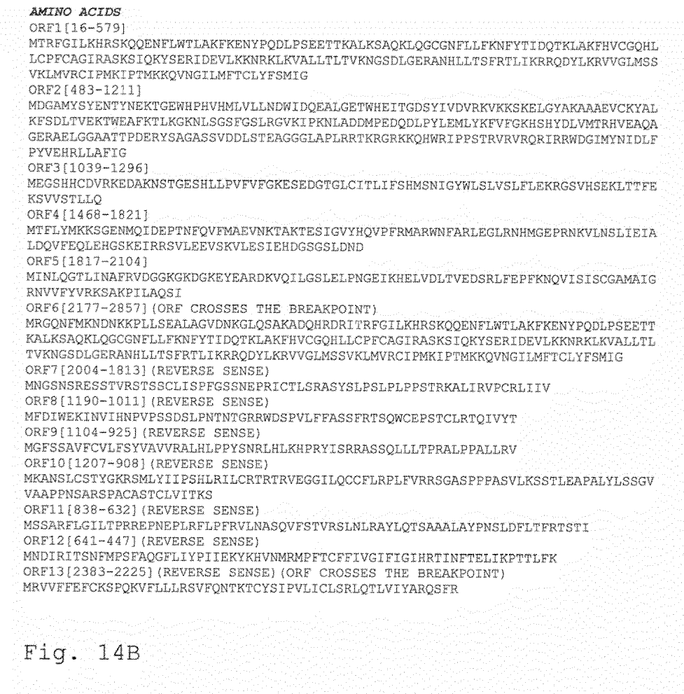

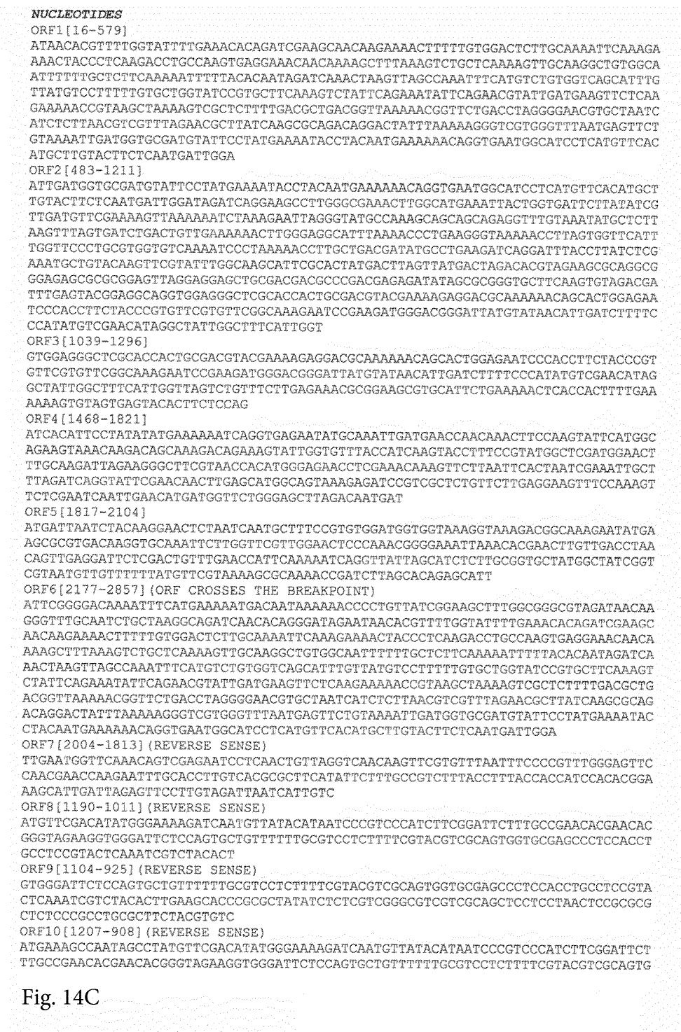

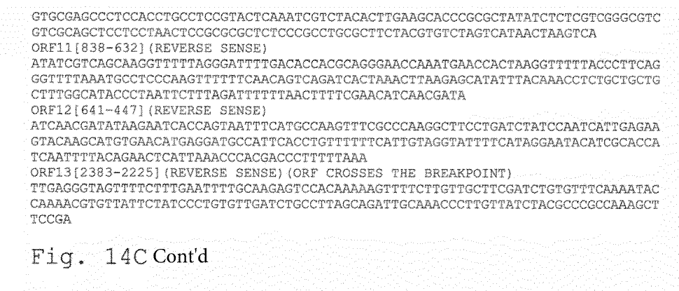

C2MI.9B.4 (FIG. 14A-14C)

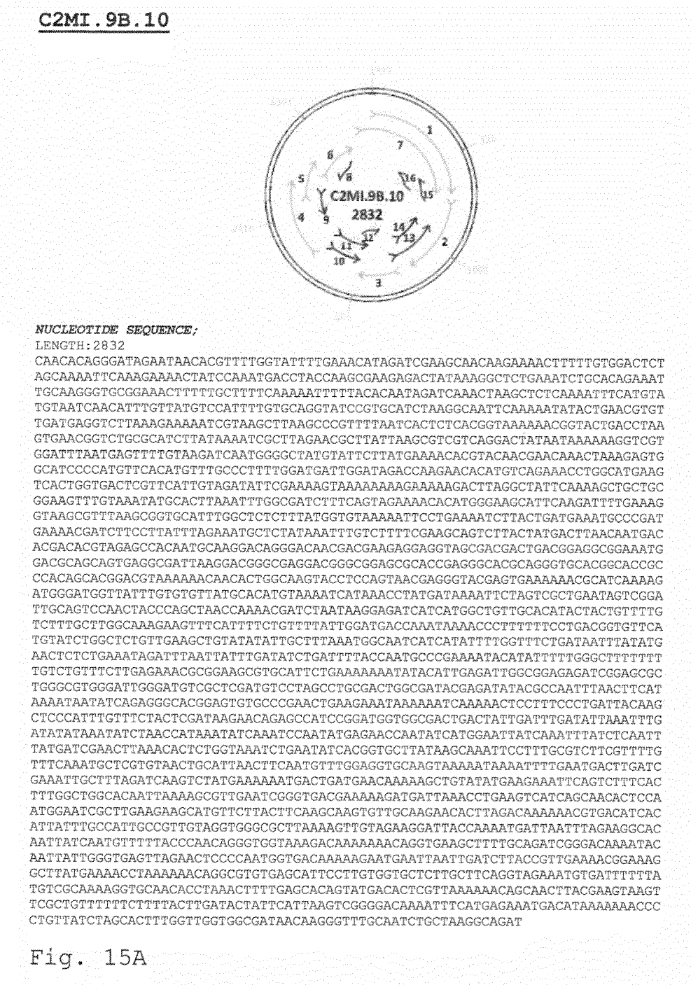

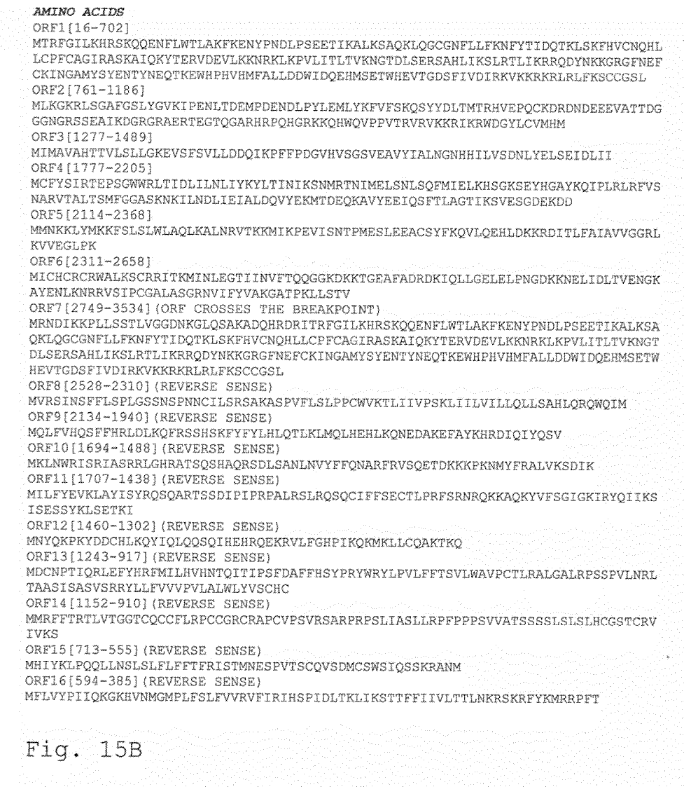

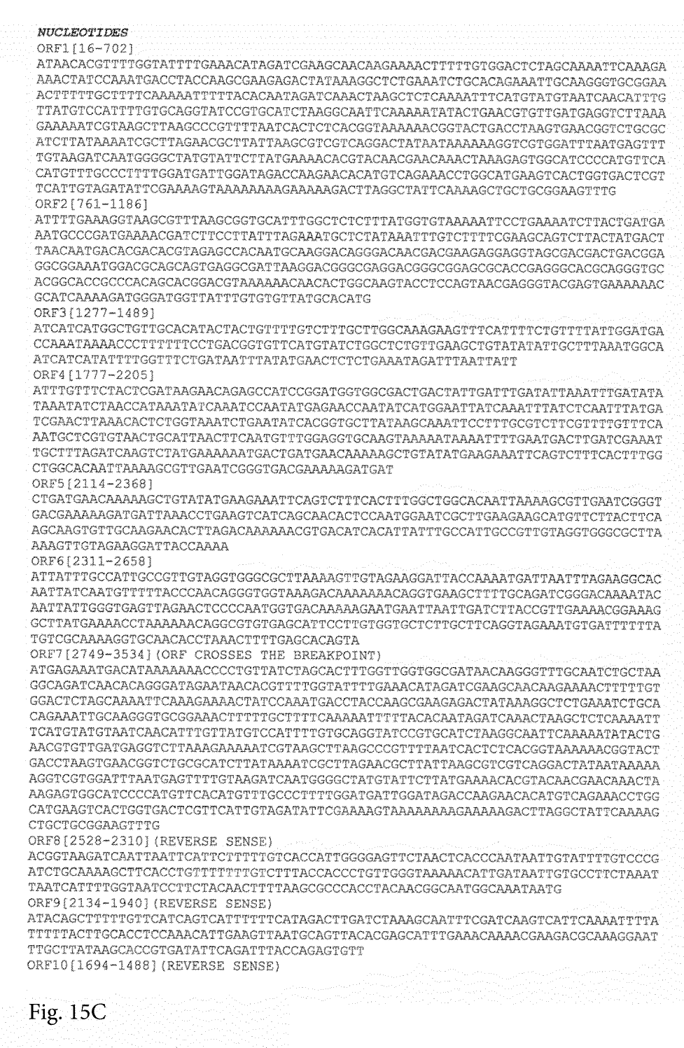

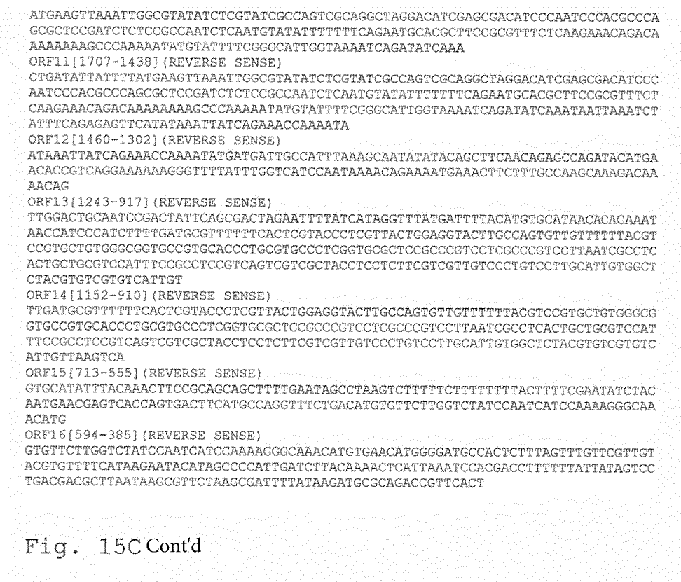

C2MI.9B.10 (FIG. 15A-15C)

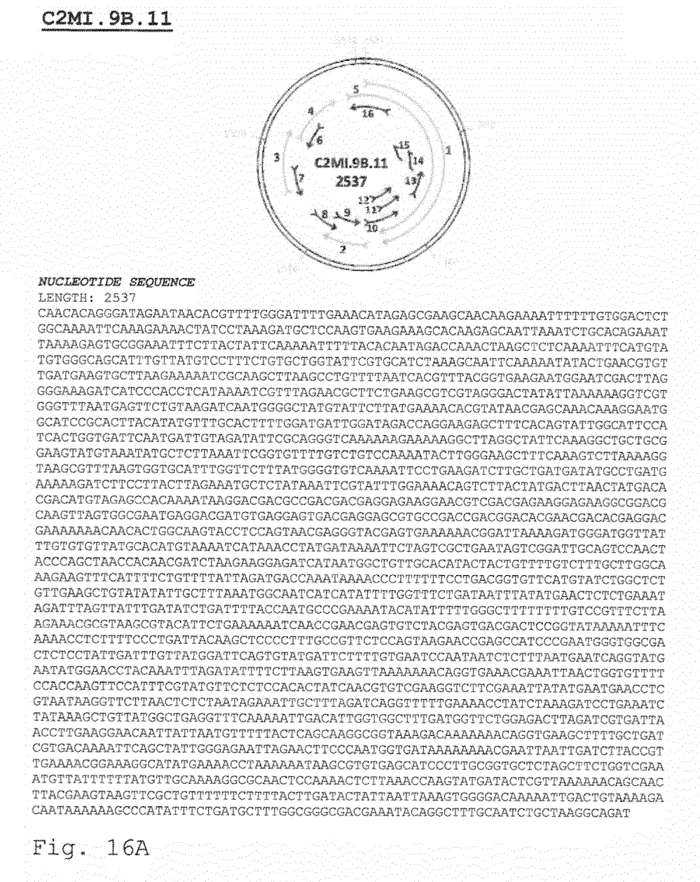

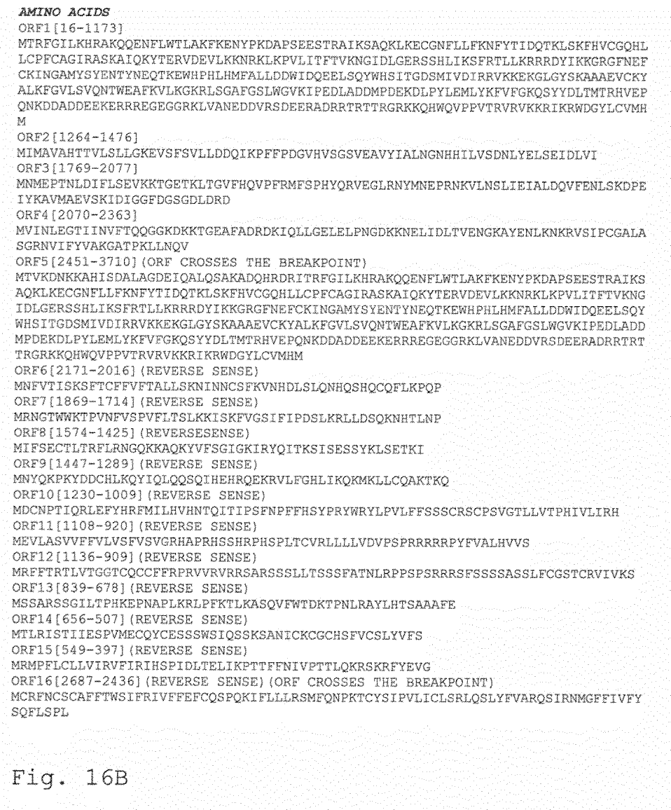

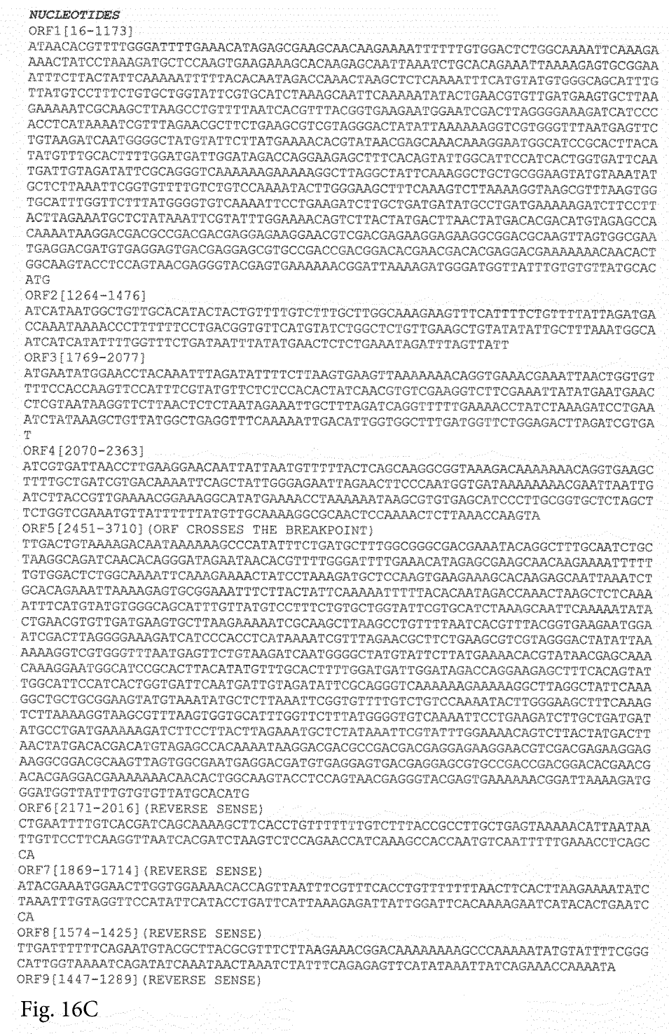

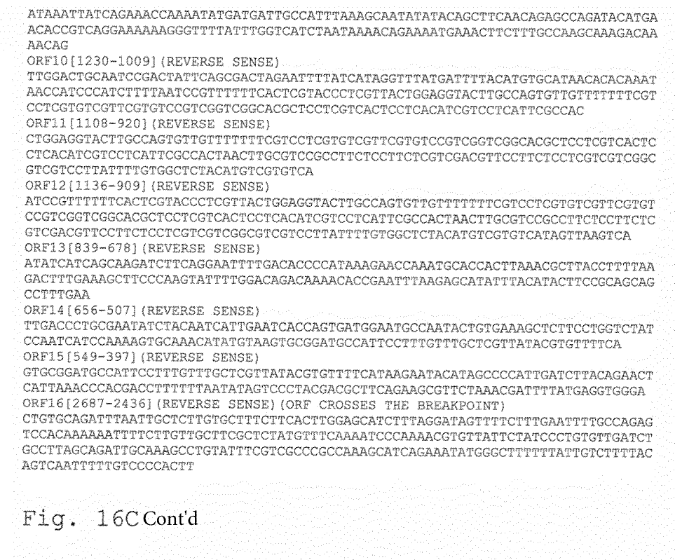

C2MI.9B.11 (FIG. 16A-16C)

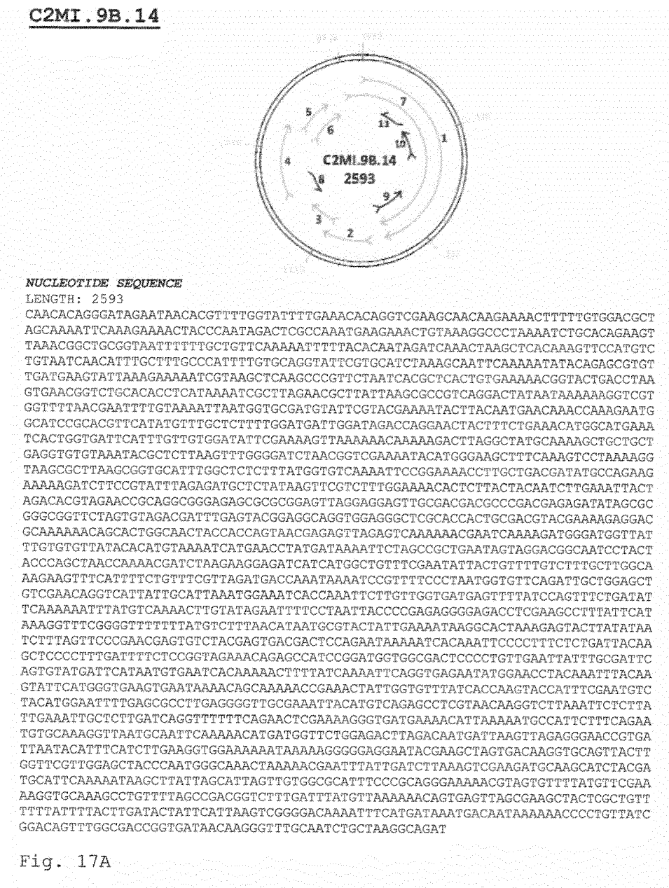

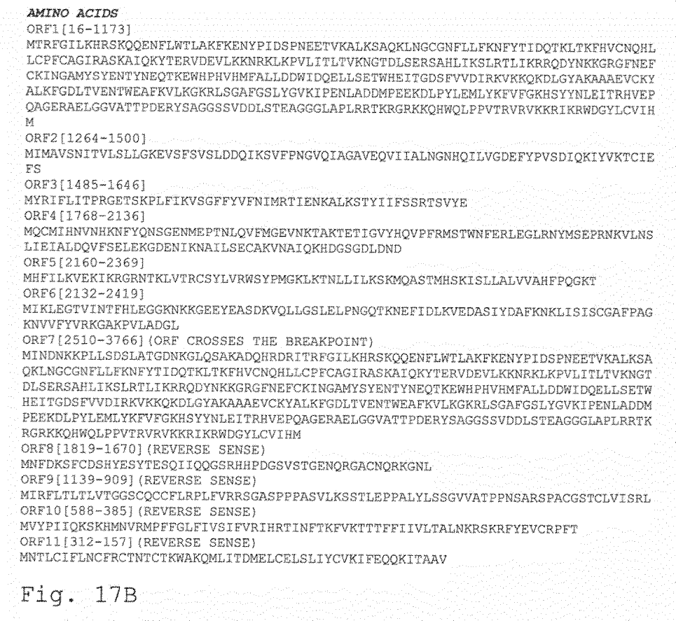

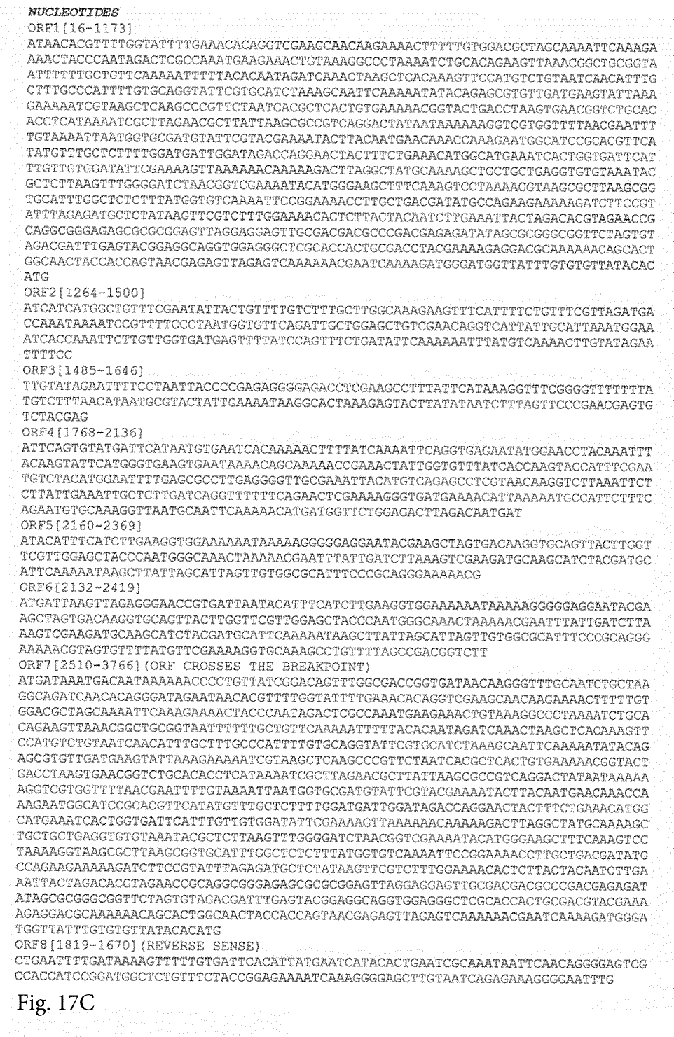

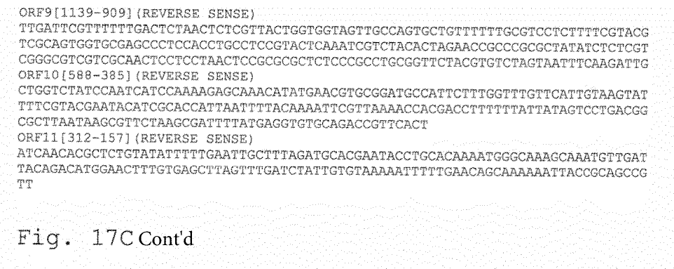

C2MI.9B.14 (FIG. 17A-17C)

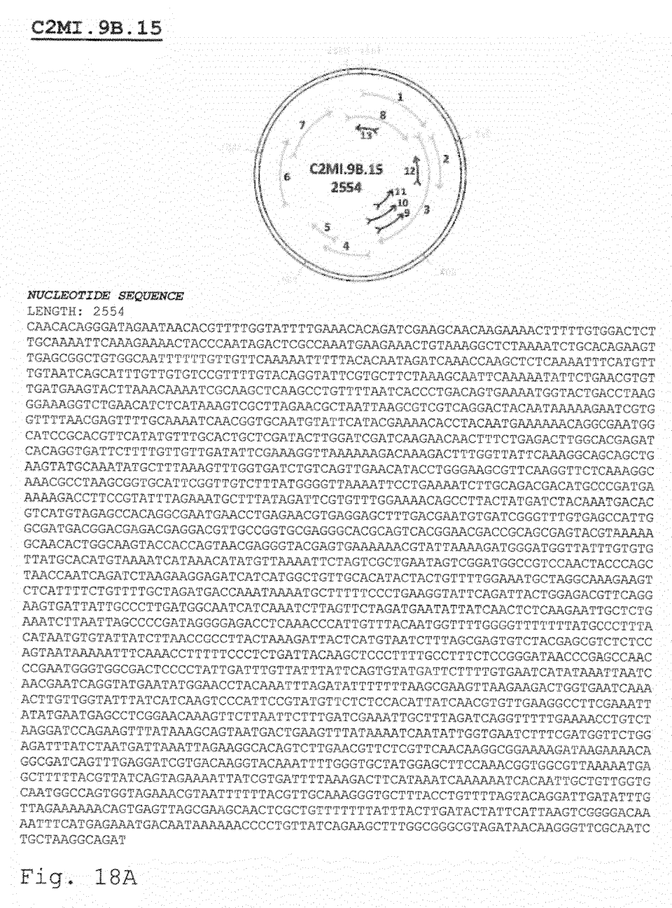

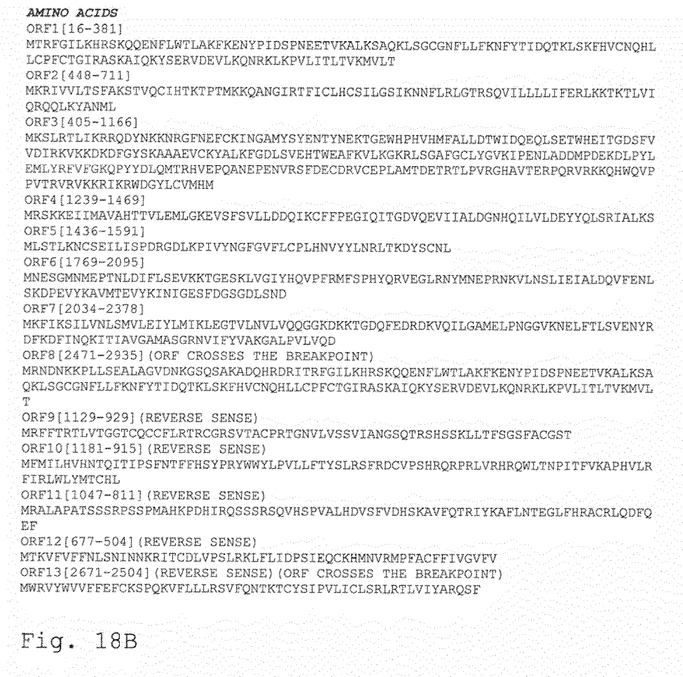

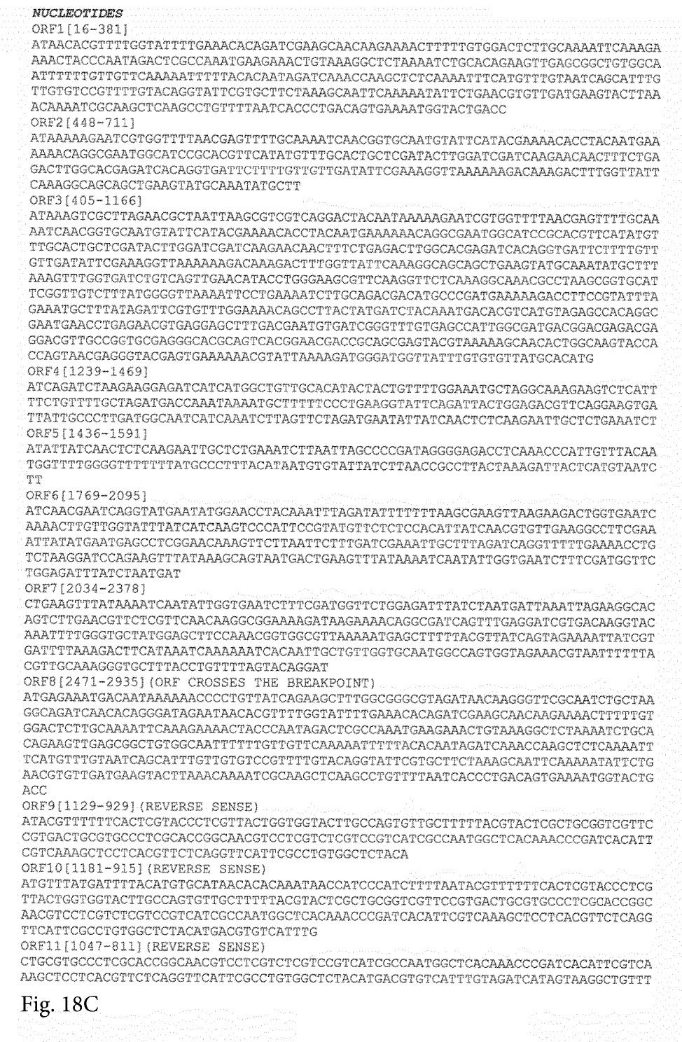

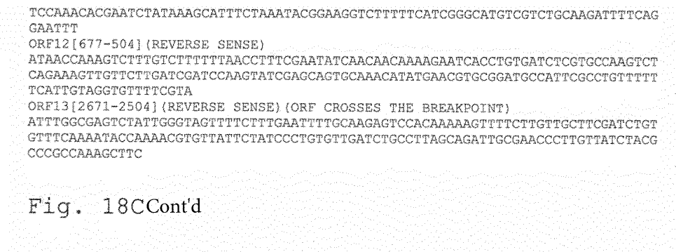

C2MI.9B.15 (FIG. 18A-18C)

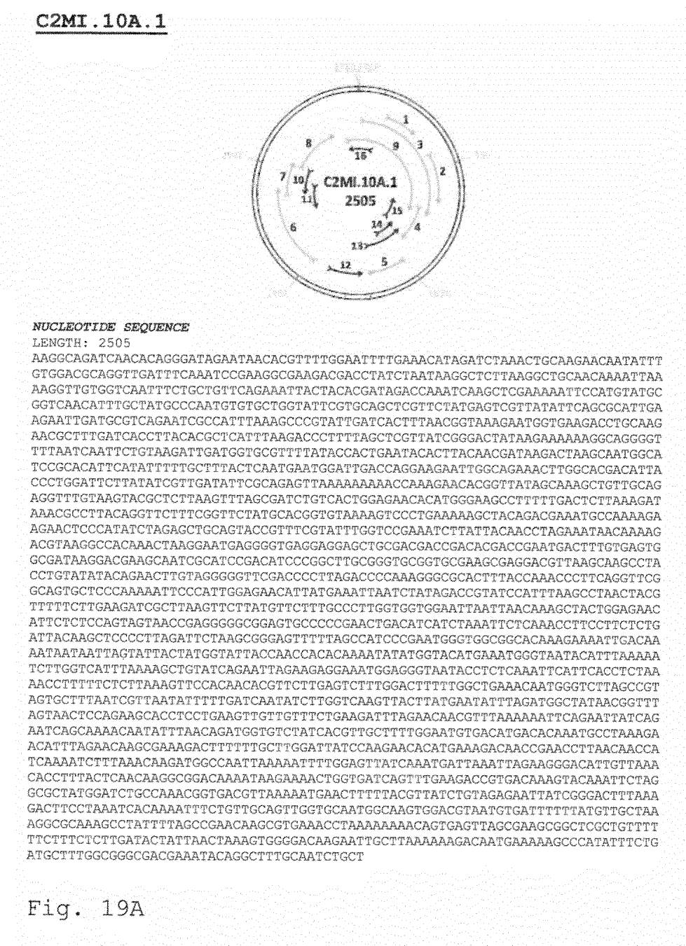

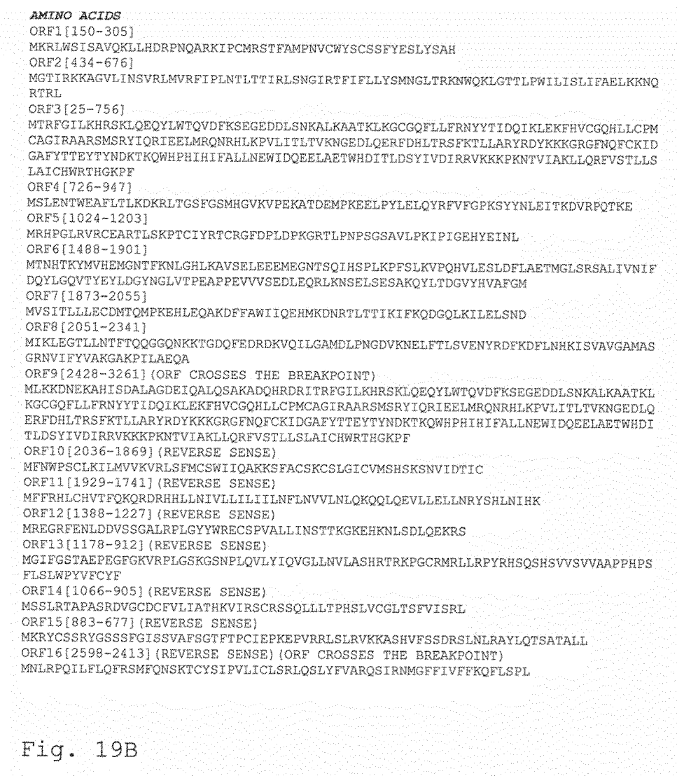

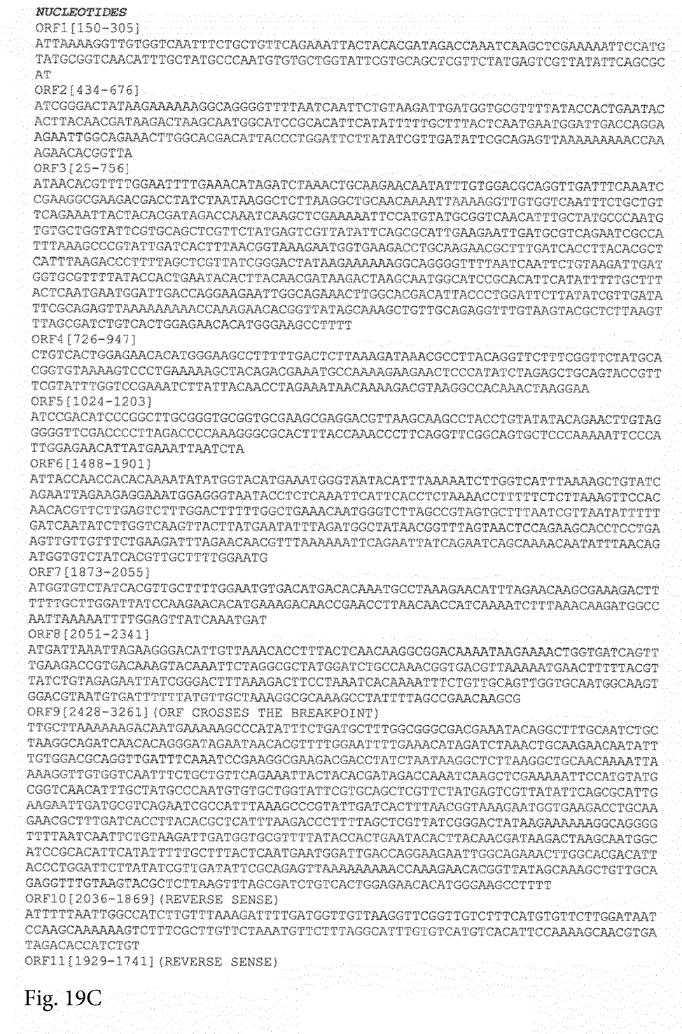

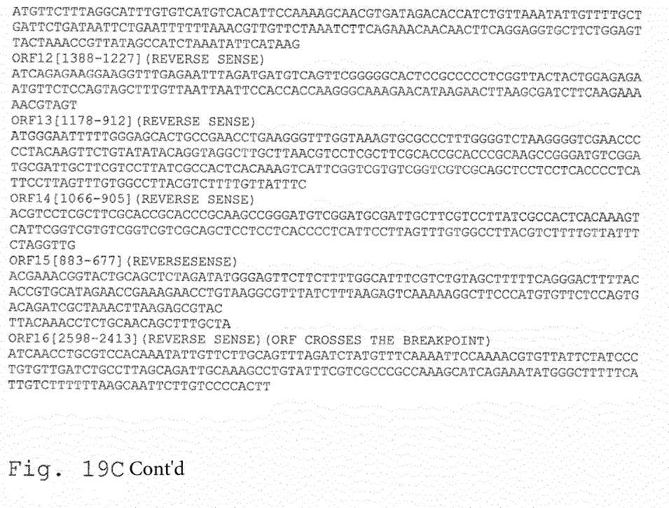

C2MI.10A.1 (FIG. 19A-19C)

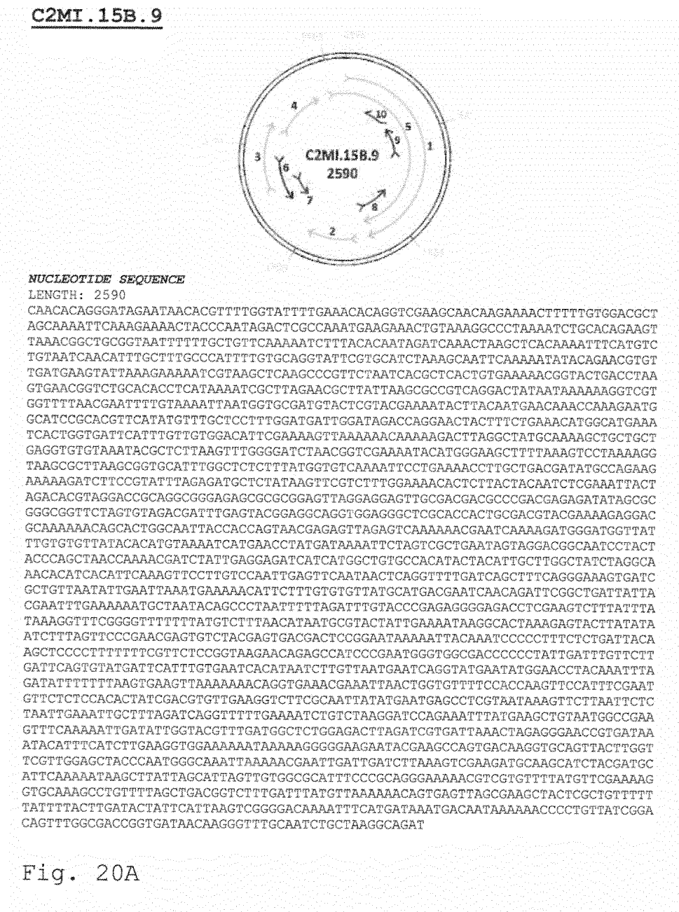

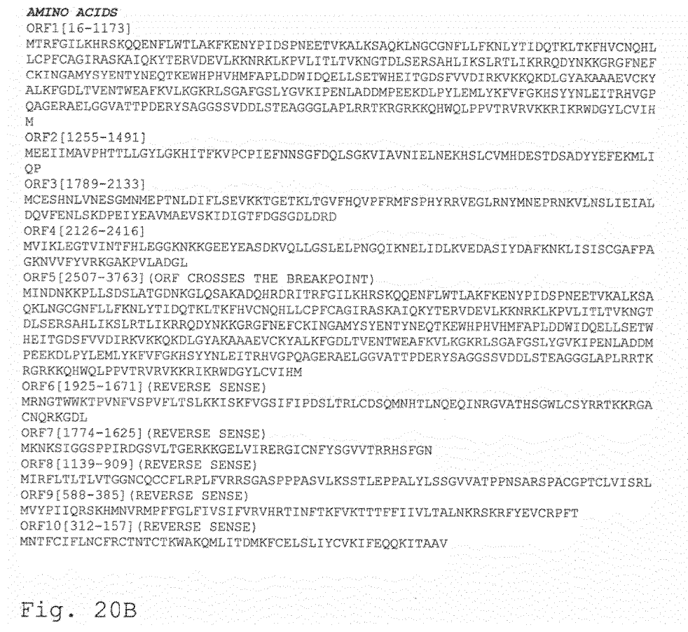

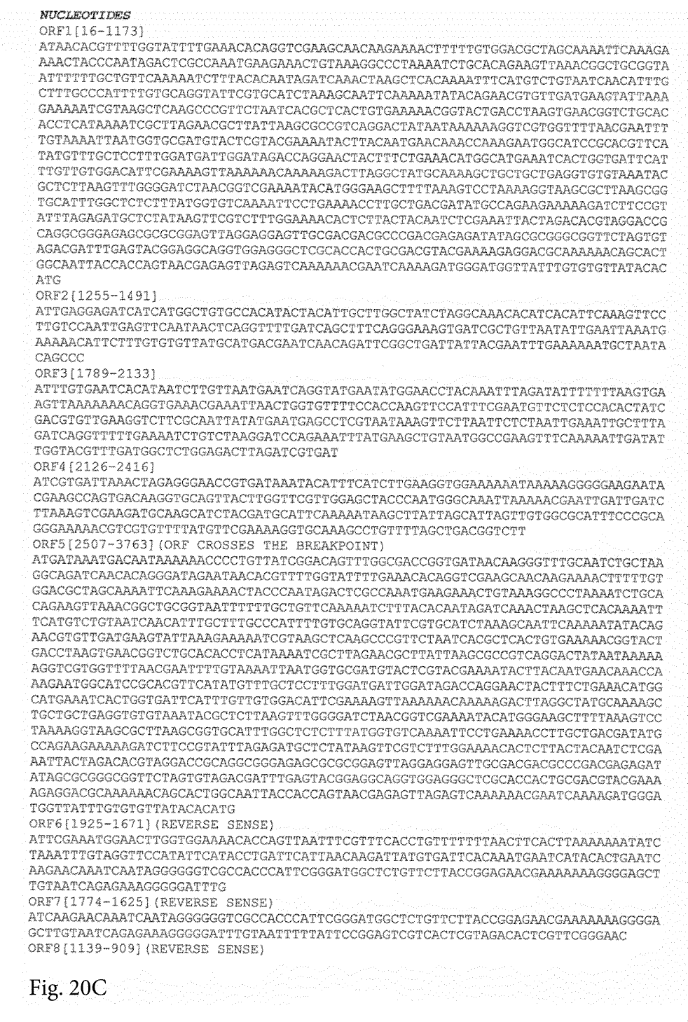

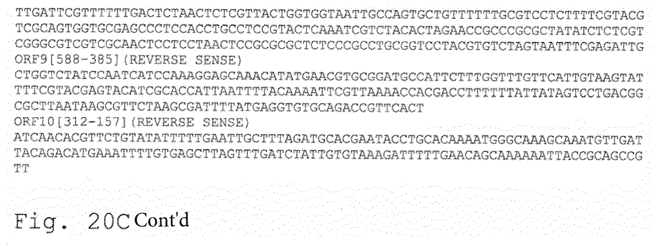

C2MI.15B.9 (FIG. 20A-20C)

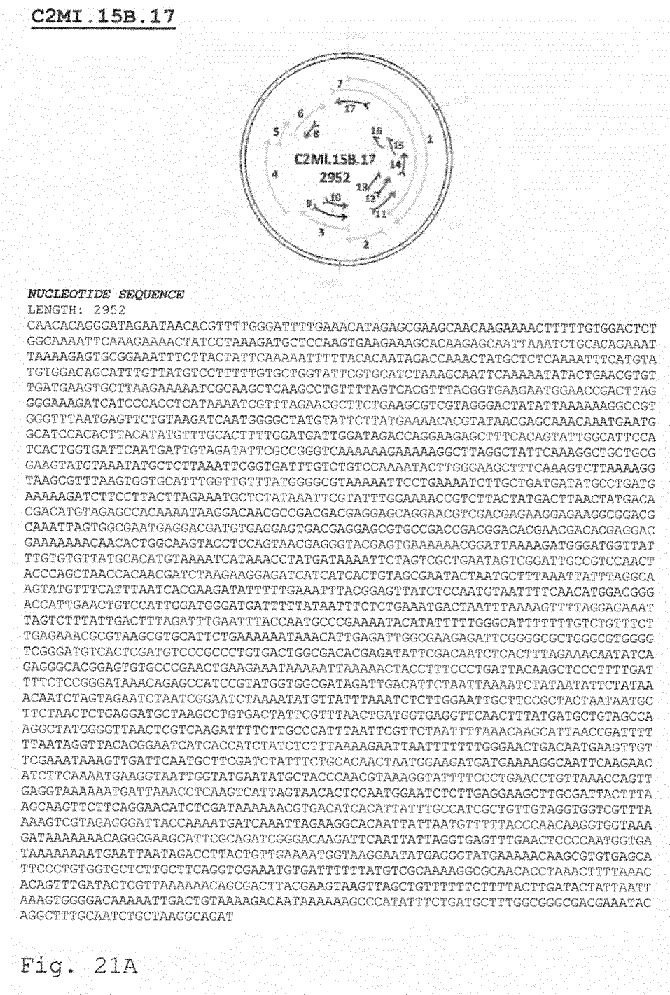

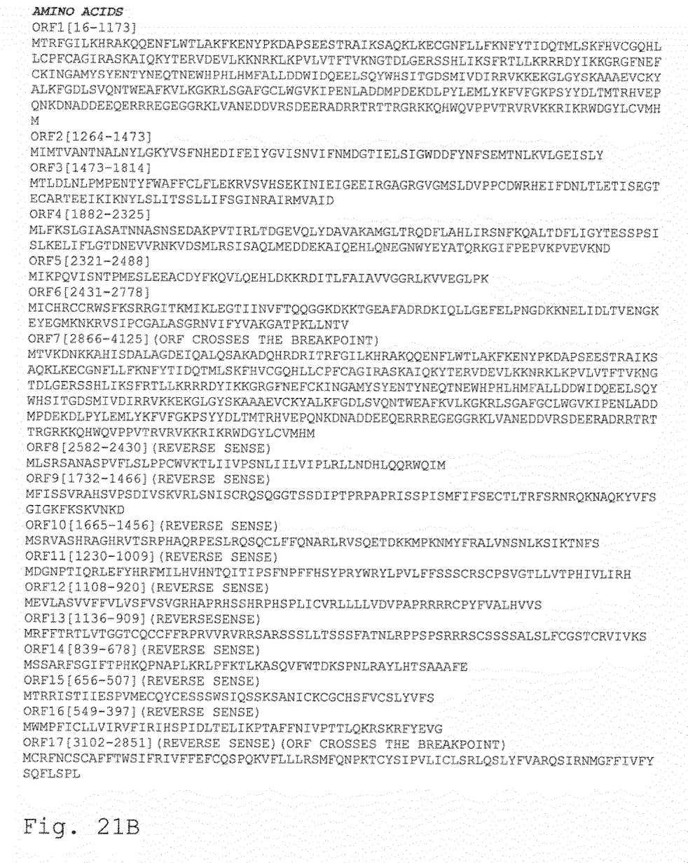

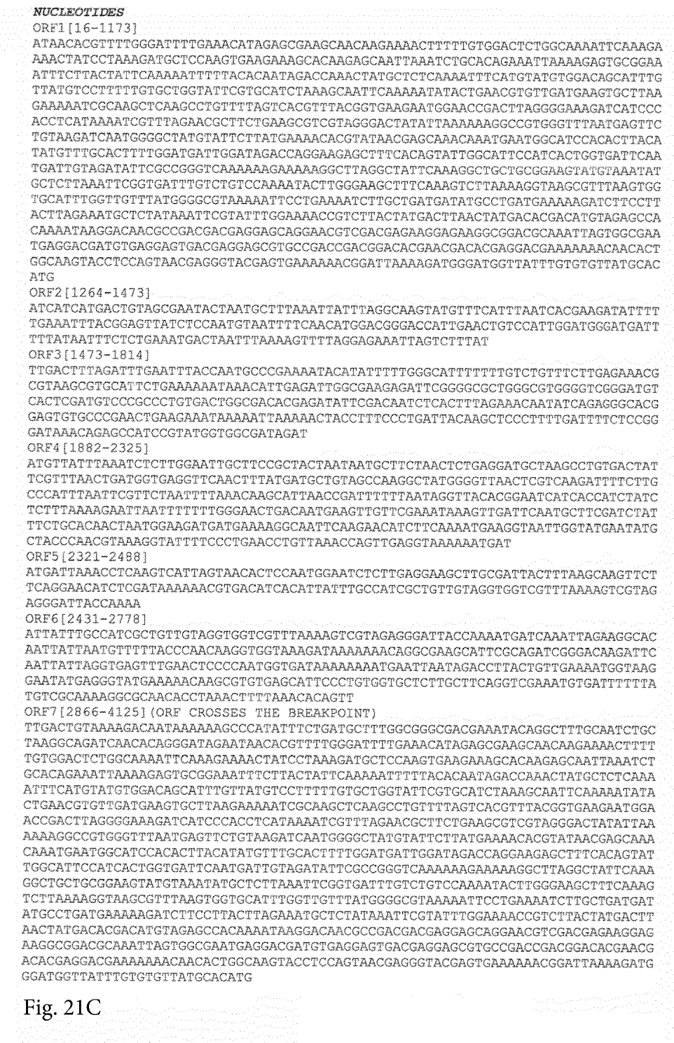

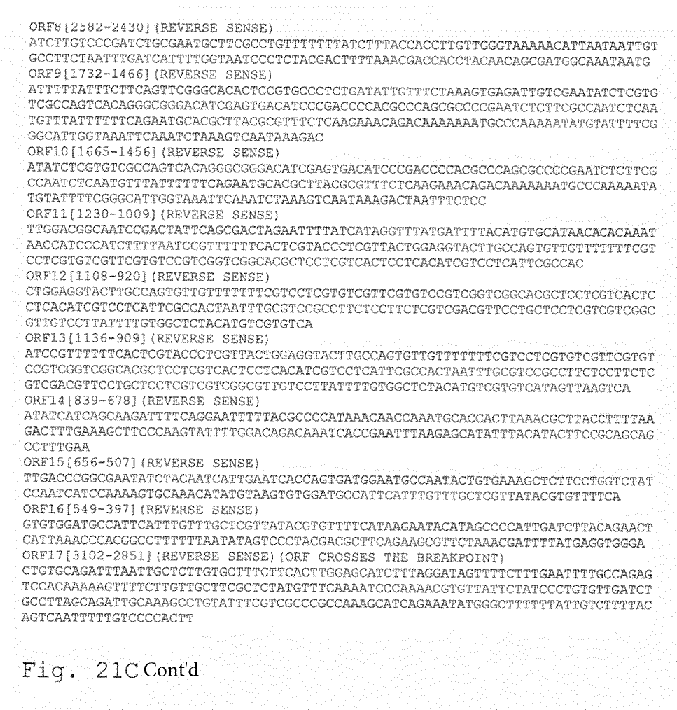

C2MI.15B.17 (FIG. 21A-21C)

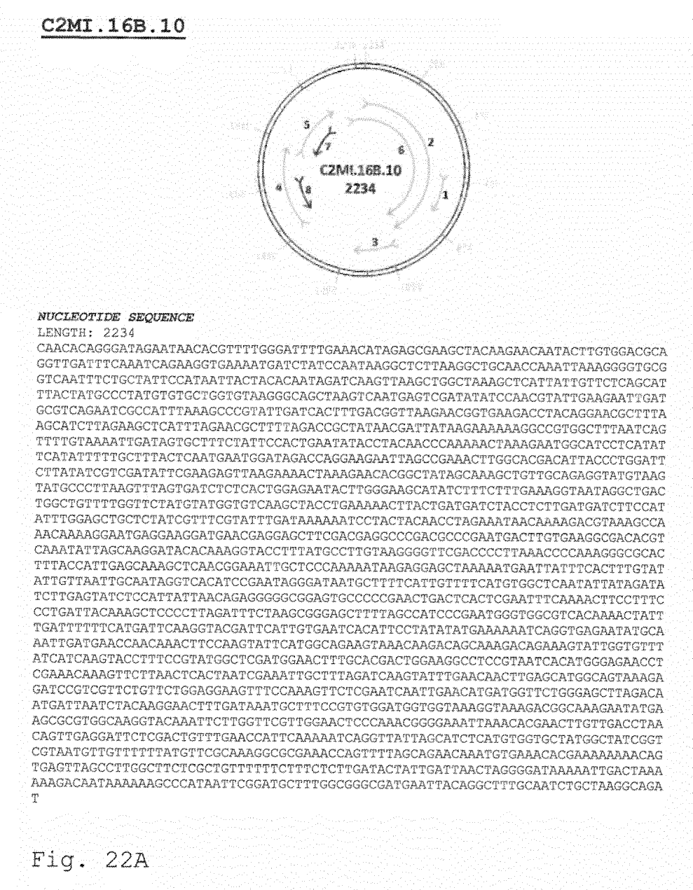

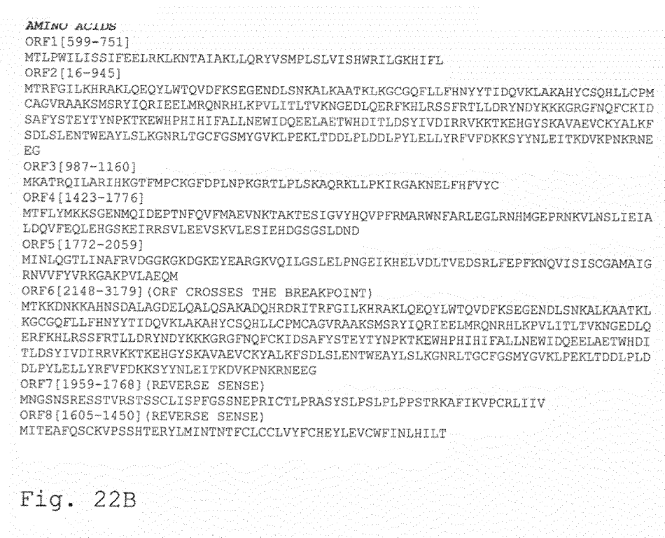

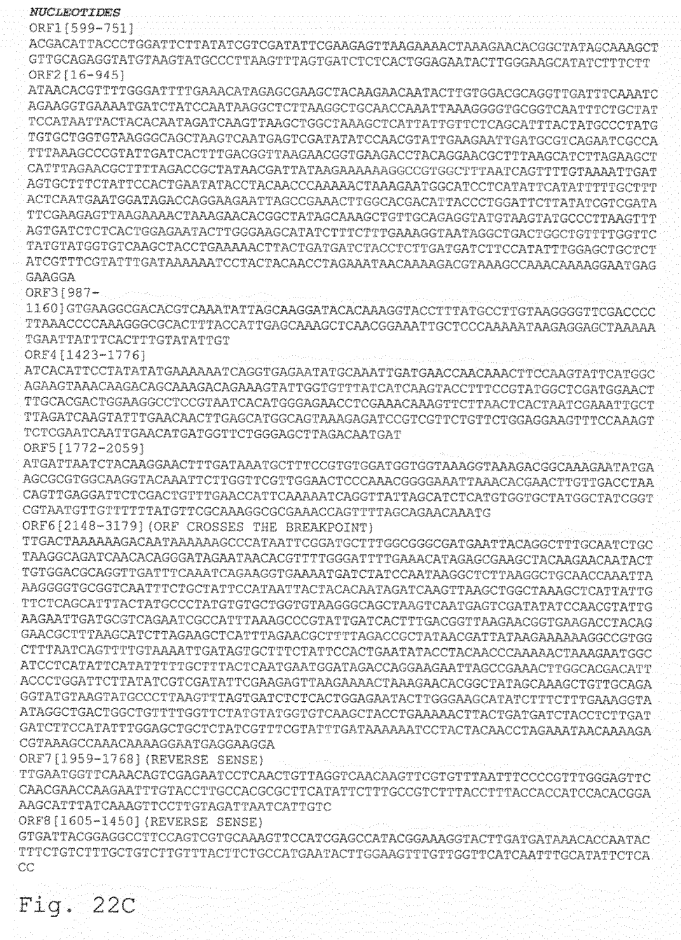

C2MI.16B.10 (FIG. 22A-22C)

### DETAILED DESCRIPTION OF THE PRESENT INVENTION

In the present application a new concept for the pathogenesis of diseases of the CNS (e.g. Multiple sclerosis MS, amyotrophic lateral sclerosis, transmissible spongiforme encephalopathies/Prion-linked diseases, Parkinson's disease, Alzheimer disease), diabetes, atherosclerosis and colon, breast and prostate cancer is presented.

For diseases of the CNS the interaction of an amplifying virus and the amplified DNA of a Bovine Meat and Milk Factor (BMMF) plays a role for their development (zur Hausen et al., 2017)

Two risk factors seem to deserve special attention: the relatively consistent results pointing to a possible role of different, predominantly herpes-group viruses, and the consumption of fresh cow milk, potentially including other dairy products. In addition, Vitamin D deficiency plays a role as a risk factor.

Apparently all Herpes virus types share two properties which seem to be relevant for the subsequent discussion:

During their persistence in a latent stage, spontaneous reactivation may occur, in part regulated by specific gene functions, partly also by epigenetic mechanisms. Reactivation may also be triggered by interaction with extracellular cytokines, such as transforming growth factor 3.

Spontaneous induction of a lytic cycle has been observed for virtually every human pathogenic Herpes virus type. The high antibody titers against Epstein-Barr virus structural proteins in EBV-positive Burkitt's lymphomas and nasopharyngeal cancers may serve here as one example. Reactivations of human Herpes virus type 6, Varicella-Zoster virus, Herpes simplex virus and others are not rare events and may affect a number of different cell types.

A second remarkable property of herpes virus infection represents the amplification of various double- or single-stranded small DNA virus genomes upon infection of cells containing such DNAs in a latent state. This has been noted for human and monkey Polyoma viruses, JC and SV40, for human and bovine Papilloma viruses, as well as for single-stranded Adeno-associated (AAV) and Anello-/TT-viruses.

Spontaneous induction of Herpes-group viruses and the amplification of latent small viral DNA form the basis for the subsequent postulation of the mechanism underlying diseases of the CNS.

A correlation between consumption of non-pasteurized cow milk and MS development has been observed, in particular when non-pasteurized cow milk was consumed in the early phase of life (zur Hausen, 2017; zur Hausen, 2018).

A role of vitamin-D deficiency has repeatedly been implicated for the initiation of MS.

A convincing relationship between vitamin D deficiency and Epstein Barr Virus reactivation originates from early studies on EBV reactivation by transforming growth factor beta (TGF-β). A serum factor, purified and labeled as Epstein-Barr virus-inducing factor (EIF) have been reported to be identical to the subsequently described TGF-β molecule. TGF-β in turn is negatively regulated by activated vitamin D receptors. This could very well explain the season-related preferential onset of MS and of exacerbations.

Thus, at least two factors have been implicated as potential etiological contributors for both diseases of the CNS (e.g. MS) and cancer (e.g. colon and breast cancer): vitamin D deficiency and the reactivation of various herpes group viruses, mainly Epstein-Barr virus (EBV), human herpes virus type 6, and varicella-zoster virus.

Thus, it is considered by the inventors that diseases of the CNS, in particular Multiple Sclerosis (MS), Parkinson, ALS or Alzheimer, result from
Latent infections of the same cell with two different infectious agents, one of them most likely a herpes-type virus (in particular EBV, HHV-6, VZV, but also HSV, HHV-7), the other one acquired by bovine milk consumption (bovine milk factor—BMF) as the first event. Each of them latently infects individual cells, but occasionally genomes of both agents occur within the same cell.
Reactivation of the herpes-type virus most frequently, but not only, Epstein Barr virus (EBV) to a lytic cycle as a second precondition. For EBV this is probably linked to increased levels of transforming growth factor R (TGF 3) which is negatively regulated by activated vitamin D receptors;
As third event, amplification of BMMF, resulting in partial suppression of Herpes-type DNA synthesis and formation of BMF particles or spreading of its nucleic acid to neighbouring cells via neuronal interconnections;
This is followed by an infection of neighbouring cells with expression of BMMF protein;
Finally, T-cell response against BMMF leads to the destruction of affected cells and in case of MS to plaque formation. This supports the clinical observation of the focal appearance of lesions, commonly starting from a central vein and the intensive localized immune response in early lesions.

The risk for insulin-dependent diabetes mellitus or atherosclerosis has been repeatedly also linked to cow milk consumption and vitamin D deficiency. The latter system seems to come particularly close to the MS situation.

This concept also may apply to other presumably autoimmune diseases and certain cancers occurring at increased frequency under conditions of vitamin D deficiency. As far as cancers are concerned, this specifically accounts for colon- and breast cancer, and possibly for ovarian, prostate, pancreatic cancer and lung cancers.

As regards colon cancer the inventors found that the uptake of BMMF (Bovine Meat and Milk Factor) agents within the first months of life either by substitution of breast-feeding during weaning by cow milk products or by the uptake of cow milk products, in general, leads to the early infection of newborns with BMMF antigens in the colon. Based on the decline of maternal antibodies and the frequently observed weakness of the immune system often coupled with induction of immunotolerances of the newborn during this very early period of life, these agents might either directly escape colonic immune response or a situation of immunotolerance against these agents might be induced. Within the next years or decades—depending on the immune system of the host—more and more BMMF antigens accumulate within the mesenchymal region of the lamina propria. This accumulation may be triggered also by the uptake of specific molecules that may represent receptors for BMMFs. These molecules are also taken up by consumption of cow products and are metabolized into receptors on the surface of the host cells. When a certain level of antigen is reached by continuous uptake of BMMFs in combination with focal spreading of infection, the host immune response induces a state of chronic and local inflammation producing a stable increase of reactive oxygen species (ROS) and cyclooxygenase-2 (Cox-2) which dramatically increases the probability deregulated cell proliferation with concomitant fixation of random mutations in surrounding cells induced by ROS.

Available results based on Ki67 (Ki67: proliferation marker) IHC staining show that especially the basal epithelial cells derived from epithelial stem cells located close to the endpoint of the crypts and migrating towards the distal luminal part of the crypts feature an intrinsically high proliferation enabling stochastic manifestation of mutations as a basic requirement for tumorgenesis and development of colon cancer. Thus, BMMFs represent a specific and local trigger for induction of chronic inflammation within mesenchymal tissue leading to an increase of ROS inducing proliferation and mutation in surrounding epithelial cells eventually leading to the formation of polyps as source for colon cancer.

Accordingly, the present invention relates to a C2MI polynucleic acid which may comprise:
(a) a nucleotide sequence depicted in any one of FIGS. 1(A) to 22(A);
(b) a nucleotide sequence having at least 93% identity to a nucleotide sequence of (a);
(c) a fragment of a nucleotide sequence of (a) or (b);
(d) a nucleotide sequence being complementary to a nucleotide sequence of (a), (b) or (c); or
(e) a nucleotide sequence which is redundant as a result of the degeneracy of the genetic code compared to any of the above-given nucleotide sequences.

The term “polynucleic acid” refers to a single-stranded or double-stranded nucleic acid sequence. A polynucleic acid may consist of deoxyribonucleotides or ribonucleotides, nucleotide analogues or modified nucleotides or may have been adapted for diagnostic or therapeutic purposes. A polynucleic acid may also comprise a double stranded cDNA clone which can be used, for example, for cloning purposes.

The C2MI polynucleic acids of the invention can be prepared according to well-known routine methods, for example, by (a) isolating the entire DNA or RNA from a sample, (b) detecting the CMI sequence by hybridization or PCR and (c) cloning of the C2MI sequence into a vector.

Also included within the present invention are sequence variants and recombinants of the polynucleic acid of the invention containing either deletions and/or insertions of one or more nucleotides, especially insertions or deletions of one or more codons, mainly at the terminal ends of oligonucleotides (either 3′ or 5′) but also within and which show at least 93%, 94%, 95%, 96%, 97%, 98% or 99% identity as well as recombination to said polynucleic acid sequences of the invention. Polynucleic acid sequences according to the present invention which are similar to the sequences as shown in FIGS. 1 to 22 can be characterized and isolated according to any of the techniques known in the art, such as amplification by means of sequence-specific primers, hybridization with sequence-specific probes under more or less stringent conditions, sequence determination of the genetic information of C2MI.

The present invention also provides functional fragments of the nucleotide sequences of the present invention described above that harbour a replication gene which codes for a replication protein. An autonomous replicating nucleotide sequence may comprise a nucleotide sequence of the replication gene or a fragment thereof which is capable of inducing autonomous replication. Replication protein represents an endonuclease which binds single-stranded DNA inducing a single-stranded cut at or near the origin of replication (Wolds, 1997). The skilled person can derive at such fragments capable of inducing autonomous replication without undue experimentation. Such fragments may have a length of at least 45, at least 55, or at least 65 nt.

The person skilled in the art can easily determine by using standard assays which nucleic acid sequences are related to a nucleotide sequence of FIG. 1 to 22 or are fragments thereof and have still the same function as the full length sequences.

The present invention also provides polynucleic acid sequences which are redundant as a result of the degeneracy of the genetic code compared to any of the above-given nucleotide sequences. These variant polynucleic acid sequences will thus encode the same amino acid sequence as the polynucleic acids they are derived from.

The C2MI polynucleic acids of the invention might be present as an extrachromosomal episome, might be integrated into the host's genome and/or might be linked to a host cell DNA.

The present invention also relates to an oligonucleotide primer which may comprise or consisting of part of a polynucleic acid as defined above, with said primer being able to act as primer for sequencing or specifically amplifying C2MI polynucleic acid of the invention.

The term “primer” refers to a single stranded DNA oligonucleotide sequence capable of acting as a point of initiation for synthesis of a primer extension product which is complementary to the nucleic acid strand to be copied. The length and the sequence of the primer must be such that they allow priming the synthesis of the extension products. Preferably the primer is about 5-50 nucleotides. Specific length and sequence will depend on the complexity of the required DNA or RNA targets, as well as on the conditions of primer use, such as temperature and ionic strength.

The fact that amplification primers do not have to match exactly with a corresponding template sequence to warrant proper amplification is an accepted principle. The amplification method used can be, for example, polymerase chain reaction (PCR), ligase chain reaction (LCR), nucleic acid sequence-based amplification (NASBA), transcription-based amplification system (TAS), strand displacement amplification (SDA) or amplification by means of Qb replicase or any other suitable method to amplify nucleic acid molecules using primer extension. During amplification, the amplified products can be labelled either using labelled primers or by incorporating labelled nucleotides. In a particular preferred embodiment the C2MI sequences of the present invention have been obtained by using two sets of abutting PCR primers overlapping in the highly conserved region of their Rep genes to amplify full-length circular DNA molecules. All C2MI isolates of the present invention were isolated by using a back-to-back primer pair A or B (5p->3p). The “A” or “B” in the sequence name denotes these two primer airs.

Primer Pair A:
>LSconAfwd
aaggcagatcaacacagg
>LSconArev
agcagattgcaaagcctg
Primer Pair B:
>LSconBfwd
caacacagggatagaataac
>LSconBrev
atctgccttagcagattgc

Labels may be isotopic (32P, 355, etc.) or non-isotopic (biotin, digoxigenin, etc.). The amplification reaction is repeated between 20 and 70 times, advantageously between 25 and 45 times.

Any of a variety of sequencing reactions known in the art can be used to directly sequence the genetic information and determine the ORF by translating the sequence of the sample into the corresponding amino acid sequence. Exemplary sequencing reactions include those based on techniques developed by Sanger or Maxam and Gilbert. It is also contemplated that a variety of automated sequencing procedures may be utilized when performing the subject assays including sequencing by mass spectrometry (see, for example: PCT publication WO 94/16101). It will be evident to one skilled in the art that, for example the occurrence of only two or three nucleic bases needs to be determined in the sequencing reaction.

Preferably, these primers are about 5 to 50 nucleotides long, more preferably from about 10 to 25 nucleotides. Most preferred are primers having a length of at least 13 bases.

The present invention also relates to an oligonucleotide probe which may comprise or consisting of part of a C2MI polynucleic acid as defined above, with said probe being able to act as a hybridization probe for specific detection of a C2MI polynucleic acid according to the invention.

The probe can be labelled or attached to a solid support.

The term “probe” refers to single stranded sequence-specific oligonucleotides which have a sequence which is complementary to the target sequence of a C2MI polynucleic acid to be detected.

Preferably, these probes are about 5 to 50 nucleotides long, more preferably from about 10 to 25 nucleotides. Most preferred are probes having a length of at least 13 bases.

The term “solid support” can refer to any substrate to which an oligonucleotide probe can be coupled, provided that it retains its hybridization characteristics and provided that the background level of hybridization remains low. Usually the solid substrate will be a microtiter plate, a membrane (e.g. nylon or nitrocellulose) or a microsphere (bead). Prior to application to the membrane or fixation it may be convenient to modify the nucleic acid probe in order to facilitate fixation or improve the hybridization efficiency. Such modifications may encompass homopolymer tailing, coupling with different reactive groups such as aliphatic groups, NH₂ groups, SH groups, carboxylic groups, or coupling with biotin or haptens.

The oligonucleotides according to the present invention, used as primers or probes may also contain or consist of nucleotide analogues such as phosphorothioates, alkylphosphoriates or peptide nucleic acids or may contain intercalating agents. These modifications will necessitate adaptions with respect to the conditions under which the oligonucleotide should be used to obtain the required specificity and sensitivity. However, the eventual results will be essentially the same as those obtained with the unmodified oligonucleotides.

The introduction of these modifications may be advantageous in order to positively influence characteristics such as hybridization kinetics, reversibility of the hybrid-formation, biological stability of the oligonucleotide molecules, etc.

The polynucleic acids of the invention may be comprised in a composition of any kind. Said composition may be for diagnostic, therapeutic or prophylactic use.

The present invention also relates to a recombinant expression vector which may comprise a C2MI polynucleic acid of the invention as defined above operably linked to prokaryotic, eukaryotic or viral transcription and translation control elements as well as host cells containing such vector.

The term “vector” may comprise a plasmid, a cosmid, an artificial chromosome, a phage, or a virus or a transgenic non-human animal. Examples are adenovirus, adeno-associated virus, herpes simplex virus, vaccinia, or, an RNA virus such as a retrovirus.

The term “recombinant expression” used within the context of the present invention refers to the fact that the polypeptides of the present invention are produced by recombinant expression methods be it in prokaryotes, or lower or higher eukaryotes as discussed in detail below.

The term “host cell” refers to cells which can be or have been, used as recipients for a recombinant vector or other transfer polynucleotide, and include the progeny of the original cell which has been transfected.

It is understood that the progeny of a single parental cell may not necessarily be completely identical in morphology or in genomic or total DNA complement as the original parent, due to natural, accidental, or deliberate mutation or recombination.

The term “lower eukaryote” refers to host cells such as yeast, fungi and the like. Lower eukaryotes are generally (but not necessarily) unicellular. Preferred lower eukaryotes are yeasts, particularly species within Saccharomyces, Schizosaccharomyces, Kluiveromyces, Pichia (e. g. Pichia pastoris), Hansenula (e. g. Hansenula polymorph), Schwaniomyces, Schizosaccharomyces, Yarowia, Zygosaccharomyces and the like. Saccharomyces cerevisiae, S. carlsbergensis and K. lactis are the most commonly used yeast hosts, and are convenient fungal hosts.

The term “higher eukaryote” refers to host cells derived from higher animals, such as mammals, reptiles, insects, and the like. Presently preferred higher eukaryote host cells are derived from Chinese hamster (e. g. CHO), monkey (e. g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, the 293TT cell line (Buck et al., 2004) and insectcell lines (e.g. Spodoptera frugiperda). The host cells may be provided in suspension or flask cultures, tissue cultures, organ cultures and the like. Alternatively the host cells may also be transgenic non-human animals.

The term “prokaryotes” refers to hosts such as E. coli, Lactobacillus, Lactococcus, Salmonella, Streptococcus, Bacillus subtilis, Acinetobacter or Streptomyces. Also these hosts are contemplated within the present invention.

The segment of the C2MI DNA encoding the desired sequence inserted into the vector sequence may be attached to a signal sequence. Said signal sequence may be that from a non-C2MI source, but particularly preferred constructs according to the present invention contain signal sequences appearing in the C2MI genome before the respective start points of the proteins.

Higher eukaryotes may be transformed with vectors, or may be infected with a recombinant virus, for example a recombinant vaccinia virus. Techniques and vectors for the insertion of foreign DNA into vaccinia virus are well known in the art, and utilize, for example homologous recombination. A wide variety of viral promoter sequences, possibly terminator sequences and poly(A)-addition sequences, possibly enhancer sequences and possibly amplification sequences, all required for the mammalian expression, are available in the art. Vaccinia is particularly preferred since vaccinia halts the expression of host cell proteins. For vaccination of humans the avipox and Ankara Modified Virus (AMV) are particularly useful vectors.

Also known are insect expression transfer vectors derived from baculovirus Autographa californica nuclear polyhedrosis virus (AcNPV), which is a helper-independent viral expression vector. Expression vectors derived from this system usually use the strong viral polyhedrin gene promoter to drive the expression of heterologous genes. Different vectors as well as methods for the introduction of heterologous DNA into the desired site of baculovirus are available to the person skilled in the art for baculovirus expression. Also different signals for posttranslational modification recognized by insect cells are known in the art.

The present invention also relates to a polypeptide having an amino acid sequence encoded by a C2MI polynucleic acid as defined above, or a part or an analogue thereof being substantially similar and biologically equivalent. Reference is made to FIG. 1-22 (B) und (C).

The term “polypeptide” refers to a polymer of amino acids and does not refer to a specific length of the product. Thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogues of an amino acid (including, for example, unnatural amino acids, peptide nucleic acid (PNA), etc.), polypeptides with substituted linkages, as well as other modifications known in the art, both naturally occurring and non-naturally occurring.

The polypeptides according to the present invention contain preferably at least 3, preferably 4 or 5 contiguous C2MI amino acids, 6 or 7 preferably however at least 8 contiguous C2MI amino acids, at least 10 or at least 15.

The polypeptides of the invention, and particularly the fragments, can be prepared by classical chemical synthesis. The synthesis can be carried out in homogeneous solution or in solid phase. The polypeptides according to this invention can also be prepared by means of recombinant DNA techniques.

The present invention also relates to a method for production of a recombinant polypeptide as defined above, which may comprise: (a) transformation of an appropriate cellular host with a recombinant vector, in which a polynucleic acid or a part thereof as defined above has been inserted under the control of the appropriate regulatory elements, (b) culturing said transformed cellular host under conditions enabling the expression of said insert, and (c) harvesting said polypeptide.

The present invention also relates to an antibody raised upon immunization with at least one polypeptide as defined above, with said antibody being specifically reactive with any of said polypeptides, and with said antibody being preferably a monoclonal antibody. The term “antibody”, preferably, relates to antibodies which consist essentially of pooled monoclonal antibodies with different epitopic specificities, as well as distinct monoclonal antibody preparations. Monoclonal antibodies are made from an antigen containing, e.g., a polypeptide encoded by a C2MI polynucleic acid of the invention or a fragment thereof by methods well known to those skilled in the art. As used herein, the term “antibody” (Ab) or “monoclonal antibody” (Mab) is meant to include intact molecules as well as antibody fragments (such as, for example, Fab and F(ab′)2 fragments) which are capable of specifically binding to protein. Fab and F(ab′)2 fragments lack the Fc fragment of intact antibody, clear more rapidly from the circulation, and may have less non-specific tissue binding than an intact antibody. Thus, these fragments are preferred, as well as the products of a Fab or other immunoglobulin expression library. Moreover, antibodies useful for the purposes of the present invention include chimerical, single chain, and humanized antibodies.

The present invention also relates to diagnostic and therapeutic approaches using cell-mediated immune responses.

Preferably, the antibody or antigen binding fragment thereof carries a detectable label. The antibody/fragment can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels for binding to the antibody, or will be able to ascertain such, using routine experimentation.

The present invention also relates to a diagnostic kit for use in determining the presence of a C2MI polynucleic acid or polypeptide of the invention, said kit which may comprise a primer, a probe, and/or an antibody of the invention. Said kit may have any format well known to the person skilled in the art, e.g. can be an ELISA-based kit.

The present invention also relates to a method for the detection of a C2MI polynucleic acid according to the invention present in a biological sample, which may comprise: (a) optionally extracting sample polynucleic acid, (b) amplifying the polynucleic acid as described above with at least one primer as defined above, optionally a labelled primer, and (c) detecting the amplified polynucleic acids.

The term “polynucleic acid” can also be referred to as analyte strand and corresponds to a single- or double-stranded polynucleic acid molecule.

The term “labelled” refers to the use of labelled nucleic acids. This may include the use of labelled nucleotides incorporated during the polymerase step of the amplification or labelled primers, or by any other method known to the person skilled in the art.

The present invention also relates to a method for the detection of a C2MI polynucleic acid according to the invention present in a biological sample, which may comprise: (a) optionally extracting sample polynucleic acid, (b) hybridizing the polynucleic acid as described above with at least one probe as defined above, and (c) detecting the hybridized polynucleic acids.

The hybridization and washing conditions are to be understood as stringent and are generally known in the art. However, according to the hybridization solution (SSC, SSPE, etc.), these probes should be hybridized at their appropriate temperature in order to attain sufficient specificity.

According to the hybridization solution (SSC, SSPE, etc.), these probes should be stringently hybridized at their appropriate temperature in order to attain sufficient specificity. However, by slightly modifying the DNA probes, either by adding or deleting one or a few nucleotides at their extremities (either 3′ or 5′), or substituting some non-essential nucleotides (i.e. nucleotides not essential to discriminate between types) by others (including modified nucleotides or inosine) these probes or variants thereof can be caused to hybridize specifically at the same hybridization conditions (i.e. the same temperature and the same hybridization solution). Also changing the amount (concentration) of probe used may be beneficial to obtain more specific hybridization results. It should be noted in this context, that probes of the same length, regardless of their GC content, will hybridize specifically at approximately the same temperature in TMACI solutions.

Suitable assay methods for purposes of the present invention to detect hybrids formed between the oligonucleotide probes and the C2MI polynucleic acid sequences in a sample may comprise any of the assay formats known in the art, such as the conventional dot-blot format, sandwich hybridization or reverse hybridization. For example, the detection can be accomplished using a dot blot format, the unlabelled amplified sample being bound to a membrane, the membrane being incorporated with at least one labelled probe under suitable hybridization and wash conditions, and the presence of bound probe being monitored.

An alternative and preferred method is a “reverse” dot-blot format, in which the amplified sequence contains a label. In this format, the unlabelled oligonucleotide probes are bound to a solid support and exposed to the labelled sample under appropriate stringent hybridization and subsequent washing conditions. It is to be understood that also any other assay method which relies on the formation of a hybrid between the polynucleic acids of the sample and the oligonucleotide probes according to the present invention may be used.

The present invention also relates to a method for detecting a polypeptide encoded by a C2MI polynucleic acid of the present invention or an antibody against said polypeptide present in a biological sample, which may comprise: (a) contacting the biological sample for the presence of such polypeptide or antibody as defined above, and (b) detecting the immunological complex formed between said antibody and said polypeptide.

The immunoassay methods according to the present invention may utilize antigens from different domains of the new and unique polypeptide sequences of the present invention. It is within the scope of the invention to use for instance single or specific oligomeric antigens, dimeric antigens, as well as combinations of single or specific oligomeric antigens. The C2MI antigens of the present invention may be employed in virtually any assay format that employs a known antigen to detect antibodies or cell-mediated immune responses. Thus, the present invention also encompasses the detection of cell-mediated immune responses against C2MI antigens and the application of therapeutic interferences based on cell-mediated immune responses against C2MI antigens.

Of course, an assay format that denatures the C2MI conformational epitope should be avoided or adapted. A common feature of all of these assays is that the antigen is contacted with the body component suspected of containing C2MI antibodies under conditions that permit the antigen to bind to any such antibody present in the component. Such conditions will typically be physiologic temperature, pH and ionic strength using an excess of antigen. The incubation of the antigen with the specimen is followed by detection of immune complexes comprised of the antigen.

Design of the immunoassays is subject to a great deal of variation, and many formats are known in the art. Protocols may, for example, use solid supports, or immunoprecipitation. Most assays involve the use of labeled antibody or polypeptide; the labels may be, for example, enzymatic, fluorescent, chemiluminescent, radioactive, or dye molecules. Assays which amplify the signals from the immune complex are also known; examples of which are assays which utilize biotin and avidin or streptavidin, and enzyme-labeled and mediated immunoassays, such as ELISA assays.

The immunoassay may be in a heterogeneous or in a homogeneous format, and of a standard or competitive type. In a heterogeneous format, the polypeptide is typically bound to a solid matrix or support to facilitate separation of the sample from the polypeptide after incubation. Examples of solid supports that can be used are nitrocellulose (e. g., in membrane or microtiter well form), polyvinyl chloride (e. g., in sheets or microtiter wells), polystyrene latex (e. g., in beads or microtiter plates, polyvinylidine fluoride (known as Immunolon), diazotized paper, nylon membranes, activated beads, and Protein A beads. The solid support containing the antigenic polypeptides is typically washed after separating it from the test sample, and prior to detection of bound antibodies. Both standard and competitive formats are known in the art.

In a homogeneous format, the test sample is incubated with the combination of antigens in solution. For example, it may be under conditions that will precipitate any antigen-antibody complexes which are formed. Both standard and competitive formats for these assays are known in the art.

In a standard format, the amount of C2MI antibodies in the antibody-antigen complexes is directly monitored. This may be accomplished by determining whether (labelled) anti-xenogeneic (e. g. anti-human) antibodies which recognize an epitope on anti-C2MI antibodies will bind due to complex formation. In a competitive format, the amount of C2MI antibodies in the sample is deduced by monitoring the competitive effect on the binding of a known amount of labelled antibody (or other competing ligand) in the complex.

Complexes formed which may comprise anti-C2MI antibody (or in the case of competitive assays, the amount of competing antibody) are detected by any of a number of known, depending on the format. For example, unlabeled C2MI antibodies in the complex may be detected using a conjugate of anti-xenogeneic Ig complexed with a label (e. g. an enzyme label).

In an immunoprecipitation or agglutination assay format the reaction between the C2MI antigens and the antibody forms a network that precipitates from the solution or suspension and forms a visible layer or film of precipitate. If no anti-C2MI antibody is present in the test specimen, no visible precipitate is formed.

There currently exist three specific types of particle agglutination (PA) assays. These assays are used for the detection of antibodies to various antigens when coated to a support. One type of this assay is the hemagglutination assay using red blood cells (RBCs) that are sensitized by passively adsorbing antigen (or antibody) to the RBC. The addition of specific antigen/antibodies present in the body component, if any, causes the RBCs coated with the purified antigen to agglutinate.

To eliminate potential non-specific reactions in the hemagglutination assay, two artificial carriers may be used instead of RBC in the PA. The most common of these are latex particles.

The solid phase selected can include polymeric or glass beads, nitrocellulose, microparticles, microwells of a reaction tray, test tubes and magnetic beads. The signal generating compound can include an enzyme, a luminescent compound, a chromogen, a radioactive element and a chemiluminescent compound. Examples of enzymes include alkaline phosphatase, horseradish peroxidase and beta-galactosidase. Examples of enhancer compounds include biotin, anti-biotin and avidin. Examples of enhancer compounds binding members include biotin, anti-biotin and avidin.

The above methods are useful for evaluating the risk of developing diseases e.g. cancer or a disease of the CNS, due to the cllevayius effects of the presence of a subgenomic C2MI polynucleotide sequence by itself or linked to a particular host gene or gene fragment within the patient's cells and allow taking appropriate counter measures.

Thus, the present invention also relates to an antisense oligonucleotide or iRNA specific for the C2MI virus polynucleic acid of the invention.

The generation of suitable antisense oligonucleotides or iRNAs includes determination of a site or sites within the C2MI polynucleic acid for the antisense interaction to occur such that the desired effect, e.g., inhibition of expression of the polypeptide, will result. A preferred intragenic site is (a) the region encompassing the translation initiation or termination codon of the open reading frame (ORF) of the gene or (b) a region of the mRNA which is a “loop” or “bulge”, i.e., not part of a secondary structure. Once one or more target sites have been identified, oligonucleotides are chosen which are sufficiently complementary to the target, i.e., hybridize sufficiently well and with sufficient specificity, to give the desired effect. In the context of this invention, “hybridization” means hydrogen bonding, which may be Watson-Crick, Hoogsteen or reversed Hoogsteen hydrogen bonding, between complementary nucleoside or nucleotide bases. “Complementary” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound does not need to be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., in the case of therapeutic treatment.

“Oligonucleotide” (in particular in the context of antisense compounds) refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. While antisense oligonucleotides are a preferred form of the antisense compound, the present invention comprehends other oligomeric antisense compounds, including but not limited to oligonucleotide mimetics such as are described below. The antisense compounds in accordance with this invention comprise from about 6 to about 50 nucleobases (i.e. from about 6 to about 50 linked nucleosides). Particularly preferred antisense compounds are antisense oligonucleotides, even more preferably those which may comprise from about 15 to about 25 nucleobases. Antisense compounds include ribozymes, external guide sequences (EGS), oligonucleotides (oligozymes), and other short catalytic RNAs or catalytic oligonucleotides which hybridize to the target nucleic acid and inhibit its expression. The antisense compounds also include an iRNA which may comprise a sense sequence and an antisense sequence, wherein the sense and antisense sequences form an RNA duplex and wherein the antisense sequence may comprise a nucleotide sequence sufficiently complementary to the nucleotide sequence of a C2MI polynucleic acid of the present invention.

Alternatively, the invention provides a vector allowing to transcribe an antisense oligonucleotide of the invention, e.g., in a mammalian host. Preferably, such a vector is a vector useful for gene therapy. Preferred vectors useful for gene therapy are viral vectors, e.g. adenovirus, adeno-associated virus, herpes simplex virus, vaccinia, or, an RNA virus such as a retrovirus. Preferably, the retroviral vector is a derivative of a murine or avian retrovirus. Examples of such retroviral vectors which can be used in the present invention are: Moloney murine leukemia virus (MoMuLV), Harvey murine sarcoma virus (HaMuSV), murine mammary tumor virus (MuMTV) and Rous sarcoma virus (RSV). Most preferably, a non-human primate retroviral vector is employed, such as the gibbon ape leukemia virus (GaLV), providing a broader host range compared to murine vectors. Since recombinant retroviruses are defective, assistance is required in order to produce infectious particles. Such assistance can be provided, e.g., by using helper cell lines that contain plasmids encoding all of the structural genes of the retrovirus under the control of regulatory sequences within the LTR. Suitable helper cell lines are well known to those skilled in the art. Said vectors can additionally contain a gene encoding a selectable marker so that the transduced cells can be identified. Moreover, the retroviral vectors can be modified in such a way that they become target specific. This can be achieved, e.g., by inserting a polynucleotide encoding a sugar, a glycolipid, or a protein, preferably an antibody. Those skilled in the art know additional methods for generating target specific vectors. Further suitable vectors and methods for in vitro- or in vivo-gene therapy are described in the literature and are known to the persons skilled in the art; see, e.g., WO 94/29469 or WO 97/00957. The C2MI polynucleotide sequences of the invention may also serve as a suitable vector itself, either composed solely of rearranged C2MI sequences or of chimeric C2MI host cell DNA sequences. In addition, the nucleotide sequences of the invention may be used for the construction of artificial chromosomes.

In order to achieve expression only in the target organ, the DNA sequences for transcription of the antisense oligonucleotides can be linked to a tissue specific promoter and used for gene therapy. Such promoters are well known to those skilled in the art.

Within an oligonucleotide structure, the phosphate groups are commonly referred to as forming the internucleoside backbone of the oligonucleotide. The normal linkage or backbone of RNA and DNA is a 3′ to 5′ phosphodiester linkage. Specific examples of preferred antisense compounds useful in the present invention include oligonucleotides containing modified backbones or non-natural internucleoside linkages. Oligonucleotides having modified backbones include those that retain a phosphorus atom in the backbone and those that do not have a phosphorus atom in the backbone. Modified oligonucleotide backbones which can result in increased stability are known to the person skilled in the art, preferably such modification is a phosphorothioate linkage.

A preferred oligonucleotide mimetic is an oligonucleotide mimetic that has been shown to have excellent hybridization properties, and is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

Modified oligonucleotides may also contain one or more substituted or modified sugar moieties. Preferred oligonucleotides comprise one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. A particularly preferred modified sugar moiety is a 2′-O-methoxyethyl sugar moiety.

Antisense-oligonucleotides of the invention may also include nucleobase modifications or substitutions. Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine etc., with 5-methylcytosine substitutions being preferred since these modifications have been shown to increase nucleic acid duplex stability.

Another modification of the oligonucleotides of the invention involves chemically linking to the oligonucleotide one or more moieties or conjugates which enhance the activity, cellular distribution or cellular uptake of the oligonucleotide. Such moieties include lipid moieties such as a cholesterol moiety, cholic acid, a thioether, a thiocholesterol, an aliphatic chain, e.g., dodecandiol or undecyl residues, a phospholipid, a polyamine or a polyethylene glycol chain, or adamantane acetic acid, a palmityl moiety, or an octadecylamine or hexylamino-carbonyl-oxycholesterol moiety.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers.

The present invention also relates to a pharmaceutical composition which may comprise an antibody or antisense oligonucleotide of the invention and a suitable excipient, diluent or carrier. Preferably, in a pharmaceutical composition, such compound as described above is combined with a pharmaceutically acceptable carrier. “Pharmaceutically acceptable” is meant to encompass any carrier, which does not interfere with the effectiveness of the biological activity of the active ingredient and that is not toxic to the host to which it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and the active compound can be administered to the subject at an effective dose.

An “effective dose” refers to an amount of the active ingredient that is sufficient to prevent the disease or to affect the course and the severity of the disease, leading to the reduction or remission of such pathology. An “effective dose” useful for treating and/or preventing these diseases or disorders may be determined using methods known to one skilled in the art.

Administration of the suitable compositions may be effected by different ways, e.g. by intravenous, intraperitoneal, oral, subcutaneous, intramuscular, topical or intradermal administration. The route of administration, of course, depends on the kind of therapy and the kind of compound contained in the pharmaceutical composition. The dosage regimen will be determined by the attending physician and other clinical factors. As is well known in the medical arts, dosages for any one patient depends on many factors, including the patient's size, body surface area, age, sex, the particular compound to be administered, time and route of administration, the kind of therapy, general health and other drugs being administered concurrently.

In a preferred embodiment of the present invention, the disease that can be prevented/treated is cancer, preferably breast cancer, ovarian cancer, lung cancer, prostate cancer, pancreatic cancer, Hodgkin's disease, colorectal cancer or colon cancer or a disease of the CNS, preferably Alzheimer's disease or multiple sclerosis (MS), amyotrophic lateral sclerosis, Parkinson's disease, or transmissible spongiforme encephalopathies/Prion-linked diseases. In addition, due to a similarity of risk factors between MS and diabetes mellitus, the latter condition is also included. Furthermore, atherosclerosis is included. The terms “cancer” and “disease of the CNS” also comprise early stages of said diseases.

The present invention also relates to a vaccine for immunizing a mammal against a C2MI infection, which may comprise at least one C2MI polypeptide or C2MI polynucleic acid as defined above or corresponding VLP (virus-like particle) or peptide/protein/DNA complexes, in a pharmaceutically acceptable carrier. It also involves molecular and immunological tests in animals (in particular cattle) and within their products (e.g. milk and dairy products).

It may also include small chemicals for targeted therapy derived from the analysis of structural components of these agents.

A “vaccine” is an immunogenic composition capable of eliciting protection against C2MI, whether partial or complete. A vaccine may also be useful for treatment of an already infected individual, in which case it is called a therapeutic vaccine.

The term “therapeutic” refers to a composition capable of treating C2MI infection or diseases linked to this infection. The term “effective amount” refers to an amount of epitope-bearing polypeptide sufficient to induce an immunogenic response in the individual to which it is administered, or to otherwise detectably immunoreact in its intended system (e. g., immunoassay). Preferably, the effective amount is sufficient to effect treatment, as defined above. The exact amount necessary will vary according to the application. For vaccine applications or for the generation of polyclonal antiserum/antibodies, for example, the effective amount may vary depending on the species, age, and general condition of the individual, the severity of the condition being treated, the particular polypeptide selected and its mode of administration, etc. Effective amounts will be found within a relatively large, non-critical range. An appropriate effective amount can be readily determined using routine experimentation. Preferred ranges of proteins for prophylaxis of C2MI caused diseases are 0.01 to 100 μg/dose, preferably 0.1 to 50 μg/dose. Several doses may be needed per individual in order to achieve a sufficient immune response and subsequent protection against a C2MI infection and a C2MI linked disease, respectively.

Pharmaceutically acceptable carriers include any carrier that does not itself induce the production of antibodies harmful to the individual receiving the vaccine. Suitable carriers are typically large, slowly metabolized macromolecules such as proteins, polysaccharides, polylactic acids, polyglycolic acids, polymeric amino acids, and amino acid copolymers. Such carriers are well known to those of ordinary skill in the art.

Preferred adjuvants to enhance effectiveness of the composition include, but are not limited to: aluminium hydroxide (alum), N-acetyl-muramyl-L-threonyl-D-isoglutamine (thr-MDP) as found in U.S. Pat. No. 4,606,918, N-acetyl-normuramyl-L-alanyl-D-isoglutamine (nor-MDP), N-acetylmuramyl-L-alanyl-D-isoglutaminyl-L-alanine-2-(1′-2′-dipalmitoyl sn-glycero-3-hydroxy-phosphoryloxy)-ethylamine (MTP-PE) and RIBI, which contains three components extracted from bacteria, monophosphoryl lipid A, trehalose dimycolate, and cell wall Skeleton (MPL+TDM+CWS) in a 2% squalene/Tween 80 emulsion. Any of the 3 components MPL, TDM or CWS may also be used alone or combined 2 by 2. Additionally, adjuvants such as Stimulon (Cambridge Bioscience, Worcester, Mass.) or SAF-1 (Syntex) may be used. Further, Complete Freund's Adjuvant (CFA) and Incomplete Freund's Adjuvant (IFA) may be used for non-human applications and research purposes.

The immunogenic compositions typically will contain pharmaceutically acceptable vehicles, such as water, saline, glycerol, ethanol, etc. Additionally, auxiliary substances, such as wetting or emulsifying agents, pH buffering substances, preservatives, and the like, may be included in such vehicles.

Typically, the immunogenic compositions are prepared as injectables, either as liquid solutions or suspensions. Solid forms suitable for solution in, or suspension in, liquid vehicles prior to injection may also be prepared. The preparation also may be emulsified or encapsulated in liposomes for enhanced adjuvant effect. The proteins may also be incorporated into Immune Stimulating Complexes together with saponins, for example Quil A (ISCOMS).

Immunogenic compositions used as vaccines comprise a “sufficient amount” or “an immunologically effective amount” of the proteins of the present invention, as well as any other of the above mentioned components, as needed. “Immunologically effective amount” means that the administration of that amount to an individual, either in a single dose or as part of a series, is effective for treatment, as defined above. This amount varies depending upon the health and physical condition of the individual to be treated, the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials. Usually the amount will vary from 0.01 to 1000 □g/dose, more particularly from 0.1-100 μg/dose.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

The following examples are intended to illustrate, but not to limit the invention. While such examples are typical of those that might be used, other methods known to those skilled in the art may alternatively be utilized.

### EXAMPLES

### Commercial Assays

NucleoSpin® Gel and PCR Clean up Macherey-Nagel
Invitrogen™ TA Cloning™ Kits with pCR™ 2.1 Vector, without Competent Cells Invitrogen
Invitrogen™ TA Cloning™ Kit, with pCR™2.1 Vector and One Shot™ TOP10F′ Chemically Competent E. coli Invitrogen
Invitrogen™ One Shot™ TOP10F′ Chemically Competent E. coli Invitrogen
AccuPrep Plasmid Mini Extraction Kit Bioneer
JeneJet Plasmid Miniprep Kit Thermo Scientific
Plasmid maxi Kit Qiagen

### Chemicals:

TaKaRa LA Taq® DNA Polymerase with GC Buffer TaKaRa
Sodium chloride Sigma-Aldrich
Yeast Extract Gerbu
Trypton/Pepton from Casein Roth
phi29 DNA Polymerase New England Biolabs M02695
dNTPs Takara '4030
Exo-resistant Random primer Thermo Fisher Scientific S0181

### Example 1: DNA Extraction

DNA (16 samples from cow milk and cow milk products) was extracted with phenol/chloroform as previously described (Whitley et al., 2014). Samples were diluted 1:1 with proteinase K buffer (0.2M Tris-HCL, pH7.5, 25 mM EDTA, 0.3M NaCl, 2% SDS) and digested overnight with proteinase K (final concentration 1 mg/ml, Sigma-Aldrich P6556) at 37° C. Ethanol precipitation followed after the phenol/chloroform extraction. Samples 5-16 were subsequently subjected to an additional purification using a Nucleospin Gel and PCR Clean up Kit according to the manufacturer's protocol (Machery and Nagel, 740.609.250). DNA was finally eluted in 5 mM Tris-HCl, pH 8.0.

### Example 2: Rolling Circle Amplification

Rolling circle amplification was performed as described by Funk et al, 2014. Template DNA (50 ng) was incubated in a total volume of 10 μl (1× phi29 DNA polymerase buffer) with 25 μM Exo-resistant Random primer at 95° C. for 3 min followed by cooling on ice. This sample was diluted to 20 μl by adding BSA (0.4 mg/ml), 0.75 mM dNTPs each and 10U phi29 DNA Polymerase in 1× phi29 DNA polymerase buffer. Incubation followed at 30° C. for 18 h and 10 minutes at 65° C. before stopping the reaction at 15° C.

### Example 3: Polymerase Chain Reaction

A 3 μl template from the RCA amplified DNA was used in the polymerase chain reaction (PCR) using TAKARA Taq enzyme and the accompanying solutions according to the manufacturer's protocol (LA TAKARA RR02AG). The respective abutting primers were added at a final concentration of 0.2 mM each. Primers were designed based on the conserved regions after aligning HCBI1, HCBI2 and HCBI7 for the BMMF2 group and for the BMMF1 group based on the conserved region of the replication gene.

PCR was performed by using touchdown protocols specific for the respective primer pairs:

MBB2 primers for the BMMF1 group:

Mbb2 (MSBI1.176bbF296 forward)-
5′-TGCAGAAATTGCCCCTCGACT-3′
and
Mbb2 (MSBI1.176bbR295 reverse)-
5′-AACAATGGGGAAGAAGTCAAAGG-3′.

The first round of 5 cycles amplification was performed at 30 seconds melting at 94° C., 1 minute annealing at 64° C. and 2 minutes elongation at 72° C., followed by a second round of 5 cycles using 62° C. annealing and a third round of 30 cycles using 60° C. for annealing. The final round of 10 minutes at 72° C. followed.

Amplification for the BMMF2 group was performed as described for the BMMF1 group using the MBB2 primers, except that the touchdown temperatures were 58° C., 56° C. and 54° C. for the LSconA primer and 56° C., 54° C. and 52° C. for the LSconB primer and the elongation time was prolonged to 3 minutes at 72° C. per cycle.

The abutting primer pairs used for the BMMF2 group were:

LSConA5p (forward)-
5′- AAGGCAGATCAACACAGG-3′
and
LSconA3p (reverse)-
5′-AGCAGATTGCAAAGCCTG-3′.
LSconB5p (forward)-
5′-CAACACAGGGATAGAATAAC-3′
and
LSconB3p (reverse)-
5′-ATCTGCCTTAGCAGATTG C-3′.

PCR products were separated by gel electrophoresis, visible fragments excised and DNA extracted using the Nucleospin Gel and PCR Clean up Kit according to the manufacturer's protocol (Machery and Nagel, 740.609.250). DNA was finally eluted in 5 mM Tris-HCl, pH 8.0.

Eluted DNA was ligated and cloned into pCR2.1 using the TA-Cloning Kit and transformed into E. coli—all according to the manufacturer's protocol (Invitrogen™ TA Cloning™ Kit, with pCR™2.1 Vector and One Shot™ TOP10F′ Chemically Competent E. coli (Invitrogen K203040).

Sequencing of all clones was performed by GATC.

### Example 4: In Silico Analyses

All sequences obtained were compared to sequences available in Genbank and the putative protein sequences to swissprot and swissprot_splicevar.

Predicted genes were obtained with the gene identification program GENSCAN which was developed Chris Burge from Stanford University (Burge and Karlin, 1997). It analyzes genomic DNA sequences from human, other vertebrates, invertebrates and plants.

Phylogenetic trees were developed using maximum likelihood based on aligned (MUSCLE algorithm) sequences—both full-length nucleotide genomes as well as the core region of Rep.

### REFERENCES

Bertini A, Poirel L, Mugnier P D, Villa L, Nordmann P, Carattoli A. Characterization and PCR-based replicon typing of resistance plasmids in Acinetobacter baumannii. Antimicrob Agents Chemother. 2010; 54:4168-4177. doi: 10.1128/AAC.00542-10. Biagini P. Bacterial plasmid-related DNA sequences isolated from plasma of patients in maintenance hemodialysis (unpublished).
Boer D R, Ruiz-Masó J A, Rueda M, Petoukhov M V, Machón C, Svergun D I, Orozco M1, del Solar G, Coll M. Conformational plasticity of RepB, the replication initiator protein of promiscuous streptococcal plasmid pMV158. Sci Rep. 2016; 6:20915. doi: 10.1038/srep20915.
Boer D R, Ruíz-Masó J A, López-Blanco J R, Blanco A G, Vives-Llàcer M, Chacón P, Usón I, Gomis-Rüth F X, Espinosa M, Llorca O, del Solar G, Coll M. Plasmid replication initiator RepB forms a hexamer reminiscent of ring helicases and has mobile nuclease domains. EMBO J. 2009; 28:1666-78. doi: 10.1038/emboj.2009.125
Briddon R W, Martin D P, Roumagnac P, Navas-Castillo J, Fiallo-Olivé E, Moriones E, Lett J-M, Zerbini F M, Varsani A. Alphasatellitidae: a new family with two subfamilies for the classification of geminivirus- and nanovirus-associated alphasatelllites. Arch Virol. 2018; doi.org/10.1007/s00705-018-3854-2.
Burge C, Karlin S. Prediction of complete gene structures in human genomic DNA. J Mol Biol 1997; 268:78-94. (Genscan)
Curinha Al, Oliveira Braz S, Pereira-Castro I, Cruz A, Moreira A. Implications of polyadenylation in health and disease. Nucleus. 2014; 5(6):508-19. doi: 10.4161/nucl.36360.
Denoeud F1, Henriet S, Mungpakdee S, Aury J M, Da Silva C, Brinkmann H, Mikhaleva J, Olsen L C, Jubin C, Cañestro C, Bouquet J M, Danks G, Poulain J, Campsteijn C, Adamski M, Cross I, Yadetie F, Muffato M, Louis A, Butcher S, Tsagkogeorga G, Konrad A, Singh S, Jensen M F, Huynh Cong E, Eikeseth-Otteraa H, Noel B, Anthouard V, Porcel B M, Kachouri-Lafond R, Nishino A, Ugolini M, Chourrout P, Nishida H, Aasland R, Huzurbazar S, Westhof E, Delsuc F, Lehrach H, Reinhardt R, Weissenbach J, Roy S W, Artiguenave F, Postlethwait J H, Manak J R, Thompson E M, Jaillon O, Du Pasquier L, Boudinot P, Liberles D A, Volff I N, Philippe H, Lenhard B, Roest Crollius H, Wincker P, Chourrout D. Plasticity of animal genome architecture unmasked by rapid evolution of a pelagic tunicate. Science. 2010 Dec. 3; 330(6009):1381-5
de Villiers E M, Kimmel R, Leppik L, Gunst K. Intragenomic rearrangement in TT viruses: a possible role in the pathogenesis of disease. Curr Top Microbiol Immunol 2009; 331:91-107.Review.
de Villiers E M, Borkosky S S, Kimmel R, Gunst K, Fei J W. The diversity of torque teno viruses: in vitro replication leads to the formation of additional replication-competent subviral molecules. J Virol. 2011; 85(14):7284-7295. doi: 10.1128/JVI.02472-10. Epub 2011 May 18.
Eilebrecht S, Hotz-Wagenblatt A, Sarachaga V, Burk A, Falida K, Chakraborty D, Nikitina E, Tessmer C, Whitley C, Sauerland C, Gunst K, Grewe I, Bund T. Expression and replication of virus-like circular DNA in human cells. Sci Rep. 2018; 8:2851.
Falida K, Eilebrecht S, Gunst K, Zur Hausen H, de Villiers E M. Isolation of Two Virus-Like Circular DNAs from Commercially Available Milk Samples. Genome Announc. 2017; 5(17). pii: e00266-17. doi: 10.1128/genomeA.00266-17.
Fernandes M A, Verstraete S G, Phan T G, Deng X, Stekol E, LaMere B, Lynch S V, Heyman M B, Delwart E. Enteric Virome and Bacterial Microbiota in Children with Ulcerative Colitis and Crohn's Disease. J Pediatr Gastroenterol Nutr. 2018 Aug. 30. doi: 10.1097/MPG.0000000000002140. [Epub ahead of print]
Fondi M, Bacci G, Brilli M, Papaleo M C, Mengoni A, Vaneechoutte M, Dijkshoorn L, Fani R. Exploring the evolutionary dynamics of plasmids: the Acinetobacter pan-plasmidome. BMC Evol Biol. 2010; 10:59. doi: 10.1186/1471-2148-10-59.
Fournier PE1, Vallenet D, Barbe V, Audic S, Ogata H, Poirel L, Richet H, Robert C, Mangenot S, Abergel C, Nordmann P, Weissenbach J, Raoult D, Claverie J M. Comparative genomics of multidrug resistance in Acinetobacter baumannii. PLoS Genet. 2006; 2(1):e7. Epub 2006 Jan. 13.
Funk M, Gunst K, Lucansky V, Müller H, zur Hausen H, de Villiers E M. Isolation of protein-associated circular DNA from healthy cattle serum. Genome Announc 2014; 2:e00846-14.
Gomis-Rüth F X, Solá M, Acebo P, Párraga A, Guasch A, Eritja R, González A, Espinosa M, del Solar G, Coll M. The structure of plasmid-encoded transcriptional repressor CopG unliganded and bound to its operator. EMBO J. 1998 Dec. 15; 17(24):7404-15.
Gronenborn B, Randles J W, Knierim D, Barrière Q, Vetten H J, Warthmann N, Cornu D, Sileye T, Winter S, Timchenko T. Analysis of DNAs associated with coconut foliar decay disease implicates a unique single-stranded DNA virus representing a new taxon. Sci Rep. 2018; 8(1):5698. doi: 10.1038/s41598-018-23739-y.
Gunst K, Zur Hausen H, de Villiers E M. Isolation of bacterial plasmid-related replication-associated circular DNA from a serum sample of a multiple sclerosis patient. Genome Announc. 2014; 2(4). pii: e00847-14. doi: 10.1128/genomeA.00847-14.
Hamidian, M., Kenyon, J. J., Holt, K. E., Pickard, D., Hall, R. M. A conjugative plasmid carrying the carbapenem resistance gene blaOXA-23 in AbaR4 in an extensively resistant GC1 Acinetobacterbaumannii isolate. J. Antimicrob. Chemother. 2014; 69: 2625-2628.
Harding C M, Hennon S W, Feldman M F. Uncovering the mechanisms of Acinetobacter baumannii virulence. Nat Rev Microbiol. 2018; 16:91-102. doi: 10.1038/nrmicro.2017.148. Epub 2017 Dec. 18.
Hassan I, Orillio A F, Fiallo-Olive E, Briddon R W, Navas-Castillo J. Infectiviy, effects on helper viruses and whitefly transmission of the deltasatellites associated with sweepoviruses (genus Begomovirus, family Geminiviridae). 2016; Sci Rep. 6: 30204.
Jelcic I, Hotz-Wagenblatt A, Hunziker A, zur Hausen H, de Villiers E M. Isolation of multiple TT virus genotypes from spleen biopsy tissue from a Hodgkin's disease patient: genome reorganization and diversity in the hypervariable region. J Virol 2004; 78:7498-74507.
Jones D. T., Taylor W. R., and Thornton J. M. (1992). The rapid generation of mutation data matrices from protein sequences. Computer Applications in the Biosciences 8: 275-282.
Jørgensen TS, Xu Z, Hansen M A, Sørensen SJ, Hansen L H. Hundreds of circular novel plasmids and DNA elements identified in a rat cecum metamobilome. PLoS One. 2014; 9e87924.
Jorgensen T J, Hansen M A, Xu Z, Tabak M A, Sorensen, S J, Hansen, L H. Plasmids, circular viruses and viroids from rat gut. (unpublished).
Kazlauskas D, Dayaram A, Kraberger S, Goldstein S, Varsani A, Krupovic M. Evolutionary history of ssDNA bacilldnaviruses features horizontal acquisition of the capsid gene from ssRNA nodaviruses. Virology 2017; 504: 114-121.
Kazlauskas D, Varsani A, Krupovic M. Pervasive chimerism in the replication-associated proteins of uncultured single-stranded DNA viruses. Viruses 2018; 10: 187; doi:10.3390/v10040187.
Kowarsky M, Camunas-Soler J, Kertesz M, De Vlaminck I, Koh W, Pan W, Martin L, Neff N F, Okamoto J, Wong R J, Kharbanda S, El-Sayed Y, Blumenfeld Y, Stevenson D K, Shaw G M, Wolfe N D, Quake S R. Numerous uncharacterized and highly divergent microbes which colonize humans are revealed by circulating cell-free DNA. PNAS USA 2017; 114:9623-9628. doi/10.1073/pnas.1707009114
Kraberger S, Argüello-Astorga G R, Greenfield L G, Galilee C, Law D, Martin D P, Varsani A. Characterisation of a diverse range of circular replication-associated protein encoding DNA viruses recovered from a sewage treatment oxidation pond. Infect Genet Evol. 2015; 31:73-86.
Krupovic M. Networks of evolutionary interactions underlying the polyphyletic origin of ssDNA viruses. Curr Opin Virol. 2013; 3: 578-586.
Krupovic M, Zhi N, LiJ, Hu G, Koonin E V, WEong S, Shevchenko S, Zhao K, Young N S. Multiple layers of chimerism in a single-stranded DNA discovered by deep sequencing. Genome Biol Evol. 2015; 7:993-1001.
Lamberto I, Gunst K, Müller H, Zur Hausen H, de Villiers E M. Mycovirus-like DNA virus sequences from cattle serum and human brain and serum samples from multiple sclerosis patients. Genome Announc. 2014; 2(4). pii: e00848-14. doi: 10.1128/genomeA.00848-14.
Lean S S, Yeo C C. Small, Enigmatic Plasmids of the Nosocomial Pathogen, Acinetobacter baumannii: Good, Bad, Who Knows? Front Microbiol. 2017; 8:1547. doi: 10.3389/fmicb.2017.01547. eCollection 2017.
Legoff J, Resche-Rigon M, Bouquet J, Robin M, Naccache S N, Mercier-Delarue S, Federman S, Samayoa E, Rousseau C, Piron P, Kapel N, Simon F, Socié G, Chiu C Y. The eukaryotic gut virome in hematopoietic stem cell transplantation: new clues in enteric graft-versus-host disease. Nat Med. 2017; 23: 1080-1085.
Leppik L, Gunst K, Lehtinen M, Dillner J, Streker K, de Villiers E M. In vivo and in vitro intragenomic rearrangement of TT viruses. J Virol. 2007; 81(17):9346-9356. Epub 2007 Jun. 27.
Longkumer T, Kamireddy S, Muthyala V R, Akbarpasha S, Pitchika G K, Kodetham G, Ayaluru M, Siddavattam D. Acinetobacter phage genome is similar to Sphinx 2.36, the circular DNA copurified with TSE infected particles. Sci Rep. 2013; 3:2240. doi: 10.1038/srep02240.
Manuelidis L. Nuclease resistant circular DNAs copurify with infectivity in scrapie and CJD. J Neurovirol. 2011; 17(2):131-45.
Miras M, Miller W A, Truniger V, Aranda M A. Non-canonical translation in plant RNA viruses. Front. Plant Sci. 8:494 doi: 10.3389/fpls.2017.00494. eCollection 2017. Review.
Moustafa A, Xie C, Kirkness E, Biggs W, Wong E, Turpaz Y, Bloom K, Delwart E, Nelson K E, Venter J C, Telenti A. The blood DNA virome in 8,000 humans. PLoS Pathog. 2017; 13:e1006292. doi: 10.1371/journal.ppat.1006292. eCollection 2017 March
Peretti A, FitzGerald P C, Bliskovsky V, Buck C B. Hamburger polyomaviruses. J Gen Virol. 2015; 96: 833-839.
Priebe S D, Lacks S A. Region of the Streptococcal plasmid pMV158 required for conjugative mobilization. J Bateriol 1989; 171: 4778-4784.
Rosario K, Duffy S, Breitbart M. A field guide to eukaryotic circular single-stranded DNA viruses: Insights gained from metagenomics. Arch Virol. 2012; 157: 1851-1871.
Rosario K, Schenck R O, Harbeitner R C, Lawler S N, Breitbart M. Novel circular single-stranded DNA viruses identified in marine invertebrates reveal high sequence diversity and consistent predicted intrinsic disorder patterns within putative structural proteins. Front Microbiol. 2015; 6:696. doi: 10.3389/fmicb.2015.00696. eCollection 2015.
Roux S, Enault F, Bronner G, Vaulot D, Forterre P, Krupovic M. Chimeric viruses blur the borders between the major groups of eukaryotic single-stranded DNA viruses. Nature Commun. 2013; 4:2700.
Ruiz-Maso J A, Leungo L M, Moreno-Cordoba I, Diaz-Orejas R, del Solar G.
Successful establishment of plasmids R1 and pMV158 in a new host requires the relief of the transcriptional repression of their essential rep genes. Frontiers in Microbiol 2017; 8: 2367. Doi: 10.3389/fmicb.2017.02367.
Sadeghi M, Kaupusinsky B, Yugo D M, Phan T G, Deng X, Kanevsky I, Opriessnig T, Woolums A R, Hurley D J, Meng X J, Delwart E. Virome of US bovine calf serum. Biologicals 2017; 46: 64-67.
Salto I P, Torres Tejerizo G, Wibberg D, Pühler A, Schlüter A, Pistorio M. Comparative genomic analysis of Acinetobacter spp. plasmids originating from clinical settings and environmental habitats. Sci Rep. 2018 May 17; 8:7783. doi: 10.1038/s41598-018-26180-3.
Stedman K M. Deep Recombination: RNA and ssDNA Virus Genes in DNA Virus and Host Genomes. Annu Rev Virol. 2015 November; 2(1):203-17. doi: 10.1146/annurev-virology-100114-055127. Review.
Tamura K., Stecher G., Peterson D., Filipski A., and Kumar S. (2013). MEGA6: Molecular Evolutionary Genetics Analysis version 6.0. Molecular Biology and Evolution30: 2725-2729.
Varsani A, Krupovic M. Smacoviridae: a new family of animal-associated single-stranded DNA viruses. Arch Virol. 2018; doi.org/10.1007/s00705-018-3820-z.
Wang H, Li S, Mahmood A, Yang S, Wang X, Shen Q, Shan T, Deng X, Li J, Hua X, Cui L, Delwart E, Zhang W. Plasma virome of cattle from forest region revealed diverse small circular ssDNA viral genomes. Virology Journal 2018; 15:11.
Wawrzyniak P, Plucienniczak G, Bartosik D. The different faces of rolling-circle replication and its multifunctional initiator proteins. Frontiers in Microbiol. 2017; 8: 2353. doi: 10.3389/fmicb.2017.02353.
Whitley C, Gunst K, Müller H, Funk M, zur Hausen H, de Villiers E-M. Novel replication-compotent circular DNA molecules from healthy cattle serum and milk and multiple sclerosis-affected human brain tissue. Genome Announc. 2014; 2(4). pii: e00849-14.
zur Hausen H. Red meat consumption and cancer: Reasons to suspect involvement of bovine infectious factors in colorectal cancer. Int J Cancer 2012; 130:2475-83.
zur Hausen H. What do breast and CRC cancers and MS have in common? Nature Rev Clinical Oncology 2015; 12:569-570.
zur Hausen H, de Villiers E M. Cancer “causation” by infections—individual contributions and synergistic networks. Semin Oncol. 2014; 41(6):860-875. doi: 10.1053/j.seminoncol.2014.10.003. Epub 2014 Oct. 29.
zur Hausen H, de Villiers E M. Dairy cattle serum and milk factors contributing to the risk of colon and breast cancers. Int J Cancer 2015; 137:959-967
zur Hausen H, Bund T, de Villiers E M. Infectious agents in bovine red meat and milk and their potential role in cancer and other chronic diseases. Curr Topics Microbiol Immunol 2017; Mar.28. doi: 10.1007/82_2017_3. [Epub ahead of print]
zur Hausen H, Bund T, de Villiers E M. Specific Nutritional Infections Early in Life as Risk Factors for Human Colon and Breast Cancers Several Decades Later. Int J Cancer. 2018 Sep. 24. doi: 10.1002/ijc.31882. [Epub ahead of print]
The invention is further described by the following numbered paragraphs:

1. A method for isolating DNA from a cow milk sample, comprising the steps of
(a) phenol-chloroform extraction of DNA from the cow milk sample followed by ethanol precipitation,
(b) performing rolling circle amplification (RCA) with the DNA extracted in step (a),
(c) inverted PCR using 2 abutting primer pairs A and B selected from Primer pair A:

>LSconAfwd:
aaggcagatcaacacagg
>LSconArev:
Agcagattgcaaagcctg
Primer Pair B:
>LSconBfwd:
caacacagggatagaataac
>LSconBrev:
atctgccttagcagattgc

2. A C2MI polynucleic acid obtainable by the method of paragraph 1 comprising:
(a) a nucleotide sequence depicted in any one of FIGS. 1(A) to 22(A);
(b) a nucleotide sequence having at least 93% identity to a nucleotide sequence of (a);
(c) a fragment of a nucleotide sequence of (a) or (b);
(d) a nucleotide sequence being complementary to a nucleotide sequence of (a), (b) or (c); or
(e) a nucleotide sequence which is redundant as a result of the degeneracy of the genetic code compared to any of the above-given nucleotide sequences.

3. An oligonucleotide probe comprising part of a C2MI polynucleic acid of paragraph 2, said probe being capable of acting as a hybridization probe for specific detection of the nucleic acid of a certain C2MI isolate containing a nucleotide sequence of paragraph 2.

4. An expression vector comprising a C2MI polynucleic acid of paragraph 2 or 3 operably linked to prokaryotic, eukaryotic or viral transcription and translation control elements.

5. A host cell transformed or modified with an expression vector according to paragraph 4.

6. A polypeptide being encoded by a C2MI polynucleic acid of paragraph 2.

7. An antibody or antigen binding fragment thereof specifically binding to a polypeptide of paragraph 6.

8. Use of a probe according to paragraph 3, a polypeptide of paragraph 6, or an antibody or fragment thereof according to paragraph 7 for the preparation of a diagnostic composition for the diagnosis of a predisposition or an early stage of cancer, a disease of the CNS, atherosclerosis or diabetes.

9. Use according to paragraph 8, wherein the cancer is breast cancer, ovarian cancer, lung cancer, prostate cancer, colorectal cancer or colon cancer, and the disease of the CNS is Multiple sclerosis MS, amyotrophic lateral sclerosis, transmissible spongiforme encephalopathies/Prion-linked diseases, Parkinson's disease or Alzheimer disease.

10. A method for the detection of a C2MI polynucleic acid according to paragraph 2 in a biological sample, comprising: (a) optionally extracting sample polynucleic acid, (b) hybridizing the polynucleic acid as described above with at least one probe according to paragraph 3, optionally a labelled probe, and (c) detecting the hybridized polynucleic acid.

11. A method for detecting a polypeptide of paragraph 6 or an antibody of paragraph 7 present in a biological sample, comprising: (a) contacting the biological sample for the presence and/or concentration of such polypeptide or antibody as defined above, and (b) detecting the immunological complex formed between said antibody and/or said polypeptide.

12. An antisense oligonucleotide reducing or inhibiting the expression of a C2MI polynucleic acid of paragraph 2 or a vector containing said antisense oligonucleotide.

13. A pharmaceutical composition comprising the antibody or antigen binding fragment thereof of paragraph 7 or the antisense oligonucleotide of paragraph 12 and a suitable pharmaceutical carrier.

14. A vaccine comprising a C2MI polynucleic acid of paragraph 2 or a polypeptide according to paragraph 6, preferably wherein the vaccine comprises a VLP or protein/DNA or polypeptide/DNA complex or specific proteins or attenuated infectious agents.

15. Use of a C2MI polynucleic acid of paragraph 2 as a lead component for the development of a medicament for prevention or treatment of cancer, a disease of the CNS, atherosclerosis or diabetes, preferably wherein the cancer is breast cancer, ovarian cancer, lung cancer, prostate cancer, colorectal cancer or colon cancer and the disease of the CNS is Multiple sclerosis MS, amyotrophic lateral sclerosis, transmissible spongiforme encephalopathies/Prion-linked diseases, Parkinson's disease or Alzheimer disease.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for isolating DNA from a cow milk sample, comprising the steps of
(a) phenol-chloroform extraction of DNA from the cow milk sample followed by ethanol precipitation,
(b) performing rolling circle amplification (RCA) with the DNA extracted in step (a),
(c) inverted PCR using 2 abutting primer pairs A and B selected from
Primer pair A:
>LSconAfwd:
aaggcagatcaacacagg
>LSconArev:
Agcagattgcaaagcctg
Primer Pair B:
>LSconBfwd:
caacacagggatagaataac
>LSconBrev:
atctgccttagcagattgc

2. A C2MI polynucleic acid obtained by the method of claim 1 comprising:
(a) a nucleotide sequence depicted in any one of FIGS. 1(A) to 22(A);
(b) a nucleotide sequence having at least 93% identity to a nucleotide sequence of (a);
(c) a fragment of a nucleotide sequence of (a) or (b);
(d) a nucleotide sequence being complementary to a nucleotide sequence of (a), (b) or (c); or
(e) a nucleotide sequence which is redundant as a result of the degeneracy of the genetic code compared to any of the above-given nucleotide sequences.

3. An oligonucleotide probe comprising part of a C2MI polynucleic acid of claim 2, said probe being capable of acting as a hybridization probe for specific detection of the nucleic acid of a certain C2MI isolate containing a nucleotide sequence of claim 2.

4. An expression vector comprising a C2MI polynucleic acid of claim 2 operably linked to prokaryotic, eukaryotic or viral transcription and translation control elements.

5. A host cell transformed or modified with an expression vector according to claim 4.

6. A polypeptide encoded by the C2MI polynucleic acid of claim 2.

7. A diagnostic composition for the diagnosis of a predisposition or an early stage of cancer, a disease of the CNS, atherosclerosis or diabetes comprising the probe of claim 3.

8. A diagnostic composition for the diagnosis of a predisposition or an early stage of cancer, a disease of the CNS, atherosclerosis or diabetes comprising the polypeptide of claim 6.

9. The composition of claim 7, wherein the cancer is breast cancer, ovarian cancer, lung cancer, prostate cancer, colorectal cancer or colon cancer, and the disease of the CNS is Multiple sclerosis MS, amyotrophic lateral sclerosis, transmissible spongiforme encephalopathies/Prion-linked diseases, Parkinson's disease or Alzheimer disease.

10. The composition of claim 8, wherein the cancer is breast cancer, ovarian cancer, lung cancer, prostate cancer, colorectal cancer or colon cancer, and the disease of the CNS is Multiple sclerosis MS, amyotrophic lateral sclerosis, transmissible spongiforme encephalopathies/Prion-linked diseases, Parkinson's disease or Alzheimer disease.

11. A method for detecting the C2MI polynucleic acid according to claim 2 in a biological sample, comprising: (a) optionally extracting sample polynucleic acid, (b) hybridizing the polynucleic acid as described above with at least one probe of claim 3, optionally a labelled probe, and (c) detecting the hybridized polynucleic acid.

12. A method for detecting the polypeptide of claim 6 present in a biological sample, comprising: (a) contacting the biological sample for the presence and/or concentration of such polypeptide, and (b) detecting the immunological complex formed between said antibody and/or said polypeptide.

13. An antisense oligonucleotide reducing or inhibiting the expression of a C2MI polynucleic acid of claim 2 or a vector containing said antisense oligonucleotide.

14. A pharmaceutical composition comprising the antisense oligonucleotide of claim 13 and a suitable pharmaceutical carrier.

15. A vaccine comprising the C2MI polynucleic acid of claim 2 or a polypeptide according to claim 6.

16. A vaccine comprising the polypeptide according to claim 6.

17. The vaccine of claim 15, wherein the vaccine comprises a VLP or protein/DNA or polypeptide/DNA complex or specific proteins or attenuated infectious agents.

18. The vaccine of claim 16, wherein the vaccine comprises a VLP or protein/DNA or polypeptide/DNA complex or specific proteins or attenuated infectious agents.
